(19) United States
(12) Patent Application Publication
(10) Pub. No.: US 2021/0186454 A1
(43) Pub. Date: Jun. 24, 2021

(54) ANATOMICAL LOCATOR TAGS AND USES

(71) Applicant: Kambiz Behzadi, Pleasanton, CA (US)

(72) Inventors: Kambiz Behzadi, Pleasanton, CA (US); Michael E. Woods, Brisbane, CA (US)

(73) Assignee: Kambiz Behzadi, Pleasanton, CA (US)

(21) Appl. No.: 16/945,908

(22) Filed: Aug. 2, 2020

Related U.S. Application Data
(63) Continuation of application No. 16/819,092, filed on Mar. 14, 2020, now abandoned.
Application No. 16/819,092 is a continuation-in-part of application No. 16/375,736, filed on Apr. 4, 2019.
Application No. 16/375,736 is a continuation-in-part of application No. 16/030,603, filed on Jul. 9, 2018, now abandoned.
Application No. 16/030,603 is a continuation-in-part of application No. 15/716,533, filed on Sep. 27, 2017.
Application No. 15/716,533 is a continuation-in-part of application No. 15/687,324, filed on Aug. 25, 2017.
Application No. 15/687,324 is a continuation of application No. 15/284,091, filed on Oct. 3, 2016, now Pat. No. 10,441,244.
Application No. 15/284,091 is a continuation-in-part of application No. 15/234,782, filed on Aug. 11, 2016, now Pat. No. 10,912,655.
Application No. 15/234,782 is a continuation-in-part of application No. 15/202,434, filed on Jul. 5, 2016, now abandoned.
Application No. 15/716,533 is a continuation-in-part of application No. 15/284,091, filed on Oct. 3, 2016, now Pat. No. 10,441,244.
Application No. 15/284,091 is a continuation-in-part of application No. 15/234,782, filed on Aug. 11, 2016, now Pat. No. 10,912,655.
Application No. 15/234,782 is a continuation-in-part of application No. 15/202,434, filed on Jul. 5, 2016, now abandoned.
Application No. 16/030,603 is a continuation-in-part of application No. 15/284,091, filed on Oct. 3, 2016, now Pat. No. 10,441,244.
Application No. 16/819,092 is a continuation-in-part of application No. 15/592,229, filed on May 11, 2017, now Pat. No. 10,849,766.
Application No. 15/592,229 is a continuation-in-part of application No. 15/284,091, filed on Oct. 3, 2016, now Pat. No. 10,441,244.
(60) Provisional application No. 62/743,042, filed on Oct. 9, 2018.
(60) Provisional application No. 62/742,851, filed on Oct. 8, 2018.
(60) Provisional application No. 62/651,077, filed on Mar. 31, 2018.
(60) Provisional application No. 62/277,294, filed on Jan. 11, 2016.
(60) Provisional application No. 62/355,657, filed on Jun. 28, 2016.
(60) Provisional application No. 62/353,024, filed on Jun. 21, 2016.

(51) Int. Cl. A61B 7/02; A61F 2/34; A61F 2/46

(52) U.S. Cl. CPC A61B 7/023; A61B 2090/064; A61F 2/468; A61F 2/34

(57) ABSTRACT

A system and method for providing a set of anatomical subdermal tags configured to form part of a local positioning system (in contrast to an operating room-wide global reference system) used in obscured visualization/localization of anatomical structures, locations, and components, as well as visualization/localization/orientation of implant(s) into referenced anatomical structures.

### CROSS REFERENCE TO RELATED APPLICATIONS

This application is a Continuation-in-part of application Ser. No. 16/819,092 filed on Mar. 14, 2020; application Ser. No. 16/819,092 is a Continuation-in-part of application Ser. No. 16/375,736 filed on Apr. 4, 2019; application Ser. No. 16/375,736 claims the benefit of U.S. Provisional Application 62/743,042 filed on Oct. 9, 2018; application Ser. No. 16/375,736 claims the benefit of U.S. Provisional Application 62/742,851 filed on Oct. 8, 2018; application Ser. No. 16/375,736 is a Continuation-in-part of application Ser. No. 16/030,603 filed on Jul. 9, 2018; application Ser. No. 16/030,603 claims the benefit of U.S. Provisional Application 62/651,077 filed on Mar. 31, 2018; application Ser. No. 16/030,603 is a Continuation-in-part of application Ser. No. 15/716,533 filed on Sep. 27, 2017; application Ser. No. 15/716,533 is a Continuation-in-part of application Ser. No. 15/687,324 filed on Aug. 25, 2017; application Ser. No. 15/687,324 is a Continuation of application Ser. No. 15/284,091 filed on Oct. 3, 2016; application Ser. No. 15/284,091 is a Continuation-in-part of application Ser. No. 15/234,782 filed on Aug. 11, 201; application Ser. No. 15/234,782 is a Continuation-in-part of application Ser. No. 15/202,434 filed on Jul. 5, 2016; application Ser. No. 15/202,434 claims the benefit of U.S. Provisional Application 62/277,294 filed on Jan. 11, 2016; application Ser. No. 15/234,782 claims the benefit of U.S. Provisional Application 62/355,657 filed on Jun. 28, 2016; application Ser. No. 15/234,782 claims the benefit of U.S. Provisional application 62/353,024 filed on Jun. 21, 2016; application Ser. No. 15/716,533 is a Continuation-in-part of application Ser. No. 15/284,091 filed on Oct. 3, 2016; application Ser. No. 15/716,533 is a Continuation-in-part of application Ser. No. 15/234,782 filed on Aug. 11, 2016; application Ser. No. 15/716,533 is a Continuation-in-part of application Ser. No. 15/202,434 filed on Jul. 5, 2016; application Ser. No. 16/030,603 is a Continuation-in-part of application Ser. No. 15/284,091 filed on Oct. 3, 2016; application Ser. No. 16/819,092 is a Continuation-in-part of application Ser. No. 15/592,229 filed on May 11, 2017; and application Ser. No. 15/592,229 is a Continuation-in-part of application Ser. No. 15/284,091 filed on Oct. 3, 2016; and all of the these identified applications, including direct and indirect parent applications, are hereby expressly incorporated by reference thereto in their entireties for all purposes.

### FIELD OF THE INVENTION

The present invention relates generally to in situ sensing or monitoring of tissue structures, implants into such structures, and surgical procedures and devices associated with preparation and operation on such structures, as well as relating generally to orthopedic surgical systems and procedures employing a prosthetic implant for, and more specifically, but not exclusively, to joint replacement therapies such as total hip replacement including controlled installation and positioning of the prosthesis such as during replacement of a pelvic acetabulum with a prosthetic implant, and relates generally to installation of a prosthesis, and more specifically, but not exclusively, to improvements in prosthesis placement and positioning, and relates generally to force measurement systems such as may be used in these systems and methods which may evaluate a quality of installation, and generally to assessing a quality of an installation of an implant structure installed in a body, and more specifically, but not exclusively, to quantitative assessment of prosthesis press-fit fixation into a bone cavity, for example, assessment of press-fit fixation of an acetabular cup into a prepared (e.g., relatively under-reamed acetabulum) bone cavity, assessment of connective tissue installation and repair.

### BACKGROUND OF THE INVENTION

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also be inventions.

Total hip replacement refers to a surgical procedure where a hip joint is replaced using a prosthetic implant. There are several different techniques that may be used, but all include a step of inserting an acetabular component into the acetabulum and positioning it correctly in three dimensions (along an X, Y, and Z axis).

In total hip replacement (THR) procedures there are advantages to patient outcome when the procedure is performed by a surgeon specializing in these procedures. Patients of surgeons who do not perform as many procedures can have increased risks of complications, particularly of complications arising from incorrect placement and positioning of the acetabular component.

The incorrect placement and positioning may arise even when the surgeon understood and intended the acetabular component to be inserted and positioned correctly. This is true because in some techniques, the tools for actually installing the acetabular component are crude and provide an imprecise, unpredictable coarse positioning outcome.

Some techniques may employ automated and/or computer-assisted navigation tools, for example, x-ray fluoroscopy or computer guidance systems. There are computer assisted surgery techniques that can help the surgeon in determining the correct orientation and placement of the acetabular component. However, current technology provides that at some point the surgeon is required to employ a hammer/mallet to physically strike a pin or alignment rod. The amount of force applied and the location of the application of the force are variables that have not been controlled by these navigation tools. Thus even when the acetabular component is properly positioned and oriented, when actually impacting the acetabular component into place the actual location and orientation can differ from the intended optimum location and orientation. In some cases the tools used can be used to determine that there is, in fact, some difference in the location and/or orientation. However, once again the surgeon must employ an impacting tool (e.g., the hammer/mallet) to strike the pin or alignment rod to attempt an adjustment. However the resulting location and orientation of the acetabular component after the adjustment may not be, in fact, the desired location and/or orientation. The more familiar that the surgeon is with the use and application of these adjustment tools can reduce the risk to a patient from a less preferred location or orientation. In some circumstances, quite large impacting forces are applied to the prosthesis by the mallet striking the rod; these forces make fine tuning difficult at best and there is risk of fracturing and/or shattering the acetabulum during these impacting steps.

Earlier patents issued to the present applicant have described problems associated with prosthesis installation, for example acetabular cup placement in total hip replacement surgery. See U.S. Pat. Nos. 9,168,154 and 9,220,612, which are hereby expressly incorporated by reference thereto in their entireties for all purposes. Even though hip replacement surgery has been one of the most successful operations, it continues to be plagued with a problem of inconsistent acetabular cup placement. Cup mal-positioning is the single greatest cause of hip instability, a major factor in polyethylene wear, osteolysis, impingement, component loosening and the need for hip revision surgery.

These incorporated patents explain that the process of cup implantation with a mallet is highly unreliable and a significant cause of this inconsistency. The patents note two specific problems associated with the use of the mallet. First is the fact that the surgeon is unable to consistently hit on the center point of the impaction plate, which causes undesirable torques and moment arms, leading to mal-alignment of the cup. Second, is the fact that the amount of force utilized in this process is non-standardized.

Traditionally these methods do not have any clear understanding of the forces, including magnitude and direction, involved in installing a prosthesis. A surgeon often relies on qualitative factors from tactile and auditory senses. Consequently, the surgeon is left somewhat haphazardly and variably relying on two different fixation methods (e.g., pins and press-fit) without knowing how or why.

In these patents there is presented a new apparatus and method of cup insertion which uses an oscillatory motion to insert the prosthesis. Prototypes have been developed and continue to be refined, and illustrate that vibratory force may allow insertion of the prosthesis with less force, as well, in some embodiments, of allowing simultaneous positioning and alignment of the implant.

There are other ways of breaking down of the large undesirable, torque-producing forces associated with the discrete blows of the mallet into a series of smaller, axially aligned controlled taps, which may achieve the same result incrementally, and in a stepwise fashion to those set forth in the incorporated patents, (with regard to, for example, cup insertion without unintended divergence).

There are two problems that may be considered independently, though some solutions may address both in a single solution. These problems include i) undesirable and unpredictable torques and moment arms that are related to the primitive method currently used by surgeons, which involves manually banging the mallet on an impaction plate mated to the prosthesis and ii) non-standardized and essentially uncontrolled and unquantized amounts of force utilized in these processes.

Total hip replacement has been one of the most successful orthopedic operations. However, as has been previously described in the incorporated applications, it continues to be plagued with the problem of inconsistent acetabular cup placement. Cup mal-positioning is a significant cause of hip instability, a major factor in polyethylene wear, osteolysis, impingement, component loosening, and the need for hip revision surgery.

Solutions in the incorporated applications generally relate to particular solutions that may not, in every situation and implementation, achieve desired goal(s) of a surgeon.

There are various sensing/monitoring/auditing/transacting/guiding systems that may be used over a course of preparation and installation of a prosthesis, for example an acetabular cup. These systems may detect various parameters such as an orientation angle of the prosthesis at any given time. These systems may provide a set of periodic snapshots in time over the course of the procedure, but they do not provide true realtime continuous data over the installation procedure. That is, a surgeon may employ a system to measure an orientation before striking an acetabular cup using a mallet and tamp, and may employ a system to measure an orientation after striking the acetabular cup. But these systems do not provide an orientation measurement (and in most cases no measurement of any information) during the strike. That is, the surgeon often measures, strikes, remeasures, restrikes, and repeats until the surgeon decides to stop. For a conventional system in which the surgeon manually swings the mallet and the installation model includes a sequence of discrete impulses from the mallet, this paradigm is understandable.

Some conventional systems may describe some measurements as “real time” but those systems are real time in the sense that the measurements are taken in the operating room during a procedure. The actual system does not provide realtime measurement during the actual insertion event.

In the incorporated applications, alternatives to the manual swinging of the mallet are described and in these systems the conventional measurement paradigm may be unnecessarily restrictive.

There are generally three types of fixation in orthopedic surgery: 1. Press fit fixation (commonly used in arthroplasty) 2. Screw fixation (used in fracture and arthroplasty) 3. Bone cement (mostly used in arthroplasty).

Initial stability of metal backed acetabular components is an important factor in an ultimate success of cement-less hip replacement surgery. The press fit technique, which involves impaction of an oversized (relative to a prepared cavity in an acetabulum) porous coated acetabular cup into an undersized cavity (relative to the prosthesis to be installed) of bone produces primary stability through cavity deformation and frictional forces, and has shown excellent long term results. This press fit technique avoids use of screw fixation associated with risk of neurovascular injury, fretting and metallosis, and egress of particulate debris and osteolysis.

However, it has been difficult to assess a primary implant stability due to complex nature of bone-implant interface, or to evaluate an optimal press fit fixation. The initial interaction of the implant with bone is due the circumferential surface interference at the aperture transitioning to compression of the cavity with deeper insertion. A compromise exists between seating the cup enough to get sufficient primary stability and avoiding fracture of bone. There is no quantitative method in current clinical practice to assess the primary stability of the implant, with surgeons relying solely on their qualitative proprioceptive senses (tactile, auditory, and visual) to determine point of optimal press fit fixation.

Four factors associated with difficulty obtaining optimal press fit fixation: i) no current method exists to gauge the resulting stress field in bone during the impaction of an oversized implant; ii) the material properties of bone (bone density) vary significantly based on age and sex of the patient, and are unknown to the surgeon; iii) current mallet based techniques for impaction do not allow surgeons to control (quantify and increment) the magnitude of force using in installation; and iv) surgeons are charged with the difficult task of: a) applying and modulating magnitude of force; b) deciding when to stop application of force; and c) assessing a quality of press fit fixation all simultaneously in their “mind's eye” during the process of impaction.

A significance of this problem on patients, medical practice and economy is great. Although Total Hip Replacement (THR) is widely recognized as a successful operation, 3 to 25% of operations fail requiring revision surgery. Aseptic loosening of press fit THR components is one of the most common causes of failure at 50% to 90% and closely associated with insufficient initial fixation. Inadequate stabilization may lead to late presentation of aseptic loosening due to formation of fibrous tissue and over stuffing the prosthesis may lead to occult and/or frank peri-prosthetic fractures. The cost of poor initial press fit fixation resulting from (loosening, occult fractures, subsidence, fretting, metallosis, and infections) maybe under reported however estimated to be in tens of billions of dollars. Over 400,000 total hip replacements are done in US every year, over 80% of which are done by surgeons who do less than ten per year. The limitations of this procedure produce frustration and anxiety for surgeons, physical and emotional pain for patients, at great costs to society.

Initial implant fixation can be measured by pullout, lever out, and torsional test in vitro; however, these methods have minimal utility in a clinical setting in that they are destructive. Vibration analysis, where secure and loose implants can be distinguished by the differing frequency responses of the implant bone interface, has been successfully employed in evaluating fixation of dental implants however, this technology has not been easily transferable to THR surgery, and currently has no clinical utility.

In clinical practice, surgeons err on the side of not overstuffing the prosthesis which leads to a smaller under ream (or line to line ream) and screw fixation with attendant risks.

Finally, several visual tracking methods (Computer Navigation, Fluoroscopy, MAKO Robotics) are utilized to assess the depth of cup insertion during impaction in order to guide application of force; however, these techniques, from and engineering perspective, are considered to be open loop, where the feedback response to the surgeon is not a force (sensory) response, and therefore does not provide any information about the stress response of the cavity.

Injury to connective tissue is common, particularly for those that are physically active. A common type of injury among certain sports and activities is the ACL injury. A healing potential of a ruptured ACL has been poor, and reconstruction of the ACL is often required for return to activity and sports. Various types of tendon grafts are used to reconstruct the ACL including allograft and autograft tissues. In general, bony tunnels are created in the tibia and femur and a variety of fixation devices are used to fix a graft that has been pulled into the knee joint, within the tunnels, to the tibia and femur. Various types of fixation are utilized to fix the graft to the bone tunnels. These fixation methods broadly categorized into cortical suspensory button fixation vs. aperture interference screw fixation.

A system and method may be useful to quantitatively assess a press fit value (and provide a mechanism to evaluate optimal quantitative values) of any implant/bone interface regardless the variables involved including bone site preparation, material properties of bone and implant, implant geometry and coefficient of friction of the implant-bone interface without requiring a visual positional assessment of a depth of insertion.

What may be useful is a system and method for allowing any surgeon, including those surgeons who perform a fewer number of a replacement procedure as compared to a more experienced surgeon who performs a greater number of procedures, to provide an improved likelihood of a favorable outcome approaching, if not exceeding, a likelihood of a favorable outcome as performed by a very experienced surgeon with the replacement procedure, such as by understanding the prosthesis installation environment (e.g., cup/cavity interface) and to provide intelligent and interactive tools and methods to standardize the installation process and provide feedback regarding a quality of insertion.

What may be useful is a solution that improves in situ sensing of components, tools, and processes for tissue repair options.

In many procedures, a surgeon may be required to rely on subjective anatomical references for assessing a quality of a procedure (e.g., a leg length and/or offset during hip replacement uses a an imprecise and non-repeatable assessment for a trochanter or teardrop) to gauge proper post-surgical leg length and offset. Addressing this subjectively to allow use of objective and repeatable references may be helpful, as well as providing a local positioning system (in contrast to a global/operating room wide) common reference for a surgical procedure.

### BRIEF SUMMARY OF THE INVENTION

Disclosed is a solution that improves in situ sensing of components, tools, and processes for tissue repair options, a system and method for quantitatively assessing a press fit value (and provide a mechanism to evaluate optimal quantitative values) of any implant/bone interface regardless the variables involved including bone site preparation, material properties of bone and implant, implant geometry and coefficient of friction of the implant-bone interface without requiring a visual positional assessment of a depth of insertion, and a system, method, and computer program product for allowing any surgeon, including those surgeons who perform a fewer number of a replacement procedure as compared to a more experienced surgeon who performs a greater number of procedures, to provide an improved likelihood of a favorable outcome approaching, if not exceeding, a likelihood of a favorable outcome as performed by a very experienced surgeon with the replacement procedure, such as by understanding the prosthesis installation environment (e.g., cup/cavity interface) and to provide intelligent and interactive tools and methods to standardize the installation process and provide feedback regarding a quality of insertion/installation. A system and method for providing a set of anatomical subdermal tags configured to form part of a local positioning system (in contrast to an operating room-wide global reference system) used in obscured visualization/localization of anatomical structures, locations, and components, as well as visualization/localization/orientation of implant(s) into referenced anatomical structures.

The following summary of the invention is provided to facilitate an understanding of some of the technical features related to installation of an acetabular cup prosthesis into a relatively undersized prepared cavity in an acetabulum (e.g., a press fit fixation procedure), and is not intended to be a full description of the present invention. A full appreciation of the various aspects of the invention can be gained by taking the entire specification, claims, drawings, and abstract as a whole. The present invention is applicable to other press fit fixation systems, including installation of different prostheses into different locations, and installation of other structures into an elastic substrate.

The following summary of the invention is also provided to facilitate an understanding of some of technical features related to total hip replacement, and is not intended to be a full description of the present invention. A full appreciation of the various aspects of the invention can be gained by taking the entire specification, claims, drawings, and abstract as a whole. The present invention is applicable to other surgical procedures, including replacement of other joints replaced by a prosthetic implant in addition to replacement of an acetabulum (hip socket) with an acetabular component (e.g., a cup). Use of pneumatic and electric motor implementations have both achieved a proof of concept development. Provision of anatomical tags as described or suggested herein for enhancement of an accuracy and quality of various surgical procedures can assist a surgeon perform more efficient and superior surgeries.

Also disclosed is a system and method for an improved connective tissue repair option that reduces disadvantages of conventional fixation options. The following summary of the invention is provided to facilitate an understanding of some of the technical features related to connective tissue preparation and repair systems and methods, and is not intended to be a full description of the present invention. A full appreciation of the various aspects of the invention can be gained by taking the entire specification, claims, drawings, and abstract as a whole. The present invention is applicable to other connective tissue repair systems and methods in addition to repair of an anterior cruciate ligament (ACL) injury including other connective tissue repairs using a suspensory-type or aperture-type solution.

Some embodiments of the proposed technology may enable a standardization of: a) application of force; and b) assessment of quality of fixation in joint replacement surgery, such that surgeons of all walks of life, whether they perform five or 500 hip replacements per year, will produce consistently superior/optimum/perfect results with respect to press fit fixation of implants in bone.

From the surgeon perspective this standardization process will level the playing field between the more and less experienced surgeons, leading to less stress and anxiety for the surgeons affecting their mental wellness. From the patient perspective there will be a decrease in the number of complications and ER admissions leading to decrease in morbidity and mortality. From an economic perspective there will be a significant cost savings for the government and insurance companies due to a decrease in the number of readmissions and revision surgery's, particularly since revision surgery in orthopedics accounts for up to 30% of a 50-billion-dollar industry.

To address this deficiency, some embodiments and related applications have considered a novel means of accessing and processing various force responses of bone (Invasive Sensing Mechanism) and propose that this mechanism can guide application of force to the bone cavity, to obtain optimal press fit technologically without reliance on surgeon's proprioception. There are several possible outcomes of this proposal, if validated, including that it may make joint replacement surgery a significantly safer operation leading to less morbidity and complications, readmissions, and revision surgery; resulting in great benefits to patients, surgeons and society in general.

Some embodiments of the present invention relate to non-interventional in situ communication of data to an extracorporeal data accessing system, such as intracorporeal data provided to a surgeon during a surgery procedure (e.g., a total hip replacement) relating to one or both of leg length or leg offset. Current systems have a surgeon stop the surgery and take and evaluate an image or use mechanical measurement systems (e.g., calipers) for inaccurately assessing leg length/offset. Having this information provided in continuously in realtime/near realtime, or sufficiently updated automatically that the surgeon is not required to intervene to initiate/receive/calculate these numbers.

An embodiment of the present invention may include a series of operations for installing a prosthesis into a relatively undersized cavity prepared in a portion of bone, including communicating, using an installation agency, a quantized applied force to a prosthesis being press-fit into the cavity; monitoring a rigidity metric and an elasticity metric of the prosthesis with respect to the cavity (some embodiments do this in real-time or near real-time without requiring imaging or position-determination technology); further processing responsive to the rigidity and elasticity metrics, including continuing to install the prosthesis at present level of applied force while monitoring the metrics when the metrics indicate that installation change is acceptable and a risk of fracture remains at an acceptable level, increasing the applied force and continuing applying the installation agency while monitoring the metrics when the metrics indicate that installation change is minimal and a risk of fracture remains at an acceptable level, or suspending operation of the installation agency when the metrics indicate that installation change is minimal when a risk of fracture increases to an unacceptable level. Some embodiments may determine rigidity/elasticity from position, or vibration spectrum in air (sound) or bone. In some embodiments, while rigidity and elasticity may be determined in several different ways, some of which are disclosed herein, some implementations may determine a quantitative assessment responsive to evaluations of both responsive rigidity and elasticity factors during controlled operation of an insertion agency communicating an application force to a prosthesis (best fixation short of fracture—BFSF). BFSF may be related to one or both of these rigidity and elasticity factors.

An apparatus for insertion of a prosthesis into a cavity formed in a portion of bone, the prosthesis relatively oversized with respect to the cavity, including an insertion device providing an insertion agency to the prosthesis, the insertion agency operating over a period, the period including an initial prosthesis insertion act with the insertion device and a subsequent prosthesis insertion act with the insertion device; and a system physically coupled to the insertion device configured to provide a parametric evaluation of an extractive force of an interface between the prosthesis and the cavity during the period, the parametric evaluation including an evaluation of a set of factors of the prosthesis with respect to the cavity, the set of factors including one or more of a rigidity factor, an elasticity factor, and a combination of the rigidity factor and the elasticity factor.

A method for an insertion of an implant into a cavity in a portion of bone, the cavity relatively undersized with respect to the implant, including a) providing, using a device, an implant insertion agency to the implant to transition the implant toward a deepen insertion into the cavity; and b) predicting, responsive to the implant insertion agency, a press-fit fixation of the implant at an interface between the implant and the cavity during the providing of the implant insertion agency.

An impact control method for installing an implant into a cavity in a portion of bone, the cavity relatively undersized with respect to the implant, including a) imparting a first initial known force to the implant; b) imparting a first subsequent known force to the implant, the first subsequent known force about equal to the first initial force; c) measuring, for each the imparted known force, an Xth number measured impact force; d) comparing the Xth measured impact force to the Xth−1 measured impact force against a predetermined threshold for a threshold test; and e) repeating steps b)-d) as long as the threshold test is negative.

A method for an automated installation of an implant into a cavity in a portion of bone, including a) initiating an application of an installation agency to the implant, the installation agency including an energy communicated to the implant moving the implant deeper into the cavity in response thereto; b) recording a set of measured response forces responsive to the installation agency; c) continuing applying and recording until a difference in successive measured responses is within a predetermined threshold to estimate no significant displacement of the implant at the energy as the implant is installed into the cavity; d) increasing the energy; e) repeating steps b)-c) until a plateau of the set of the measured response forces; and f) terminating steps b)-e) when a steady-state is detected.

A method for insertion of a prosthesis into a cavity formed in a portion of bone, the prosthesis relatively oversized with respect to the cavity, including a) applying an insertion agency to the prosthesis, the insertion agency operating over a period, the period including an initial prosthesis insertion act with the insertion device and a subsequent prosthesis insertion act with the insertion device; and b) providing a parametric evaluation of an extractive force of an interface between the prosthesis and the cavity during the period, the parametric evaluation including an evaluation of a set of factors of the prosthesis with respect to the cavity, the set of factors including one or more of a rigidity factor, an elasticity factor, and a combination of the rigidity factor and the elasticity factor.

An apparatus for installing a prosthesis into a relatively undersized prepared cavity in a portion of a bone, including a force applicator operating an insertion agency for installing the prosthesis into the cavity; a force transfer structure, coupled to the force applicator and to the prosthesis, for conveying an application force F1 to the prosthesis, the application force F1 derived from the insertion agency; a force sensing system determining a force response of the prosthesis at an interface of the prosthesis and the cavity, the force response responsive to the application force F1; and a controller, coupled to force applicator and to the force sensing system, the controller setting an operational parameter for the insertion agency, the operational parameter establishing the application force F1, the controller responsive to the force response to establish a set of parameters including one or more of a rigidity metric, an elasticity metric, and combinations thereof.

A method for installing a prosthesis into a relatively undersized cavity prepared in a portion of bone, including a) communicating an application force F1 to the prosthesis; b) monitoring a rigidity factor and an elasticity factor of the prosthesis within the cavity during application of the application force F1; c) repeating a)-b) until the rigidity factor meets a first predetermined goal; d) increasing, when the rigidity factor meets the predetermined goal, the application force F1; e) repeating a)-d) until the elasticity factor meets a second predetermined goal; and f) suspending a) when the elasticity factor meets the first goal and the rigidity factor meets the second goal.

An acetabular cup for a prepared cavity in a portion of bone, including a generally hemispherical exterior shell portion defining a generally hemispherical interior cavity; and a snubbed polar apex portion of the generally hemispherical exterior shell portion without degradation of the generally hemispherical interior cavity producing a polar gap within the prepared cavity when fully seated.

An implant for a prepared cavity in a portion of bone, including an exterior shell portion having an interior cavity; and a snubbed polar apex portion of the exterior shell portion without degradation of the interior cavity producing a polar gap within the prepared cavity when fully seated.

An apparatus for insertion of a prosthesis into a cavity formed in a portion of bone, the prosthesis relatively oversized with respect to the cavity, including means for applying an insertion agency to the prosthesis, the insertion agency operating over a period, the period including an initial prosthesis insertion act with the insertion device and a subsequent prosthesis insertion act with the insertion device; and means, physically coupled to the insertion device, for determining a parametric evaluation of an extractive force of an interface between the prosthesis and the cavity during the period, the parametric evaluation including an evaluation of a set of factors of the prosthesis with respect to the cavity, the set of factors including one or more of a rigidity factor, an elasticity factor, and a combination of the rigidity factor and the elasticity factor.

An embodiment may include a graft platform (e.g., a table or stage) that is specially configured for pre-repair preparation of a connective tissue graft. This structure temporarily compresses and/or tensions (e.g., stretches) the connective tissue graft which temporarily reduces its outer perimeter (e.g., for a circular graft this may refer to a radius/circumference of the graft) appropriately in advance of installation. After installation, the connective tissue graft naturally expands towards its original unreduced perimeter in situ which may apply high compressive forces at a ligament/bone interface within bone tunnels through, or into, which the reduced graft had been installed.

An embodiment for a graft platform includes a graft compression system. A graft compression system may be implemented in many different ways—it may include a support for a pair of stages that may be coupled together via an optional controllable separation mechanism that controls a distance between these stages. Each stage may include a gripping system that provides compression to reduce and/or profile the perimeter. The compression system may include one or both of these compressive mechanisms: (a) grip and stretch, and/or (b) grip and squeeze.

This may increase the possibility of the more natural “direct-type” tendon to bone healing which decreases risks of repair failures that arise from “indirect-type” healing.

This may allow a surgeon to use repair procedures that preserve more bone. These procedures often include preparing the tunnels in the bone and allowing for use of a reduced perimeter graft allows the surgeon to prepare smaller radius tunnels or to improve graft repair strength of conventionally-sized tunnels, at the surgeon's discretion. More options allow the surgeon to provide better customized solutions to the patience.

An embodiment of the present invention may include a graft-preparation table that includes a pair of relatively-moveable stages (e.g., a distance between these stages is variable). Each stage may be provided with a compressive structure that secures the graft. The stage may compress the graft by direct compression through application of force(s) on the perimeter and/or indirect compression by tensioning the graft such as by stretching the graft through pulling.

Method and Apparatus Claims for creation of Non-cylindrical, asymmetric, conical, frustum like, profiled, curvilinear tunnels for ACL reconstruction (as well as other ligaments in other joints), in which a natural mechanical resistance to pull out is produced for a decompressing and/or expanding compressed connective tissue graft by the inherent asymmetric shape of the tunnel (A) using existing 3D sculpting or existing robotic techniques and/or new bone preparation techniques.

Method and Apparatus for creation of ACL (PCL, MPFL, MCL, LCL) ligament bone tunnels without the use of a pre-determined guide wire and over drilling technique.

Method and Apparatus for correlating precisely or matching precisely (e.g., to within 1 mm) the length of ACL graft with the length of bony tunnels+intra articular ACL, when using robotic or 3D bone sculpting techniques, instead of guide wire and over drill techniques.

Method and Apparatus for producing the environment which allows a “biologic press fit” fixation, where high tendon-bone interface forces are achieved with a passively or actively decompressing/expanding (previously compressed) ACL graft, which may be used with or without suspensory cortical fixation and with or without mechanical foreign body (e.g., screw-less) fixation.

Method and Apparatus for delivery of various biological growth factors within a compressed ACL graft to enhance tendon bone healing with direct type and/or indirect type healing at the interface (angiogenesis and osteogenesis) with or without suspensory cortical fixation and with or without mechanical foreign body (e.g., screw-less) fixation.

Method and Apparatus for embedding sensors (biologic and/or electronic) within the substance of ACL (and other ligament) grafts to assess (A) intra-tunnel interface forces (pressures), in order to determine if/when interface forces are adequate (high) enough for direct type and/or indirect type healing (B) intra-articular ligament tensile and shear forces (within the notch) to determine failure mechanisms and maximal load to failure in the case of re injury or re rupture.

Method and Apparatus for pre-compressing and shipping pre-compressed connective tissue graft, including use of a sheathing system having one or more layers, those layers may include: structural elements to maintain compression until pre-operative preparation; time-delaying materials/construction for manipulation of active/passive decompression/expansion; inclusion of biologic sensors; and/or inclusion of biologic growth/healing/bone or tissue conditioning factors to promote a desired outcome with the installation of the decompressing/expanding compressed graft within a prepared bone tunnel.

Method and Apparatus for embedding a set of one or more prosthetic elements inside a connective tissue graft (conventional or pre-compressed) and securing/deploying/installing a prosthetically-enhanced natural connective tissue within a prepared bone tunnel for fixation, the fixation may include the passive/active decompression/expansion of a pre-compressed prosthetically-enhanced connective tissue graft, the enhancement including a set of one or more natural, synthetic, and/or hybrid materials having a material property different from natural connective tissue.

Method and Apparatus for deploying expansion structures within a natural connective tissue graft, initiating and manipulating enlargement of those expansion structures to actively expand the natural connective tissue graft; and including a prosthetic element, such as described in claim 8, as part of or cooperative with the deployed expansion structures.

Certain ones of the disclosed concepts involve creation of a system/method/tool/gun that vibrates an attached prosthesis, e.g., an acetabular cup. The gun would be held in a surgeon's hands and deployed. It would use a vibratory energy to insert (not impact) and position the cup into desired alignment (using current intra-operation measurement systems, navigation, fluoroscopy, and the like).

In one embodiment, a first gun-like device is used for accurate impaction of the acetabular component at the desired location and orientation.

In another embodiment, a second gun-like device is used for fine-tuning of the orientation of the acetabular component, such as one installed by the first gun-like device, by traditional mallet and tamp, or by other methodology. However the second gun-like device may be used independently of the first gun-like device for adjusting an acetabular component installed using an alternate technique. Similarly the second gun-like device may be used independently of the first gun-like device, particularly when the initial installation is sufficiently close to the desired location and orientation. These embodiments are not necessarily limited to fine-tuning as certain embodiments permit complete re-orientation. Some implementations allow for removal of an installed prosthesis.

Another embodiment includes a third gun-like device that combines the functions of the first gun-like device and the second gun-like device. This embodiment enables the surgeon to accurately locate, insert, orient, and otherwise position the acetabular component with the single tool.

Another embodiment includes a fourth device that installs the acetabular component without use of the mallet and the rod, or use of alternatives to strike the acetabular component for impacting it into the acetabulum. This embodiment imparts a vibratory motion to an installation rod coupled to the acetabular component that enables low-force, impactless installation and/or positioning.

An embodiment of the present invention may include axial alignment of force transference, such as, for example, an axially sliding hammer moving between stops to impart a non-torqueing installation force. There are various ways of motivating and controlling the sliding hammer, including a magnitude of transferred force. Optional enhancements may include pressure and/or sound sensors for gauging when a desired depth of implantation has occurred.

Other embodiments include adaptation of various devices for accurate assembly of modular prostheses, such as those that include a head accurately impacted onto a trunnion taper that is part of a stem or other element of the prosthesis.

Additional embodiments of the present invention may include a hybrid medical device that is capable of selectively using vibratory and/or axial-impacts at various phases of an installation as required, needed, and/or desired by the surgeon during a procedure. The single tool remains coupled to the prosthesis or prosthesis component as the surgeon operates the hybrid medical device in any of its phases, which include a pure vibratory mode, a pure axial mode, a blended vibratory and impactful mode. The axial impacts in this device may have sub-modes: a) unidirectional axial force-IN, b) unidirectional axial force-OUT, or c) bidirectional axial force.

An embodiment of the present invention may include true realtime sensing before, during, and after a procedure. These procedures may benefit from this invasive sensing (sensing during preparation of bone, during installation of a prosthesis, and during assembly of a modular prosthesis) and not just periodic static snapshots. The invasive sensing may employ force sensing directly, or may employ acceleration, vibration, or acoustic sensing in addition to, or in lieu of, force sensing.

A positioning device for an acetabular cup disposed in a bone, the acetabular cup including an outer shell having a sidewall defining an inner cavity and an opening with the sidewall having a periphery around the opening and with the acetabular cup having a desired abduction angle relative to the bone and a desired anteversion angle relative to the bone, including a controller including a trigger and a selector; a support having a proximal end and a distal end opposite of the proximal end, the support further having a longitudinal axis extending from the proximal end to the distal end with the proximal end coupled to the controller, the support further having an adapter coupled to the distal end with the adapter configured to secure the acetabular cup; and a number N, the number N, an integer greater than or equal to 2, of longitudinal actuators coupled to the controller and disposed around the support generally parallel to the longitudinal axis, each the actuator including an associated impact head arranged to strike a portion of the periphery, each impact head providing an impact strike to a different portion of the periphery when the associated actuator is selected and triggered; wherein each the impact strike adjusts one of the angles relative to the bone.

An installation device for an acetabular cup disposed in a pelvic bone, the acetabular cup including an outer shell having a sidewall defining an inner cavity and an opening with the sidewall having a periphery around the opening and with the acetabular cup having a desired installation depth relative to the bone, a desired abduction angle relative to the bone, and a desired anteversion angle relative to the bone, including a controller including a trigger; a support having a proximal end and a distal end opposite of said proximal end, said support further having a longitudinal axis extending from said proximal end to said distal end with said proximal end coupled to said controller, said support further having an adapter coupled to said distal end with said adapter configured to secure the acetabular cup; and an oscillator coupled to said controller and to said support, said oscillator configured to control an oscillation frequency and an oscillation magnitude of said support with said oscillation frequency and said oscillation magnitude configured to install the acetabular cup at the installation depth with the desired abduction angle and the desired anteversion angle without use of an impact force applied to the acetabular cup.

An installation system for a prosthesis configured to be implanted into a portion of bone at a desired implantation depth, the prosthesis including an attachment system, including an oscillation engine including a controller coupled to a vibratory machine generating an original series of pulses having a generation pattern, said generation pattern defining a first duty cycle of said original series of pulses; and a pulse transfer assembly having a proximal end coupled to said oscillation engine and a distal end, spaced from said proximal end, coupled to the prosthesis with said pulse transfer assembly including a connector system at said proximal end, said connector system complementary to the attachment system and configured to secure and rigidly hold the prosthesis producing a secured prosthesis with said pulse transfer assembly communicating an installation series of pulses, responsive to said original series of pulses, to said secured prosthesis producing an applied series of pulses responsive to said installation series of pulses; wherein said applied series of pulses are configured to impart a vibratory motion to said secured prosthesis enabling an installation of said secured prosthesis into the portion of bone to within 95% of the desired implantation depth without a manual impact.

A method for installing an acetabular cup into a prepared socket in a pelvic bone, the acetabular cup including an outer shell having a sidewall defining an inner cavity and an opening with the sidewall having a periphery around the opening and with the acetabular cup having a desired installation depth relative to the bone, a desired abduction angle relative to the bone, and a desired anteversion angle relative to the bone, including (a) generating an original series of pulses from an oscillation engine; (b) communicating said original series of pulses to the acetabular cup producing a communicated series of pulses at said acetabular cup; (c) vibrating, responsive to said communicated series of pulses, the acetabular cup to produce a vibrating acetabular cup having a predetermined vibration pattern; and (d) inserting the vibrating acetabular cup into the prepared socket within a first predefined threshold of the installation depth with the desired abduction angle and the desired anteversion angle without use of an impact force applied to the acetabular cup.

This method may further include (e) orienting the vibrating acetabular cup within the prepared socket within a second predetermined threshold of the desired abduction angle and within third predetermined threshold of the desired anteversion angle.

A method for inserting a prosthesis into a prepared location in a bone of a patient at a desired insertion depth wherein non-vibratory insertion forces for inserting the prosthesis to the desired insertion depth are in a first range, the method including (a) vibrating the prosthesis using a tool to produce a vibrating prosthesis having a predetermined vibration pattern; and (b) inserting the vibrating prosthesis into the prepared location to within a first predetermined threshold of the desired insertion depth using vibratory insertion forces in a second range, said second range including a set of values less than a lowest value of the first range.

An embodiment may include a force sensing system within the BMD tools with capacity to measure the force experienced by the system (mIF) (Within the tool) and calculate the change in mIF with respect to time, number of impacts, or depth of insertion. This system provides a feedback mechanism through the BMD tools, for the surgeon, as to when impaction should stop, and or if it should continue. This feedback mechanism can be created by measuring and calculating force, acceleration or insertion depth. In some implementations, an applied force is measured (TmIF) and compared against the mIF in any of several possible ways and an evaluation is made as to whether the prosthesis has stopped moving responsive to the applied forces. There are different implications depending upon where in the installation process the system is operating. In other implementations, the applied force is known or estimated and then the mIF may need to be measured.

An aspect of the present invention is use of a special version of this system to map out ranges of parameters for different prosthesis/cavity interactions to allow better understanding of typical or applicable curve for a particular patient with a particular implant procedure.

A force sensing system for a medical device tools with capacity to measure the force experienced by the system (mIF)−(Within the tool) and calculate a change in mIF with respect to time, number of impacts, or depth of insertion, wherein this system provides a feedback mechanism through the device, for the surgeon, as to when impaction should stop, and/or whether it should continue while assessing a risk of too early suspension with poor seating or too late when bone fracture risk is high and wherein this feedback mechanism can be created by measuring and calculating force, acceleration or insertion depth, among other variables.

An apparatus, including a medical device operating over a continuous period including an initial act with the medical device to a subsequent act with the medical device; and a microelectromechanical (MEM) sensing system physically coupled to the medical device configured to provide a realtime parametric evaluation over the period.

A tool for inserting a prosthesis into a portion of a bone, including a shaft receiving an agency configured for an insertion of the prosthesis into the bone using the shaft; and a first sensor providing a feedback of a response of the bone to the agency during the insertion.

A method for inserting a prosthesis into a portion of a bone, including using a shaft to receive an agency configured for an insertion of the prosthesis into the bone using the shaft; and providing, using a first sensor, a feedback of a response of the bone to the agency during the insertion.

An embodiment of the present invention may include a system for a body, the system providing extracorporeal data regarding a status within the body, including a set of tags, each tag configured for access outside of the body; a set of fixators, each fixator associated with one of the tags of the set of tags and configured to fix the associated tag to a portion of tissue within the body; and a tag interface, disposed outside the body and in wireless communication with the set of tags, configured to wirelessly access the set of tags; and wherein the set of tags is configured to produce collectively the status; and wherein the system is configured to obtain the status from the set of tags.

A method for a body, the method providing extracorporeal data regarding a status within the body, including fixing a set of tags to a portion of tissue within the body, each tag configured for access outside of the body wherein each tag includes an associated fixator configured to fix the associated fixator to unique locations on the portion of tissue; accessing extracorporeally and wirelessly the set of tags using a tag interface; and wherein the set of tags is configured to produce collectively the status; and wherein the method is configured to obtain the status from the set of tags.

A method for evaluating a parameter of a body during a joint repair having a first implant disposed within a first bone inside the body, the first implant including a component configured to engage a second disposed within a second bone inside the body, the method including fixing a first tag to a first location on the first bone; fixing a second tag to a second location on the second bone; accessing extracorporeally the tags using a tag interface outside the body to produce the parameter. In some cases the joint repair includes a THA procedure, wherein the first bone includes a pelvis, wherein the second bone includes a femur, and wherein the parameter includes one of a leg length or a leg offset.

A method for screw/sensors to passively communicate with an external microchip/microcontroller (in the OR space, table pad, clamp to OR bed, optionally disposable) to determine changes in leg length, offset and angle between the screw/sensors.

Other embodiments may include a method for fixing a set of tags to bone (within, surface, or protruding). A method for continuous monitoring of leg length and offset (and angular changes in total joint surgery (THR), without the need for establishment of a (3-dimensional coordinate system in the OR space) or use of fluoroscopy. A method for screw/sensors (and embedded microcontrollers) to actively communicate with each other to determine changes in distance and angle between the two sensors in (and during) pre and post implantation phase of joint replacement surgery. A method for screw/sensors to passively communicate with an external microchip/microcontroller (in the OR space, table pad, clamp to OR bed, disposable) to determine changes in leg length, offset and angle between the screw/sensors. A method that allows real-time live monitoring of changes in length and angle between two fixed points about a joint non-stop during the whole (not part) course of the operation. A method that generally requires no more than 2 minutes of added time to the surgery that allows continuous awareness of changes in leg length and offset throughout the course of the operation.

A system for a body, the system providing extracorporeal data regarding a status of an anatomical structure within the body, including: a set of subdermal reference tags; a tag interface, disposed outside the body and in communication with the set of tags, configured to support a local positioning system; and wherein the local positioning system assists in location of an implement relative to one or more anatomical elements of the anatomical structure; and wherein the local positioning system is obscured from direct line of sight access.

The system wherein the anatomical structure includes a pelvis and a femur coupled to the pelvis, a knee joint, shoulder, or other anatomical location of interest.

The system wherein the set of tags include one or more devices selected from group consisting of a passive sensor, an active sensor, a passive tag, an active tag, a passive reference, an active reference, and combinations thereof.

A system wherein the anatomical structure includes a knee joint.

A method for continuous monitoring of a position of an ACL guide with respect to ACL ligament bundles, without a need for establishment of a (multi-dimensional coordinate system (2D/3D) in the OR space) or use of operating room imagers.

A system developing/presenting/implementing a local positioning system LPS (without any requirement, though optional in some capacity, to establish a global 3D coordinate system of the operating room (OR) space) for monitoring/setting bone cutting and implant positioning/fitting (prosthesis and ligaments) activities in orthopedic surgery, comprising of reference tags capable to emitting and sensing (electronic and electromagnetic) information while embedded in bone and implants, which in some cases may be performed in realtime or near realtime.

A system developing a local positioning system (LPS) as an ‘assistive positioning system’ that provides quick and accurate positional cues to the surgeons for ligament and implant positioning without requiring an addition of: (a) large bulky equipment (e.g., expensive computers, portable imagers, and robots) sterile covered and intrusively introduced into the operative theatre; (b) a need to perform multiple extra steps (continual data input) during the flow of the operation, to obtain positional data (data output).

A system of establishing an LPS for arthroplasty and ligament reconstruction surgery where positional information is created by a collective activity of reference tags, beacons, nodes, sensors attached to tissue (e.g., subdermal bone or connective tissue) and implants: (a) where self-operating (emitter/sensing activities) of the reference tags collectively produce positional information regarding anatomical landmarks of interest (bony prominences, ligament footprints) and implant position live and in real time during the surgical process; (b) without interruption of flow of operation to input data in order to receive output data; and (c) without a need for interruptive steps during a flow of a surgical procedure, typically required for imaging/navigation/robotic systems beholding to line of sight issues.

An assistive ligament positioning guide system for ACL reconstruction where an internal (within the joint) location of ligament footprints are determined by external anatomical, including subdermal tags, cues provided by reference tag LPS system.

An assistive implant position system for orthopedic arthroplasty where the positioning of implants is guided by reference tag activated LPS system.

An ACL positioning guide configured for standardization of tunnel placement, where the exact position of the chosen tunnels with respect to surrounding anatomical landmarks can be measured and documented.

A method allowing a universal data collection process, conducive to data analysis and cognitive technologies of quantified, by use of an LPS used during a procedure, including an assessment of reasons for degradation or failing of the one or more components used in the procedure.

An ACL positioning guide with capacity to customize (patient specific) ACL reconstruction wherein a reproduction of a patient's own exact anatomical ligament footprints is substantially recreated by use of an LPS.

An LPS system for total hip arthroplasty allowing for real-time, live monitoring of implant position without the need for large computers/robots inserted (in sterile cover) within the operative field.

An LPS system for total hip arthroplasty allowing for realtime/near realtime, live monitoring of implant position without a requirement for a surgeon to manually input multiple data points in order to obtain output data regarding a position of the implants.

An LPS system for arthroplasty procedures (e.g., hip, knee, and shoulder), where exact leg length and offset changes are monitored live and presented realtime/near realtime to a surgeon, without additional surgeon involvement or surgeon actions.

An ACL positioning guide system configured for exact identification of a patient's own anteromedial and posterolateral ligament bundle foot prints for ACL reconstruction.

A method of automatically postoperatively monitoring arthroplasty implants over time with subdermal reference tags for subsidence and loosening.

A system and method developing an autonomous local positioning system to be utilized in implant and ligament positioning during orthopedic surgery, that can be established purely by mathematical and algorithmic calculations without a need for imaging studies and camera, vision, optical and line of sight monitoring.

A system and method for pre-operatively installing a set of primary subdermal tags for surgical preplanning, including imaging, configuring a virtual component installed in a surgical procedure, wherein the set of primary subdermal tags are configured as part of a local positioning system locating an actual component corresponding to the virtual component installed during the surgical procedure, wherein a set of secondary subdermal tags may be installed during the procedure for enhancement/completion of the local positioning system.

A flexible foundation supporting a preinstalled set of reference tags, some or all of which may have a predetermined relative location with respect to each other, for installation as part of a local positioning system used in a surgical procedure.

Any of the embodiments described herein may be used alone or together with one another in any combination. Inventions encompassed within this specification may also include embodiments that are only partially mentioned or alluded to or are not mentioned or alluded to at all in this brief summary or in the abstract. Although various embodiments of the invention may have been motivated by various deficiencies with the prior art, which may be discussed or alluded to in one or more places in the specification, the embodiments of the invention do not necessarily address any of these deficiencies. In other words, different embodiments of the invention may address different deficiencies that may be discussed in the specification. Some embodiments may only partially address some deficiencies or just one deficiency that may be discussed in the specification, and some embodiments may not address any of these deficiencies.

Other features, benefits, and advantages of the present invention will be apparent upon a review of the present disclosure, including the specification, drawings, and claims.

### BRIEF DESCRIPTION OF THE DRAWINGS

The accompanying figures, in which like reference numerals refer to identical or functionally-similar elements throughout the separate views and which are incorporated in and form a part of the specification, further illustrate the present invention and, together with the detailed description of the invention, serve to explain the principles of the present invention.

FIG. 1 illustrates a smart tool for prosthesis installation;

FIG. 2 illustrates an identification of forces in a press fit fixation installation of a prosthesis;

FIG. 3 illustrates a set of relationships between measured impact force (e.g., F5), number of impacts (NOI), cup insertion (CI), and impact energy Joules (J);

FIG. 4 illustrates a relationship of force in bone (e.g., F5) and cup insertion (CI) for 1.0 Joules (J);

FIG. 5 illustrates a relationship of force in bone (e.g., F5) and cup insertion (CI) for 1.8 Joules (J);

FIG. 6 illustrates a relationship between a rate of insertion (1/NOI), extractive force (e.g., F4), and impact energy;

FIG. 7 illustrates a relationship between maximum applied force (e.g., F1) and cup insertion (CI);

FIG. 8 illustrates a relationship between maximum applied force (e.g., F1) and an extractive force (e.g., F4);

FIG. 9 illustrates a representative force response for incrementing impact energies;

FIG. 10 illustrates a comparison of a quantitative system versus a qualimetric system for evaluating a real time non-visually tracked press fit fixation;

FIG. 11-FIG. 14 illustrate a set of rigidity metric measurements;

FIG. 11 illustrates a comparison of F5 to F1;

FIG. 12 illustrates a comparison of ΔF5 to a predetermined threshold (e.g., 0.0);

FIG. 13 illustrates a comparison of F2 to F1;

FIG. 14 illustrates a comparison of ΔF2 to a predetermined threshold (e.g., 0.0);

FIG. 15 illustrates an evolution of an acetabular cup consistent with improving press fit fixation;

FIG. 16 illustrates a particular embodiment of a BMDx force sensing tool;

FIG. 17 illustrates an example of suspensory cortical fixation;

FIG. 18 illustrates an example of aperture interference screw fixation;

FIG. 19 illustrates an example of a native connective tissue graft;

FIG. 20 illustrates an example of a compressed connective tissue graft that may result from a pre-operative compressive treatment of the native connective tissue graft of FIG. 19;

FIG. 21 illustrates a perspective view of a graft platform;

FIG. 22 illustrates a side view of the graft platform of FIG. 21 with repositioned stages;

FIG. 23 illustrates a sectional view of a pair of collets gripping the native connective tissue graft of FIG. 19;

FIG. 24 illustrates an end view of FIG. 7;

FIG. 25 illustrates an end view similar to FIG. 8 but after lateral compression to produce the compressed connective tissue graft of FIG. 20;

FIG. 26 illustrates a perspective view of a collet of the graft platform;

FIG. 27 illustrates an end view of the collet of FIG. 26;

FIG. 28 illustrates a side sectional view of the collet of FIG. 26;

FIG. 29-FIG. 30 illustrates a reconstruction of an ACL in a pair of cylindrical bone tunnels;

FIG. 29 illustrates pre-expansion of a compressed ACL graft;

FIG. 30 illustrates a post-expansion of the compressed ACL graft;

FIG. 31-FIG. 32 illustrates a reconstruction of an ACL into a pair of profiled bone tunnels;

FIG. 31 illustrates pre-expansion of a compressed ACL graft;

FIG. 32 illustrates a post-expansion of the compressed ACL graft;

FIG. 33 illustrates different conforming expansions of a compressed ACL graft, dependent upon a preparation of a bone tunnel;

FIG. 34 illustrates a preparation of a profiled bone tunnel by an automated surgical apparatus;

FIG. 35 illustrates an allograft system including a pre-compressed allograft with a sheathing subsystem having an outer sheath and an inner sheath;

FIG. 36 illustrates an allograft system including a pre-compressed allograft with a prosthesis subsystem having at least one connective tissue prosthetic element; and

FIG. 37 illustrates an allograft system including a pre-compressed allograft with an expansion subsystem having at least one expansion element.

FIG. 38-FIG. 46 illustrate aspects of biologic installation structures including a set of sensors;

FIG. 38 illustrates a general biosensor;

FIG. 39 illustrates a point-of-care (PI-POCT) diagnostic device;

FIG. 40 illustrates an implementation of force/displacement sensing with interference fit fixation;

FIG. 41 illustrates an implementation of an aseptic loosening sensing, linear variable displacement transformers (LVDT), with interference fit fixation;

FIG. 42 illustrates a biosensor integrated microelectronic sensor;

FIG. 43 illustrates a system for assessing metallosis and trunnionosis;

FIG. 44 illustrates a system for assessing optimal press fit in ligament reconstruction;

FIG. 45 illustrates a system for assessing poor healing of a reconstructed ligaments;

FIG. 46 illustrates a system for assessing various failure modes of a reconstructed ligament grafts;

FIG. 47 illustrates a first gait reaction force over time for a step;

FIG. 48 illustrates a second gait reaction force over time for a step;

FIG. 49 illustrates a biologic sensing architecture;

FIG. 50 illustrates a set of “cup prints” for a number of interactions between a cup and a cavity;

FIG. 51 illustrates a particular one representative cup print;

FIG. 52 illustrates a controlled modulated installation force envelope;

FIG. 53 illustrates an example installation force envelope that is representative of use of a mallet in its production;

FIG. 54 illustrates an example installation force envelope that is representative of use of a BMD3 in its production;

FIG. 55 illustrates an example installation force envelope that is representative of use of a BMD4 in its production;

FIG. 56-FIG. 59 relate to a vibratory Behzadi Medical Device (BMD3);

FIG. 56 illustrates a representative installation system;

FIG. 57 illustrates a disassembly of the representative installation system of FIG. 56;

FIG. 58 illustrates a first disassembly view of the pulse transfer assembly of the installation system of FIG. 56;

FIG. 59 illustrates a second disassembly view of the pulse transfer assembly of the installation system of FIG. 56;

FIG. 60 illustrates an embodiment for a sliding impact device having a pressure sensor to provide feedback and attachment of an optional navigation device;

FIG. 61 illustrates a Force Resistance (FR) curve;

FIG. 62-FIG. 63 illustrate a general force measurement system for understanding an installation of a prosthesis into an installation site (e.g., an acetabular cup into an acetabulum during total hip replacement procedures);

FIG. 62 illustrates an initial engagement of a prosthesis to a cavity when the prosthesis is secured to a force sensing tool;

FIG. 63 illustrates a partial installation of the prosthesis of FIG. 62 into the cavity by operation of the force sensing tool;

FIG. 64 illustrates a generalized FR curve illustrating various applicable forces implicated in operation of the tool in FIG. 62 and FIG. 63;

FIG. 65-FIG. 70 illustrate a first specific implementation of the system and method of FIG. 62-FIG. 64;

FIG. 65 illustrates a representative plot of insertion force for a cup during installation;

FIG. 66 illustrates a first particular embodiment of a BMDX force sensing tool;

FIG. 67 illustrates a graph including results of a drop test over time;

FIG. 68 illustrates a graph of measured impact force as impact energy is increased;

FIG. 69 illustrates a discrete impact control and measurement process; and

FIG. 70 illustrates a warning process; and

FIG. 71-FIG. 76 illustrate a second specific implementation of the system and method of FIG. 62-FIG. 64;

FIG. 71 illustrates a basic force sensor system for controlled insertion;

FIG. 72 illustrates an FR curve including TmIF and mIF as functions of displacement;

FIG. 73 illustrates a generic force sensor tool to access variables of interest in FIG. 72;

FIG. 74 illustrates a B-cloud tracking process using TmIF and MIF measurements;

FIG. 75 illustrates a control system for the “controlled action” referenced in FIG. 74;

FIG. 76 illustrates possible B-cloud regulation strategies;

FIG. 77 illustrates a generalized BMD including realtime invasive sense measurement;

FIG. 78-FIG. 79 illustrate an alternative general force measurement system for understanding an installation of a prosthesis into an installation site (e.g., an acetabular cup into an acetabulum during total hip replacement procedures);

FIG. 78 illustrates an initial engagement of a prosthesis to a cavity when the prosthesis is secured to a force sensing system;

FIG. 79 illustrates a partial installation of the prosthesis of FIG. 78 into the cavity by operation of the force sensing tool; and

FIG. 80 illustrates a conventional sensing implementation system implementing a set of Schantz screws fixated to bone, which may be coupled, directly or indirectly, to a clamp, sensor, marker, reference base or optical tracker;

FIG. 81 illustrates two fixed points on the pelvis and femur that may be used to measure changes in leg length (y-axis) and offset (x-axis);

FIG. 82-FIG. 86 illustrate various types of tags, such as anchors, screws, barbs, hooks, and threaded pins armed with sensors;

FIG. 82 illustrates a tag having a body coupled to an active/passive device;

FIG. 83 illustrates a tag having a body coupled to an active/passive device;

FIG. 84 illustrates a tag having a body coupled to an active/passive device;

FIG. 85 illustrates a tag having a body coupled to an active/passive device; and

FIG. 86 illustrates a tag having a body coupled to an active/passive device;

FIG. 87 illustrates various types of sensors incorporated within anchors and applied to pelvis and femur to allow real time evaluation of leg length and offset changes during THR;

FIG. 88 illustrates simple measurement of “offset” or delta X in the X axis;

FIG. 89 illustrates simple measurement of “leg length” or delta Y in the Y axis;

FIG. 90 illustrates integrated circuits and microelectronics incorporated within the anchor-sensors calculating change (delta) in the Y and X planes between the two anchor-sensors simultaneously through mathematical and geometric algorithms;

FIG. 91 illustrates threaded screw sensor similar to {rotator cuff anchors} rapidly applied and removed with a drill or hand screwdriver;

FIG. 92 illustrates an anchor-sensor;

FIG. 93 illustrates a Schantz screw-sensor which may be permanent or absorbable;

FIG. 94 illustrates a set of alternative set of reference tag attachment modalities; and

FIG. 95-FIG. 97 illustrate a set of implementations for systems for realtime externally accessed intracorporeal reference tags;

FIG. 95 illustrates a first implementation of a system for realtime externally accessed intracorporeal reference tags;

FIG. 96 illustrates a second implementation of a system for realtime externally accessed intracorporeal reference tags;

FIG. 97 illustrates a third implementation of a system for realtime externally accessed intracorporeal reference tags;

FIG. 98-FIG. 116 illustrate additional uses and implementations of anatomical locator tags;

FIG. 98 illustrates femoral and tibial attachment sites (footprints) of the anterior cruciate ligament anteromedial (AM) and posterolateral (PL) bundles;

FIG. 99 illustrates a conventional anterior cruciate ligament tibial ACL guide;

FIG. 100 illustrates a spatial relationship of three anatomical tags to the anteromedial AM bundle attachment of ACL on tibia;

FIG. 101 illustrates a spatial relationship between three anatomical tags on the tibia and the anteromedial (AM) and posterolateral (PM) bundle attachments sites (footprints) of ACL on the tibia;

FIG. 102 illustrates a spatial relationship between three anatomical tags on the femur and the anteromedial (AM) and posterolateral (PM) bundle attachments sites (footprints) of ACL on the femur;

FIG. 103 illustrates a three-dimensional spatial relationship between three anatomical reference tags on the femur and femoral anteromedial (AM) tunnel, and a three-dimensional spatial relationship between three reference tags on the tibia and the tibial anteromedial (AM) tunnel;

FIG. 104 illustrates a tibial ACL guide positioned to determine an eventual tunnel and footprint placement;

FIG. 105 illustrates an LPS-ACL guide all-in-one unit using the installed anatomical reference tags;

FIG. 106 illustrates an alternative LPS-ACL guide with separate monitor display using the installed anatomical reference tags;

FIG. 107 illustrates an LPS-ACL guide sensor tip indicating a discrepancy between its position and the AM bundle attachment site (improperly positioned footprint) of ACL, indicated by star off center from bulls' eye;

FIG. 108 illustrates the LPS-ACL guide sensor tip properly positioned hovering of FIG. 107 over the AM bundle attachment site (footprint) of ACL, indicated by star centered over the bulls eye;

FIG. 109 illustrates a determination of time of flight Measurements for distance—speed of light and sound are known;

FIG. 110 illustrates an example of distance and angular relationship measurements between the anatomical reference tags and desired insertion sites, in this example the footprint of the anteromedial AM bundle of ACL on the tibia;

FIG. 111 illustrates an LPS-ACL guide using a set of anatomical sensors using a distributed magnetic local positioning system (DMLP) to identify insertion sites;

FIG. 112 illustrates a preparation system using the anatomical reference tags;

FIG. 113 illustrates a reference virtual implant position;

FIG. 114 illustrates an LPS measurement of actual implant position(s) using installed anatomical reference tags;

FIG. 115 illustrates a generalized BMD including an anatomical tag local positioning system; and

FIG. 116 illustrates a generalized BMD including an anatomical tag installation function.

### DETAILED DESCRIPTION OF THE INVENTION

Embodiments of the present invention provide a system and method for quantitatively assessing a press fit value (and provide a mechanism to evaluate optimal quantitative values) of any implant/bone interface regardless the variables involved including bone site preparation, material properties of bone and implant, implant geometry and coefficient of friction of the implant-bone interface without requiring a visual positional assessment of a depth of insertion. The following description is presented to enable one of ordinary skill in the art to make and use the invention and is provided in the context of a patent application and its requirements.

Embodiments of the present invention provide a system and method for allowing any surgeon, including those surgeons who perform a fewer number of a replacement procedure as compared to a more experienced surgeon who performs a greater number of procedures, to provide an improved likelihood of a favorable outcome approaching, if not exceeding, a likelihood of a favorable outcome as performed by a very experienced surgeon with the replacement procedure, such as by understanding the prosthesis installation environment (e.g., cup/cavity interface) and to provide intelligent and interactive tools and methods to standardize the installation process and provide feedback regarding a quality of insertion/installation. The following description is presented to enable one of ordinary skill in the art to make and use the invention and is provided in the context of a patent application and its requirements.

Various modifications to the preferred embodiment and the generic principles and features described herein will be readily apparent to those skilled in the art. Thus, the present invention is not intended to be limited to the embodiment shown but is to be accorded the widest scope consistent with the principles and features described herein.

### Definitions

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

The following definitions apply to some of the aspects described with respect to some embodiments of the invention. These definitions may likewise be expanded upon herein.

As used herein, the term “or” includes “and/or” and the term “and/or” includes any and all combinations of one or more of the associated listed items. Expressions such as “at least one of,” when preceding a list of elements, modify the entire list of elements and do not modify the individual elements of the list.

As used herein, the singular terms “a,” “an,” and “the” include plural referents unless the context clearly dictates otherwise. Thus, for example, reference to an object can include multiple objects unless the context clearly dictates otherwise.

Also, as used in the description herein and throughout the claims that follow, the meaning of “in” includes “in” and “on” unless the context clearly dictates otherwise. It will be understood that when an element is referred to as being “on” another element, it can be directly on the other element or intervening elements may be present therebetween. In contrast, when an element is referred to as being “directly on” another element, there are no intervening elements present.

As used herein, the term “set” refers to a collection of one or more objects. Thus, for example, a set of objects can include a single object or multiple objects. Objects of a set also can be referred to as members of the set. Objects of a set can be the same or different. In some instances, objects of a set can share one or more common properties.

As used herein, the term “adjacent” refers to being near or adjoining. Adjacent objects can be spaced apart from one another or can be in actual or direct contact with one another. In some instances, adjacent objects can be coupled to one another or can be formed integrally with one another.

As used herein, the terms “connect,” “connected,” and “connecting” refer to a direct attachment or link. Connected objects have no or no substantial intermediary object or set of objects, as the context indicates.

As used herein, the terms “couple,” “coupled,” and “coupling” refer to an operational connection or linking. Coupled objects can be directly connected to one another or can be indirectly connected to one another, such as via an intermediary set of objects.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent of the given numeric value provided such a deviation does not alter the end function or result of the value. Therefore, a value of about 1% can be construed to be a range from 0.9% to 1.1%.

As used herein, the terms “substantially” and “substantial” refer to a considerable degree or extent. When used in conjunction with an event or circumstance, the terms can refer to instances in which the event or circumstance occurs precisely as well as instances in which the event or circumstance occurs to a close approximation, such as accounting for typical tolerance levels or variability of the embodiments described herein.

As used herein, the terms “optional” and “optionally” mean that the subsequently described event or circumstance may or may not occur and that the description includes instances where the event or circumstance occurs and instances in which it does not.

As used herein, the term “bone” means rigid connective tissue that constitute part of a vertebral skeleton, including mineralized osseous tissue, particularly in the context of a living patient undergoing a prosthesis implant into a portion of cortical bone. A living patient, and a surgeon for the patient, both have significant interests in reducing attendant risks of conventional implanting techniques including fracturing/shattering the bone and improper installation and positioning of the prosthesis within the framework of the patient's skeletal system and operation.

As used herein, the term “size” refers to a characteristic dimension of an object. Thus, for example, a size of an object that is spherical can refer to a diameter of the object. In the case of an object that is non-spherical, a size of the non-spherical object can refer to a diameter of a corresponding spherical object, where the corresponding spherical object exhibits or has a particular set of derivable or measurable properties that are substantially the same as those of the non-spherical object. Thus, for example, a size of a non-spherical object can refer to a diameter of a corresponding spherical object that exhibits light scattering or other properties that are substantially the same as those of the non-spherical object. Alternatively, or in conjunction, a size of a non-spherical object can refer to an average of various orthogonal dimensions of the object. Thus, for example, a size of an object that is a spheroidal can refer to an average of a major axis and a minor axis of the object. When referring to a set of objects as having a particular size, it is contemplated that the objects can have a distribution of sizes around the particular size. Thus, as used herein, a size of a set of objects can refer to a typical size of a distribution of sizes, such as an average size, a median size, or a peak size.

As used herein, the term “mallet” or “hammer” or similar refers to an orthopedic device made of stainless steel or other dense material having a weight generally a carpenter's hammer and a stonemason's lump hammer.

As used herein, the term “impact force” for impacting an acetabular component (e.g., an acetabular cup prosthesis) includes forces from striking an impact rod multiple times with the orthopedic device that are generally similar to the forces that may be used to drive a three inch nail into a piece of lumber using the carpenter's hammer by striking the nail approximately a half-dozen times to completely seat the nail. Without limiting the preceding definition, a representative value in some instances includes a force of approximately 10 lbs./square inch.

As used herein, the term “realtime” sensing means sensing relevant parameters (e.g., force, acceleration, vibration, acoustic, and the like) during processing (e.g., installation, reaming, cutting) without stopping or suspending processing for visual evaluation of insertion depth of a prosthesis into a prepared cavity.

As used herein, the term “implant” means, unless the context clearly indicates otherwise, an expansive collection of structures designed and intended to be installed into tissue or bone of a body such as a living body or cadaver and includes prostheses, implants, grafts, and the like.

As used herein, the term “tag” means, unless the context clearly indicates otherwise, a structure fixed, often temporarily but not required, to a portion of tissue. This structure may be a passive or wholly/partially active element and may interact with other tags with at least one interactive tag of a set of tags in wireless extracorporeal interaction with a tag interface disposed outside a body including the portion of tissue. A passive tag is unpowered and responds to some stimulus or signal to wirelessly communicate a status to the tag interface. An active tag is powered, such as by a battery or wirelessly provided power, and may wirelessly transmit data regarding the status to the tag interface. A tag may include a reference (e.g., an imaging opaque structure), sensor, a transmitter, a transceiver, a ranging system, and the like, and may employ different technologies such as electromagnetics, magnetics, sound (e.g., ultrasound), and the like.

As used herein, the term “obscure” or “obscured” means, unless the context clearly indicates otherwise, a fixation of one or more of the interactive tags at a location in which a wholly or partially opaque barrier is disposed between the interactive tag and the tag interface that would degrade or disrupt conventional line-of-sight navigation structures. For example, with an interactive tag fixed to tissue within a body, an overlying dermis layer, or other tissue or structure, may obscure such an interactive tag when the dermis, tissue, or structure extends between a line-of-sight, for visual systems, between the interactive tag and the tag interface.

As used herein, the term “non-interventional” means, unless the context clearly indicates otherwise, operation without active configuration or manipulation by the surgeon. Conventional THA procedures include multiple interventional assessments of length and/or offset such as by preparing and operating imaging equipment that is brought into the operating room for imaging and then removed from the operating room after imaging and then the image is processed and reviewed. Another interventional process includes the surgeon operating a manual measurement device, e.g., calipers, each time the surgeon desires information about current leg length or leg offset. In contrast, some embodiments of the present invention may provide non-interventional realtime or near realtime assessment of leg length or leg offset without having the surgeon interrupt the THA with imaging or with operating the measurement device. Some advance pre-operative configuration is acceptable, such as fixation of the set of set of tags at desired locations with respect to one or more portions of tissue of a body.

The following description relates to improvements in a wide-range of prostheses installations into live bones of patients of surgeons. The following discussion focuses primarily on total hip replacement (THR) in which an acetabular cup prosthesis is installed into the pelvis of the patient. This cup is complementary to a ball and stem (i.e., a femoral prosthesis) installed into an end of a femur engaging the acetabulum undergoing repair.

Embodiments of the present invention may include one of more solutions to the above problems. U.S. Pat. No. 9,168,154, expressly incorporated by reference thereto in its entirety for all purposes, includes a description of several embodiments, sometimes referred to herein as a BMD3 device, some of which illustrate a principle for breaking down large forces associated with the discrete blows of a mallet into a series of small taps, which in turn perform similarly in a stepwise fashion while being more efficient and safer. The BMD3 device produces the same displacement of the implant without the need for the large forces from the repeated impacts from the mallet. The BMD3 device may allow modulation of force required for cup insertion based on bone density, cup geometry, and surface roughness. Further, a use of the BMD3 device may result in the acetabulum experiencing less stress and deformation and the implant may experience a significantly smoother sinking pattern into the acetabulum during installation. Some embodiments of the BMD3 device may provide a superior approach to these problems, however, described herein are two problems that can be approached separately and with more basic methods as an alternative to, or in addition to, a BMD3 device. An issue of undesirable torques and moment arms is primarily related to the primitive method currently used by surgeons, which involves manually banging the mallet on the impaction plate. The amount of force utilized in this process is also non-standardized and somewhat out of control.

With respect to the impaction plate and undesirable torques, an embodiment of the present invention may include a simple mechanical solution as an alternative to some BMD3 devices, which can be utilized by the surgeon's hand or by a robotic machine. A direction of the impact may be directed or focused by any number of standard techniques (e.g., A-frame, C-arm or navigation system). Elsewhere described herein is a refinement of this process by considering directionality in the reaming process, in contrast to only considering it just prior to impaction. First, we propose to eliminate the undesirable torques by delivering the impacts by a sledgehammer device or a (hollow cylindrical mass) that travels over a stainless rod.

As noted in the background, the surgeon prepares the surface of the hipbone which includes attachment of the acetabular prosthesis to the pelvis. Conventionally, this attachment includes a manual implantation in which a mallet is used to strike a tamp that contacts some part of the acetabular prosthesis. Repeatedly striking the tamp drives the acetabular prosthesis into the acetabulum. Irrespective of whether current tools of computer navigation, fluoroscopy, robotics (and other intra-operative measuring tools) have been used, it is extremely unlikely that the acetabular prosthesis will be in the correct orientation once it has been seated to the proper depth by the series of hammer strikes. After manual implantation in this way, the surgeon then may apply a series of adjusting strikes around a perimeter of the acetabular prosthesis to attempt to adjust to the desired orientation. Currently such post-impaction result is accepted as many surgeons believe that post-impaction adjustment creates an unpredictable and unreliable change which does not therefore warrant any attempts for post-impaction adjustment.

In most cases, any and all surgeons including an inexperienced surgeon may not be able to achieve the desired orientation of the acetabular prosthesis in the pelvis by conventional solutions due to unpredictability of the orientation changes responsive to these adjusting strikes. As noted above, it is most common for any surgeon to avoid post-impaction adjustment as most surgeons understand that they do not have a reliable system or method for improving any particular orientation and could easily introduce more/greater error. The computer navigation systems, fluoroscopy, and other measuring tools are able to provide the surgeon with information about the current orientation of the prosthesis during an operation and after the prosthesis has been installed and its deviation from the desired orientation, but the navigation systems (and others) do not protect against torsional forces created by the implanting/positioning strikes. The prosthesis will find its own position in the acetabulum based on the axial and torsional forces created by the blows of the mallet. Even those navigation systems used with robotic systems (e.g., MAKO) that attempt to secure an implant in the desired orientation prior to impaction are not guaranteed to result in the installation of the implant at the desired orientation because the actual implanting forces are applied by a surgeon swinging a mallet to manually strike the tamp.

A Behzadi Medical Device (BMD) is herein described and enabled that eliminates this crude method (i.e., mallet, tamp, and surgeon-applied mechanical implanting force) of the prosthesis (e.g., the acetabular cup). A surgeon using the BMD is able to insert the prosthesis exactly where desired with proper force, finesse, and accuracy. Depending upon implementation details, the installation includes insertion of the prosthesis into patient bone, within a desired threshold of metrics for insertion depth and location) and may also include, when appropriate and/or desired, positioning at a desired orientation with the desired threshold further including metrics for insertion orientation). The use of the BMD reduces risks of fracturing and/or shattering the bone receiving the prosthesis and allows for rapid, efficient, and accurate (atraumatic) installation of the prosthesis. The BMD provides a viable interface for computer navigation assistance (also useable with all intraoperative measuring tools including fluoroscopy) during the installation as a lighter more responsive touch may be used.

The BMD encompasses many different embodiments for installation and/or positioning of a prosthesis and may be adapted for a wide range of prostheses in addition to installation and/or positioning of an acetabular prosthesis during THR, including examples of a device, which may be automated, for production and/or communication of an installation agency to a prosthesis.

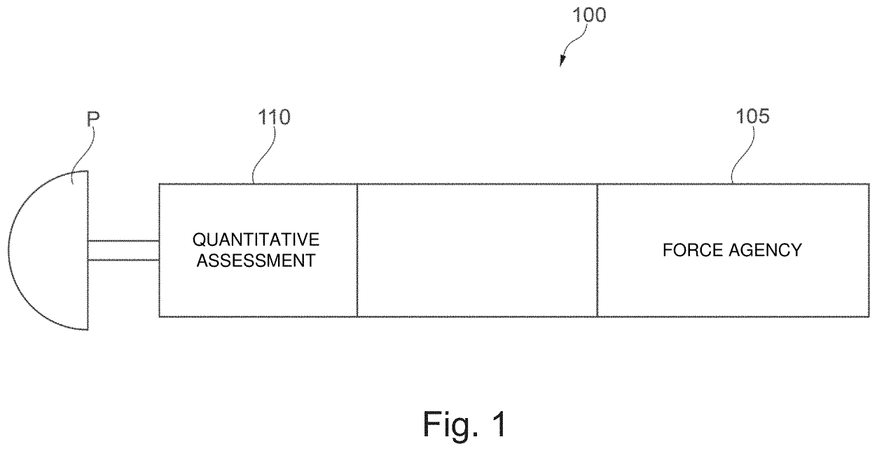

FIG. 1 illustrates a smart tool 100 for prosthesis installation, including structures and methods for operation of a force agency 105 and a responsive quantitative assessment 110 with respect to installation of a prosthesis P (e.g., an acetabular cup) into a prepared cavity in a portion of bone (e.g., an acetabulum). Agency 105 may include several different types of force applicators, including vibratory insertion agencies and/or controlled impaction agencies and/or constant applied force and/or other force profile as described in the incorporated patents and applications. Quantitative assessment 110 may include a processor and sensors for evaluating parameters and functions as described herein including a rigidity metric and an elasticity metric, for press-fit fixation of prosthesis P, such as in realtime or near-realtime operation of force agency 105.

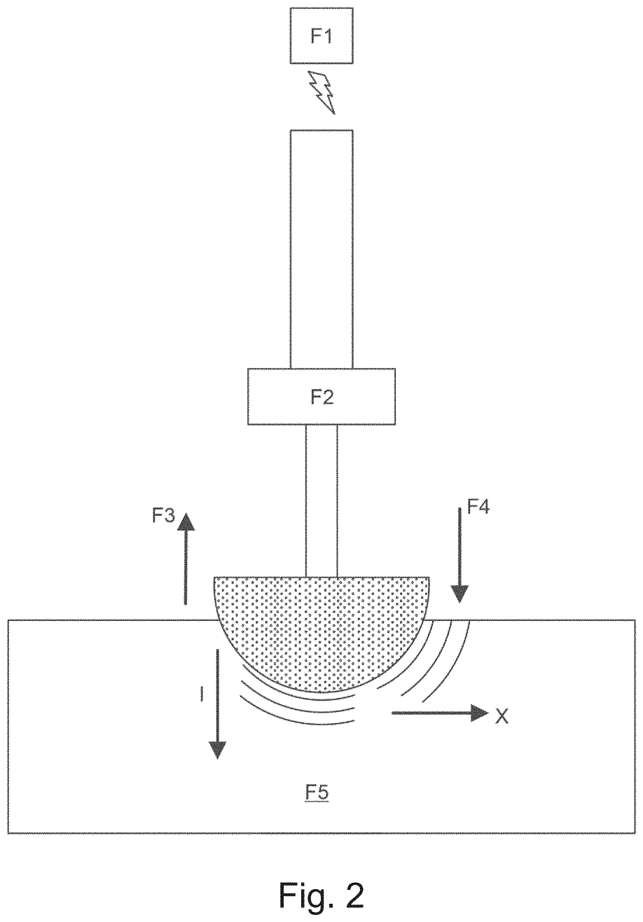

FIG. 2 illustrates an identification of forces in a press fit fixation installation of a prosthesis. These forces, as illustrated, include F1 (applied force), F2 (responsive force in smart tool), F3 (resistive force to installation), F4 (axial extractive force), and/or F5 (force in bone substrate). There may be other forces that may be measured or determined to be correlated, responsive, and/or related to these forces. In some circumstances, multiple related or correlated forces may be “fused” into a fusion force that provides a robust evaluation of the component forces, with any appropriate individual weightings of component forces in the fused force. That is some embodiments, a press-fit fixation may be assessed based upon contributions from multiple forces fused together rather than evaluations of individual forces or derivatives thereof.

When press fitting an acetabular component into an undersized cavity, one may expect to encounter three regions with distinct characteristics: (a) poor seating and poor pull out force; (b) deep insertion and good pull out force; and (c) full insertion which may also have strong fixation but includes higher (and possibly much higher) risk of fracture.

Some embodiments may exhibit relationships between extraction force (F4) and cup insertion CI with respect to similarity and proportionality to a standard stress/strain curve of material deformation.

While two collisions occur during the process of prosthesis impaction into bone in some embodiments for each force application, a proximal collision is usually elastic and typically presents a maximum value of F1 for any given impact energy E of the force application. A distal collision is conversely initially inelastic and progresses to an elastic state as insertion no longer occurs. In some experiments, force measurements in the impaction rod (F2) and bone (F5) may represent the distal collision.

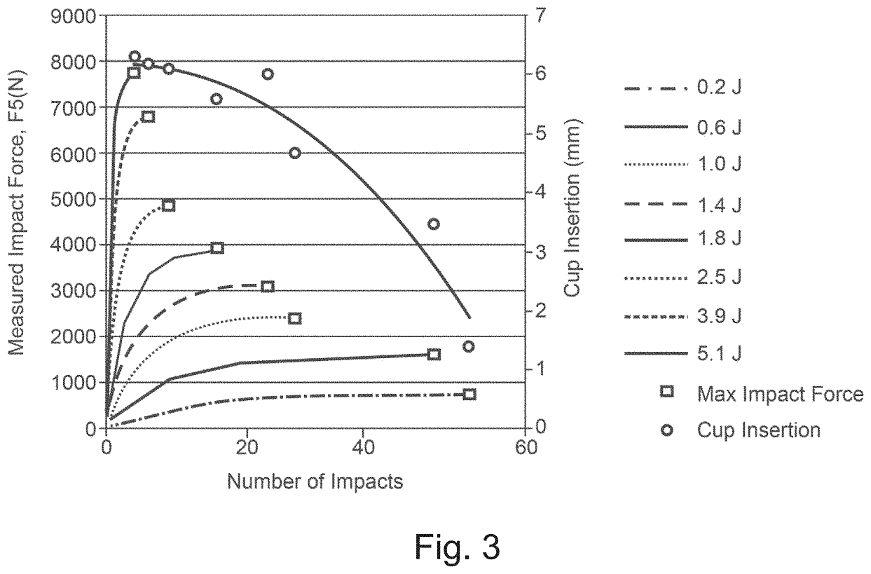

FIG. 3 illustrates a set of relationships between measured impact force (e.g., F2, F3, and/or F5 and/or derivatives and/or combinations thereof), number of impacts (NOI), cup insertion (CI), and impact energy Joules (J). Experiments in the study of vibratory insertion of orthopedic implants [Published Patent App. Invasive Sensing Mechanism: Pub No. 20170196506, incorporated herein by reference in its entirety for all purposes] where an oversized acetabular prosthesis, Zimmer Continuum Cup (62 mm) was inserted into an undersized (61 mm) bone substitute cavity (20 lbs. Urethane foam), using three different insertion techniques including controlled impaction, vibratory insertion, and constant insertion. The forces at play were considered in FIG. 2. An 8900 N force gauge was placed within the polyurethane sample to measure forces in the cavity F5.

With the controlled impaction technique we tested eight-drop heights producing a range of impact energies from 0.2 J to 5.0 J corresponding to impact forces ranging from 550 N to 8650 N. Five replications were performed for each height, with a total sample population of 40 units. For each sample, impacts were repeated at a selected drop height until implant displacement between impacts were within the measurement error of 0.05 mm. Peak impact force in bone F5, total cup insertion CI, and number of impacts NOI to full insertion were recorded for each sample. Cup stability was measured by axial extraction force by means of a pull test using Mark 10 M5-100 test stand and force gauge. The results are shown in Table I.

TABLE I
Drop Test Results

| | | Maximum | | | |
|---|---|---|---|---|---|
| Drop | Impact | Impact Force | Mean | Cup | Extraction |
| Height | Energy | in bone F5 | Number of | Insertion | Force F4 |
| (mm) | (J) | (N) | Impacts | (mm) | (N) |
| 10 | 0.2 | 774 | 52 | 1.4 | 71 |
| 30 | 0.6 | 1641 | 47 | 3.5 | 258 |
| 50 | 1.0 | 2437 | 27 | 4.7 | 480 |
| 70 | 1.4 | 3104 | 23 | 6.0 | 676 |
| 90 | 1.8 | 3927 | 16 | 5.6 | 765 |
| 130 | 2.5 | 4870 | 9 | 6.1 | 827 |
| 200 | 3.9 | 6814 | 6 | 6.2 | 849 |
| 260 | 5.1 | 7757 | 4 | 6.3 | 867 |

These data indicate that every level of impact energy is associated with a final depth of cup insertion CI, a plateauing of the force response in bone F5 to an asymptote, and a certain rate of insertion inversely related to the number of impacts NOI required for insertion. As an example, it took 4 impacts for a maximum applied force of 7757 N to insert the cup 6.3 mm, whereas it took 52 impacts for a maximum applied force of 774 N to insert the cup 1.4 mm.

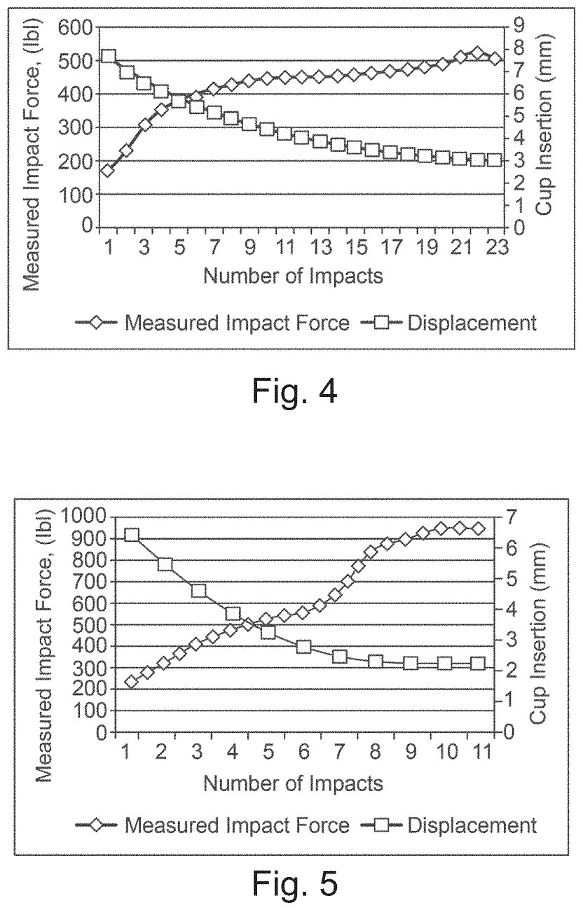

FIG. 4 illustrates a relationship of force in bone (e.g., F5) and cup insertion (CI) for 1.0 Joules (J) and FIG. 5 illustrates a relationship of force in bone (e.g., F5) and cup insertion (CI) for 1.8 Joules (J). A decaying of the force response in bone F5 to an asymptote (when ΔF5 approaches 0) could be used as a parametric value guiding incremental application of energy to obtain optimal press fit fixation of implants. This phenomena is identified herein as the rigidity factor (or rigidity metric) which appears to reach a maximum for any given impact energy.

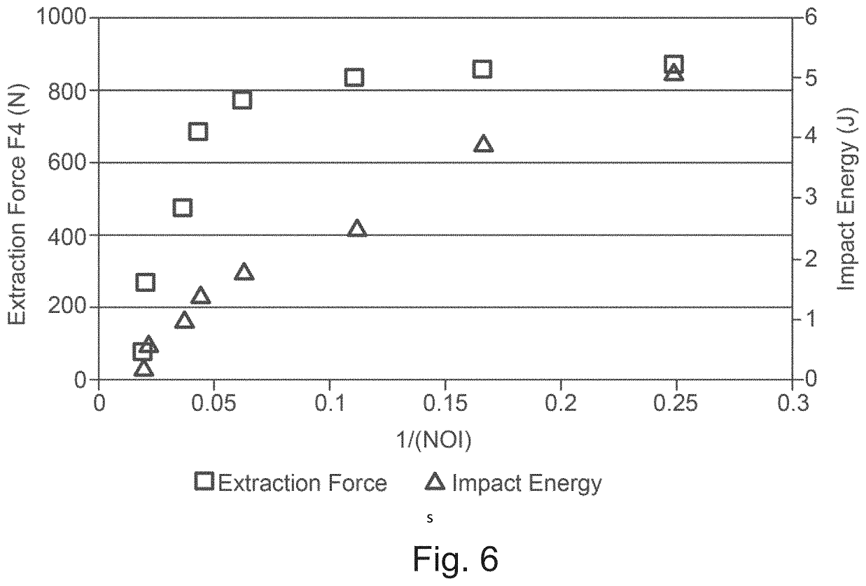

FIG. 6 illustrates a relationship between a rate of insertion (1/NOI), extractive force (e.g., F4), and impact energy. A direct relationship was observed between rate of insertion, inversely related to number of impacts NOI, and the extractive force F4, and this phenomenon is termed an elasticity factor (or elasticity metric), which appears to provide a real-time estimation of the extractive force of the implant/bone interface, as well as an indirect measure of the elastic/plastic behavior of the aperture of bone. A decaying rate of insertion is considered and appears inversely related to a number of impacts and suggests an ultimate stress point of the cavity aperture.

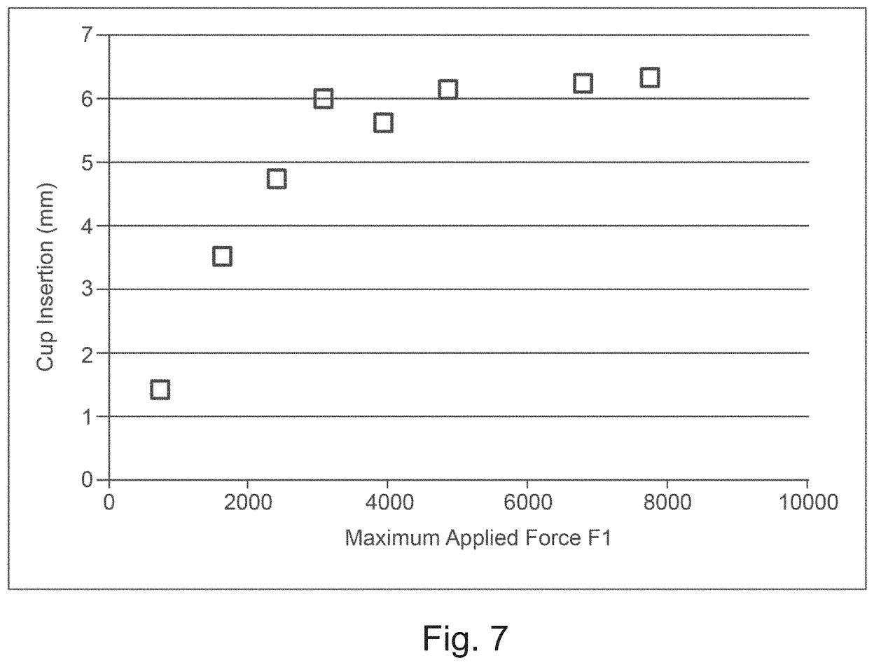

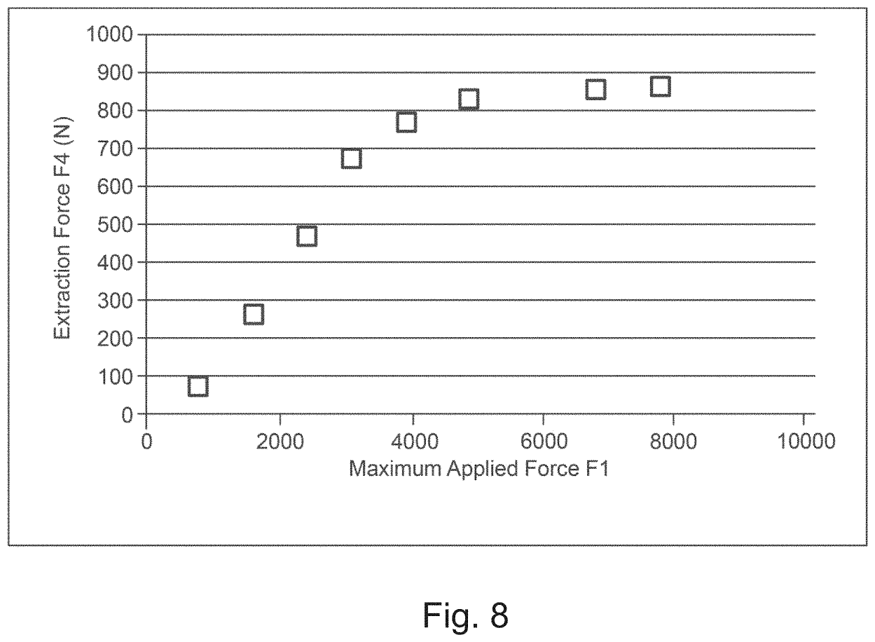

FIG. 7 illustrates a relationship between maximum applied force (e.g., F1) and cup insertion (CI) and FIG. 8 illustrates a relationship between maximum applied force (e.g., F1) and an extractive force (e.g., F4). The relationships of applied force F1 and cup insertion CI as well as applied force F1 and extractive force F4 were evaluated and showed characteristic non-linear curves.

Of note was the observation that an inflection point or (range) exists above which increased applied force F1 (impact energies) did not appear to provide any meaningful increase in cup insertion CI or extraction force F4. As example 1.8 joules of impact energy produced 5.6 mm (89%) of cup insertion CI and 827 N (88%) of extraction force F4. An additional 3.3 joules of impact energy was required for a marginal insertion gain of 0.7 mm and extraction force gain of 102 N.

Questions were posed as to how much force is required for optimal press fit fixation? Does the insistence to fully seat the cup work against the patients and surgeon? Do surgeons risk fracturing the acetabulum in the desire to fully seat the cup? The existence of polar gaps in acetabular press fit fixation have been clinically studied and shown no adverse outcomes.

It was contemplated that a point or (a small range), defined by the parametric values above, exists which could produce the best fixation short of fracture (BFSF) and an embodiment may propose BFSF as an ideal endpoint for all press fit joint replacement surgery. BFSF may, in some situations, act not only as a point of optimal press fit, but also define a sort of speed limit or force limit for the surgeon.

In this application an embodiment may develop a method described as the invasive sensing mechanism (ISM), by which the end point BFSF can be defined in four chosen systems. Additionally, an embodiment may develop an Automatic Intelligent Prosthesis Installation Device (AI-PID) that can quantitatively access this point. The following concept is proposed for a fixation algorithm to achieve BFSF for any implant/cavity interface. (A Double Binary Decision)

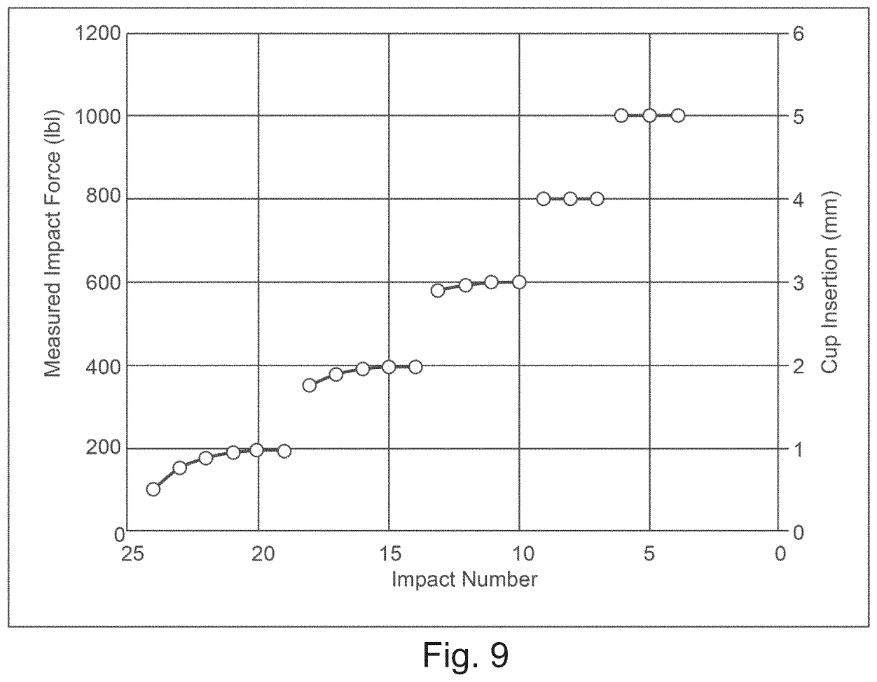

FIG. 9 illustrates a representative force response for incrementing impact energies. The rigidity factor represented by plateauing levels of force in bone (e.g., F5) can be used to guide incremental increase in impact energy J. For any impact energy J, as the force in bone plateaus to a maximum, no further insertion is occurring; a decision can be made as to whether impact energy should be increased or not. This is the first binary decision. The elasticity factor represented by the speed of insertion of an implant (e.g., inversely related to number of impacts (NOI) required for insertion) can be used to guide the surgeon as to whether application of force should continue or not. This is the second binary decision. Two binary decisions for BFSF which may not include full seating.

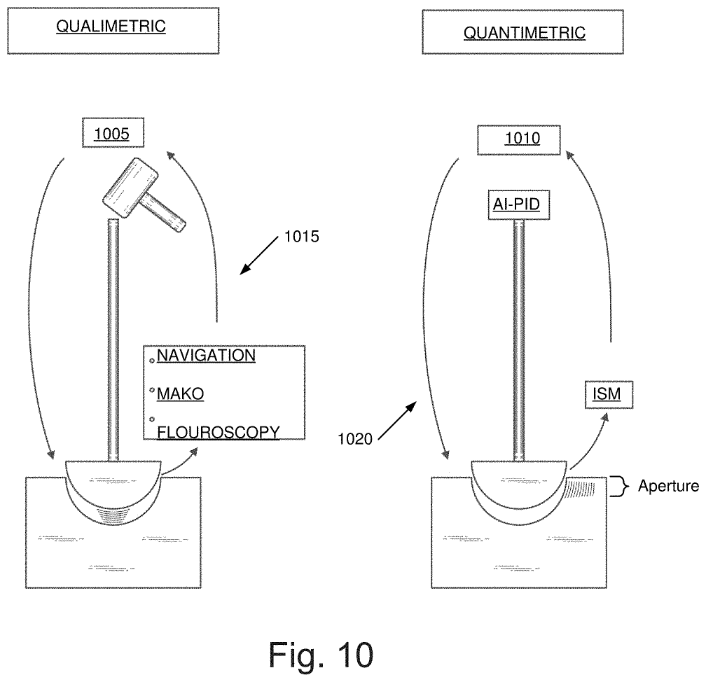

FIG. 10 illustrates a comparison of a quantimetric system (including a measured quantitative determination/use of BFSF) versus a qualimetric system (typically based on a visual qualitative assessment of a depth of insertion) for evaluating a real time non-visually tracked press-fit fixation. An invasive sensing mechanism (ISM) and an automatic intelligent prosthesis installation device (AI-PID) may standardize an application of force and an assessment of a measured quality of fixation in joint replacement surgery, through exploitation of the relationships between the force responses in the installation tool, bone and the interface.

The qualimetric system includes various visual tracking mechanisms (e.g., computer navigation, MAKO assistant, fluoroscopy, and the like) in which an uncontrolled force is applied manually such as by a mallet 1005. The quantitative system operates an insertion agency 1010 which enables application of controlled forces (e.g., force vectors of controlled direction and/or controlled magnitude). The insertion agency may involve ISM which, in some implementations, may assess the stress response of bone at the implant/bone interface as opposed to qualimetric discussed in the above paragraph that does visual tracking.

The qualimetric system includes a striking-evaluation system 1015 in which a mallet strikes a rod which drives a prosthesis into a prepared cavity. The surgeon then qualitatively assesses the placement using secondary cues (audio, tactile, visual imaging) to estimate a quality of insertion and assume a quality of fixation. This cycle of strike and assess continues until the surgeons stop, often wondering whether stopping is appropriate and/or whether they have struck the rod too many times/too hard.

In contrast, a quantitative cycle 1020 in the quantimetric system includes operation of an insertion agency, measurement of force response(s) to determine elastic and rigidity factors, and use these factors to determine whether to continue operation and whether to modify the applied force from the insertion agency. The quantitative system assumes BFSF and optimal press-fit fixation relies primarily on a cavity aperture of a relatively oversized prosthesis/relatively undersized cavity which provides a contact area around a “rim” of the cavity where bone contacts, engages, and fixates the prosthesis. A depth of the aperture region may depend upon a degree of lateral compression of the prepared bone as the prosthesis is installed.

The parametric values of the quantimetric system provide meaningful actionable information to surgeons as to when to increment the magnitude of force, and as to when to stop application of force. Additionally, surgeons currently utilize qualitative means (auditory and tactile senses) as well as auxiliary optical tracking means (fluoroscopy, navigation) to assess the depth of insertion and estimate a quality of fixation during press fit arthroplasty. Application of force to achieve press fit fixation is uncontrolled and based on human proprioceptive and auxiliary optical tracking means. The optimal endpoint for press fit fixation remains undefined and elusive.

An embodiment may include development of a reliable quantitative technique for real-time intra-operative determination of optimal press fit, and the development of a smart tool to obtain this point automatically. The ability to base controlled application of force for installation of prosthesis in joint replacement surgery on the force response of the implant/bone interface is an innovative concept allowing a quantimetric evaluation of the implant/bone interface.

An embodiment for a quantimetric system may include a hand-held tool (See, e.g., FIG. 1) that can produce impact energies of the necessary magnitude and accuracy. A variety of actuation methods can be used to create controlled impacts, including pneumatic actuators, electro magnetics actuators, or spring-loaded masses. An example implementation using pneumatic, vibratory, motorized, controlled, or other actuation The device shall have industry standard interfaces in order to allow for use with a variety of cup models.

A slide hammer pneumatic prototype is created to allow precise and incremental delivery of energy E. It is equipped with inline force sensors in order to measure resulting forces F1 and F2 and controlled by integrated electronics that provides analysis of F1, F2, ΔF2, number of impacts, and impact energy E. Programed algorithms based on the double binary system described herein will produce successive impacts of a known energy, making two simultaneous binary decisions before each impact: (a) modify energy or not; and (b) apply energy or not. These two binary decisions will be based on parametric values produced by the control electronics, which provides an essential feedback of the implant/bone interface, and the elastic response of bone at the aperture. The following algorithm provides a basic example of the double binary decision making process.

A method for assessing a seatedness and quality of press fit fixation includes a series of operations for installing a prosthesis into a relatively undersized cavity prepared in a portion of bone, including communicating, using an installation agency, a quantized applied force to a prosthesis being press-fit into the cavity; monitoring a rigidity metric and an elasticity metric of the prosthesis with respect to the cavity (some embodiments do this in real-time or near real-time without requiring imaging or position-determination technology); further processing responsive to the rigidity and elasticity metrics, including continuing to install the prosthesis at present level of applied force while monitoring the metrics when the metrics indicate that installation change is acceptable and a risk of fracture remains at an acceptable level, increasing the applied force and continuing applying the installation agency while monitoring the metrics when the metrics indicate that installation change is minimal and a risk of fracture remains at an acceptable level, or suspending operation of the installation agency when the metrics indicate that installation change is minimal when a risk of fracture increases to an unacceptable level.

1. Apply energy E1 and measure F2, number of impacts (NOI), ΔF2.

2. Monitor F2 over number of impacts (NOI), and/or monitor ΔF2 as it approaches zero.

3. When ΔF2 approaches zero, insertion is not occurring for that particular energy E1. If NOI required to achieve this point is sufficiently large (low speed of insertion) as determined by the control algorithm, then E1 is increased to E2

4. Continue steps 1 through 3 until the NOI required for ΔF2 to approach zero is sufficiently small (high speed of insertion) as determined by the control algorithm.

5. The smart tool may be implemented so it will not generate automated impacts after this level is reached. Additional increase in energy E is not recommended but can be produced manually or after a considered override by the surgeon. For example, it may be that no more than one incremental manual increase is recommended or established as a best practice.

Validation of the tool may be performed by comparing the quality of insertion (extractive force F4) produced by AI-PID with those produced by a mallet and standard impaction techniques. Specifically, the two distinct endpoints of (i) BFSF (achieved through AI-PID) and (ii) full seating (achieved through mallet strikes) will be compared to determine differences in the extractive force F4 and fracture incidence. A risk benefit analysis will be done to determine whether additional impacts and insertion beyond BFSF provided any significant value as to implant stability, or conversely led to increased incidence of fracture of the cavity. (As noted herein, it may be the case that BFSF may be achieved without full seating, a stated goal of many conventional procedures.)

It is anticipated that the measurements of F2, and ΔF2 and its comparative analysis with respect to number of impacts NOI will provide a principled and organized process for application of energy to achieve a desired endpoint of fixation BFSF. We expect that the first order relationship of ΔF2 will provide the information as to whether, for any particular level of applied energy, insertion is occurring or not; providing a guidance as to whether applied energy should be increased. We expect the rate of ΔF2 decay to zero will provide information about elastic/plastic behavior of the aperture, indicating when the maximum strain X, normal force FN, and extractive force F4 at the aperture of the bone cavity have been achieved. We anticipate reproducing the results of phase I aim 1, namely that there is a strong correlation between pull force F4 and rate of decay of ΔF2, that an inflection point exists in the elasticity factor, beyond which addition of impact energy will lead to marginal gains in extraction force F4 and depth of insertion, mitigating against goal of full seating as the best policy.

We have indicated that the grasp of bone (bone substitute) on an implant at the aperture can be modeled in some cases by formula such as FN*Us where FN represents the normal forces at the interface, and Us represents the coefficient of static friction. FN is estimated by Hooke's Law and is represented by K·X, where K represents the material properties of bone including the elastic and compressive moduli and X represents the difference in diameter between the implant and the cavity. We note that the value of K can vary dramatically between different ages and sexes. We anticipate this tool to be capable of automatically producing the proper amount of impact energy E, cup insertion CI, stretch on bone X, normal force FN, and extractive force F4 to achieve optimal press fit for patients of various ages and sexes, eliminating an over reliance on surgeon senses and experience.

Having access to this interface sensing phenomena, an embodiment may develop a simple controlled impaction process that allows the surgeon to quantize the impact energy, and deliver it in a controlled and modulatable fashion based on the above two parametric value representing the stress/strain behavior of bone. Some embodiments may develop the concept of controlled force application based on an evaluation of the interface force phenomena (forces felt at the prosthesis/cavity interface). This is in stark contradistinction of uncontrolled application of force with a mallet based on a VISUAL assessment/tracking of the depth of prosthesis insertion (MAKO, all navigation techniques, Fluoroscopy, Nikou—a navigation technique).

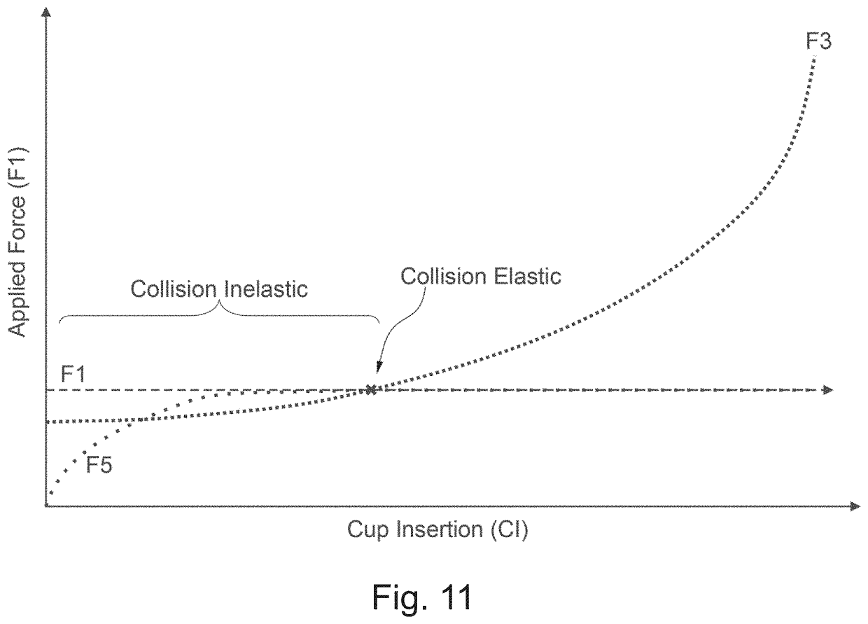

There may be many different ways to assess rigidity factor and to assess an elasticity factor. FIG. 11-FIG. 14 illustrates F2 approaching F1 and F5 approaching F1, as well as (ΔF2 approaching 0) and (ΔF5 approaching 0). Additional non-illustrated ways include F3 approaching F1 and ΔF3 approaching 0). As noted herein, data fusion may produce a fusion variable that can measure, evaluate, or indicate rigidity and/or elasticity. For example, one or more of F2, F3, and F5, appropriately weighted, may be fused into a variable that may be used such as by comparing to F1 or delta fused variable compared to a threshold value (such as zero).

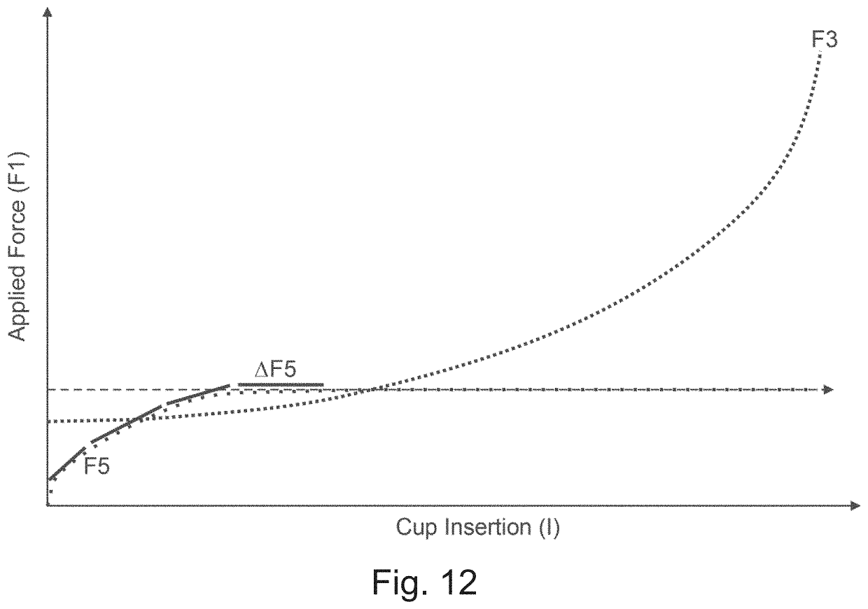

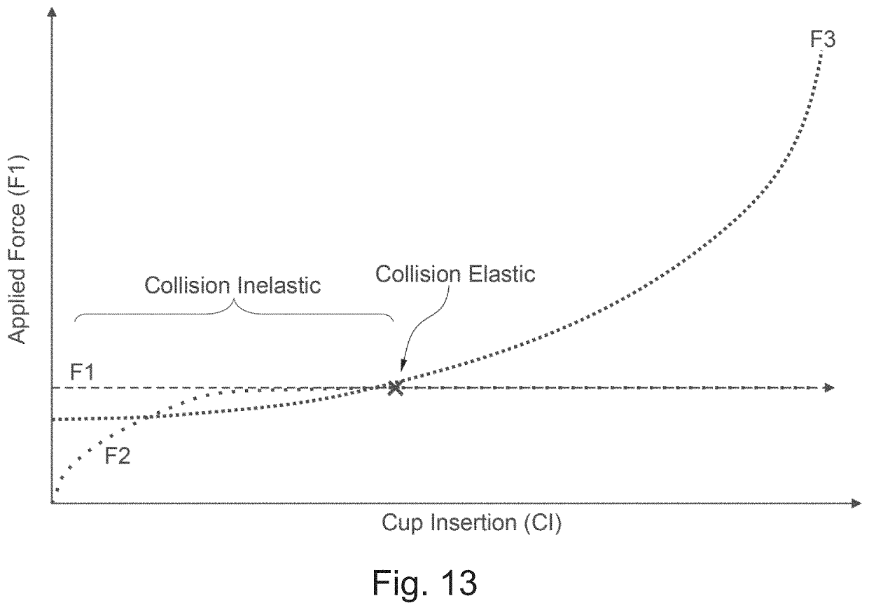

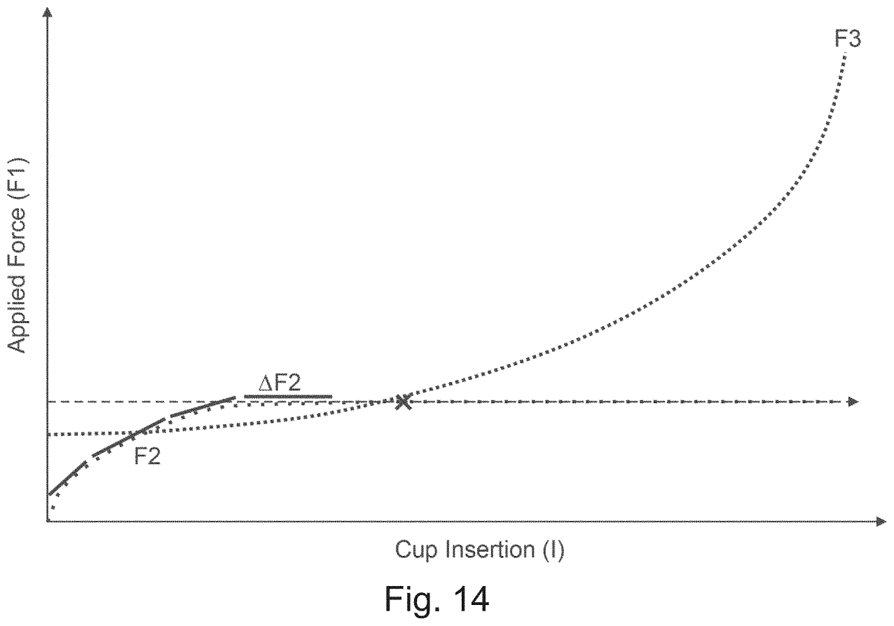

FIG. 11-FIG. 14 illustrate a set of rigidity metric measurements that may be used in the methods and systems described herein. FIG. 11 illustrates a comparison of F5 to F1; FIG. 12 illustrates a comparison of ΔF5 to a predetermined threshold (e.g., 0.0); FIG. 13 illustrates a comparison of F2 to F1; and FIG. 14 illustrates a comparison of ΔF2 to a predetermined threshold (e.g., 0.0).

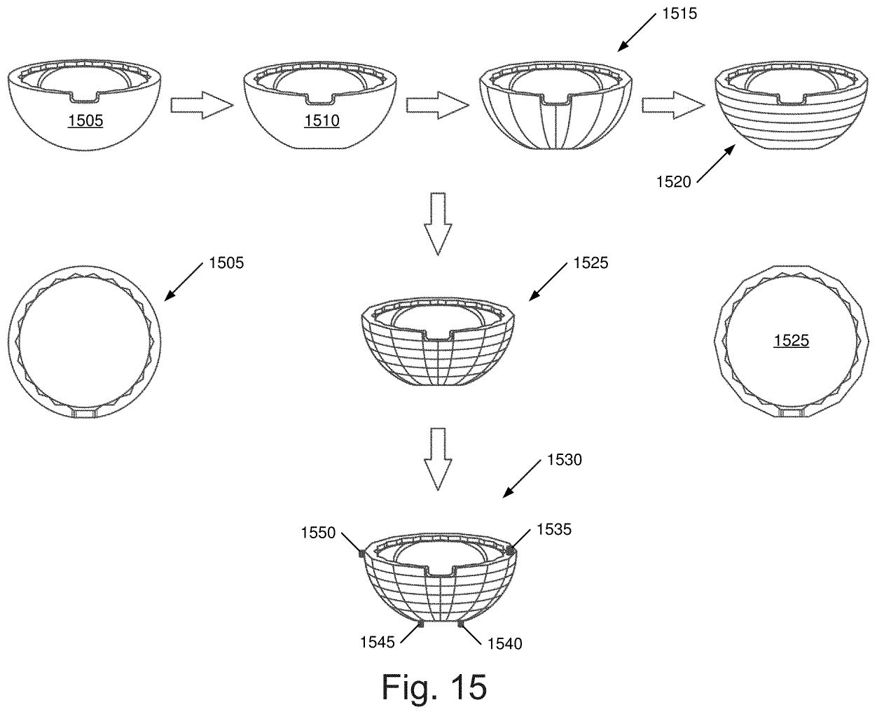

FIG. 15 illustrates a possible evolution of an acetabular cup 1505 consistent with improving press fit fixation. As noted, a conventional acetabular cup for an implant includes a hemispherical outer surface designed to be installed/impacted into a prepared bone cavity (also hemispherical produced from a generally hemispherical reamer for example).

Different stages of evolution illustrate possible improvements to prosthesis embodiments that are responsive to assumptions and embodiments of the present invention. An assumption of some conventional systems is that full depth of insertion results in a maximum extractive press fit fixation. In contradiction to this assumption, it may be the case that embodiments of the present invention achieve maximum/optimal press fit fixation (BFSF) short of full insertion (i.e., intentional presence of a polar gap).

There may be advantages to reducing polar gaps, and rather than full insertion, a modification to the prosthesis may include a truncated hemisphere (snub nosed) cup 1510. There is a desire to reduce insertion forces while maximizing press fit fixation. Evolution of the prosthesis may incorporate several different ideas, including asymmetric deformation control using a truncated cup with longitudinally extending ribs 1515 and laterally extending planks 1520—the combination of ribs and planks cup 1525 may produce an asymmetric deformation to improve installation (such as making it easier to install and more difficult to remove). Further, a perimeter of an improved cup may include a discrete polygon having many sides. The reduced surface area contacting the prepared cavity may reduce force needed to install while the vertices of the polygon may provide sufficient press-fit fixation. Cup 1525 may include tuned values of the snub, different stiffnesses of ribs and planks, a perimeter configuration of the regular/irregular non-hemispherical polygonal outer surface. These vertices themselves may be angular and/or rounded, based upon design goals of a particular implementation of an embodiment to achieve the desired trade-offs of installation efficiency and press-fit fixation to improve the possibility of achieving BFSF.

These concepts have implications on how the acetabular (all press fit prosthesis) prosthesis are made. If it holds true that the dome of the cup mostly acts like a wedge to cause fracture, it may be best to eliminate the dome (flatten the cup) and change the geometry of the cup to be more like a frustum polygon with an N number of sides, or a hemisphere with a blunted dome.

A. With the ability to provide a proportional amount of force for any particular (implant/bone) interface, we can expect to use just the right amount of force for installation of the prosthesis (not too much and not too little). Additionally we have previously in U.S. patent application Ser. No. 15/234,927, expressly incorporated herein, discussed methods to manufacture prosthesis with an inherent tendency for insertion, MECHANICAL ASSEMBLY INCLUDING EXTERIOR SURFACE PREPARATION. Specifically, we have descried the concept of two-dimensional stiffness incorporated within the body of the prosthesis, which would produce a bias for insertion due to the concept of undulatory motion, typically observed in Eel and fish skin.

FIG. 15 includes a side view of a prosthesis including a two-dimensional asymmetrical stiffness configuration, and illustrates a top view of prosthesis. The prosthesis may include a set of ribs and one or more planks disposed as part of a prosthetic body, represented as an alternative acetabular cup. The body may be implemented in conventional fashion or may include an arrangement consistent with prosthesis P. The ribs and plank(s) are configured to provide an asymmetric two-dimensional (2D) stiffness to body that may be more conducive to transmission of force and energy through the longitudinal axis of the cup as opposed to circumferentially. Ribs are longitudinally extending inserts within body (and/or applied to one or more exterior surfaces of body). Plank(s) is/are laterally extending circumferential band(s) within body (and/or applied to one or more exterior surfaces of body). For example, planks may be “stiffer” than ribs (or vice-versa) to produce a desired asymmetric functional assembly that may provide for an undulatory body motion as it is installed into position.

Based on our understanding of the acetabular prosthesis/bone interface in our Invasive sensing studies in one or more incorporated patent applications and in conjunction with the incorporated '927 application of MECHANICAL ASSEMBLY INCLUDING EXTERIOR SURFACE PREPARATION, we anticipate that the prosthesis of the future may have different characteristics.

A. The acetabular component may be shaped more like a frustum with Nth (e.g., 160 sides) and an amputated dome. The snubbed dome of the new prosthesis would not engage the acetabular fossa (Cotyloid fossa) allowing the new prosthesis fully to engage the stronger acetabular walls/rim (constituted by the ilum, ischium and pubic bones). This shape of prosthesis avoids the possibility of a wedge type fracture which can be produced by the dome of a hemispherical implant.

B. Each angle of the frustum may produce longitudinal internal rib extending from the rim distally, (developed within the structure of the prosthesis by additive manufacturing by controlling the material properties of crystalline metal), that is more flexible than the horizontal stiffer planks that extend from the rim to the snub distally in a circumferential fashion. (See the incorporated '927 application). This shape of prosthesis will have a strong bias for insertion due to undulatory motion, and will require less force for installation.

Permanent or Removable Sensors on the surface of the Prosthesis.

A. As described herein, in some experiments that when F2 approaches F1, that in fact F1=F2=F3=F5. That is, when the implant/bone collision becomes elastic, the resistive force at the interface F3 and the forces felt in bone F5 can be inferred from applied force F1 and force felt in tool F2. This can provide the surgeon valuable information about the forces she is imparting to the bone. We also contemplate that F3 and F5 can be directly measured by application of mechanical and biologic sensors directly on a sensing prosthesis 1530. We believe given the mass production and ubiquitously available sensors, at some point, the prosthesis of the future would be equipped with its own sensor (biologic and or mechanical) to convey to the surgeon the forces being imparted into the bone, to prevent excessive forces on bone, as well as to prevent loose fitting prosthesis. Sensors on the applied on the surface of the prosthesis to measure interface or dome pressure (F3 or F5) can be permanent or removable i.e., a slot on the side of the prosthesis can allow incorporation of a small sliding sensor to provide information about the interface to the system. Examples of incorporated sensors, one or more which may be used, may include an internal sensor 1535, a mechanical sensor 1540, a biologic sensor 1545, and an external sensor 1550.

B. Data Fusion of F2, F5, F3 for most sensitive evaluation of stress response of Bone at the Implant Bone Interface—multiple parameters are weighted and merged or fused that may provide a robust parameter offering improved performance over reliance on a single parameter.

2. Application of Force Based on a Sensory (not Visual) Evaluation of Implant/Bone Interface.

A. For years surgeons have applied uncontrolled force to impact prosthesis into bone, and have assessed the quality of insertion by human visual, tactile and auditory means. More recently surgeons have begun to use visual tracking means such as fluoroscopy, computer navigation (including Nikou), and MAKO techniques to assess depth of insertion. We are the first to suggest that the application of force for installation of prosthesis should be predicated on the force sensing activity of the prosthesis/bone interface. This is a new technique that predicates application of force for installation of prosthesis to be based (NOT VISUAL TRACKING MEANS—depth of insertion) but rather (FORCE SENSING MEANS OF THE INTERFACE—proof resilience). This is a novel concept that will eliminate too tight and too loose press fit fixation of all prosthesis, and associated problems such as subsidence, loosening, and infection.

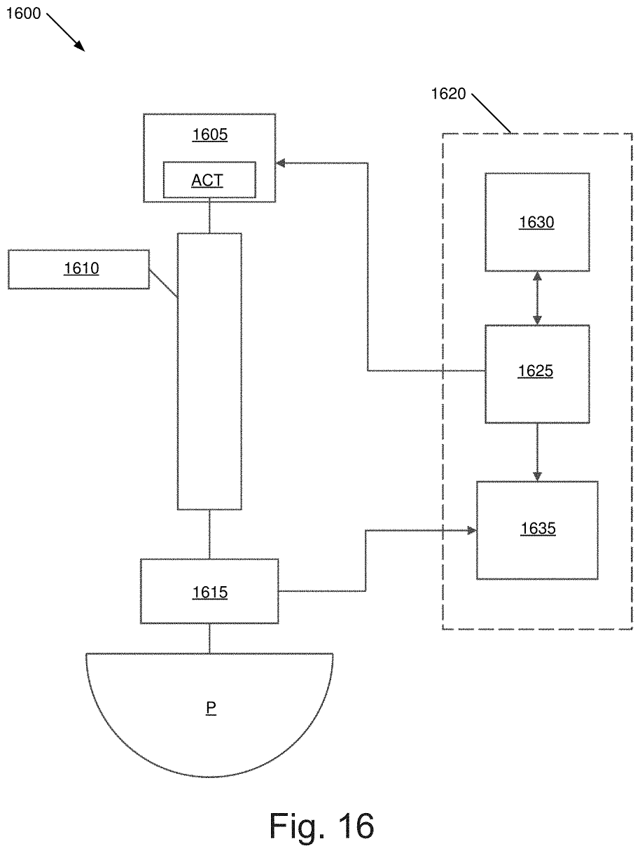

FIG. 16 illustrates a particular embodiment of a BMDx force sensing tool 1600. Tool 1600 allows indirect measurement of a rate of insertion of an acetabular cup and may be used to control the impact force being delivered to a prosthesis based upon control signals and the use of features described herein. Tool 1600 may include a controllable force applicator (e.g., an actuator) 1605, an impaction transfer structure 1610 (e.g., impaction rod), and a force sensor 1615.

Applicator 1605 may include a force sensor to measure/determine F1 (in some cases applicator 1605 may be designed/implemented to apply a predetermined and known a priori force.

Structure 1610 transfers force as an insertion agency (for prosthesis implant applications) to prosthesis P and system 1615 measures a realtime (or near realtime) force response of prosthesis P to the insertion agency while it is being implanted into the implant site. There are many different possible force response mechanisms as described herein. For example, F2, F3, F5, and first/second order derivatives and combinations thereof as noted herein. In some cases, system 1615 may include in-line or external sensor(s) associated with or coupled to structure 1610. In other cases, some embodiments of system 1615 may include sensor(s) associated with the bone or cavity or other aspect of the cavity, prosthesis, cavity/prosthesis interface or other force response parameter. System 1615, as noted herein, may include multiple concurrent sensors from different area including one or more of tool, prosthesis and bone/cavity.

One representative method for force measurement/response would employ such a tool 1600. Similar to the impaction rod currently used by surgeons, tool 1600 may couple to an acetabular cup (prosthesis P) using an appropriate thread at the distal end of structure 1610. Applicator 1605 may couple to a proximal end of structure 1610, and create an insertion agency (e.g., controlled and reproducible impacts) that would be applied to structure 1610 and connected cup P. A magnitude of the impact(s) would be controlled by the surgeon through a system control 1620, for example using an interface such as a dial or other input mechanism on the device, or directly by the instrument's software. System control 1620 may include a microcontroller 1625 in two-way communication with a user interface 1630 and receiving inputs from a signal conditioner 1635 receiving data from force sensing system 1615. Controller 1625 is coupled to actuator 1605 to set a desired impact profile including a set of force applications that may change over time as described herein.

System 1615 may be mounted between structure 1610 and acetabular cup P. System 1615 may be of a high enough sampling rate to capture the peak force generated during an actuator impact. It is known that for multiple impacts of a given energy, the resulting forces increase as the incremental cup insertion distance decreases/

This change in force given the same impact energy may be a result of the frictional forces between cup P and surrounding bone of the installation site. An initial impact may have a slow deceleration of the cup due to its relatively large displacement, resulting in a low force measurement. The displacement may decrease for subsequent impacts due to the increasing frictional forces between the cup and bone, which results in faster deceleration of the cup (the cup is decelerating from the same initial velocity over a shorter distance). This may result in an increase in force measurement for each impact. A maximum force for a given impact energy may be when the cup P can no longer overcome, responsive to a given impact force from the actuating system, the resistive (e.g., static friction) forces from the surrounding bone. This results in a “plateau”, where any subsequent impact will not change either the insertion of cup P or the force measured.

In some embodiments, this relationship may be used to “walk up” the insertion force plot, allowing tool 1600 to find the “plateau” of larger and larger impact energies. By increasing the energy, the relationship between measured impact force and cup insertion should hold until the system reaches a non-linear insertion force regime. When the non-linear regime is reached, a small linear increase in impact energy will not overcome the higher static forces needed to continue to insert the cup. This will result in an almost immediate steady state for the measured impact force (mIF of a force application X is about the same as MIF of a force application X+1).

A procedure for automated impact control/force measurement may include: a) Begin operation of an insertion agency with a static, low energy; b) Record the measured force response (MIF); c) continue operation of the insertion agency until the difference in measured impact force approaches zero (dMIF=>0), inferring that the cup is no longer displacing; d) increase the energy of the operation of the insertion agency by a known, relatively small amount; and e) repeat operation of the modified insertion agency until plateau and increasing energy in a fashion (e.g., a linear manner) until a particular plateau patterning is detected. Instead, an increase in energy results in a “step function” in recorded forces, with an immediate steady-state. The user could be notified of each increase in energy, allowing a decision by the surgeon to increase the resulting impact force.

A goal of a validated ISM concept is to produce a sophisticated tool for a surgeon that provides automatic, intelligent prosthesis installation, with the capacity to provide access to an optimal best fixation short of fracture (BFSF) endpoint inherent in any implant/cavity system. This tool will allow surgeons of all walks of life, regardless of level of experience, to obtain the best possible press fit fixation of any cup/cavity system, without fear of too loose or tight press fit, as well as obviating the need for screw fixation with all its attendant problems.

The tool may include a handheld pneumatic instrument with a sliding mass component. It may have the following features: 1) ability to deliver precisely controlled axial impacts of known impact energy E, 2) ability to increase or modify applied force (F1) over the course of use, 3) ability to acquire the resulting F1, F2, F3, and F5 for each impact, 4) ability to automatically control the application of impact energy to optimally seat an acetabular cup (implant) using the algorithms determined in Phase I, 5) communicate data pertaining to ISM and BFSF to the surgeon, 6) allow for manual override and selection of impact energy by the surgeon.

Actuators of applicator 1605 may include a one or more of a wide variety of devices (or combinations thereof), including pneumatic actuators, electro-magnetic actuators, spring-loaded masses, and the like.

The device may have industry standard interfaces in order to allow for use with a variety of cup models. For the example implementation, the impact energy is controlled through a piston actuation control mechanism and by additional adjustments of sliding mass and travel distance. Once a final actuation method is selected, a working prototype will be designed and fabricated to allow for controlled insertion of acetabulum cups.

The instrument may be equipped with inline force sensors and wireless connectivity in order to determine resulting forces F1, F2, F3, F5 within the system. Applied force F1 and felt force within the tool (F2) will be measured using internal sensors, whereas the forces felt in bone (F5) and at the implant/bone interface (F3) will be measured separately with appropriately placed sensors in the system and the data conveyed to the central processing unit (CPU) through wireless (intranet) systems.

The tool will be controlled by integrated electronics that provide analysis of the inter-relationships between F1, F2, F3, F5 with respect to number of impacts (NOI) to full insertion, and impact energy. The magnitude of the impacts will be controlled by a CPU (FIG. 16) and associated software, where the system control may include a microcontroller in two-way communication with a user interface and receive inputs from a signal conditioner, which receives data (directly or indirectly) from the sensors within the system. The microcontroller will be coupled to the actuator to set a desired impact energy and run a fixation algorithm to obtain endpoint BFSF.

Programmed algorithms based on the binary decision system described in Phase I Specific Aim #1 will produce successive impacts of known energy, making two simultaneous decisions before each impact: 1. Continue applying force or not, and if so, then 2. Increase energy or not. These binary decisions will be based on parametric values produced by the control electronics, which provide essential feedback of the implant/bone interface, and the elastic response of bone at the aperture. The following algorithm provides a basic example of the binary “fixation algorithm” to be incorporated in the control mechanism: (i) apply energy E1 and measure F2, NOI, ΔF2; (ii) monitor F2 over NOI, and/or monitor ΔF2 as it approaches 0; (iii) when ΔF2 approaches 0, insertion is not occurring for that particular energy E1. If NOI required to achieve this point is sufficiently large (low rate of insertion), as determined by the control algorithm, then E1 is increased to E2; (iv) continue steps (i) through (iii) until the NOI required for ΔF2 to approach 0 is sufficiently small (high rate of insertion), as determined by the control algorithm; (v) the sophisticated tool will not generate automated impacts after this level is reached. Additional increase in energy E is not recommended but can be produced manually at the surgeon's discretion. No more than one incremental manual increase is recommended.

As noted earlier, our preliminary data indicate that force measurements directly at the interface (F3), and in bone (F5) will show similar trends and characteristics as F2, such that although independent, they may be considered redundant, complimentary and/or cooperative. We expect to be able to incorporate these data into an independent system architecture and utilize existing data fusion algorithms to potentially produce a higher resolution evaluation of the stress (force) field around the implant/bone interface than with each individual sensor alone.

Validation of the tool will be performed at Excelen and at the University of Minnesota Department of Engineering by comparing the quality of insertion (extractive force F4) produced by AI-PID—which automatically achieves endpoint BFSF—with the quality produced by a mallet and standard impaction techniques accomplished by a board certified orthopedic surgeon blinded to the study. Specifically, the two distinct endpoints of 1. BFSF (achieved through AI-PID) and 2. Full Seating (achieved through mallet strikes) will be compared to determine differences in F4 and fracture incidence. All parameters associated with these two endpoints will be compared and evaluated. Specifically, a risk benefit analysis will be performed to determine whether higher impact energies were required to obtain full seating, and if so, whether the additional impacts provided any significant value as to CI or F4, and whether there was any increase in fracture incidence (failure of the cavity) with either technique.

Interpretation of Results:

Measurements of F2 and ΔF2 and their first and second order derivatives and comparative analysis with respect to NOI to insertion may provide a principled and organized process for application of energy to achieve the desired optimal endpoint BFSF. It is anticipated that the second order relationship of ΔF2 to NOI, alternatively stated as the rate of decay of ΔF2 (how fast ΔF2 approaches 0) may provide an evaluation of elastic/plastic deformation and also contribute to achieving BFSF.

Biology of Graft Healing

Tendon graft healing to a bone tunnel is one important factor affecting a success of a reconstructed ACL. An unruptured ACL attaches to bone through “direct” type insertion, which has a highly differential morphology including four specific zones: tendon, fibrocartilage, mineralized fibrocartilage, bone. This small 1 mm zone plays an important mechanical role in allowing progressive distribution of tensile loads from the tendon (ligament) to subchondral bone.

A reconstructed ACL may sometimes attach to bone in a different fashion called “indirect” type insertion, which has a significantly simpler ultrastructure. Indirect insertion involves anchoring of the tendon (ligament) into bone without the intervening fibrocartilaginous zones (non-mineralized and mineralized fibrocartilage). These fibers represent the type of anchoring that occurs between periosteum and bone referred to as Sharpey fibers. The design of this type of insertion allows for micro motion at the insertion site. It is not as efficient as the “direct” type insertion in allowing transition of mechanical forces from ligament to bone.

Problem—Suspensory Cortical Fixation Versus Aperture Interference Screw Fixation

There are broadly two types of fixation: suspensory cortical fixation and aperture interference screw fixation. There is general consensus that there are advantages and disadvantages to each method of fixation.

Suspensory Cortical Fixation

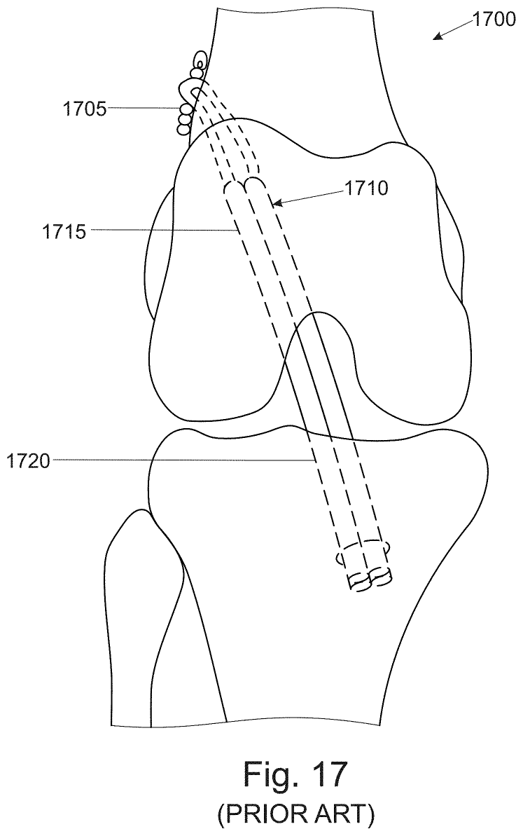

FIG. 17 illustrates an example of suspensory cortical fixation 1700. Fixation 1700 includes an endobutton 1705 supporting a graft 1710 through a femoral tunnel 1715 and a tibial tunnel 1720.

Advantages of fixation 1700 may include one or more of: (a) allows circumferential 360 degree contact between tendon and bone (maximized surface area contact for tendon to bone healing); (b) easier operation to perform; (c) less damage to bone and tendon at the time of surgery (less invasive—bone and tendon sparing); and (d) strong fixation.

Disadvantages of fixation 1700 may include one or more of: (a) allows micro motion at the aperture, including (i) bungee effect (lengthwise micro motion), (ii) windshield wiper (side-to-side micro motion), and/or (iii) increased propensity for increased risk of poor healing such as tunnel widening; (b) low tendon to bone compression forces at the interface (less than ideal healing: always heals with “indirect” type healing (Sharpey Fibers, no transitional zone of mineralized and non-mineralized fibrocartilage).

Aperture Interference Screw Fixation

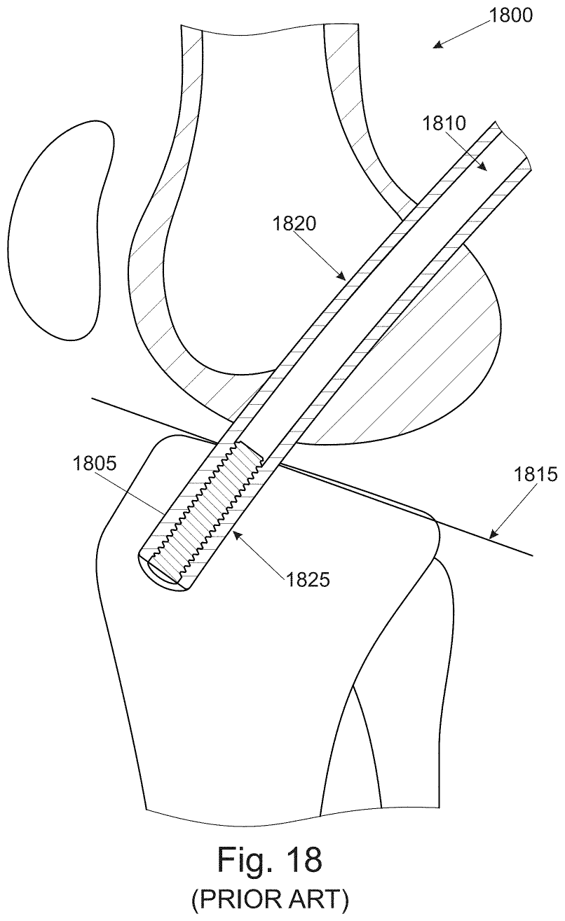

FIG. 18 illustrates an example of aperture interference screw fixation 1800. Fixation 1800 includes an interference screw 1805 attached to a graft 1810 that has the relationship illustrated between a tibial plateau 1815 and Blumensaat's line 1820 along with a tibial tunnel 1825 wherein screw 1805 is applied.

Advantages of fixation 1800 may include one or more of: (a) significantly higher compression forces between tendon/bone interface (by an order of magnitude) relative to fixation 1700; (b) rigid fixation with minimal or no micro motion in the bone tunnel; (c) ideal healing—graft 1810 heals to bone by “direct” type insertion with much higher specialization of the tendon bone interface, allowing for progressive force transfer from tendon to bone (formation of the four zones: tendon, fibrocartilage, mineralized fibrocartilage, bone); and (d) faster healing.

Disadvantages of fixation 1800 may include one or more of: (a) significant tissue damage to the graft and bone with interference screw fixation (weakening of the early fixation period—6 to 10 weeks); (b) loss of circumferential contact between tendon and bone, compromising maximal contact area between tendon and bone by at least 50%; and (c) inflammatory and cellular reaction to foreign body within the tunnel causing tunnel widening and cyst formation.

The present invention may be useful for a wide-range of connective tissue grafts used in a wide-range of repair techniques. With this understanding, to simplify the discussion a particular type of graft used in a particular type of repair technique: an ACL graft used for repair of a ruptured ACL.

The knee is a simple hinge joint at the connection point between the femur and tibia bones. It is held together by several important ligaments. The most important of these to the knee's stability is the Anterior Cruciate Ligament (ACL). The ACL attaches from the front part of the tibia to the back part of the femur. The purpose of this ligament is to keep the tibia from sliding forward on the femur. For this reason, the ACL is most susceptible to injury when rotational or twisting forces are placed on the knee. Although this can happen during a contact injury many ACL tears happen when athletes slow down and pivot or when landing from a jump.

After the ACL is torn the knee is less stable and it becomes difficult to maintain a high level of activity without the knee buckling or giving way. It is particularly difficult to perform the repetitive cutting and pivoting that is required in many sports.

Regardless of how the ACL is torn a physician will work with their patient to determine what the best course of treatment will be. In the case of an isolated ACL tear (no other ligaments are involved) the associated pain and dysfunction may often be successfully treated with rest, anti-inflammatory measures, activity modification and Physical Therapy. After the swelling resolves and range of motion and strength is returned to the knee a decision can be made as to how to proceed. Many people elect to use a sports brace and restrict their activity rather than undergo surgery to reconstruct the ACL. When a non-surgical approach is taken the patient must understand that it is imperative that she or he maintain good strength in her or his leg and avoid sports or activities that require pivoting or cutting. When conservative measures are unsuccessful in restoring function the patient and their physician may elect to have the torn ligament reconstructed.

ACL reconstruction surgery is not a primary repair procedure. This means that the ligament ends cannot simply be sewn back together. The new ACL must come from another source and grafted into place in the knee. There are a few different options as to what tissue is used for the ACL graft (three most common sources include patella tendon, hamstring tendon, and cadaver tendon) and each patient should consult with his or her surgeon to determine the best choice. During the procedure a set of tunnels are drilled within the tibia and femur and the new ACL graft is passed into these tunnels and anchored into place. Some or all of this anchoring, in embodiments of the present invention, occur by use of an in situ decompression of a compressed end portion of the ACL graft within a prepared tunnel.

The ACL graft includes a highly hydrated and compressible tissue. As observed by applicant, a diameter of a typical ACL graft may be compressed, for example by up to 2 to 4 millimeters, with special techniques that can be employed just prior to installation. The native ACL graft can be manipulated (e.g., compressed and/or stretched) to produce a manipulated ACL graft that has a smaller diameter than the native ACL graft. For this discussion, the native ACL graft may include a 10 millimeter diameter while the manipulated ACL graft may include a 7 millimeter diameter.

The manipulated ACL may subsequently be implanted at a significantly compressed diameter than its original form (i.e. 7 mm instead of 10 mm) and allowed to expand, in a delayed fashion, within bone tunnels formed and used during the repair procedure, producing high contact forces at an interface between the manipulated ACL graft and the bone of the tunnel (e.g., a tendon/bone interface).

This repair may be accomplished with all the positive attributes of suspensory cortical and aperture fixation and without any of the negative attributes of the two fixation methods.

This method of “biological press fit” fixation does not have the negative attributes of interference screw fixation including: without the use of an interference screw and its attendant negative attributes including: (i) damage to the graft and bone; (ii) loss of circumferential contact; and (iii) foreign material within the tunnels causing late inflammatory and destructive reactions in bone. Similarly, the “biological press fit” fixation dos not have the negative attributes of suspensory cortical fixation including: (i) micro motion at the aperture causing bungee (lengthwise micro motion) and windshield wiper (side-to-side micro motion) effects, (ii) increasing risk of tunnel widening; and (iii) low tendon-bone interface compression forces leading to “indirect” type healing (Sharpey Fibers, with no transitional zone of mineralized and non-mineralized fibrocartilage, for specialized transfer of force).

An embodiment of the present invention may allow all the positives attributes of both suspensory cortical and aperture fixation. “Biologic press fit” fixation may embody all the positive attributes of suspensory cortical fixation including: (i) circumferential 360-degree contact between tendon and bone (maximized surface area contact for tendon to bone healing); (ii) easier operation to perform; (iii) less damage to bone and tendon at the time of surgery (less invasive—bone and tendon sparing); (iv) strong fixation. “Biological press fit” fixation similarly may embody all the positive attributes of aperture fixation including: (i) significantly higher compression forces between tendon/bone interface; (ii) rigid fixation with minimal or no micro motion in the bone tunnel; (iii) ideal healing—by “direct” type insertion with specialization of the tendon bone interface, allowing for progressive force transfer from tendon to bone (formation of the four zones: tendon, fibrocartilage, mineralized fibrocartilage, bone); and (iv) faster healing.

The combination of factors noted above are believed to allow high interference forces that may be obtained soon after implantation (including decompression of manipulated ACL graft within a portion of a one tunnel), these interference forces due to the in situ decompression of the manipulated ACL graft, without interference of foreign material within the tunnels.

Some embodiments may include application of one or more remotely-readable biological sensors to the manipulated ACL graft. The sensors may, for example, include a capacity to measure contact forces at the tendon/bone interface of the expanding manipulated ACL graft within a tunnel. These sensors may be applied to the ACL graft as part of the preparation or provided to the surgeon prior to compression. There may be various uses of this/these sensor(s), in order to assess compressive forces produced at the tendon/bone junction at time zero and over defined periods of time.

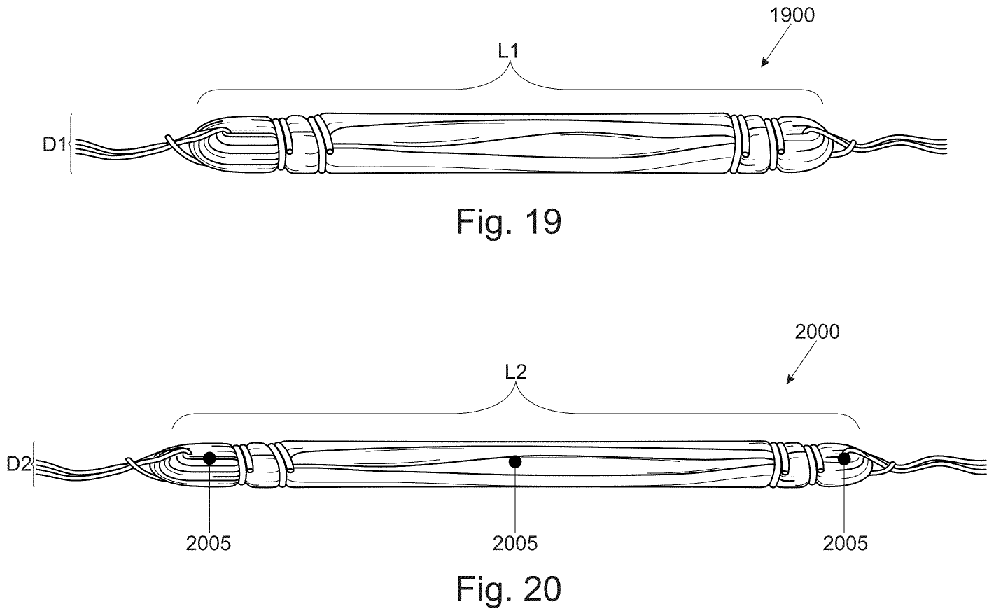

FIG. 19 illustrates an example of a native connective tissue graft 1900. Graft 1900 is provided with predetermined general dimensions, including a length L1 and a diameter D1. For example, for an ACL reconstruction, graft may have L1 about 90-180 millimeters (determined by patient anatomy) and D1 about 10 millimeters.

FIG. 20 illustrates an example of a compressed connective tissue graft 2000 that may result from a pre-operative compressive treatment of native connective tissue graft 1900. Graft 2000 includes a length L2 that may be about greater than or equal to L1 and further includes a diameter D2 that is less than D1. One or more remotely-readable biologic sensors 2005 may be included with graft 2000.

Sensor(s) 2005 may be included as part of graft 1900 (pre-manipulation) or may be applied to a surface of graft 2000 or bulk-integrated into a body of graft 2000 as part of, or attendant to, pre-reconstruction preparation of graft 2000.

Sensor(s) 2005 may be used for different purposes to assess a quality of various aspects of the reconstruction procedure. For example, a compression reading at one or more interfaces between one or more end portions of graft 2000 within the bone tunnel into which graft 2000 was installed may be used to measure healing and fixation. A sensor 2005 disposed outside of a tunnel between the femur and the tibia may include a stress-strain gauge to understand the potentially rupturing forces that the patient applies to the reconstructed ACL graft (after surgery) in the course of their activities. Readings may be taken immediately after installation and then at various subsequent times to assess a magnitude of the graft/bone interface at that/those portion(s). The readings may indicate that healing is progressing (and some metric of how well the healing has progressed), healing has largely completed past a predetermined threshold, or that there may be some complication in the healing process.

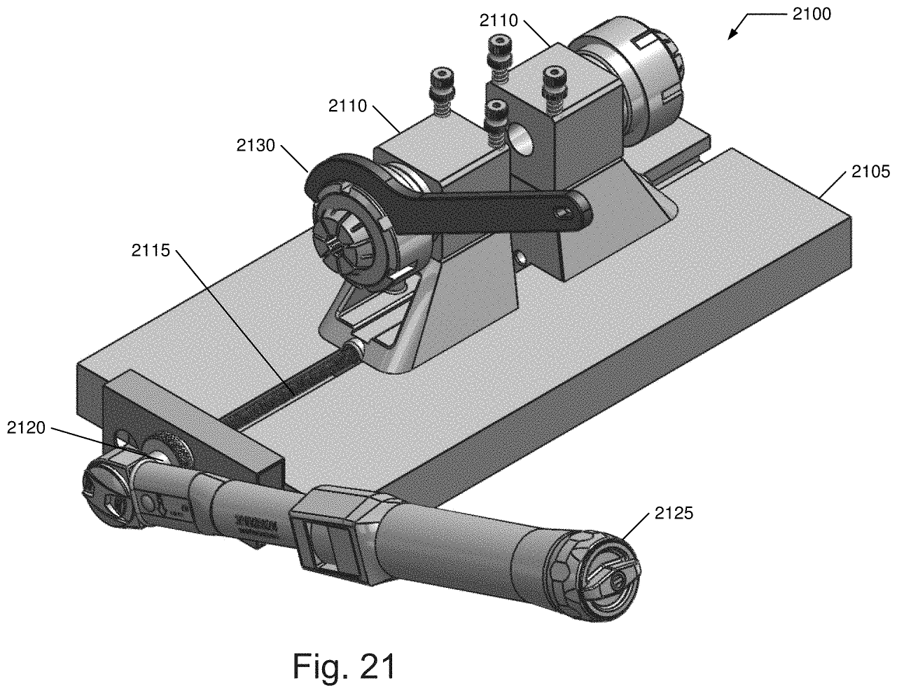

FIG. 21 illustrates a perspective view of a graft platform 2100. Platform 2100 may include a table 2105 supporting a pair of moveable sleeve housings 2110. Housings 2110 move relative to each other (one or both housings 2110 may move). Movement may be controlled by a drive rod 2115 having a knob 2120. Knob 2120 may be turned using a torque wrench 2125 to understand how much force is being used to separate housings 2110. One may want to be sure that not too small or too large force is used in separating housings 2110 as this influences an amount of tension/deformation to any graft being manipulated by platform 2100.

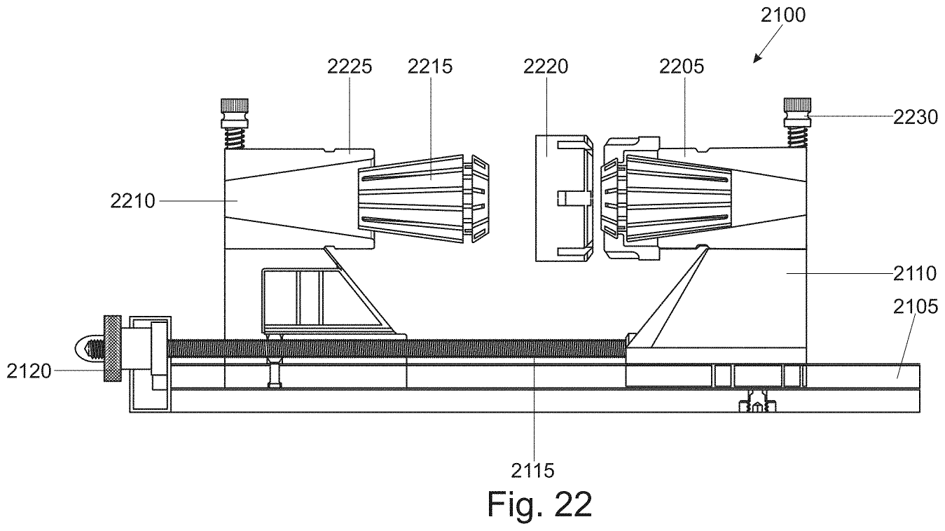

Each housing 2110 supports a graft sleeve that defines a conical internal sleeve structure into which a collet chuck is introduced and upon which a collet nut is threaded over the collet chuck within the internal sleeve structure using complementary threaded portions of an end of the graft sleeve. A wrench 2130 may be used to tighten the collet nut onto the graft sleeve. One or more suture holders may be used to support graft 1900 when initially installed into graft platform 2100. For purposes of this illustration FIG. 21, sleeve housings 2110 are shown facing away from each, while in actual operation housings 2110 are reversed as illustrated in FIG. 22.

FIG. 22 illustrates a side view of graft platform 2100 with repositioned housings 2110 to face each other. Platform 21500 includes a graft sleeve 2205 coupled to housing 2110. Each graft sleeve 2205 defines a conical internal sleeve structure 2210 into which a collet chuck 2215 is positioned. A threaded collet nut 2220 is positioned over collet chuck 2215 and is installed onto sleeve 2205 by use of a threaded end 2225 of graft sleeve 2205. Each graft sleeve 2205 includes one or more suture holders 2230.

In operation, graft 1900 is installed into graft platform 2100 with each sleeve 2205 gripping one end. There are different possible operational modes for graft platform 2100 to compress graft 1900 and produce graft 2000, depending upon the procedure agreed upon by the patient and surgeon.

Graft platform 2100 may compress some or all of graft 1900 by applying equal lateral compressive forces along its length (by appropriate positioning and tightening of collet chucks 2225 into structures 2210 using nut 2220 and/or separating housings 2110 from each other using knob 2120 to rotate rod 2115.

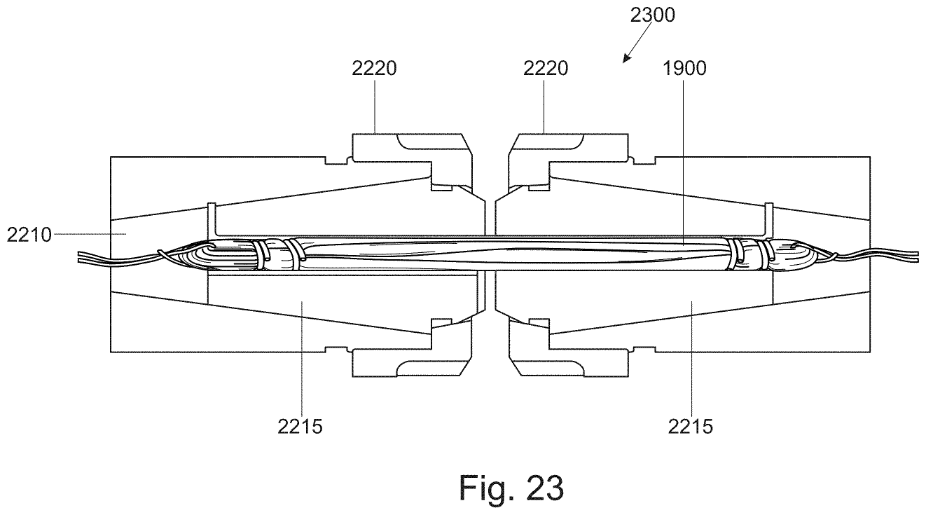

FIG. 23 illustrates a sectional view 2300 of a pair of collet chucks 2215 of platform 2100 gripping native connective tissue graft 1900 by being forced into structure 2210. Each collet chuck 2215 includes a longitudinal tunnel having a variable diameter. That diameter is greatest when it is initially installed into structure 2210. As nut 2220 is tightened, such as with wrench 2130, the corresponding chuck 2215 is forced deeper into conical structure 2210 which decrease the diameter of the longitudinal tunnel. Decreasing the longitudinal tunnel while a portion of graft 1900 is installed is one manner by which lateral compressive forces may be applied to that portion of graft 1900 (which decreases the diameter of that portion of graft 1900). Chuck 1915 may be designed to have a physically-determined minimum diameter to help ensure that graft 1900 is not excessively compressed.

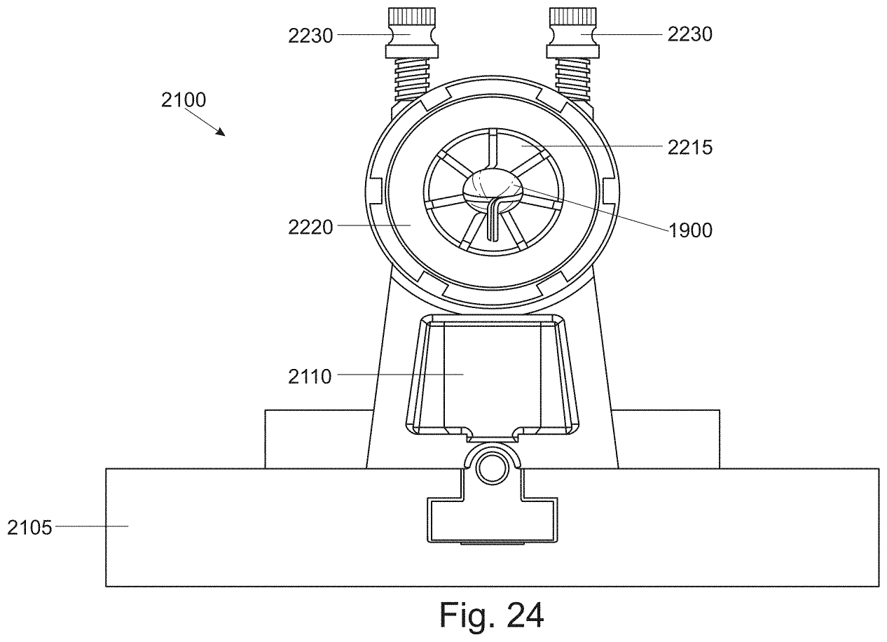

FIG. 24 illustrates an end view of FIG. 23 in the context of platform 2100. In this view, chuck 2215 is in the initial or “open” state. Each collet chuck includes a number of tabs arrayed around the longitudinal tunnel, and in the open state, these tabs are separated. Forcing chuck 2215 into structure 2210 by turning nut 2220 moves these tabs closer together to narrow the longitudinal tunnel and to thereby compress graft 1900.

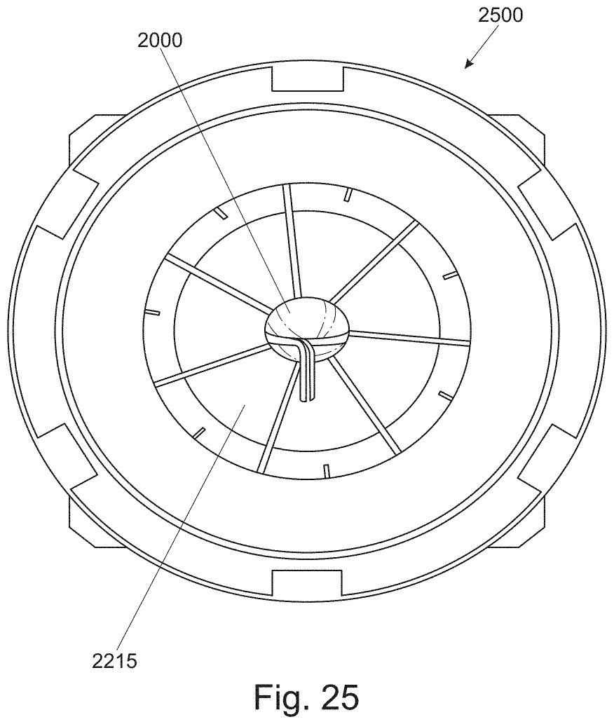

FIG. 25 illustrates an end view 2500 similar to FIG. 24 but after lateral compression (e.g., longitudinal tunnel of chuck 2215 closed) to produce compressed connective tissue graft 2000. In FIG. 25 the tabs of chuck 2215 are closed/touching which produces the smallest diameter longitudinal tunnel. This is in contrast to FIG. 24 where the tabs are separated and define a larger diameter longitudinal tunnel.

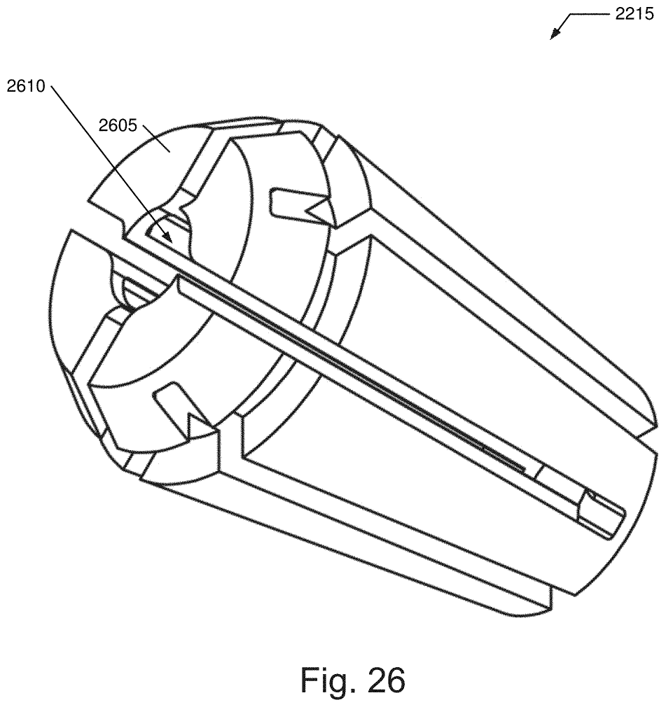

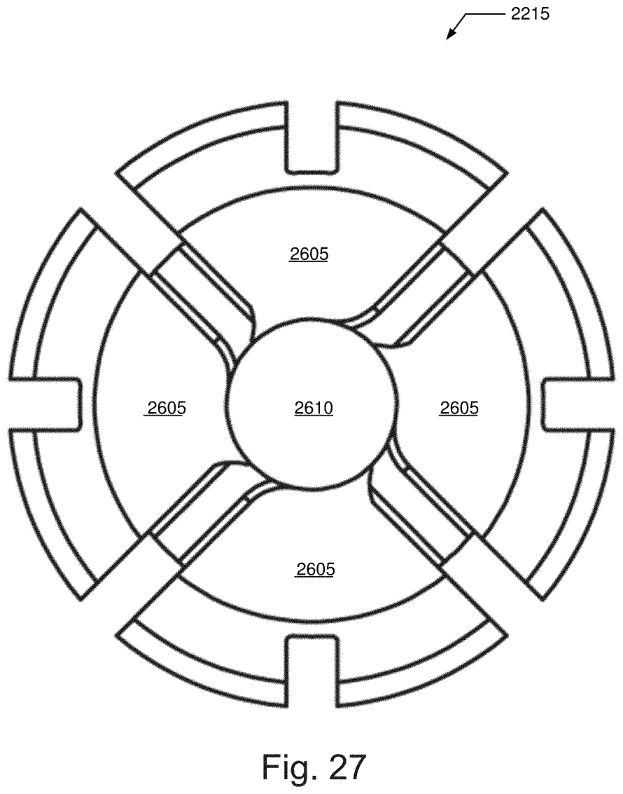

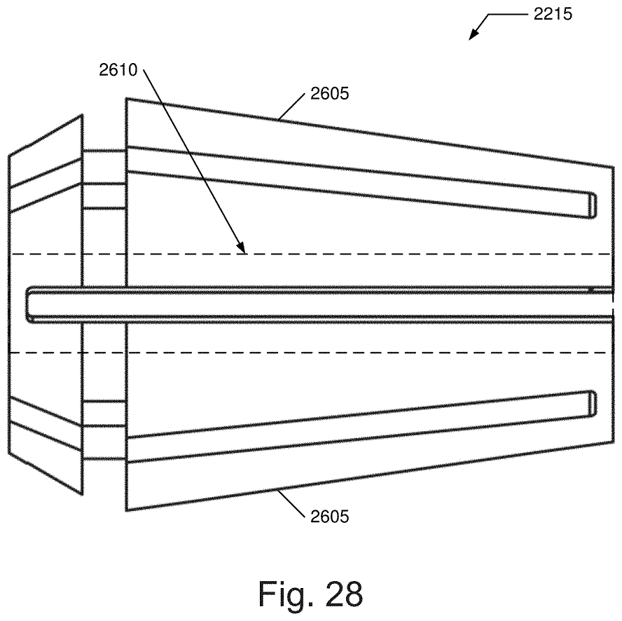

FIG. 26-FIG. 28 illustrate details of collet chuck 2215. FIG. 26 illustrates a perspective view of collet chuck 2215 of graft platform 2100; FIG. 27 illustrates an end view of collet chuck 2215 of FIG. 26; and FIG. 28 illustrates a side sectional view of collet chuck 2215 of FIG. 26. Collet chuck 2215 includes an N number, N≥2, of moveable tabs 2605 that collectively define a longitudinal tunnel 2610. N may be any integer two or greater and may often be an even number, for example N is an element of the set {2, 3, 4, 5, 6, 7, 8, 9, 10, 11, 12, . . . } depending upon various design considerations in compressing and shaping an outer perimeter of graft 1900 to produce graft 2000. In FIG. 26-FIG. 28, N=4. In FIG. 24 and FIG. 25, N=7.

In operation, platform 2100 may include one or more different modalities for decreasing D1 of graft 1900 and providing D2 of graft 2000 that may be significantly smaller. One modality includes inserting all or portion of graft 1900 into one or both collet chucks 2215 (chucks 2215 may have a length to accommodate the intended use. A single chuck 2215 that is long enough may compress an entire length of graft 1900. The tightening of the collet nut while some of all of graft 1900 is disposed inside the longitudinal tunnel of the corresponding collet chuck will compress D1 of graft 1900 to D2 of graft 2000.

In other embodiments, a portion of each end, up to one-half for example, of graft 1900 is installed into each of two opposing collet chucks on platform 2100. That end portion in each collet chuck may then be compressed by tightening the corresponding collet nut. In this example, one-half of graft 1900 is compressed by each stage. Variations are possible, such as where ⅓ of graft 1900 is installed into one chuck and the remainder ⅔ of graft 1900 installed into the other chuck. This allows for each end or portion of each end to be compressed to different diameters (the compressed diameter of one end may be different than the compressed diameter of the opposite end). Some procedures or protocols may be advantaged by producing differently sized or profiled tunnels in the different bones—one tunnel size or profile in a femur and a different one in the tibia for example. Some embodiments of the present invention allow for this as necessary or desired.

Another possible modality for decreasing D1 of graft 1900 is to use platform 2100 to grip ends of graft 1900 in each housing and then to use the drive rod to separate the housings. By using the torque wrench, an operator understands how much tension is applied to graft 1900 intermediate the gripped ends which tensions, stretches, and thins the intermediate portion. The degree of thinning of this intermediate portion is dependent upon the force applied and the tensile and compressive moduli (mechanical properties) of graft 1900. As long as the thinning occurs in the elastic deformation range, there will be a tendency for the intermediate portion thinned this way to return towards a thicker instance. The graft may also exhibit elastic and/or inelastic behavior frequently described in solids, where a subset of viscoelastic materials have a unique equilibrium configuration and ultimately recover fully after removal of a transient load, such that after being squeezed, they return to their original shape, given enough time. The transient strain is recoverable after the load or deformation is removed. Time scale for recovery may be short, or it may be so long as to exceed the observer's patience.

In some embodiments, it is thus possible to produce a diameter profile over a length L2 of graft 2000. Typically graft 2000 includes a single diameter D1 over the entire length L1. However, embodiments of the present invention may tailor each end or portion thereof with a desired diameter (the same or different from the other end) and with a desired diameter for the intermediate portion that is the same or different from either or both ends. Some amount of each end, and the intermediate portion, may have its diameter be relatively independently controlled. Any end or intermediate portion may have a greater or lesser diameter than another part of graft 2000. The intermediate portion may have the same, larger, or smaller diameter than one or both end portions. The same is true of each end relative to the other end and the intermediate portion.

In the above discussion, the grafts and tunnels, and structures complementary thereto have been described as generally elongate circular cross-sectional structures (e.g., cylindrical tunnels). This is because the current procedures provide for drilling tunnels in the implicated bones and the drilling produces generally circular cross-sectional tunnels. In general all ACL reconstructive techniques, whether performed arthroscopically or open, utilize the particular technique of initially proposing the tibial and femoral tunnels with a “guide wire”, which is drilled in the desired position, and after confirmation, over-drilled with a cannulated drill bit to produce a perfect cylindrical tunnel.

In some instances, it may be possible to produce tunnels in the bones, possibly utilizing different techniques and completely different technologies, with the tunnels having other than circular (e.g., cylindrical) cross-sections. Perhaps healing and recovery may be better achieved with a generally elliptical cross-section tunnel such as a frustrum (e.g., of a pyramid or cone or other closed three-dimensional cavity volume), a rectilinear cross-section tunnel, or a tunnel that has a varying diameter over its length. In some cases, a bone preparation tool may include a LASER, a 3-dimensional (3D) bone sculpting tool, or robotic instruments to define a desired regular/irregular/symmetric/asymmetric tunnel that varies from a same-sized cylindrical bore (iv) typically produced in the femur and the tibia for current ACL reconstructive techniques.

An advantage of some embodiments of the present invention when installing a compressed graft 2000 into any of these alternative types of tunnels (as well as the cylindrical bores from a drill) is that the graft 2000 may selectively expand to fill any variable profile of the tunnel in the femur and tibia.

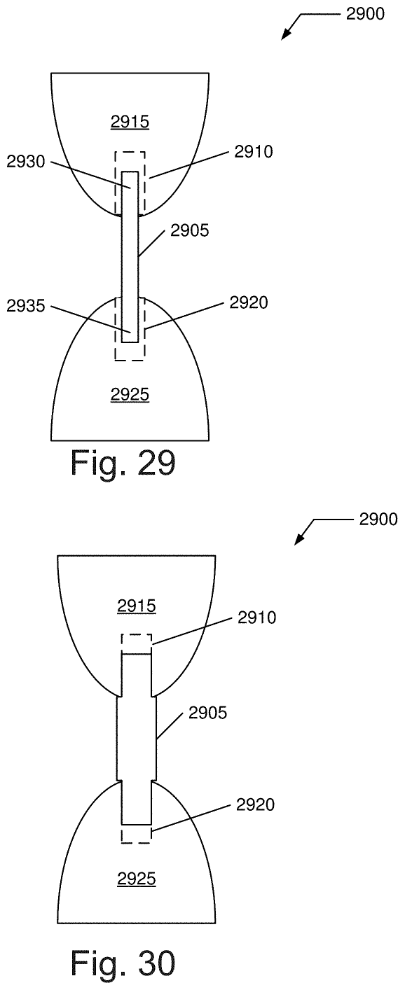

FIG. 29-FIG. 30 illustrate a reconstruction 2900 of an ACL in a pair of cylindrical bone tunnels. FIG. 29 illustrates pre-expansion of a compressed ACL graft 2905, such as an appropriately sized embodiment of graft 2000 in FIG. 20 and FIG. 30 illustrates a post-installation-expansion of compressed ACL graft 2905. A bone tunnel 2910 is prepared (e.g., profiled, sculpted, processed) in a portion of a femur 2915 and a bone tunnel 2920 is prepared in a portion of an adjacent tibia 2925. There may be several ways to prepare these bone tunnels, such as by installing a guide wire along a desired path and then using a cannulated drill bit to follow the guide wire to the desired depth. For example, these tunnels may have a diameter of about 9 millimeters and ACL graft may have an uncompressed diameter of about 10 millimeters and a compressed diameter of about 6-8 millimeters. With these dimensions, the compressed ACL graft may easily be installed into a prepared bone tunnel and an uncompressed ACL graft may produce significant lateral frictional forces holding it in place as the healing occurs and natural fixation completes itself to bond the uncompressed ACL graft into the prepared bone tunnels (with or without external fixation devices or structures).

After decompression of compressed ACL graft 2905 (in FIG. 30) the expanded ACL graft 2905 tightly fills each bone tunnel as it conforms to the cross-section profile (e.g. circle for a cylindrical bone tunnel). A diameter/profile of bone tunnel 2910 need not, but may be, the same as a diameter/profile of bone tunnel 2920. As long as portions of the diameters of the bone tunnels where the ACL graft is to be bonded (e.g., openings of the bone tunnels) are smaller than an original unexpanded diameter of the compressed ACL graft, temporary press-fit fixation from the decompression of the installed graft will secure the decompressing ACL graft into the bone tunnels and provide the advantages noted herein.

Once the bone tunnels are prepared, a first end 2930 of compressed ACL graft 2905 is installed into bone tunnel 2910 and a second end 2935 of compressed ACL graft 2905 is installed into bone tunnel 2920. As compressed graft decompresses it expands towards its original pre-compressed shape unless constrained (by a bone tunnel side wall for example).

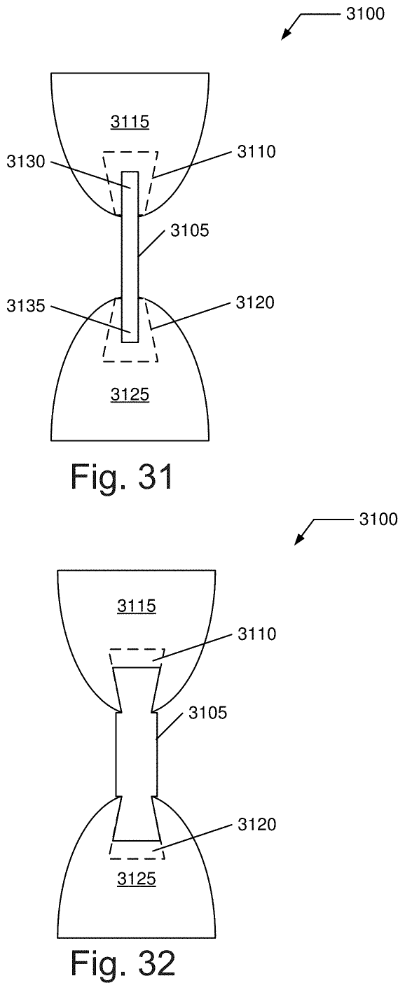

FIG. 31-FIG. 32 illustrate an alternative reconstruction 3100 of an ACL into a pair of profiled bone tunnels. FIG. 31 illustrates pre-expansion of a compressed ACL graft 3105 (which may be similar to ACL graft 2905 in FIG. 29), such as an appropriately sized embodiment of graft 2000 in FIG. 20 and FIG. 30 illustrates a post-installation-expansion of compressed ACL graft 3105.

Alternative reconstruction 3100 is similar to reconstruction 2900 with the exception of the shape of the bone tunnels (and consequently the manner of the formation of the profiled bone tunnels in FIG. 31 and FIG. 32. The noted characteristic of the conforming decompression of a compressed ACL graft 3105 is used in this alternative to expand into specially profiled bone tunnels that may have a number of shapes where an opening profile is purposefully and significantly smaller than a cavity profile deeper into the bone.

A profiled bone tunnel 3110 is prepared in a portion of a femur 2915 and a profiled bone tunnel 3120 is prepared in a portion of an adjacent tibia 2925. There may be several ways to prepare these profiled bone tunnels, such as by use of a surgical robot or three-dimensional bone sculpting, or LASER such as laser ablation of bone as described herein, for example in the discussion of FIG. 34 below. For example, these profiled tunnels may be generally shaped as a frustum have a narrower opening diameter of about 8 millimeters, a wider base diameter of about 9-10 millimeters, and the ACL graft may have an uncompressed diameter of about 10 millimeters and a compressed diameter of about 6-7 millimeters. With these dimensions, the compressed ACL graft may easily be installed into a prepared bone tunnel and a decompressing ACL graft, when decompressed, may produce significant frictional and mechanical forces (e.g., normal forces) holding it in place as the healing occurs and natural fixation completes itself to bond the uncompressed ACL graft into the prepared bone tunnels (with or without external fixation devices or structures).

After decompression of compressed ACL graft 3105 (in FIG. 31) the expanded ACL graft 3105 tightly fills each profiled bone tunnel as it conforms to the cross-section profile (e.g. circle for a cylindrical frustum bone tunnel). A shape of profiled bone tunnel 3110 need not, but may be, the same shape as the shape of profiled bone tunnel 3120. As long as portions of the diameters of the profiled bone tunnels where the ACL graft is to be bonded (e.g., openings of the bone tunnels) are smaller than an original unexpanded diameter of the compressed ACL graft, temporary “biologic press-fit” and mechanical fixation from the decompressing ACL graft will secure the ACL graft into the profiled bone tunnels and provide the advantages noted herein along with improved resistance to pull-out.

Once the bone tunnels are profiled, a first end 3130 of compressed ACL graft 3105 is installed into bone tunnel 3110 and a second end 3135 of compressed ACL graft 3105 is installed into bone tunnel 3120. As compressed graft decompresses it expands towards its original pre-compressed shape unless constrained (by a bone tunnel profiled side wall for example).

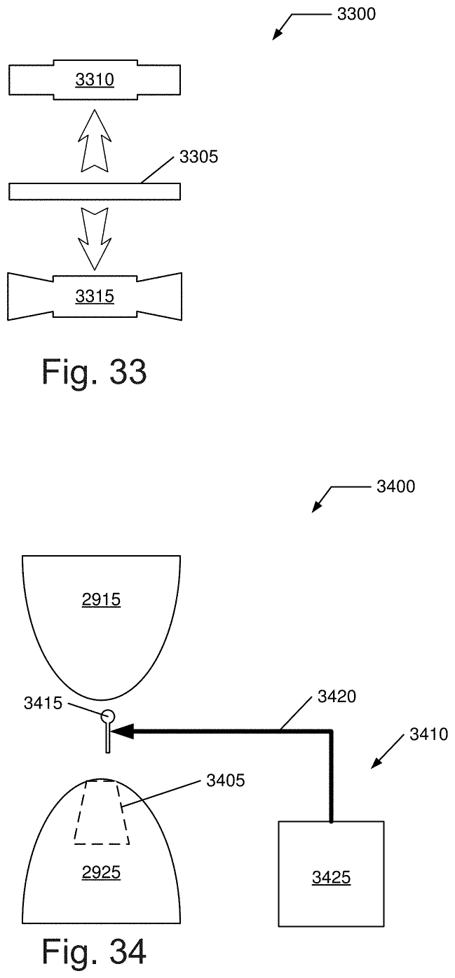

FIG. 33 illustrates different conforming expansions 3300 of a compressed ACL graft 3305, dependent upon a preparation of a bone tunnel and represent the examples from FIG. 29-FIG. 32. For example, when compressed ACL graft 3305 is installed into cylindrical bone tunnels (a simple example of a profiled bone tunnel), its decompression results in an uncompressed graft similar to graft 3310. When compressed ACL graft 3305 is installed into “inverted frustum” profiled bone tunnels (e.g., as illustrated in FIG. 31 and FIG. 32), its decompression results in an uncompressed graft similar to graft 3315.

FIG. 34 illustrates a bone profiling apparatus 3400 for a preparation of a profiled bone tunnel 3405 by an automated or semi-automated (constrained surgeon manipulation) surgical apparatus 3410. In FIG. 34, apparatus 3410 has produced a first profiled bone tunnel 3405 in tibia 2925 and is preparing to produce a second profiled bone tunnel in femur 2915. Apparatus 3410 includes a bone preparation implement 3415 having a mechanical coupling 3420 (direct or indirect) between a controller 3425 (e.g., a stored program computing system including processor executing instructions from a memory including a user interface to set user options and parameters).

There are automated assistive surgical devices which may fill the role of apparatus 3410, such as robotic assisted surgical platforms (e.g., MAKO, da Vinci, Verb, Medtronic, TransEnterix, Titan Medical systems, NAVIO blue belt, and the like). These platforms provide positional control/limitation of surgical implements operated by a surgeon, such that the robotic tools (some of which utilize custom software and CT data) resist the movements by the surgeon that may attempt to deviate from a planned procedure, bone preparation, or other processing. These platforms are often installed into a known reference frame shared by the patient so precise position control/limitation may be imposed. Installing bone preparation tool 3415 (e.g., a high-speed rotating burr or the like) the surgeon may operate the platform to form a precisely profiled bone tunnel as described herein (e.g., first profiled tunnel 3405). A profiled tunnel may be initiated from a bit-prepared cylindrical tunnel and then profiled from there or apparatus 3410 may prepare the entirety of the profiled bone tunnel.

Further, current ACL techniques require that the surgeon estimate the length of the graft to fit the combined length of the tibial and femoral tunnels plus the intra-articular length of the ACL graft, housed in the notch. Despite best efforts mismatches between the length of the graft and the tunnels is not infrequent, which adversely affects the outcome. The use of automated surgical devices noted above has the advantage of providing the exact lengths of the tibial and femoral tunnels as well as the intra-articular length of the ACL graft within the notch. These techniques allow bone resection of any profile with varying trajectories and depths based on planned procedure, for example to within a millimeter. The tunnel lengths can be determined pre-operatively or intraoperatively and correlated with the length and diameter of the prepared allograft. Growth factors can be applied to pre-prepared allograft with external of and/or internal sheaths, or to auto-grafts prepared at the time of surgery.

Apparatus 3410 may be used to produce internal ridges, dimples, or other irregularities in the lateral wall of a bone tunnel (profiled or “conventional” cylindrical tunnel). The uncompressing ACL graft will fill these irregularities which may further promote fixation and healing.

Described above are embodiments (apparatus and methods) for production of a compressed connective tissue graft. Such a graft may be prepared from patient or may be provided separately (e.g., a frozen pre-prepared allograft) that may be sized and compressed.

An embodiment of the present invention includes off-site advance preparation of compressed connective tissue graft that are shipped and stored in the compressed state. They may be frozen in the compressed state sufficiently partially thawed at the time of installation to allow appropriate decompression in situ. It may be that the pre-compressed allograft is delivered in a peel pack while freeze dried in the compressed state. The allograft is removed from the packaging and the surgeon will have some time for installation before it decompresses. In some cases, the allograft's decompression is accelerated by saline solution. Exposure of the compressed allograft to body fluids in the bone tunnels may also accelerate the decompression for fixation into the bone tunnel.

In other embodiments, a protective sheath may be provided that is installed after compression to maintain the connective tissue graft in the compressed state. Removal of the sheath allows for decompression. The sheath may be dissolvable in body fluids and installation into a bone tunnel begins the dissolution and decompression.

The sheath may be provided as a two-part element: an outer protective film prevents decompression and an inner layer that may temporarily inhibit decompression during the installation process. When ready to install, the outer layer is removed and the connective tissue graft (with inner layer) is inserted into the bone tunnel. Alternatively, the outer and inner sheaths of compressed ACL prepared grafts can be embedded with a combination of biological growth factors including the TGF family, bone morphogenic proteins (BMP), insulin like growth factors, matrix metalloproteinases, fibroblast growth factors, vascular endothelial growth factors, platelet derived growth factors, and or other stem cell derived growth factors (including epithelial and mesenchymal stromal cells), which alone or in combination can significantly improve healing of tendon to bone, promoting angiogenesis and osteogenesis at the tendon-bone interface after ACL reconstruction. The sheaths may also include other allogenic sources of growth factors such as amniotic membrane products and the like.

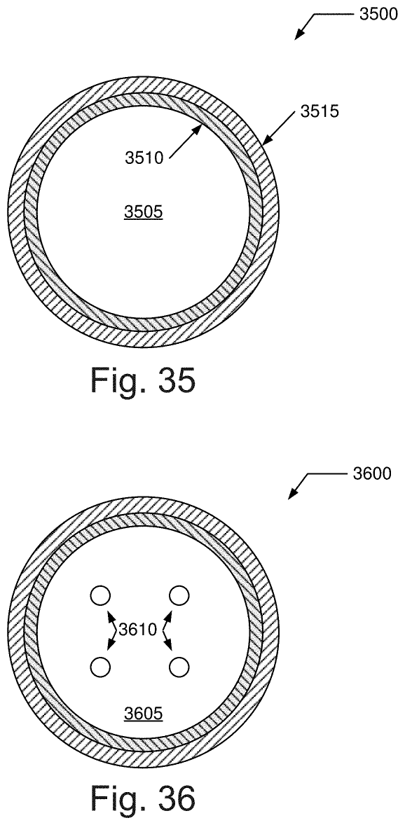

FIG. 35 illustrates an allograft system 3500 including a pre-compressed allograft 3505 with a sheathing subsystem having one or more sheaths (e.g., an inner sheath 3510 and an outer sheath 3515). The sheathing subsystem may accomplish one or more functions depending upon implementation, to achieve desired goals as described herein. Those goals may include a number of functions, such as maintaining a pre-compressed allograft 3505 in its compressed mode until installed into a prepared bone tunnel for decompression as described herein. Other functions include enhancing preservation of sterility and delivery of growth factors into the bone tunnel at the graft/tunnel interface.

FIG. 36 illustrates an allograft system 3600 including a pre-compressed allograft 3605 with an embedded prosthesis subsystem having at least one connective tissue prosthetic element 3610 that runs a length. There is a history of development and investigation of synthetic ACL grafts but have generally not proven to be successful. There are a number of problems of a pure synthetic connective tissue graft, including a) breakdown of the synthetic material with exposure in the joint that too often leads to synovitis and arthritis due to existence of the foreign material in joints; and b) not finding a synthetic graft that has equivalent material properties of connective tissue. There is not complete agreement on the mechanical properties needed or desired for such a synthetic graft: some materials discuss a “stiffness” of the synthetic material. However, it may be the case that a graft that has the similar “toughness properties” of native ACL may be preferable: i.e., more ductile than brittle (i.e. a larger plastic range).

Allograft system 3600 is believed to address some of these drawbacks as it is a hybrid system: native connective tissue on the outside with an embedded prosthetic element(s) inside. Illustrated is embedding the prosthetic elements inside a pre-compressed allograft as described herein. Some embodiments may embed these synthetic elements within a conventional allograft and use an alternative fixation method.

The one or more prosthetic elements may each include single strands of suitable material (e.g., natural and/or synthetic material) or may include a weave of such materials (including composite weaves of multiple different materials). The one or more embedded prosthetic elements do not provide for intra-articular bone exposure.

When embedded into a pre-compressed, the expansion fixation of the decompressing allograft into a bone tunnel secures the prosthetic elements along with the outer native decompressed graft.

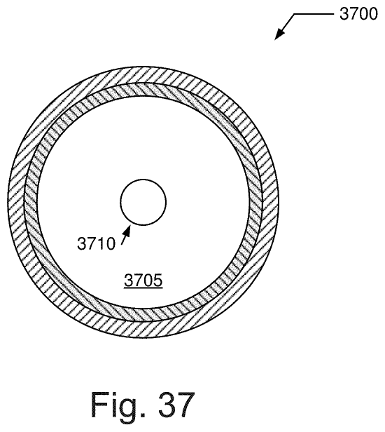

FIG. 37 illustrates an allograft system 3700 including a pre-compressed allograft 3705 with a expansion subsystem having at least one expansion element 3710 disposed in one or more portions that are to be expanded. These portions may be one or both end portions and/or middle portion. In some cases, the at least one expansion element may run a length of the compressed allograft. In many embodiments, the enlargement of a pre-compressed allograft has been described as a generally passive process in which a compressed allograft is allowed to decompress. It is the case that under some circumstances that natural connective tissue may expand somewhat when subjected to bodily fluids or pre-operative fluid baths (e.g., saline solution) for thawing an often-frozen allograft.

Allograft system 3700 includes an active expansion system which expands compressed native connective tissue. Expansion may be accomplished by use of the at least one embedded expansion element 3710. This at least one expansion element 3710 may be embedded into a pre-compressed allograft as described herein or embedded into a conventional allograft. In some implementations, the at least one expansion element 3710 may be part of, included within, integrated with, or provided as part of at least synthetic prosthetic element as illustrated in FIG. 37. For example, a structure may have a dual-use of providing the synthetic prosthetic element and the expansion element.

System 3700 introduces the concept of “internal expandable structures (e.g., tubes) for screw-less interference fixation of pre-compressed ACL grafts (it being noted that herein that these expandable structures may be used with conventional allografts and/or with conventional fixation methods).

One method to increase tendon/bone interface pressures (in lieu of interference screws) is a new concept of introducing expandable tubes, cages or stents within the ends of the allografts, and allowing the tube, cage or stent to expand passively or actively, to subsequently increase graft bone interface pressures to assure “direct” type fixation.

The material for the “intra graft tubes” can be synthetic non-absorbable material such as plastic and or polyester or similar material; or absorbable material.

Absorbable material could be polymer based as in polylactide (PLLA), polyglycolic acid (PGA), copolymers (PGA/PLA) poly paradiaxanone, and various stereoisomers of lactic acid, along with various bio-composite materials including a mix of polymers noted above plus calcium phosphate etc. Alternatively absorbable material could be magnesium alloy based with similar functionality where the material absorbs over time (e.g., over three months).

The expansion of the tubes may occur passively over defined period of time or actively. Active expansion can be done by balloon expansion after implantation of the graft, similar to what is done with balloon expandable stents in vascular procedures, where inflation of a balloon within the tube expands the tube inside the graft to increase intra graft pressure on the graft/bone interface, without any contact of the tube (whether bio absorbable or synthetic) with the tendon/bone interface. This concept theoretically eliminates the current problem of screw breakdown and release of inflammatory cytokines associated with tunnel widening and poor graft healing. Active expansion can also occur by “unsheathing the tube” or “pulling a rip cord” immediately after implantation of the graft, which is also done in vascular procedures.

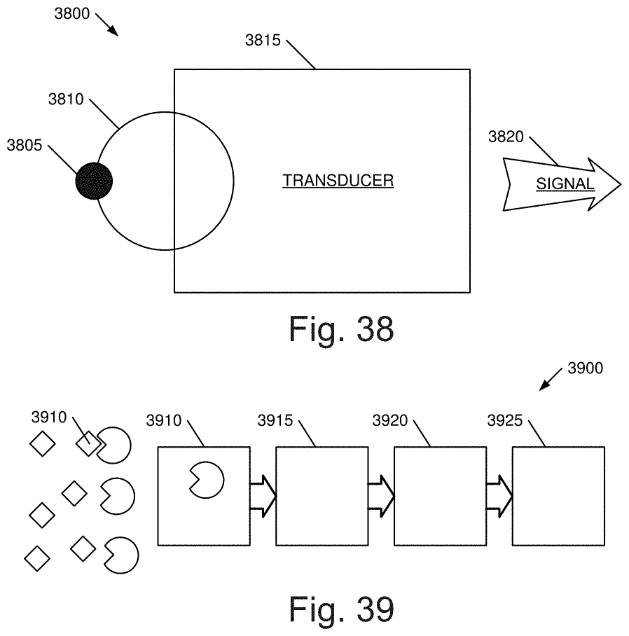

FIG. 38-FIG. 46 illustrate aspects of biologic installation structures including a set of sensors. Cement-less arthroplasty has been recognized as one of the most successful operations of the 20th century providing pain relief for millions of patients suffering from osteoarthritis. However, cement-less arthroplasty is still plagued with failures related to aseptic loosening, infections, and metallosis. There has been increasing concerns regarding these failure modes as more surgeons with less experience perform an increasing proportion of these operations, leading to failure rates of as high as 25% (for example in hip replacement surgery) over the last 10 years.

Aseptic loosening in total joint arthroplasty is directly related to a lack of ability to precisely calibrate (interference fit) at the prosthesis/bone interface. It is generally known that micromotion of greater than 50 μm will lead to poor osteointegration (bone ingrowth), leading to fibrous tissue formation at the interface and eventual aseptic loosening, which accounts for 75% of total joint failures (including total hip and knee replacements). A prosthesis that is too loose-fitting may lead to fibrous tissue formation, while one that is too tight-fitting may lead to occult fracture, both scenarios subsequently lead to poor interference fit fixation and aseptic loosening. These problems have resulted in significant pain and suffering for patients, as well as producing tens of billions of dollars of additional cost to society.

Infections of artificial joints cause severe damage to patients bone and joints and are difficult to diagnose and treat. In particular, a diagnosis of an infected prosthesis installation involves a use of multiple laboratory tests including blood analysis, X-rays, MRIs, CAT scans, nuclear medicine scans, and a variety of chemical analysis performed on joint fluids. These tests individually and collectively yield poor results and are neither highly specific nor sensitive. The surgeon is frequently called upon to make a “clinical judgement” in assessment of prosthetic joint installation infections and ultimately is faced with incorporating the varied and frequently conflicting data provided through these tests.

Metallosis is recently a recognized clinical syndrome that has caused significant concern for the orthopedic community. Morse taper technology has been utilized in orthopedics to bond modular prosthesis to each other (described in U.S. patent applications (Ser. No. 15/362,675 filed 28 Nov. 2016), (Ser. No. 15/396,785 2 Jan. 2017), and (Ser. No. 14/965,851 10 Dec. 2015)). Micromotion at the modular prosthesis interface has led to production of metal debris, which through the process of Mechanically Assisted Crevice Corrosion (MACC) led to the clinical syndrome of Trunnionosis and Metallosis causing Adverse local Tissue Reactions ALTR with significant damage to bone, joints and soft tissues, as well as metal toxicity.

Current diagnostic methods for evaluation of aseptic loosening, infection and metallosis in orthopedics (especially cement-less arthroplasty) are highly inaccurate, lacking both specificity and sensitivity, often leaving the surgeons to rely on “clinical judgement” without the benefit of clear and convincing evidence.

Ligament reconstruction techniques in orthopedics similarly involve application (placement) of ligaments grafts as prosthetic devices in prepared bone tunnels. These (soft tissue prosthetic) replacements share the some of the same concerns as metal alloy prosthetic replacements including infection and loosening (graft rejection), as well as graft failures with subsequent traumatic injuries.

Dental procedures similarly involve application of prosthesis or implants into bone and can be plagued by similar problems (as in orthopedic surgery) such as aseptic loosening, infection, and metallosis. For example, an early infection of a dental implant may not be easily detectable through standard testing with X-rays and laboratory tests. When laboratory tests are negative, but the patient is symptomatic, dentists typically treat patients empirically with oral antibiotics. However, deep infections do not respond well to oral antibiotic treatment, which can lead to progression of the disease and development of antibiotic resistance. Biosensors, as described for orthopedic Prosthetic Interface Point of Care Testing PI-POCT described in the subsequent sections, have similar uses and utility in dental surgery, particularly in diagnosis of infections and implant loosening.

What is needed is in the field of orthopedic surgery, in particular in cement-less arthroplasty and ligament reconstruction techniques (essentially all aspects of orthopedic surgery where a prosthesis is introduced into the body), as well as dental surgery, is a development of prosthesis interface point-of-care (POC) testing devices which can provide diagnostic tests and ‘sensing’ in situ, directly, and at the actual site of possible pathological process, to facilitate evidence-based diagnosis. We term this phenomena Prosthetic Interface Point of Care Testing PI-POCT.

Current diagnostic methods for evaluation of failures of orthopedic arthroplasty and soft tissue prosthetic replacements are varied and expensive and collectively produce low yield. There is a need for methods to produce POC tests in orthopedic arthroplasty (and ligament reconstruction) that are affordable, user friendly, specific, sensitive, robust and equipment free.

Recent advances in biosensors, semiconductors and wireless communication techniques have attracted significant interest in multiple industries. Wireless POC devices as described herein offer an advantage of continuous monitoring of biologically and physically relevant parameters, metabolites and bio-molecules relevant to pathologic conditions such as aseptic loosening, infections, metallosis, and graft failures.

Biosensors are ubiquitous in biomedical diagnosis as well as other POC monitoring of disease, drug discovery, forensics, and biomedical research. A wide range of methods have been used for development of biosensors.

A biosensor includes two components: a bioreceptor and a transducer. In its most basic form the bioreceptor is a biomolecule that recognizes a target analyte, and a transducer converts the recognition event into a measurable signal. A uniqueness of the biosensor includes that these two components are integrated into a single sensor (unit), which measures the target analyte without use of a reagent. A simplicity and a speed of measurement requiring no specialized laboratory skills are some advantages of a biosensor.

FIG. 38 illustrates a generalized biosensor 3800. An analyte 3805 is recognized by a bioreceptor 3810 through a recognition event. The recognition event is transformed by a transducer 3815 into a signal 3820 that may be measured/quantified.

Biosensor research has experienced explosive growth over the last two decades. A modern biosensor is an analytical device that converts a biological response into a quantifiable processable signal. Biosensors are employed in disease monitoring, drug discovery, detection of pollutants and disease-causing microorganisms.

Recent advances in integrated biosensors and wireless communication have created a new breed of POC diagnostic devices which may include one or more of the following components: (a) an analyte—a substance of interest that needs detection; (b) a bioreceptor—a molecule/material/compound that specifically recognizes the analyte is known as a bioreceptor with enzymes, antibodies, DNA, RNA, aptamers, cells, receptor proteins included as examples of bioreceptors wherein an interaction of the bioreceptor with the analyte is termed bio-recognition; (c) a transducer—the transducer converts one form of energy into another, which when incorporated into a biosensor means the transducer converts the bio recognition signal into a measurable signal, which may include either an electrical signal (e-) or and optical signal; (d) a set of electronics—for example integrated circuits and wireless systems wherein the transduced signal may be processed and amplified for display; and (e) a user interface—for example an indicator or display mechanism which may involve hardware and software that interprets the results of a biosensor in a user-friendly/perceptible manner.

FIG. 39 illustrates a point-of-care (POC) diagnostic device 3900. Device 3900 is responsive to an analyte 3905 using a bioreceptor 3910. Bioreceptor 3910, in the presence of analyte 3905, produces a bio-recognition event that is converted by a transducer 3915 into a signal, sometimes referred to as signalization. The signalization is processed by a set of electronics 3920 and may be presented to a user by some type of a display 3925 or indicator or may be otherwise analyzed or incorporated into post-conversion activities of a system or process that makes incorporates device 3900.

Bioreceptor 3910 may include an enzyme, cell, aptamer, DNA, nanoparticle, and antibodies producing the bio-recognition event which may include production of light, heat, pH change, mass change, and combinations. These bio-recognition events are processed by transducer 3915 which may include a photodiode, pH electrode, quartz electrode, field-effect-transistor (FET), and the like and combinations thereof.

Transducer 3915 produces a transducer signal that is received by electronics 3920 which may convert from analog-to-digital and/or include signal conditioning structures, systems, and processes. Electronics 3920 produces a processed signal for display 3925.

In such manner biological molecules are “immobilized” (attached) on sensing electrodes for detection of a target analyte. The target analyte interacts with immobilized bioreceptors on the surface of sensing electrodes which further induces a change in an electrical signal such as conductance, current, potential, frequency, phase, amplitude, impedance or capacitance. The signal response is monitored and correlated to the concentration to the target analyte through a calibration curve.

Wireless biological electronic sensors have been created by integrating a bio-receptor sensing transducer with wireless antennas. The wireless aspect of (biological electronic systems) are classified into following categories: wireless radio frequency identification, wireless acoustic waved based biosensors, wireless magneto elastic biosensors, wireless self-powered biosensors and wireless potentiostat-based biosensors.

To develop wireless biological electronic sensors, a sensing transducer is immobilized (attached) to bioreceptor to make a biosensing transducer. This biosensing transducer is further integrated with a wireless communication element to transmit sensing signals to external receiving device.

Several types of sensing transducers have been used and include electrochemical electrodes, transistors, resistors, capacitors, surface acoustic wave electrodes, magnetic acoustic plates, magnetoelastic ribbons.

The bioreceptors mainly include catabolic based bioreceptors such as enzymes or binding/hybridization based bioreceptors such as antibodies, DNA, RNA, aptamers, peptides, or phages.

Among different type of sensing transducers, electrochemical electrodes are a basic and widely used class of transducers, majority of which are amperometry based (H₂O₂ or O₂ measurement), potentiometry based (pH or pIon measurement), or photometry based utilizing optical measurements. All of which may act to convert action of the bioreceptor molecule (a biorecognition event) into a signal.

Over time different methods of transduction have been developed and will be developed, some of which may be bio-compatible for use with a biologic structure for compatible installation in a living body. In principle any method that is affected by the biorecognition reaction can be used to generate a transduced signal and may, in some cases, be included in an embodiment of the present invention.

Piezoelectric materials and surface acoustic wave devices offer a surface that is sensitive to changes in mass. For example, piezoelectric silicon crystals called quartz crystal microbalance QCM may be used to measure very small changes in mass in the order of picograms.

Conductimetric transducers may be used when a biorecognition reaction causes a change in the dielectric measurement of the medium.

Thermometric transducers may be used when the biorecognition event is accompanied with the creation or absorption of heat.

For some implementations of the present invention, there may be advantages associated with miniaturization. Mass production has led to the development of field-effect-transistor (FET) technology for application as a transducer which may be incorporated into some biosensors as described herein. Field-effect transistors (FETs) are used extensively in semiconductor industry in memory and logic chips and respond to changes in an electric field. The construction of multi-analyte conductance biosensors and conductive polymer-based devices have been, and will be, enhanced by a rapid development of semiconductor technology and sensor integration with microelectronics devices producing FET devices.

In recent years an emerging field of nanotechnology has produced interesting materials (such as nanowires, nanotubes, nanoparticles, nanorods, thin films, graphene and graphene oxide, carbon nanotubes), all of which are increasingly being used as building blocks of biosensing techniques and new transduction technologies, advancing biosensor development.

The nanostructures sometimes are associated with extraordinary electronic properties, enhanced electron transport ability, mechanical strength, pliability and impermeability, and have found their place in several biosensors such as biological field effect transistors Bio-FET which couple a transistor device with a bio-sensitive layer that can specifically detect bio-molecules by detecting changes in electrostatic potential due to binding of analyte. Commonly used Bio-FET systems in medical diagnostics include: (ion-selective field-effect transistor ISFET and enzyme field-effect transistor EnFET).

Specifically, reducing a size of a biosensor to nanoscale may result in a better signal to noise ratio, as well as requiring smaller sample volumes for detection. In particular, in the nanoscale dimension, a surface to volume ratio of the sensing active area increases and the size of the detecting electrode and the target analyte become comparable. This may result in both better sensitivity and specificity providing the promise of single molecule detection. Nanomaterials provide new and enhanced methods of biosensing by improving sensitivity, increasing stability and shelf life, achieving better signal to noise ratio, better response time and so on, and while at the same time reducing fabrication costs, and allowing development small compact biosensing devices.

Another use of nanotechnology involves creation of nanopores and nanochannels with encapsulation techniques (lipid, hydrogel, Sol-Gel, lipid bilayers) to produce “ion channels” and to make use of a concept of transport process across appropriate membranes to create highly sensitive transduction elements.

Traditional electrochemical measuring methods (with electrodes) have largely contributed to the current advanced understanding of transduction mechanisms. Over time the integration of sensors with field-effect transistor technology (FET) and nanotechnology have produced devices that can be highly specific, sensitive and compact with low cost of fabrication. The fusion of electrochemical biosensing, nanotechnology, and field effect transistor FET technology makes this technology adaptable for point of care (POC) diagnostics in orthopedic surgery and post-operative care and monitoring.

In addition to an integration of electrochemistry with microelectronics and nanotechnology, novel and complementary biosensing techniques have emerged that provide specific additional strengths in biosensing, providing the ability to detect changes in mass and optical evanescence. For example, Electrochemical Surface-Plasmon Resonance EC-SPR and Optical Waveguide Light Mode Spectroscopy (OWLS) can be combined with electrochemical transducers to provide direct observation of changes in optics and mass absorption, in addition to electrical change. Electrochemical Quartz Crystal Microbalance (EC-QCM) uses the inherent resonance of crystals and its decrease with mass absorption to detect biological reactions.

The varied and extensive biosensing methods and techniques discussed herein will continue to develop more sophistication over time. The field of orthopedic surgery and post-operative care (and monitoring) has not so far benefited from PI-POCT diagnostic methods. In the discussion below various representative embodiments outline some concepts of PI-POCT diagnostics that may be utilized in orthopedic surgery and post-operative care.

Press Fit Measurement in Orthopedic Arthroplasty

As noted herein, aseptic loosening is a major cause of failure of cement-less arthroplasty. An embodiment of the present invention may make use of implantable sensors on prosthesis, to be utilized at the prosthesis/bone interface, specifically as a PI-POCT device, to provide real-time information about a quality of the interference fit of the implant into its implant location, both during installation and after implant installation. Implant PI-POCT may be accomplished with (i) pressure and force sensors; and/or (ii) distance, proximity and displacement sensors. Once an appropriate interference fit of any particular prosthesis/bone interface is determined through in vivo and in vitro studies, a calibration curve can be produced to determine how much force, pressure, distance, and displacement is necessary to obtain appropriate and optimal press fit. A biosensor, suitably positioned for permanent implantation on the surface of a prosthesis, to be engaged at a prosthesis/bone interface can provide necessary data (i.e., force and/or displacement measurement) in real-time fashion. In this way the surgeon will know immediately as to whether appropriate (optimal) interference fit fixation has been obtain at the time of implantation, and may be used for subsequent post-operative evaluation.

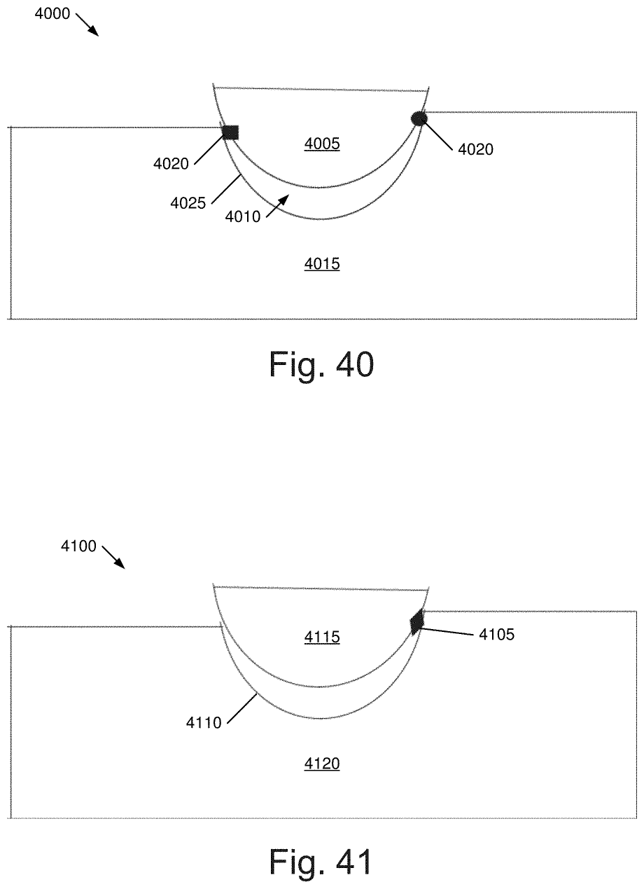

FIG. 40 illustrates an implementation of force/displacement sensing embodiment 4000 with interference fit fixation for installation of an implant 4005 into a prepared cavity 4010 in a portion of bone 4015. One or more biosensors 4020 may be installed on implant 4005 and/or at an bone/implant interface 4025. Biosensor 4020 may include a force and/or displacement transducer.

Aseptic Loosening in Orthopedic Arthroplasty

An electromechanical biosensor incorporated within a prosthesis surface at an anticipated junction of the prosthesis/bone interface can provide information regarding a loose prosthesis that is experiencing micromotion greater than 50 to 150 μm. Motion detectors such as Linear Variable Displacement Transformers LVDT applied permanently at this bone/implant interface may provide immediate PI-POCT diagnostics of a loose prosthesis.

FIG. 41 illustrates an implementation of an aseptic loosening sensing embodiment 4100, including a biosensor 4105 having an LVDT transducer, disposed at an interface 4110 of an implant 4115 and a portion of bone 4120, implant 4115 installed an interference fit fixation.

Infection in Orthopedic Arthroplasty

Infection of prosthesis with micro-organisms produces a variety of metabolic and electrochemical byproducts including pH, pIons, O₂, production of electrical currents and optical signals, as well as metabolites associated with specific infections. Common examples of substrates used to assess an infectious process include leukocyte estrace, alpha-defensing, nitrates, white blood cells, inflammatory debris to name a few. Given the advancement in biosensor technology and in particular its fusion with nanotechnology and integrated chips, it is advantageous to construct biosensors in the nanoscale with bioreceptors and transduction mechanisms that are highly specific to infectious processes. Any of the metabolites discussed above can be chosen as analytes to be detected. Bioreceptors (enzymes, antibodies, DNA, aptamers etc.) for detection the chosen analyte can be chosen and immobilized to transduction elements (capacitors, electrodes, transistors, FET, etc.), which are incorporated in integrated electronic chips with the capacity to transfer information wirelessly for interpretation and display.

In addition to monitoring the metabolites associated with infections, biosensor chip technology can directly measure the concentration of microorganisms. For example, Complementary Metal Oxide Semiconductor (CMOS) based integrated microelectrodes can be used to monitor growth of specific bacterial pathogens, such as methicillin resistant staphylococcus, which are of particular interest in orthopedics.

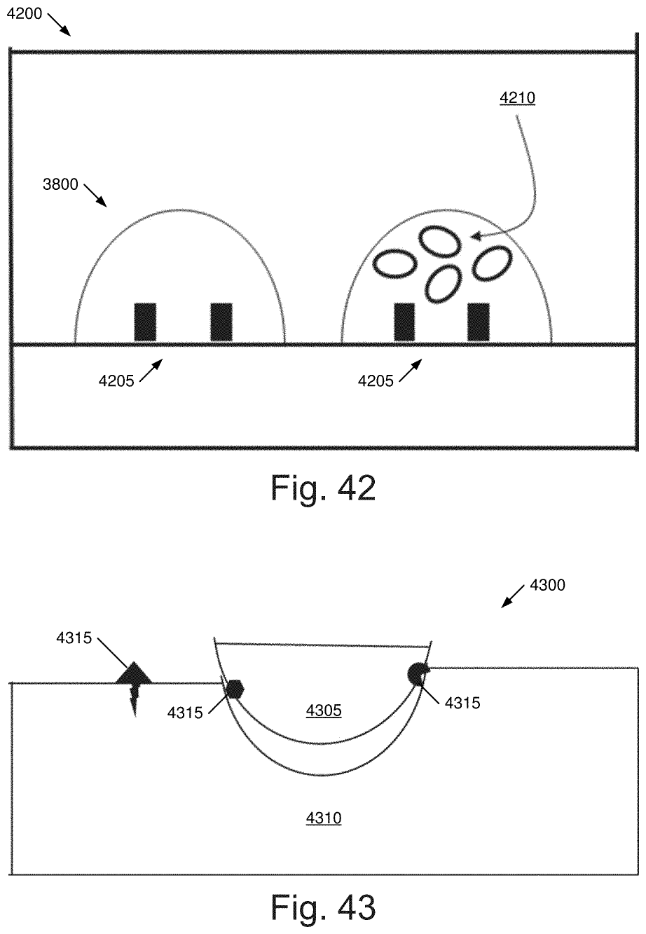

FIG. 42 illustrates a biosensor integrated microelectronic biosensor 4200 implemented in a CMOS package. Biosensor 4200 includes a set of electrodes 4205 for detection of one or more pathogens, such as bacteria 4210.

Metallosis and Trunnionosis in Orthopedic Arthroplasty

A presence of metallic debris in orthopedics is caused by micromotion between modular prosthesis. High concentrations of metal ion debris such as cobalt, chromium, titanium in the joint fluid and surrounding soft tissues occur as a result of poor interference fit between modular components. Metallosis can be a significant, and up to now, unrecognized source of inflammatory debris which can secondarily lead to loosening and infection. The current diagnostic methods for evaluation of metallosis and trunnionosis and are complex and indirect and generally result in poor yields in the early stages of the condition. The bio sensor technology noted herein may be incorporated and adapted for a PI-POCT device for detection of, immediate, and early diagnosis of metallosis and associated conditions such as Adverse Local Soft Tissue Reactions ALTR and metal toxicity. The biosensors are placed within a prosthesis or in the vicinity of the prosthesis directly embedded in bone. The analyte to be examined would be ion debris such as Cobalt, Chromium or Titanium. A variety of bioreceptors can be chosen to recognize the ion debris and proper transduction mechanisms can convert the biorecognition of metal debris into an electrical or optical signal which is wirelessly transferred for interpretation and display.

A concept of PI-POCT biosensor diagnostics for infectious conditions and metallosis in orthopedics (PI-POCT-IMO) may be included in an embodiment of the present invention, and may provide structures and methods to quickly, accurately and with high degree of specificity and sensitivity (purely evidenced based) confirm or rule out these conditions, at the same time eliminating or reducing a need for multiple expensive tests and overreliance on surgeon judgement, which frequently leads to late diagnosis and damage to the patient.

FIG. 43 illustrates a biosensing system 4300 for assessing metallosis and trunnionosis including at or near an implant 4305 installed into a portion of bone 4310. One or more biosensors 4315 for biosensing of metal debris (e.g., cobalt, chromium, and titanium) in or around implant 4305.

Optimal Press Fit in Ligament Reconstruction

Embodiments described herein may make use of permanent wireless implantable biosensors for orthopedic arthroplasty. In U.S. Patent Application CONNECTIVE TISSUE GRAFTING, U.S. Application No. 62/742,851 filed 8 Oct. 2018 and CONNECTIVE TISSUE GRAFTING, U.S. Application No. 62/743,042 filed 9 Oct. 2018, both applications are hereby expressly incorporated by reference thereto in their entireties for all purposes, embodiments were described that make use of biosensors for assessment of tension, torsion and shear force of the reconstructed ligaments.

Electromechanical biosensors can be utilized at a reconstructed ligament/bone interface, in much the same manner which was described in press fit arthroplasty embodiments described herein, to assess a pressure (force) and interference fit (displacement) at this ligament/bone junction, to assure proper and optimal interference fit is obtained at the time of implantation. Increased interfacial pressures between graft and tunnel may lead to direct type healing which is preferred over indirect type healing.

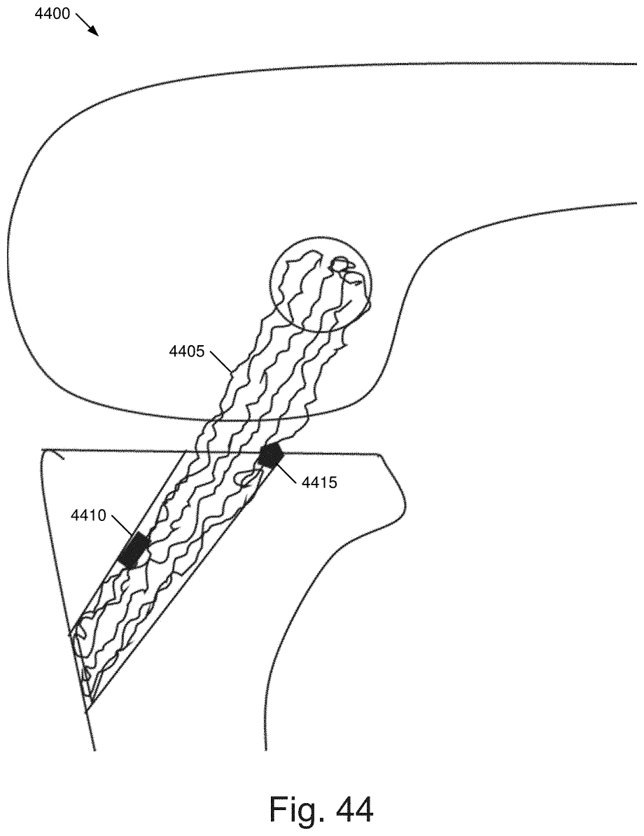

FIG. 44 illustrates a system 4400 for assessing optimal press fit in ligament reconstruction. An installed reconstructed ligament 4405 may include one or more of a displacement biosensor 4410 and/or a force biosensor 4415.

Poor Healing of Reconstructed Ligaments to Bone Assessment

Biosensors with displacement sensors such as LVDT can assess loosening and poor adhesion of the ligament graft to bone at the ligament/bone interface by measuring excessive micromotion at the ligament bone interface.

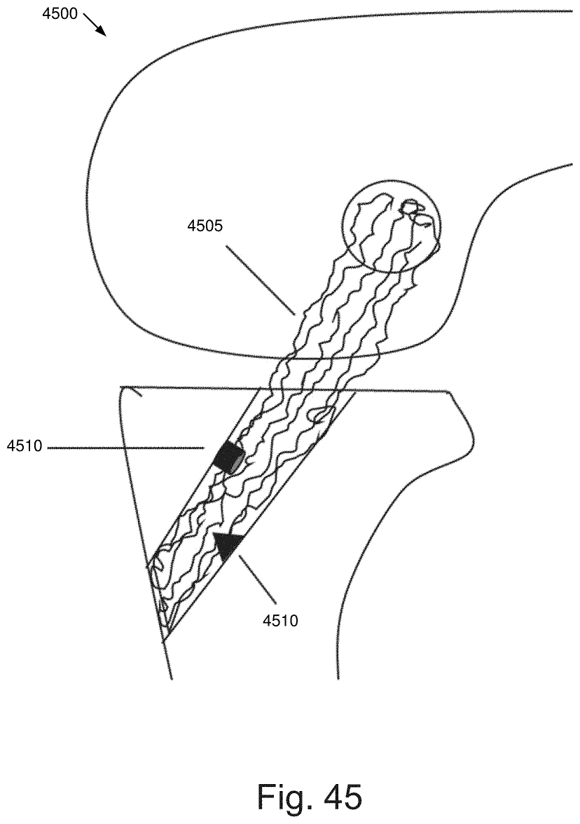

FIG. 45 illustrates a system 4500 for assessing poor healing of a reconstructed ligament 4505. Installed reconstructed ligament 4505, or a ligament/bone interface, may include one or more biosensors 4510 including an LVDT transducer.

Failure Mode Assessment of Reconstructed Ligament Grafts with PI-POCT biosensors

Electrochemical biosensors can also be used in the body of the ligament or at the tendon bone junction to evaluate a nature of damaging forces that may ultimately lead to failure of the ligament graft, which may include tension, torsion and or shear forces.

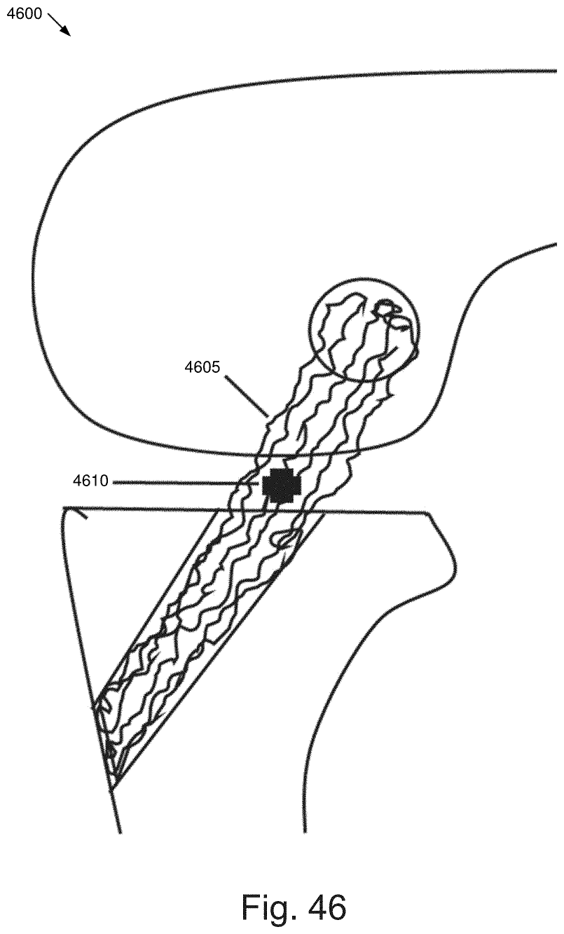

FIG. 46 illustrates a system 4600 for assessing various failure modes of a reconstructed ligament graft 4605. A biosensor 4610 may include one or more of a tension, shear, torsion, and/or displacement transducer.

In a case where a graft has failed before embarking on revision surgery. Certain causes of failure are easier to diagnose such as tunnel mal-positioning with the help of radiographic techniques such as X-ray and MRI studies. However, many times failures occur even with perfect tunnel placement. Frequently, in these scenarios the source of failure remains unknown. Repeat high force traumatic injury is one possibility and more likely in contact sports. Poor graft incorporation and healing is another possible source of failure. These scenarios can be sharply distinguished and clearly diagnosed with PI-POCT orthopedic monitoring of graft reconstructions. For example, if a graft does not heal and becomes loose over a period of 12 months (typical healing phase of an ACL graft), LVDT type biosensors employed at the time of graft implantation as PI-POCT systems may convey the information to the surgeon through wireless transmission during routine clinic visits. Alternatively, if a major traumatic event causes the rupture of a graft, a force biosensor implanted within the body of the ligament or at the ligament/bone junction may reveal the exact mechanism of injury by conveying the specific forces (tension, torsion, shear or combination thereof) involved in the ligament rupture.

The concept of post-operative monitoring of Prosthetic Interface Point of Care Testing PI-POCT naturally leads to the concept of Prophylactic Monitoring Point of Care Testing PM-POCT.

Traumatic and repetitive stress injuries in professional and recreational athletic population is very common.

Generally speaking traumatic high velocity injuries particularly in professional and collegiate athletes are more likely to be witnessed revealing the source and mechanism of injury. However, higher level understanding of these traumatic injuries can further be garnered with the use of PM-POCT devices applied to the tendons, bones and ligaments of high-level athletes to evaluate in real time the repetitive and traumatic stresses which produced a tear, rupture or failure of tissues. This ability can pin point certain biomechanical weaknesses in the athletes body (ligament, tendon, bone, muscle function and tightness) that can be addressed acutely in order to decrease the chance of major career ending injuries. In addition, gaining knowledge and the ability to accumulate data base on specific mechanisms of injury, through direct PM-POCT observation of the forces (tensile, compressive, torsional and shear or combination) involved in tissue failure, provides a level of understanding that has not been previously available. This can lead to development of better training techniques and protective orthotics for high level athletes.

Repetitive stress injuries, on the other hand are generally multifactorial, nonetheless frequently related to poor body mechanics. These include stress fractures of the lower extremity (i.e. tibia, metatarsals, calcaneus) and tendinitis problems (i.e. achilleas tendinitis and plantar fasciitis) and peripheral neuropathies (Morton's neuroma and tarsal tunnel syndrome).

As an example, poor body mechanics such as tight hip flexors and hamstrings in the proximal joints (hips) can constrain the range of motion of the lower extremity joints including hip, knee, ankle and feet, leading to stress fractures and/or tendinitis in the distal joints.

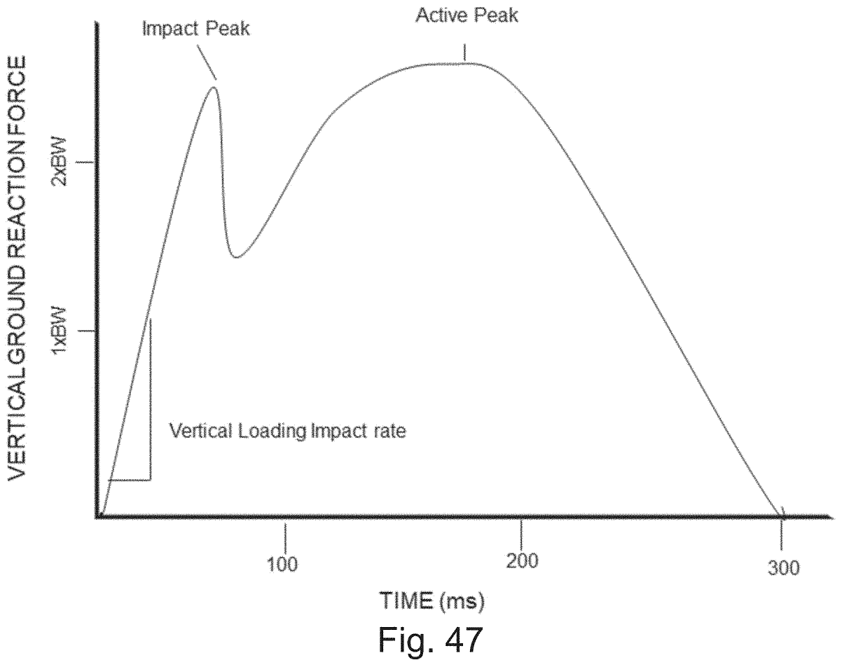

During gait cycle every time the foot lands on the ground the foot impacts the ground with certain amount of force which is countered by an equal and opposite amount of force applied by the ground to the foot called the ground reaction force GRF. The GRF has several components depending on the axis of movement being evaluated (including the x, y, and z axis). In the y axis or vertical GRF (straight up and down) motion, the foot experiences different stresses depending on whether the person strikes the ground initially with the hind foot or forefoot. A sample of the vertical GRF for a heel striker is illustrated in FIG. 47.

The Y axis is represented by body weight. The X axis is represented by milliseconds. The amount of time each foot is in contact with the ground varies for different runners but 300 ms is an average amount for a recreational runner. For a heel striker there are two distinct impacts. The impact peak which represents the initial force applied by the ground to the foot at the time of initial heel contact. The active peak which is a function of the force experienced by the foot during midstance. The slope of the impact peak (rise over run) is called vertical loading rate. The vertical loading rate represents how quickly the impact force is applied. A rapid sharp impact peak represents a large vertical load spread over a short time period. A gentler slope of indicates that the force being felt on the heel is being “diffused” or “spread” over a longer period of time.

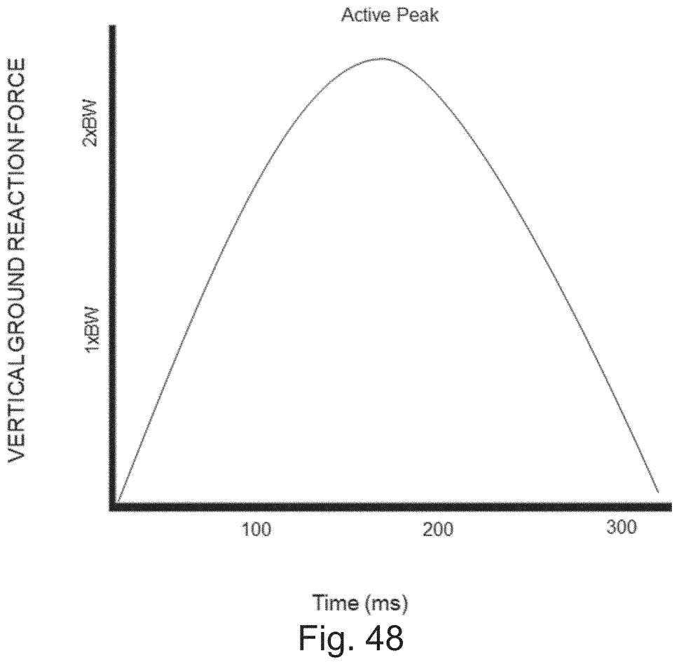

Forefoot runners, in contrast, do not have a large or significant impact peaks, illustrated in a gait chart in FIG. 48.

By eliminating the heel strike the forefoot runner has eliminated the impact peak, and the initial slope of the vertical loading rate is lower (smaller slope). The main reason for this transition is that the forefoot runner, now instead of directly impacting on the heel, has started to use the elaborate mechanical properties of the (foot/ankle) that allow absorption and release of energy and (i.e., an interplay of the arch of the foot, plantar fascia, achilleas tendon and gastric soleus muscle) to cushion the blow on the ground. The foot and ankle can collectively work as a very sophisticated shock absorber to absorb and store kinetic energy in the joints and muscles (during impact) and through and elaborate unwinding of the joints and windlass mechanism release the energy (during propulsion).

Therefore, when a runner runs with a very prominent heel strike, the natural shock absorbing mechanisms of the foot and ankle are not utilized, which leads to a “stiff system” with no compliance. This subsequently leads to increased stresses being transferred to the proximal bones and joints, which is one of the many mechanisms that leads to development of stress fractures, such ones in the tibia and calcaneus; as well as aggravation of the knee joint and development of tendon partial tears and tendinitis, such as achilleas tendinitis and plantar fasciitis.

The ability to apply, through small incisions or percutaneously, small biosensors within tendons, bones, and ligaments provides the possibility of Prophylactic Monitoring Point of Care Testing in orthopedics (PM-POCT).

It is well known that when patients generally present with early signs of tendinitis and stress fractures, that the X-rays and MRIs are typically negative and frequently provide minimal diagnostic value. The patient has a painful joint, bone or tendon (particularly with activity) and the studies are all negative. The physician typically has to make a “clinical diagnosis” of, for example, tendinitis but has no means of measuring the extent of this condition. A qualitative assessment based on experience is made. Currently there is no test that is sensitive and specific enough to diagnose or quantify “repetitive stress injuries” in the field of orthopedics.

Similar problems have arisen in repetitive stress injuries at work. In the day and age of computer science, time spent on computers and monitors has led to a significant number of upper extremity repetitive stress injuries, including tendinitis and peripheral neuropathies such as carpal tunnel syndrome and lateral epicondylitis. This has led to loss of productivity for society as well as pain and suffering for patients. There is no current method to diagnose or quantify these “work related” repetitive stress injuries at an early stage, and unfortunately, many of these patients are written off as malingerers.

PM-POCT provides the capability to apply biosensors within tendons, ligaments, and bone in order to monitor the amount of stress, micromotion, and inflammatory metabolites that typically accompany repetitive stress injuries. This capability can provide a means for early detection and correction of certain motions, positions and ergonomics that lead to these attritional injuries. The ability to collect precise data about repetitive stress injuries produces the ability to develop a database, that can be utilized to abstract formulas, algorithms and recommendations for prevention of these injuries. As well, the ability to store accumulated point of care POC information in large data bases, in combination with software development, can lead to the creation of derivative recommendations through machine learning and Artificial intelligence for injury prevention.

In the example of the heel striking runner discussed above, a biosensor applied to the calcaneus, tibia, plantar fascia, Achilleas tendon and the tarsometatarsal ligaments of the foot, with ability to measure force (loading), displacement (LVDT sensor), directionality (IMU inertial measuring units), and inflammatory metabolites (i.e., mast cells, macrophages, cytokines, chemokines, histamine, and the like) can not only detect whether microtears and inflammation are actually occurring through the (PM-POCT) process, but also determine WHY they are occurring.

In the example note above, the heel strike runner with very tight hamstring, adductor (groin muscle) and hip flexors (iliopsoas) will have a very short gait pattern (or stride length) without the ability to full flex and extend the hips producing less forward propulsion in the horizontal direction and more upward and downward motion leading to large vertical ground reaction forces GRF, and large vertical loading rate. This alteration in mechanics can clearly lead to a stress fracture of the calcaneus or tibia (and/or damage to the knee joints) for example. Similarly, any imbalance in the biomechanical function of the lower extremity musculotendinous system (typically tight and contracted muscle units) can lead to excessive loading (over repeated cycles) of certain bone and joints causing microtears, tendinitis, stress fractures and other repetitive stress injuries.

The ability to know this information through the PM-POCT process allows clinicians to make proper adjustments by focusing on the systems that are primarily responsible for causing the injury. For example, if the PM-POCT data reveal a correlation between lack of hip extension (tight iliopsoas) and excessive vertical loading rate and large impact peaks in a heel strike runner, emphasis on stretching of the hip flexors will be prescribed to decrease the chance of developing calcaneal and tibial stress fractures. Stretching of the hip flexors may be overemphasized over stretching of the adductor (groin) muscles and or other muscle groups such as the quadriceps or the Iliotibial band, particularly if these muscle groups are not excessively tight or contracted.

PM-POCT therefore allows insight to orthopedic and sports and work related repetitive stress injuries, through point of care testing, that was heretofore not conceivable and/or possible. This new capability allows early diagnosis and intervention of repetitive stress injuries, as well as a means for production of databases that can be exploited for better understanding of the musculoskeletal system mechanics and injuries through machine learning and Artificial Intelligence.

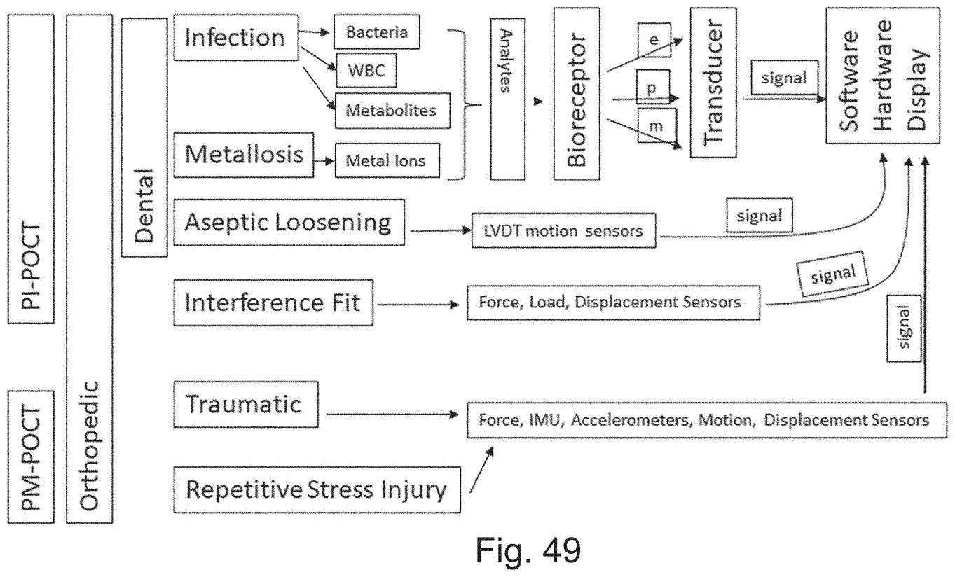

FIG. 49 illustrates a comprehensive diagram of point of care testing in orthopedic and dental surgery where PI-POCT and PM-POCT combine to provide real-time data from the immediate site of care for intra-operative decision making and post-operative monitoring of diseases, injuries, infections and implant failures.

Infection or Graft Rejection Assessment with PI-POCT Biosensors.

Similarly, to the embodiments described herein, embedded and implantable biosensors my be designed to detect infectious processes in arthroplasty for determination of infections processes in ligament reconstruction (i.e., ACL grafts) by measuring the infectious organisms directly or measuring the metabolic byproducts of the infectious condition.

The following references, expressly incorporated by reference hereto in their entireties for all purposes, support one or more of the concepts or ideas presented herein, including: 1) Udomkiat P, Dorr L D, Wan Z. Cementless hemispheric porous-coated sockets implanted with press-fit technique without screws: average ten-year follow-up. J Bone Joint Surg. 2002; 84A:1195.; 2) Takedani H, Whiteside L A, White S E, et al. The effect of screws and pegs on cementless acetabular fixation. Trans Orthop Res Soc 1991; 16:523; 3) lAhnfelt, L., P. Herberts, H. Malchau, and G. Andersson. Prognosis of total hip replacement: a swedish multicenter study of 4664 revisions. Acta Orthop. Scand. 61:2-26, 1990; 4) Corbett, K. L., E. Losina, A. A. Nti, J. J. Prokopetz, and J. N. Katz. Population-based rates of revision of primary total hip arthroplasty: a systematic review. PLoS ONE 5:e13520, 2010; 5) Huiskes, R. Failed innovation in total hip replacement: diagnosis and proposals for a cure. Acta Orthop. Scand. 64:699-716, 1993; 6) Harris, W. Aseptic loosening in total hip arthroplasty secondary to osteolysis induced by wear debris from titanium-alloy modular femoral heads. JBJS. 73:470-472, 1991; 7) Kobayashi, S., K. Takaoka, N. Saito, and K. Hisa. Factors affecting aseptic failure of fixation after primary charnley total hip arthroplasty multivariate survival analysis. JBJS. 79:1618-1627, 1997; 8) Lombardi Jr, A. V., T. Mallory, B. Vaughn, and P. Drouillard. Aseptic loosening in total hip arthroplasty secondary to osteolysis induced by wear debris from titanium-alloy modular femoral heads. JBJS. 71:1337-1342, 1989; 9) Huiskes, R. Failed innovation in total hip replacement: diagnosis and proposals for a cure. Acta Orthop. Scand. 64:699-716, 1993; 10) Clohisy, J. C., G. Calvert, F. Tull, D. McDonald, and W. J. Maloney. Reasons for revision hip surgery: a retrospective review. Clin. Orthop. Relat. Res. 429:188-192, 2004; 11) Kim, Y. S., J. J. Callaghan, P. B. Ahn, and T. D. Brown. Fracture of the acetabulum during insertion of an oversized hemispherical component. JBJS. 77:111-117, 1995; 12) Sharkey, P. F., W. J. Hozack, J. J. Callaghan, Y. S. Kim, D. J. Berry, A. D. Hanssen, and D. G. LeWallen. Acetabular fracture associated with cementless acetabular component insertion: a report of 13 cases. J. Arthro-plast.14:426-431, 1999; 13) Weeden, S. H. and W. G. Paprosky. Minimal 11-year follow-up of extensively porous-coated stems in femoral revision total hip arthroplasty. J. Arthroplast. 17:134-137, 2002; 14) Ulrich A D, Seyler T M, Bennett D, Celanois R E, Saleh K J, Thongtrangan I, Kuskowski M, Cheng E Y, Sharkey P F, Parvizi J, Stiehl J B, Mont M A. Total hip arthroplasties: What are the reasons for revision? International Orthopedics (SICOT) (2008) 32: 597-604; 15) Olory, B., E. Havet, A. Gabrion, J. Vernois, and P. Mertl. Comparative in vitro assessment of the primary stability of cementless press-fit acetabular cups. Acta Orthop. Belg. 70:31-37, 2004; 16) Meneghini, R. M., C. Meyer, C. A. Buckley, A. D. Hanssen, and D. G. Lewallen. Mechanical stability of novel highly porous metal acetabular components in revision total hip arthroplasty. J. Arthroplast. 25:337-341, 2010; 17) Fehring, K. A., J. R. Owen, A. A. Kurdin, J. S. Wayne, and W. A. Jiranek. Initial stability of press-fit acetabular components under rotational forces. J. Arthroplast 29:1038-1042, 2014; 18) Georgiou, A., and J. Cunningham. Accurate diagnosis of hip prosthesis loosening using a vibrational technique. Clin. Biomech. 16:315-323, 2001; 19) Balch C M, Freischlag J A, Shanafelt T, Stress and Burnout Among Surgeons. ARCH SURG/VOL 144 (NO. 4) April 2009; 20) Shanafelt T D, Balch C M, Bechamps G J, Tussell T, Dyrbye L, Satele D, Collicott P, Novotny P J, Sloan J, Freischlang J A Burnout and Career Satisfaction Among American Surgeons Ann Surg 2009; 250: 107-115; 21) Ulrich A D, Seyler T M, Bennett D, Celanois R E, Saleh K J, Thongtrangan I, Kuskowski M, Cheng E Y, Sharkey P F, Parvizi J, Stiehl J B, Mont M A. Total hip arthroplasties: What are the reasons for revision? International Orthopedics (SICOT) (2008) 32: 597-604; 22) Kurtz S, Ong K, Lau E, Mowat F, Halpern M, Projections of Primary and Revision Hip and Knee Arthroplasty in the United States from 2005 to 2030 JBJS (2007) Am 89: 780-785; 23) Nakasone S, Takao M, Nishii T, Sugano N, Incidence and Natural Course of Initial Polar Gaps in Birmingham Hip Resurfacing Cups. J of Arthroplasty Vol 27, (9) 1676-1682; and 24) Springer B D, Griffin W L, Fehring T K, Suarez J, Odum S, Thompson C Incomplete Seating of Press-Fit porous Coated Acetabular Components (2008) J of Arthroplasty Vol 23 (6) 121-126.

The following description relates to improvements in a wide range of prostheses installations into live bones of patients of surgeons. The following discussion focuses primarily on total hip replacement (THR) in which an acetabular cup prosthesis is installed into the pelvis of the patient. This cup is complementary to a ball and stem (i.e., a femoral prosthesis) installed into an end of a femur engaging the acetabulum undergoing repair.

Embodiments of the present invention may include one of more solutions to the above problems. The incorporated U.S. Pat. No. 9,168,154 includes a description of several embodiments, sometimes referred to herein as a BMD3 device, some of which illustrate a principle for breaking down large forces associated with the discrete blows of a mallet into a series of small taps, which in turn perform similarly in a stepwise fashion while being more efficient and safer. The BMD3 device produces the same displacement of the implant without the need for the large forces from the repeated impacts from the mallet. The BMD3 device may allow modulation of force required for cup insertion based on bone density, cup geometry, and surface roughness. Further, a use of the BMD3 device may result in the acetabulum experiencing less stress and deformation and the implant may experience a significantly smoother sinking pattern into the acetabulum during installation. Some embodiments of the BMD3 device may provide a superior approach to these problems, however, described herein are two problems that can be approached separately and with more basic methods as an alternative to, or in addition to, a BMD3 device. An issue of undesirable torques and moment arms is primarily related to the primitive method currently used by surgeons, which involves manually banging the mallet on the impaction plate. The amount of force utilized in this process is also non-standardized and somewhat out of control.

With respect to the impaction plate and undesirable torques, an embodiment of the present invention may include a simple mechanical solution as an alternative to some BMD3 devices, which can be utilized by the surgeon's hand or by a robotic machine. A direction of the impact may be directed or focused by any number of standard techniques (e.g., A-frame, C-arm or navigation system). Elsewhere described herein is a refinement of this process by considering directionality in the reaming process, in contrast to only considering it just prior to impaction. First, we propose to eliminate the undesirable torques by delivering the impacts by a sledgehammer device or a (hollow cylindrical mass) that travels over a stainless rod.

As noted in the background, the surgeon prepares the surface of the hipbone which includes attachment of the acetabular prosthesis to the pelvis. Conventionally, this attachment includes a manual implantation in which a mallet is used to strike a tamp that contacts some part of the acetabular prosthesis. Repeatedly striking the tamp drives the acetabular prosthesis into the acetabulum. Irrespective of whether current tools of computer navigation, fluoroscopy, robotics (and other intra-operative measuring tools) have been used, it is extremely unlikely that the acetabular prosthesis will be in the correct orientation once it has been seated to the proper depth by the series of hammer strikes. After manual implantation in this way, the surgeon then may apply a series of adjusting strikes around a perimeter of the acetabular prosthesis to attempt to adjust to the desired orientation. Currently such post-impaction result is accepted as many surgeons believe that post-impaction adjustment creates an unpredictable and unreliable change which does not therefore warrant any attempts for post-impaction adjustment.

In most cases, any and all surgeons including an inexperienced surgeon may not be able to achieve the desired orientation of the acetabular prosthesis in the pelvis by conventional solutions due to unpredictability of the orientation changes responsive to these adjusting strikes. As noted above, it is most common for any surgeon to avoid post-impaction adjustment as most surgeons understand that they do not have a reliable system or method for improving any particular orientation and could easily introduce more/greater error. The computer navigation systems, fluoroscopy, and other measuring tools are able to provide the surgeon with information about the current orientation of the prosthesis during an operation and after the prosthesis has been installed and its deviation from the desired orientation, but the navigation systems (and others) do not protect against torsional forces created by the implanting/positioning strikes. The prosthesis will find its own position in the acetabulum based on the axial and torsional forces created by the blows of the mallet. Even those navigation systems used with robotic systems (e.g., MAKE) that attempt to secure an implant in the desired orientation prior to impaction are not guaranteed to result in the installation of the implant at the desired orientation because the actual implanting forces are applied by a surgeon swinging a mallet to manually strike the tamp.

A Behzadi Medical Device (BMD) is herein described and enabled that eliminates this crude method (i.e., mallet, tamp, and surgeon-applied mechanical implanting force) of the prosthesis (e.g., the acetabular cup). A surgeon using the BMD is able to insert the prosthesis exactly where desired with proper force, finesse, and accuracy. Depending upon implementation details, the installation includes insertion of the prosthesis into patient bone, within a desired threshold of metrics for insertion depth and location) and may also include, when appropriate and/or desired, positioning at a desired orientation with the desired threshold further including metrics for insertion orientation). The use of the BMD reduces risks of fracturing and/or shattering the bone receiving the prosthesis and allows for rapid, efficient, and accurate (atraumatic) installation of the prosthesis. The BMD provides a viable interface for computer navigation assistance (also useable with all intraoperative measuring tools including fluoroscopy) during the installation as a lighter more responsive touch may be used.

The BMD encompasses many different embodiments for installation and/or positioning of a prosthesis and may be adapted for a wide range of prostheses in addition to installation and/or positioning of an acetabular prosthesis during THR.

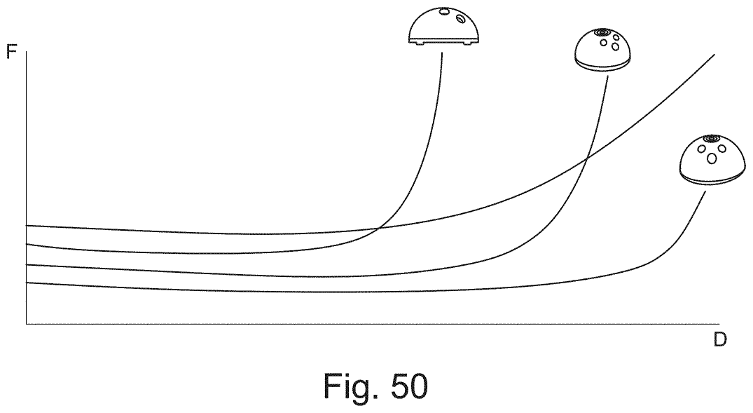

FIG. 50-55 illustrate a set of graphs of Force (y-axis) versus distance (x-axis). FIG. 50 illustrates a set of “cup prints” for a number of interactions between a cup and a cavity. Each combination of an implant (e.g., an acetabular cup) and its implant site (e.g., a reamed cavity in an acetabulum) has a resistive force (FR) that may be thought of as a particular cup print unique for that combination. FIG. 50 includes four such cup prints. Factors influencing the cup print include bone density (hard/soft), cup geometry (elliptical/spherical), cup surface preparation (e.g., roughness), and reaming preparation. Other sensors or sets of sensors may produce a more complex characteristic sensor print for processing of a prosthesis or portion of a prosthesis.

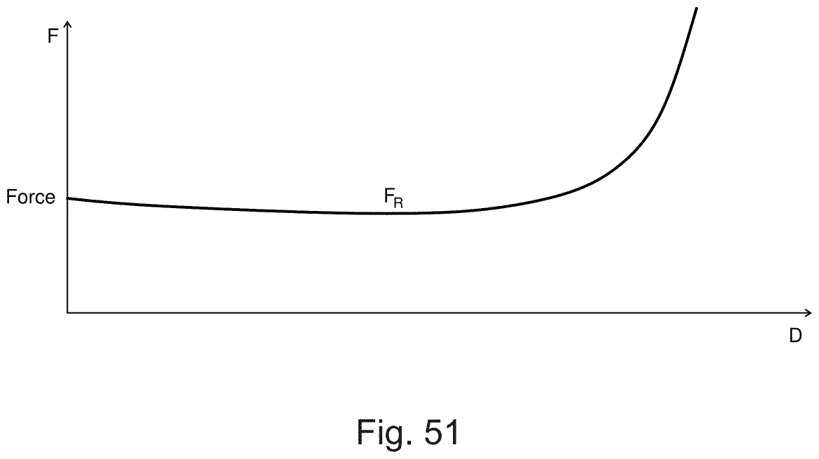

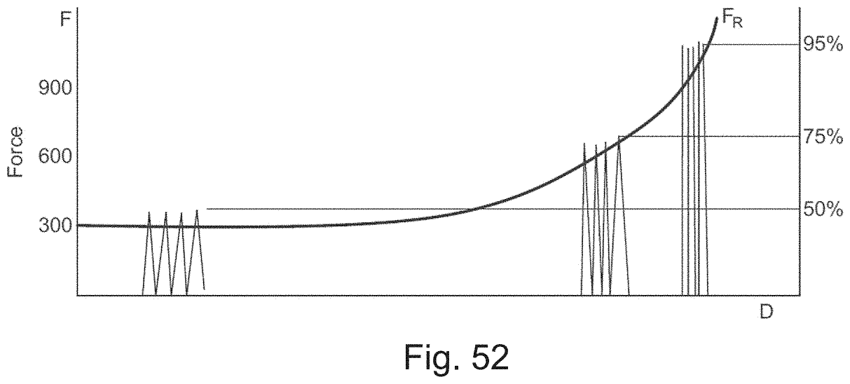

FIG. 51 illustrates a particular one representative cup print that relates to one cup/cavity interaction. FIG. 52 illustrates a controlled modulated installation force envelope superimposed over the cup print of FIG. 51. Typically the amplitude of the modulation increases as the implant is seated, with too great of force increasing a risk of fracture and tool little force increasing a risk of poor “seatedness”—a property of the implant relating to how well seated it is within its installation site.

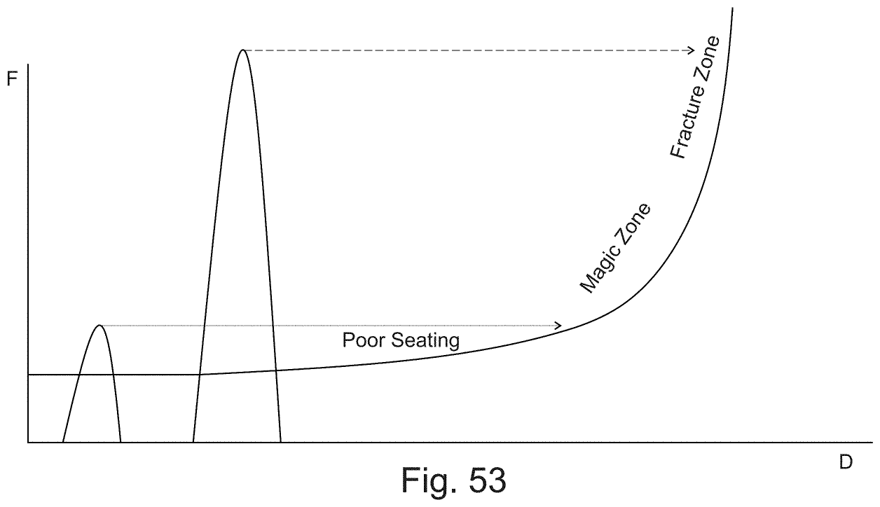

FIG. 53 illustrates an example installation force envelope that is representative of use of a mallet in its production. In this example, a surgeon “feels” and “listens” for the magic zone—adequate insertion and good pull-out force (seatedness) while being concerned with every strike that the installation site may fracture. The non-controlled mallet-applied installation force is shown superimposed over the cup print of FIG. 51.

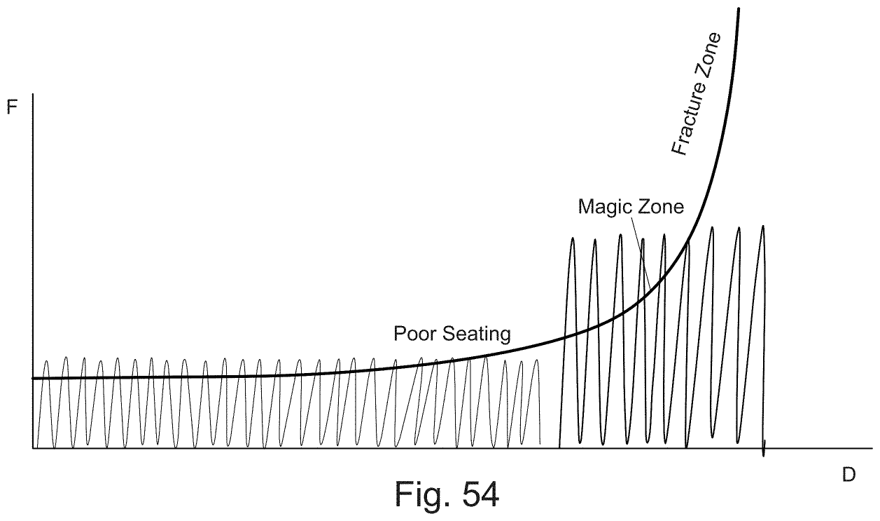

FIG. 54 illustrates an example installation force envelope that is representative of use of a BMD3 in its production. In this example, a surgeon dials into the magic zone by gradually changing the BMD3 force-applied profile. A BMD3 controlled modulated installation force envelope is shown superimposed over the cup print of FIG. 51. The surgeon is able to use a BMD3-type tool to walk the envelope (the contour of the installation force envelope) up and into the magic zone with greatly improved confidence of achieving the desired seatedness without greatly increasing a risk of fracture. Frictional forces may be decreased (effectively and realistically) at certain frequencies that may improve as the frequency increases (e.g., one to hundreds of Hertz or more, one-two kilohertz or more, and beyond to ultrasonic frequencies above two kilohertz). The reduced frictional forces may also enable easier alignment of the cup during and/or after insertion/placement.

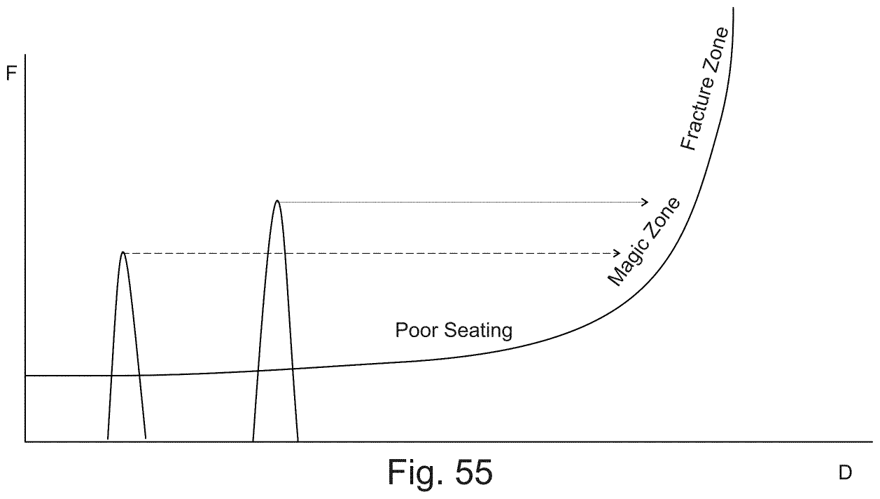

FIG. 55 illustrates an example installation force envelope that is representative of use of a BMD4 in its production. In this example, a surgeon dials into the magic zone by dialing the BMD4 force-applied profile. A BMD4 controlled modulated installation force envelope is shown superimposed over the cup print of FIG. 51. The surgeon is able to use a BMD4-type tool to dial into the magic zone (the contour of the installation force envelope) with greatly improved confidence of achieving the desired seatedness without greatly increasing a risk of fracture and while maintaining a desired alignment/positioning, for example, within the Lewinski range.

A hybrid BMD3/BMD4 embodiment may provide a hybrid controlled modulated installation force envelope that offers advantages of both BMD3 and BMD4.

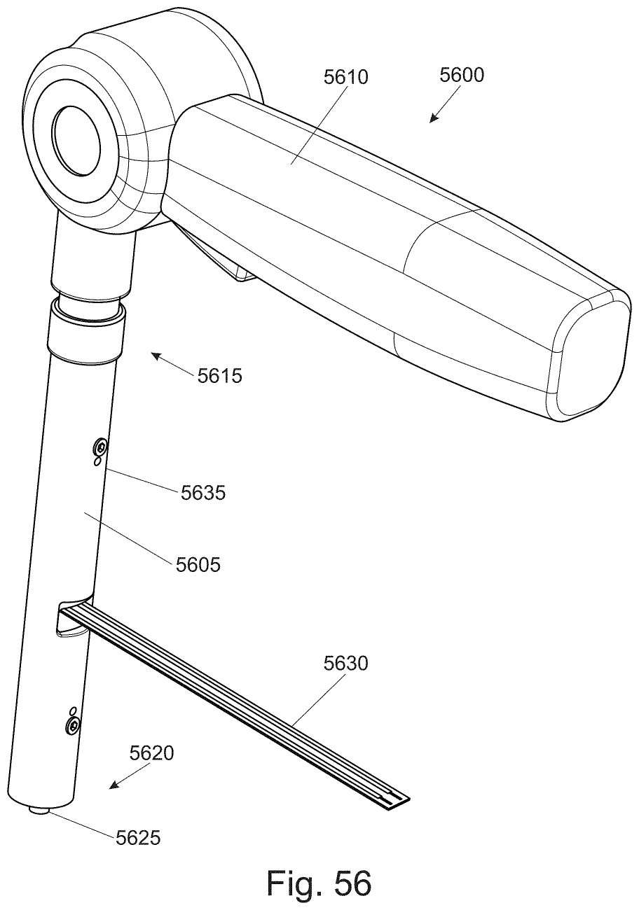

FIG. 56 illustrates a representative installation gun. The installation gun may be operable with operable using pneumatics, though other implementations may use other mechanisms including motors for creating a desired vibratory motion of prosthesis to be installed.

The installation gun may be used to control precisely one or both of (i) insertion, and (ii) abduction and anteversion angles of a prosthetic component. Installation gun 100 preferably allows both installation of an acetabular cup into an acetabulum at a desired depth and orientation of the cup for both abduction and anteversion to desired values.

The installation gun may include a controller with a handle supporting an elongate tube that terminates in an adapter that engages a cup. Operation of a trigger may initiate a motion of the elongate tube. This motion is referred to herein as an installation force and/or installation motion that is much less than the impact force used in a conventional replacement process. An exterior housing allows the operator to hold and position the prosthesis (e.g., the cup) while elongate tube moves within. Some embodiments may include a handle or other grip in addition to or in lieu of the housing that allows the operator to hold and operate installation gun without interfering with the mechanism that provides a direct transfer of installation motion to the prosthesis. The illustrated embodiment may include the prosthesis held securely by adapter allowing a tilting and/or rotation of gun about any axis to be reflected in the position/orientation of the secured prosthesis.

The installation motion includes constant, cyclic, periodic, and/or random motion (amplitude and/or frequency) that allows the operator to install the cup into the desired position (depth and orientation) without application of an impact force. There may be continuous movement or oscillations in one or more of six degrees of freedom including translation(s) and/or rotation(s) of adapter 146 about the X, Y, Z axes (e.g., oscillating translation(s) and/or oscillating/continuous rotation(s) which could be different for different axes such as translating back and forth in the direction of the longitudinal axis of the central support while rotating continuously around the longitudinal axis). This installation motion may include continuous or intermittent very high frequency movements and oscillations of small amplitude that allow the operator to easily install the prosthetic component in the desired location, and preferably also to allow the operator to also set the desired angles for abduction and anteversion.

In some implementations, the controller includes a stored program processing system that includes a processing unit that executes instructions retrieved from memory. Those instructions could control the selection of the motion parameters autonomously to achieve desired values for depth, abduction and anteversion entered into by the surgeon or by a computer aided medical computing system such as the computer navigation system. Alternatively those instructions could be used to supplement manual operation to aid or suggest selection of the motion parameters.

For more automated systems, consistent and unvarying motion parameters are not required and it may be that a varying dynamic adjustment of the motion parameters better conform to an adjustment profile of the cup installed into the acetabulum and status of the installation. An adjustment profile is a characterization of the relative ease by which depth, abduction and anteversion angles may be adjusted in positive and negative directions. In some situations these values may not be the same and the installation gun could be enhanced to adjust for these differences. For example, a unit of force applied to pure positive anteversion may adjust anteversion in the positive direction by a first unit of distance while under the same conditions that unit of force applied to pure negative anteversion may adjust anteversion in the negative direction by a second unit of distance different from the first unit. And these differences may vary as a function of the magnitude of the actual angle(s). For example, as the anteversion increases it may be that the same unit of force results in a different responsive change in the actual distance adjusted. The adjustment profile when used helps the operator when selecting the actuators and the impact force(s) to be applied. Using a feedback system of the current real-time depth and orientation enables the adjustment profile to dynamically select/modify the motion parameters appropriately during different phases of the installation. One set of motion parameters may be used when primarily setting the depth of the implant and then another set used when the desired depth is achieved so that fine tuning of the abduction and anteversion angles is accomplished more efficiently, all without use of impact forces in setting the depth and/or angle adjustment(s).

This device better enables computer navigation as the installation/adjustment forces are reduced as compared to the impacting method. This makes the required forces more compatible with computer navigation systems used in medical procedures which do not have the capabilities or control systems in place to actually provide impacting forces for seating the prosthetic component. And without that, the computer is at best relegated to a role of providing after-the-fact assessments of the consequences of the surgeon's manual strikes of the orthopedic mallet. (Also provides information before and during the impaction. It is a problem that the very act of impaction introduces variability and error in positioning and alignment of the prosthesis.

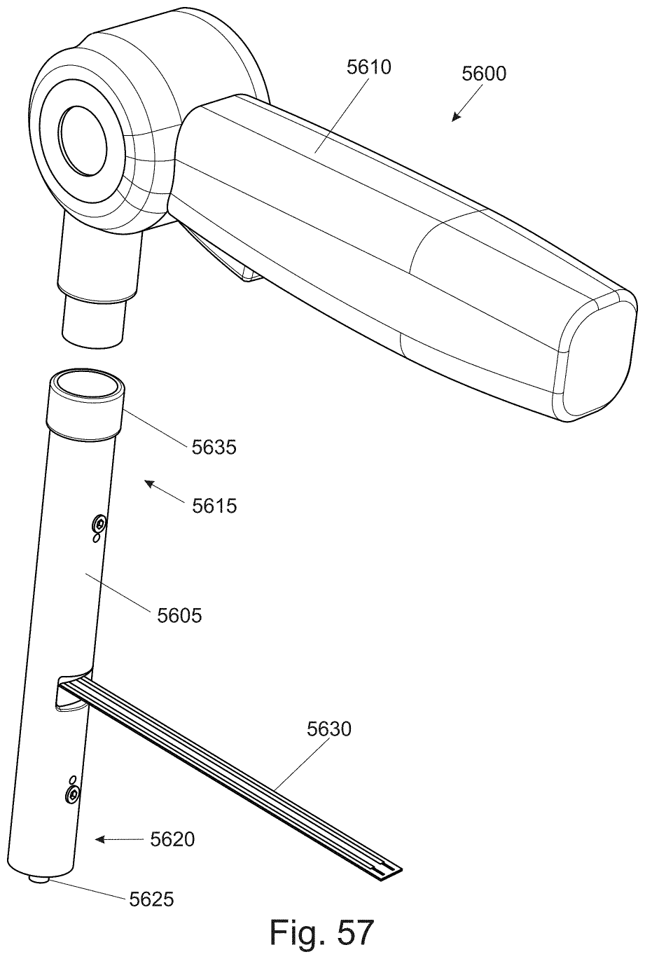

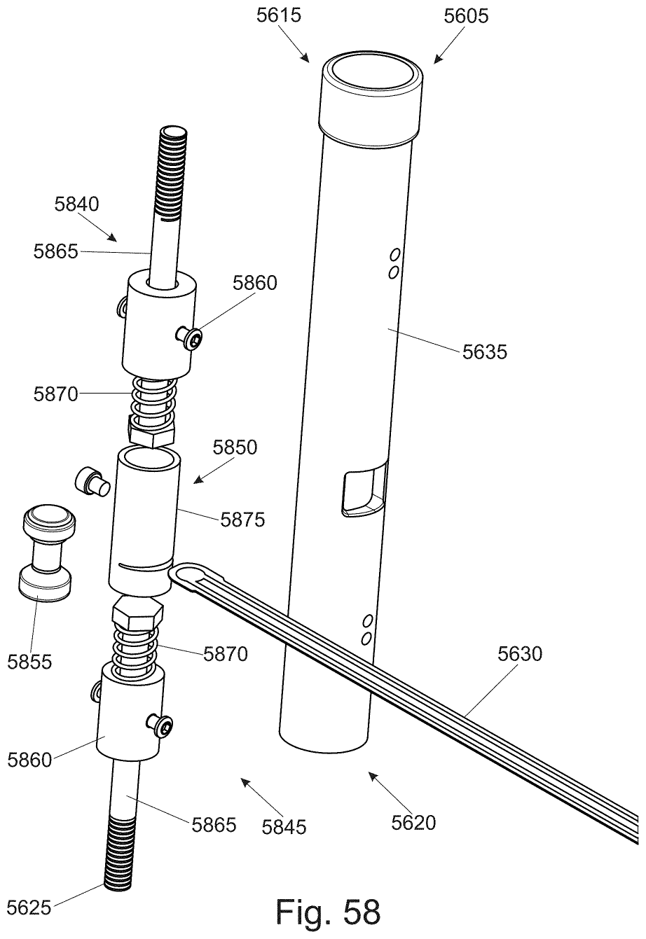

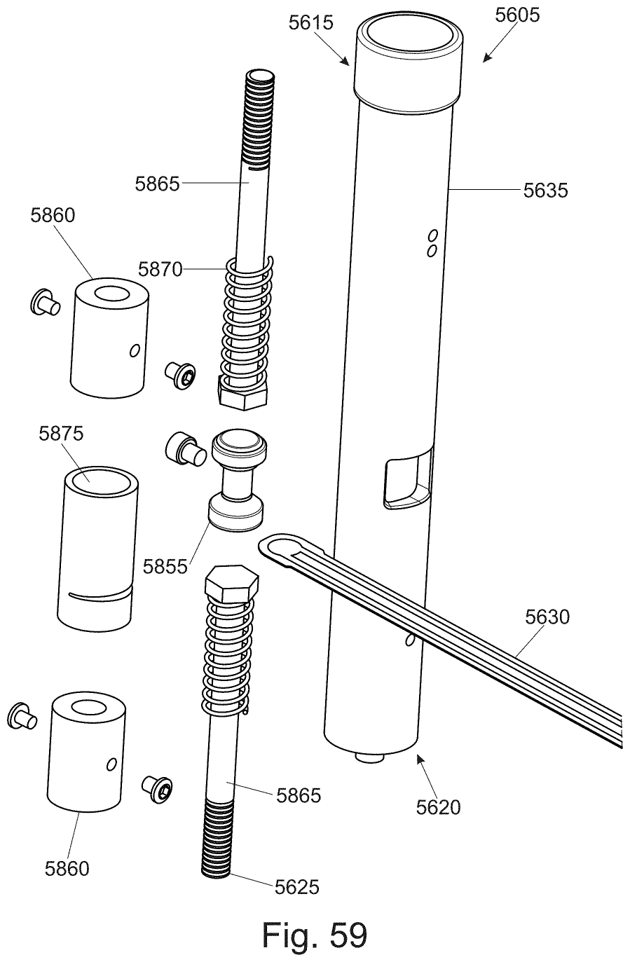

FIG. 56 illustrates a representative installation system 5600 including a pulse transfer assembly 5605 and an oscillation engine 5610; FIG. 57 illustrates a disassembly of second representative installation system 5600; FIG. 58 illustrates a first disassembly view of pulse transfer assembly 5605; and FIG. 59 illustrates a second disassembly view of pulse transfer assembly 5605 of installation system 5600.

Installation system 5600 is designed for installing a prosthesis that, in turn, is configured to be implanted into a portion of bone at a desired implantation depth. The prosthesis includes some type of attachment system (e.g., one or more threaded inserts, mechanical coupler, link, or the like) allowing the prosthesis to be securely and rigidly held by an object such that a translation and/or a rotation of the object about any axis results in a direct corresponding translation and/or rotation of the secured prosthesis.

Oscillation engine 5610 includes a controller coupled to a vibratory machine that generates an original series of pulses having a generation pattern. This generation pattern defines a first duty cycle of the original series of pulses including one or more of a first pulse amplitude, a first pulse direction, a first pulse duration, and a first pulse time window. This is not to suggest that the amplitude, direction, duration, or pulse time window for each pulse of the original pulse series are uniform with respect to each other. Pulse direction may include motion having any of six degrees of freedom—translation along one or more of any axis of three orthogonal axes and/or rotation about one or more of these three axes. Oscillation engine 5610 includes an electric motor powered by energy from a battery, though other motors and energy sources may be used.

Pulse transfer assembly 5605 includes a proximal end 5615 coupled to oscillation engine 5610 and a distal end 5620, spaced from proximal end 5620, coupled to the prosthesis using a connector system 5625. Pulse transfer assembly 5605 receives the original series of pulses from oscillation engine 5610 and produces, responsive to the original series of pulses, an installation series of pulses having an installation pattern. Similar to the generation pattern, the installation pattern defines a second duty cycle of the installation series of pulses including a second pulse amplitude, a second pulse direction, a second pulse duration, and a second pulse time window. Again, this is not to suggest that the amplitude, direction, duration, or pulse time window for each pulse of the installation pulse series are uniform with respect to each other, nor does it imply that they are non-uniform. Pulse direction may include motion having any of six degrees of freedom—translation along one or more of any axis of three orthogonal axes and/or rotation about one or more of these three axes.

For some embodiments of pulse transfer assembly 5605, the installation series of pulses will be strongly linked to the original series and there will be a close match, if not identical match, between the two series. Some embodiments may include a more complex pulse transfer assembly 5605 that produces an installation series that is more different, or very different, from the original series.

Connector system 5625 (e.g., one or more threaded studs complementary to the threaded inserts of the prosthesis, or other complementary mechanical coupling system) is disposed at proximal end 5620. Connector system 5625 is configured to secure and rigidly hold the prosthesis. In this way, the attached prosthesis becomes a secured prosthesis when engaged with connector system 5625.

Pulse transfer assembly 5605 communicates the installation series of pulses to the secured prosthesis and produces an applied series of pulses that are responsive to the installation series of pulses. Similar to the generation pattern and the installation pattern, the applied pattern defines a third duty cycle of the applied series of pulses including a third pulse amplitude, a third pulse direction, a third pulse duration, and a third pulse time window. Again, this is not to suggest that the amplitude, direction, duration, or pulse time window for each pulse of the applied pulse series are uniform with respect to each other. Pulse direction may include motion having any of six degrees of freedom—translation along one or more of any axis of three orthogonal axes and/or rotation about one or more of these three axes.

For some embodiments of pulse transfer assembly 5605, the applied series of pulses will be strongly linked to the original series and/or the installation series and there will be a close, if not identical, match between the series. Some embodiments may include a more complex pulse transfer assembly 705 that produces an applied series that is more different, or very different, from the original series and/or the installation series. In some embodiments, for example one or more components may be integrated together (for example, integrating oscillation engine 5610 with pulse transfer assembly 5605) so that the first series and the second series, if they exist independently are nearly identical if not identical).

The applied series of pulses are designed to impart a vibratory motion to the secured prosthesis that enable an installation of the secured prosthesis into the portion of bone to within 95% of the desired implantation depth without a manual impact. That is, in operation, the original pulses from oscillation engine 5610 propagate through pulse transfer assembly 5605 (with implementation-depending varying levels of fidelity) to produce the vibratory motion to the prosthesis secured to connector system 5625. In a first implementation, the vibratory motion allows implanting without manual impacts on the prosthesis and in a second mode an orientation of the implanted secured prosthesis may be adjusted by rotations of installation system 5600 while the vibratory motion is active, also without manual impact. In some implementations, the pulse generation may produce different vibratory motions optimized for these different modes.

Installation system 5600 includes an optional sensor 5630 (e.g., a flex sensor or the like) to provide a measurement (e.g., quantitative and/or qualitative) of the installation pulse pattern communicated by pulse transfer assembly 5605. This measurement may be used as part of a manual or computerized feedback system to aid in installation of a prosthesis. For example, in some implementations, the desired applied pulse pattern of the applied series of pulses (e.g., the vibrational motion of the prosthesis) may be a function of a particular installation pulse pattern, which can be measured and set through sensor 5630. In addition to, or alternatively, other sensors may aid the surgeon or an automated installation system operating installation system 5600, such as a bone density sensor or other mechanism to characterize the bone receiving the prosthesis to establish a desired applied pulse pattern for optimal installation. In some implementations, sensor 5630 measures force magnitude as part of the installation pulse pattern.

The disassembled views of FIG. 58 and FIG. 59 detail a particular implementation of pulse transfer assembly 5605, it being understood that there are many possible ways of creating and communicating an applied pulse pattern responsive to a series of generation pulses from an oscillation engine. The illustrated structure of FIG. 58 and FIG. 59 generate primarily longitudinal/axial pulses in response to primarily longitudinal/axial generation pulses from oscillation engine 5610.

Pulse transfer assembly 5605 includes an outer housing 5635 containing an upper transfer assembly 5840, a lower transfer assembly 5845 and a central assembly 5850. Central assembly 5850 includes a double anvil 5855 that couples upper transfer assembly 5840 to lower transfer assembly 5845. Outer housing 5835 and central assembly 5850 each include a port allowing sensor 5830 to be inserted into central assembly 5850 between an end of double anvil 5855 and one of the upper/lower transfer assemblies.

Upper transfer assembly 5840 and lower transfer assembly 5845 each include a support 5860 coupled to outer housing 5835 by a pair of connectors. A transfer rod 5865 is moveably disposed through an axial aperture in each support 960, with each transfer rod 5865 including a head at one end configured to strike an end of double anvil 5855 and a coupling structure at a second end. A compression spring 5870 is disposed on each transfer rod 5865 between support 5860 and the head. The coupling structure of upper transfer assembly 5840 cooperates with oscillation engine 5610 to receive the generated pulse series. The coupling structure of lower transfer assembly 5845 includes connector system 5625 for securing the prosthesis. Some embodiments may include an adapter, not shown, that adapts connector system 5625 to a particular prosthesis, different adapters allowing use of pulse transfer assembly 5605 with different prosthesis.

Central assembly 5850 includes a support 5875 coupled to outer housing 5635 by a connector and receives double anvil 5855 which moves freely within support 5875. The heads of the upper transfer assembly and the lower transfer assembly are disposed within support 5875 and arranged to strike corresponding ends of double anvil 5855 during pulse generation.

In operation, oscillation engine 5610 generates pulses that are transferred via pulse transfer assembly 5605 to the prosthesis secured by connector system 5625. The pulse transfer assembly 5605, via upper transfer assembly 5840, receives the generated pulses using transfer rod 5865. Transfer rod 5865 of upper transfer assembly 5840 moves within support 5860 of upper transfer assembly 5840 to communicate pulses to double anvil 5855 moving within support 5875. Double anvil 5855, in turn, communicates pulses to transfer rod 5865 of lower transfer assembly 5845 to produce vibratory motion of a prosthesis secured to connector system 5625. Transfer rods 5865 move, in this illustrated embodiment, primarily longitudinally/axially within outer housing 5635 (a longitudinal axis defined as extending between proximate end 5615 and distal end 5620. In this way, the surgeon may use outer housing 5635 as a hand hold when installing and/or positioning the vibrating prosthesis.

The use of discrete transfer portions (e.g., upper, central, and lower transfer assemblies) for pulse transfer assembly 5605 may allow a form of loose coupling between oscillation engine 5610 and a secured prosthesis. In this way pulses from oscillation engine 5610 are converted into a vibratory motion of the prosthesis as it is urged into the bone during operation. Some embodiments may provide a stronger coupling by directly securing one component to another, or substituting a single component for a pair of components.

The embodiment of FIG. 56 has demonstrated insertion of a prosthetic cup into a bone substitute substrate with ease and a greatly reduced force as compared to use of a mallet and tamp, especially as no impaction was required. While the insertion was taking place and vibrational motion was present at the prosthesis, the prosthesis could be positioned with relative ease by torqueing on a handle/outer housing to an exact desired alignment/position. The insertion force is variable and ranges between 20 to 800 pounds of force. Importantly the potential for use of significantly smaller forces in application of the prosthesis (in this case the acetabular prosthesis) in bone substrate with the present invention is demonstrated to be achievable.

Installation system 5600 may include an oscillation engine producing pulses at approximately 60 Hz. System 5600 operated at 60 Hz. In testing, approximately 4 seconds of operation resulted in a desired insertion and alignment of the prosthesis (meaning about 240 cycles of the oscillation engine). Conventional surgery using a mallet striking a tamp to impact the cup into place is generally complete after 10 blows of the mallet/hammer.

### Experimental

System 5600 was tested in a bone substitute substrate with a standard Zimmer acetabular cup using standard technique of under reaming a prepared surface by 1 mm and inserting a cup that was one millimeter larger. The substrate was chosen as the best option available to study this concept, namely a dense foam material. It was recognized that certain properties of bone would not be represented here (e.g. less of an ability of the bone substrate to stretch before failure).

FIG. 56 demonstrated easy insertion and positioning of the prosthetic cup within the chosen substrate. We were able to move the cup in the substrate with relative ease. There was no requirement for a mallet or hammer for application of a large impact. These experiments demonstrated that the prosthetic cups could be inserted in bone substitute substrates with significantly less force and more control than what could be done with blows of a hammer or mallet. We surmise that the same phenomena can be reproduced in human bone. We envision the prosthetic cup being inserted with ease with very little force.

Additionally, we believe that simultaneously, while the cup is being inserted, the position of the cup can be adjusted under direct visualization with any intra-operative measurement system (navigation, fluoroscopy, and the like). This invention provides a system that allows insertion of a prosthetic component with NON-traumatic force (insertion) as opposed to traumatic force (impaction).

Experimental Configuration—System 5600

Oscillation engine 5610 included a Craftsman GO Hammerhead nailed used to drive fairly large framing nails into wood in confined spaces by applying a series of small impacts very rapidly in contrast to application of few large impacts.

The bone substitute was 15-pound density urethane foam to represent the pelvic acetabulum. It was shaped with a standard cutting tool commonly used to clean up a patient's damaged acetabulum. A 54 mm cup and a 53 mm cutter were used in testing.

In one test, the cup was inserted using a mallet and tamp, with impaction complete after 7 strikes. Re-orientation of the cup was required by further strikes on an periphery of the cup after impaction to achieve a desired orientation. It was qualitatively determined that the feel and insertion were consistent with impaction into bone.

An embodiment of system 5600 was used in lieu of the mallet and tamp method. Several insertions were performed, with the insertions found to be much more gradual; allowing the cup to be guided into position (depth and orientation during insertion). Final corrective positioning is easily achievable using lateral hand pressure to rotate the cup within the substrate while power was applied to the oscillation engine.

Further testing using the sensor included general static load detection done to determine the static (non-impact) load to push the cup into the prepared socket model. This provided a baseline for comparison to the impact load testing. The prosthesis was provided above a prepared socket with a screw mounted to the cup to transmit a force applied from a bench vise. The handle of the vice was turned to apply an even force to compress the cup into the socket until the cup was fully seated. The cup began to move into the socket at about an insertion force of ˜200 pounds and gradually increased as diameter of cup inserted into socket increased to a maximum of 375 pounds which remained constant until the cup was fully seated.

Installation system 5600 was next used to install the cup into a similarly prepared socket. Five tests were done, using different frame rates and setup procedures, to determine how to get the most meaningful results. All tests used a 54 mm acetabular Cup. The oscillation engine ran at an indicated 60 impacts/second. The first two tests were done at 2,000 frames/second, which wasn't fast enough to capture all the impact events, but helped with designing the proper setup. Test 3 used the oscillation engine in an already used socket, 4,000 frames per second. Test 4 used the oscillation engine in an unused foam socket at 53 mm, 4,000 frames per second.

Test 3: In already compacted socket, the cup was pulsed using the oscillation engine and the pulse transfer assembly. Recorded strikes between 500 and 800 lbs., with an average recorded pulse duration 0.8 Ms.

Test 4: Into an unused 53 mm socket, the cup was pulsed using the oscillation engine and the pulse transfer assembly. Recorded impacts between 250 and 800 lbs., and an average recorded pulse duration 0.8 Ms. Insertion completed in 3.37 seconds, 202 impact hits.

Test 5: Into an unused 53 mm socket, the cup was inserted with standard hammer (for reference). Recorded impacts between 500 and 800 lbs., and an average recorded pulse duration 22.0 Ms. Insertion completed in 4 seconds using 10 impact hits for a total pressure time of 220 Ms. This test was performed rapidly to complete it in 5 seconds for good comparability with tests 3 and 4 used 240 hits in 4 seconds, with a single hit duration of 0.8 MS, for a total pressure time of 192 Ms.

Additionally, basic studies can further be conducted to correlate a density and a porosity of bone at various ages (e.g., through a cadaver study) with an appropriate force range and vibratory motion pattern required to insert a cup using the present invention. For example, a surgeon will be able to insert sensing equipment in patient bone, or use other evaluative procedures, (preoperative planning or while performing the procedure for example) to assess porosity and density of bone. Once known, the density or other bone characteristic is used to set an appropriate vibratory pattern including a force range on an installation system, and thus use a minimal required force to insert and/or position the prosthesis.

BMD is a “must have” device for all medical device companies and surgeons. Without BMD the Implantation problem is not addressed, regardless of the recent advances in technologies in hip replacement surgery (i.e.; Navigation, Fluoroscopy, MAKE/robotics, accelerometers/gyro meters, etc.). Acetabular component (cup) positioning remains the biggest problem in hip replacement surgery. Implantation is the final step where error is introduced into the system and heretofore no attention has been brought to this problem. Current technologies have brought significant awareness to the position of the implants within the pelvis during surgery, prior to impaction. However, these techniques do not assist in the final step of implantation.

BMD allows all realtime information technologies to utilize (a tool) to precisely and accurately implant the acetabular component (cup) within the pelvic acetabulum. BMD device coupled with use of navigation technology and fluoroscopy and (other novel measuring devices) is the only device that will allow surgeons from all walks of life, (low volume/high volume) to perform a perfect hip replacement with respect to acetabular component (cup) placement. With the use of BMD, surgeons can feel confident that they are doing a good job with acetabular component positioning, achieving the “perfect cup” every time. Hence the BMD concept eliminates the most common cause of complications in hip replacement surgery which has forever plagued the surgeon, the patients and the society in general.

It is known to use ultrasound devices in connection with some aspects of THR, primarily for implant removal (as some components may be installed using a cement that may be softened using ultrasound energy). There may be some suggestion that some ultrasonic devices that employ “ultrasound” energy could be used to insert a prosthesis for final fit, but it is in the context of a femoral component and it is believed that these devices are not presently actually used in the process). Some embodiments of BMD, in contrast, can simply be a vibratory device (non ultrasonic, others ultrasonic, and some hybrid impactful and vibratory), and is more profound than simply an implantation device as it is most preferably a positioning device for the acetabular component in THR. Further, there is a discussion that ultrasound devices may be used to prepare bones for implanting a prosthesis. BMD may address preparation of the bone in some aspects of the present invention.

Some embodiments BMD include devices that concern themselves with proper installation and positioning of the prosthesis (e.g., an acetabular component) at the time of implanting of the prosthesis. Very specifically, it uses some form of vibratory energy coupled with a variety of “realtime measurement systems” to POSITION the cup in a perfect alignment with minimal use of force. A prosthesis, such as for example, an acetabular cup, resists insertion. Once inserted, the cup resists changes to the inserted orientation. The BMDs of the present invention produce an insertion vibratory motion of a secured prosthesis that reduces the forces resisting insertion. In some implementations, the BMD may produce a positioning vibratory motion that reduces the forces resisting changes to the orientation. There are some implementations that produce both types of motion, either as a single vibratory profile or alternative profiles. In the present context for purposes of the present invention, the vibratory motion is characterized as “floating” the prosthesis as the prosthesis can become much simpler to insert and/or re-orient while the desired vibratory motion is available to the prosthesis. Some embodiments are described as producing vibrating prosthesis with a predetermined vibration pattern. In some implementations, the predetermined vibration pattern is predictable and largely completely defined in advance. In other implementations, the predetermined vibration pattern includes randomized vibratory motion in one or more motion freedoms of the available degrees of freedom (up to six degrees of freedom). That is, whichever translation or rotational freedom of motion is defined for the vibrating prosthesis, any of them may have an intentional randomness component, varying from large to small. In some cases, the randomness component in any particular motion may be large and in some cases predominate the motion. In other cases, the randomness component may be relatively small as to be barely detectable.

A tool, among others, that may support the force measurement includes an axially-impactful Behzadi Medical Device (BMD4). The BMD4 may include a moveable hammer sliding axially and freely along a rod. The rod may include a proximal stop and a distal stop. These stops that may be integrated into rod allow transference of force to rod when the hammer strikes the distal stop. At a distal end of the rod, the device includes an attachment system for a prosthesis. For example, when the prosthesis includes an acetabular cup having a threaded cavity, the attachment system may include a complementary threaded structure that screws into the threaded cavity. The illustrated design of the device allows only a perfect axial force to be imparted. The surgeon cannot deliver a blow to the edge of an impaction plate. Therefore the design of this instrument is in and of itself protective, eliminating a problem of “surgeon's mallet hitting on the edge of the impaction plate” or other mis-aligned force transference, and creating undesirable torques, and hence unintentional mal-alignment of the prosthesis from an intended position/orientation. This embodiment may be modified to include a vibratory engine as described herein.

The embodiment may include a pressure sensor to provide feedback during installation. With respect to management of the vibration/force required for some of these tasks, it is noted that with current techniques (the use of the mallet) the surgeon has no indication of how much force is being imparted onto the implant and/or the implant site (e.g., the pelvis). Laboratory tests may be done to estimate what range of force should be utilized in certain age groups (as a rough guide) and then fashioning a device 1100, for example a modified sledgehammer or a cockup gun to produce just the right amount of force and/or producing a predetermined force of a known magnitude. Typically the surgeon may use up to 2000 N to 3000 N of force to impact a cup into the acetabular cavity. Also, since some embodiments cannot deliver the force in an incremental fashion as described in association with the BMD3 device, the device may include a stopgap mechanism. Some embodiments of the BMD3 device have already described the application of a sensor in the body of the impaction rod. The device may include a system/assembly embedded in the device, for example proximate the rod near the distal end, and used to provide valuable feedback information to the surgeon. The pressure sensor can let the surgeon know when the pressures seem to have maximized, whether used for the insertion of an acetabular cup, or any other implant including knee and shoulder implants and rods used to fix tibia and femur fractures. When the pressure sensor is not showing an advance or increase in pressure readings and has plateaued, the surgeon may determine it is time to stop operation/impacting. An indicator, for example an alarm can go off or a red signal can show when maximal peak forces are repeatedly achieved. As noted above, the incorporated patents describe a presence of a pressure sensor in an installation device, the presence of which was designed as part of a system to characterize an installation pulse pattern communicated by a pulse transfer assembly. The disclosure here relates to a pressure sensor provided not to characterize the installation vibration/pulse pattern but to provide an in situ feedback mechanism to the surgeon as to a status of the installation, such as to reduce a risk of fracturing the installation site. Some embodiments may also employ this pressure sensor for multiple purposes including characterization of an applied pulse pattern such as, for example, when the device includes automated control of an impacting engine coupled to the hammer. Other embodiments of this invention may dispose the sensor or sensor reading system within a handle or housing of the device rather than in the central rod or shaft.

Previous work has sought to address the two problems noted above culminating in a series of devices identified as BMD2, BMD3, and BMD4, sometimes described herein as including or providing an insertion agency. Each of these systems attempts to address the two problems noted above with different and novel methods.

The BMD2 concept proposed a system of correcting a cup (acetabular implant) that had already been implanted in a mis-aligned position. It basically involves a gun like tool with a central shaft and peripheral actuators, which attaches to an already implanted cup with the use of an adaptor. Using computer navigation, through a series of calculations, pure points (specifically defined) and secondary points on the edge of the cup are determined. This process confers positional information to the edge of the cup. The BMD2 tool has actuators that correspond to these points on the cup, and through a computer program, the appropriate actuators impact on specific points on the edge of the cup to adjust the position of the implanted cup. The surgeon dials in the desired alignment and the BMD2 tool fires the appropriate actuators to realign the cup to the perfect position.

In BMD3, we considered that vibratory forces may be applied in a manner to disarm frictional forces in insertion of the acetabular cup into the pelvis. We asked the following questions: Is it possible to insert and position the cup into the pelvis without high energy impacts? Is it possible to insert the cup using vibratory energy? Is insertion and simultaneous alignment and positioning of the cup into the pelvis possible? BMD3 prototypes were designed and the concept of vibratory insertion was proven. It was possible to insert the cup with vibratory energy. The BMD3 principle involved the breaking down of the large momentum associated with the discrete blows of the mallet into a series of small taps, which in turn did much of the same work incrementally, and in a stepwise fashion. We considered that this method allowed modulation of force required for cup insertion. In determining the amount of force to be applied, we studied the resistive forces involved in a cup/cavity interaction. We determined that there are several factors that produce the resistive force to cup insertion. These include bone density (hard or soft), cup geometry (spherical or elliptical), and surface roughness of the cup. With the use of BMD3 vibratory insertion, we demonstrated through FEM studies, that the acetabulum experiences less stress and deformation and the cup experiences a significantly smoother sinking pattern. We discovered the added benefit of ease of movement and the ability to align the cup with the BMD3 vibratory tool. During high frequency vibration the frictional forces are disarmed in both effective and realistic ways, (see previous papers—periodic static friction regime, kinetic friction regime). We have also theorized that certain “mode shapes” (preferred directions of deformation) can be elicited with high frequency vibration to allow easy insertion and alignment of the cup. The pelvis has a resonant frequency and is a viscoelastic structure. Theoretically, vibrations can exploit the elastic nature of bone and it's dynamic response. This aspect of vibratory insertion can be used to our advantage in cup insertion and deserves further study. Empirically, the high frequency aspect of BMD3 allows easy and effortless movement and insertion of the cup into the pelvis. This aspect BMD3 is clinically significant allowing the surgeon to align the cup in perfect position while the vibrations are occurring.

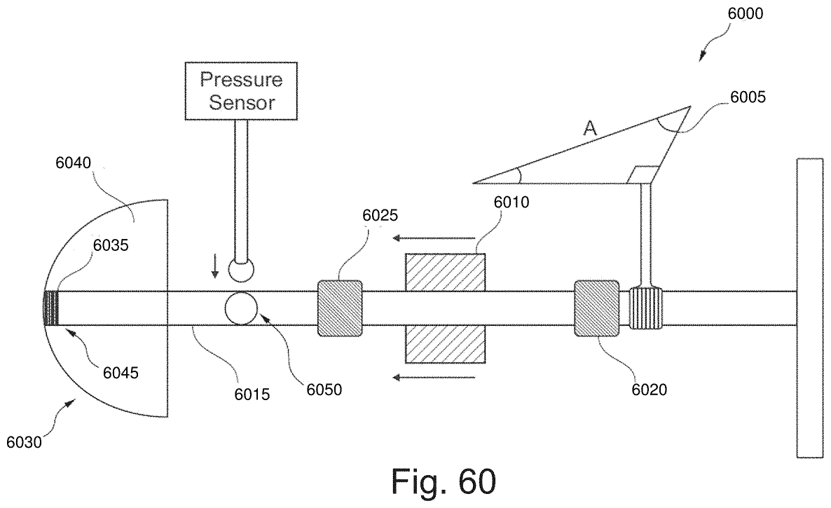

The BMD4 idea was described to address the two initial problems (uncontrolled force and undesirable torques) in a simpler manner. The undesirable torque and mis-alignment problem from mallet blows were neutralized with the concept of the “slide-hammer” which only allows axial exertion of force. With respect to the amount of force, BMD4 allowed the breaking down of the large impaction forces (associated with the use of the mallet) into quantifiable and smaller packets of force. The delivery of this force occurs through a simple slide-hammer, cockup gun, robotic tool, electric or pneumatic gun (all of which deliver a sliding mass over a central coaxial shaft attached to the impaction rod and cup. In the BMD4 paper we described two “stop gap” mechanisms to protect the pelvis from over exertion of force. We described a pressure sensor in the shaft of the BMD4 tool that monitors the force pressure in the (tool/cup system)—see, for example, FIG. 60. This force sensor would determine when the pressure had plateaued indicating the appropriate time to stop the manual impacts. We also described a pitch/sound sensor in the room, attached to the gun or attached to the pelvis that would assess when the pitch is not advancing, alerting the surgeon to stop applying force. These four aspects of BMD4 (coaxially of the gun, quantification and control of the force, a force sensor, a sound sensor) are separated and independent functions which could be used alone or in conjunction with each other.

We also recommended that BMD4's (coaxiality and force control function) and BMD3's (vibratory insertion) be utilized for application of femoral and humeral heads to trunnions, to solve the trunnionosis problem.

Materials and Methods: During our development, we evaluated different aspects of the BMD3 and BMD4 prototypes. With BMD3 concept we sought to study several aspects of vibratory insertion:

1. The ultimate effect of frequency on cup insertion

2. The range of impact forces achievable with vibratory insertion.

3. The effect of frequency and vibratory impaction forces on cup insertion and (extraction forces measured to assess the quality of insertion).

With Respect to BMD4 we studied the various aspects of “controlled impaction” utilizing Drop Tests (dynamic testing) and Instron Machine (static testing) to determine the behavior of cup/cavity interaction.

Results:

BMD3

Preliminary results suggest that vibratory insertion of the cup into a bone substitute is possible. It is clear that vibratory insertion at higher frequencies allow easy insertion and alignment of the cup in bone.

It is unclear as to how much higher frequencies contribute to the depth and quality of insertion, as measured by the extraction force, particularly as the cup is inserted deeper into the substrate.

We determined that with vibrational insertion, the magnitude of impaction force is limited and dependent on other mechanical factors such as frequency of vibration and the dwell time. So far 400 lbs. of force has been achieved with the BMD/BE prototype, 250 lbs. of force have been achieved with the auto hammer prototype, and 150 lbs. of force have been achieved by the pneumatic prototype. Further work is underway to determine the upper limit of achievable forces with the Vibrational tools.

During our study of Vibrational insertion, we also discovered that vibrational insertion can be unidirectional or bidirectional. For insertion of the cup into a substrate it was felt that unidirectional vibratory insertion (in a positive direction) is ideal. We discovered that unidirectional vibratory withdrawal and bidirectional vibration have other applications such as in revision surgery, preparation of bone, and for insertion of bidirectional prosthetic cups. The directionality of the BMD3 vibratory prototype and its applications will be further discussed in additional applications.

BMD4

With respect to controlled impacts we sought to understand the cup/cavity interaction in a more comprehensive way. We wanted to discover the nature of the resistive forces involved in a cup/cavity interaction. We felt it was necessary for us to know this information in order to be able to produce the appropriate amount of force for both BMD3 “vibratory insertion” and BMD4 “controlled impaction”. We proposed and conducted dynamic Drop tests and static Instron tests to evaluate the relationship between the cup and the cavity. Instron testing is underway and soon to be completed. The drop tests were conducted using a Zimmer continuum 62 mm cup and 20 lbs. urethane foam. Multiple drop tests were conducted at various impaction forces to evaluate the relationship between applied force (TMIF) and displacement of the cup, and the quality of insertion (Extraction Force). We discovered that for insertion of a cup into a cavity the total resistive force can be generally represented by an exponential curve. We have termed this resistive force the FR, which is determined by measuring the relationship of applied force (TMIF) and cup insertion for any particular (cup/cavity) system. FR is a function of several factors including the spring like quality of bone which applies a compressive resistive force (Hooke's law F=kx) to the cup, the surface roughness's of the cup, an amount of under reaming, and the geometry of the cup (elliptical v spherical).

Definitions: FR=Force Resistance (total resistive force to cup insertion over full insertion of the cup into bone substitute); TMIF=Theoretical Maximum Impact Force (external force applied to the system) to accomplish cup insertion; and mIF=measured Impact Force (force measured within the system) (as measured on the BMD3 and BMD4) tools.

| | |
|---|---|
| BMD/BE | vibratory prototype |
| Auto hammer | vibratory prototype |
| Pneumatic | vibratory prototype |

Evaluation of the drop test data reveals a nonlinear (exponential) curve that represents FR. We contemplated that the cup/cavity system we used (62m Continuum cup and 20 lb. urethane foam) has a specific profile or “cup print”, and that this profile was important to know in advance so that application of force can be done intelligently.

We observed the general shape of FR to be non-linear with three distinct segments to the curve, which we have termed A, B, and C. In section A the resistive force is low (from 100 to 350 lbs.) with a smaller slope. In section A, if an applied force (TMIF) greater than this FR is applied, it can produces up to 55% cup insertion and 30% extraction force. A TMIF that is tuned to cross FR at the A range is at risk for poor seating and pull out. In section B the resistive forces range from 500 lbs to 900 lbs. The slope rises rapidly and is significantly larger than in section A (as expected in an exponential curve). In section B, if a TMIF greater than this FR is applied, it can produce between 74% to 90% cup insertion and between 51% to 88% extraction force. We name this section the “B cloud”, to signify that the applied force (TMIF) should generally be tuned to this level to obtain appropriate insertion with less risk for fracture and or pull out, regardless of whether the TMIF is applied by a BMD3 or BMD4 tool. In section C the curve asymptotes, with small incremental increase in cup insertion and large increases in extraction force. The clinical value of the higher extraction force is uncertain with increased risk of fracture. A TMIF that is tuned to cross the FR at the C range is high risk for fracture and injury to the pelvis.

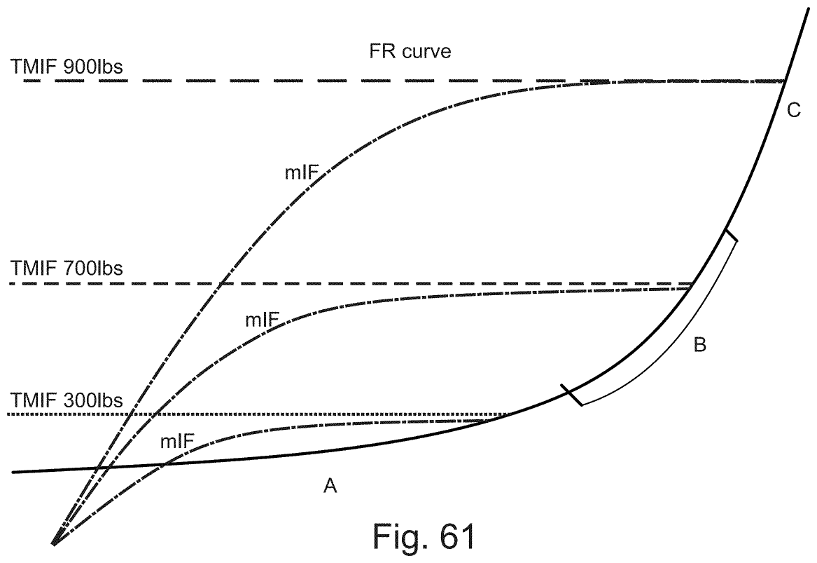

FIG. 60 relates to a Behzadi Medical Device (BMDX) which may combine vibratory and axial impactful forces from BMD3 and BMD4 among other options; and FIG. 61 illustrates a Force Resistance (FR) curve for various experimental configurations, for example, force as a function of distance or displacement.

FIG. 60 illustrates an embodiment 6000 of a BMD including a pressure sensor 6005 to provide feedback during installation. With respect to management of the force required for some of these tasks, it is noted that with current techniques (the use of the mallet) the surgeon has no indication of how much force is being imparted onto the implant and/or the implant site (e.g., the pelvis). Laboratory tests may be done to estimate what range of force should be utilized in certain age groups (as a rough guide) and then fashioning a device 6000, for example a modified sledgehammer or cockup gun to produce just the right amount of force. Typically the surgeon may use up to 2000 N to 3000 N of force to impact a cup into the acetabular cavity. Also, since some embodiments cannot deliver the force in an incremental fashion as described in association with the BMD3 device, device 6000 includes a stopgap mechanism. Some embodiments of the BMD3 device have already described the application of a sensor in the body of the impaction rod. Device 6000 includes system/assembly 6005 embedded in device 6000, for example proximate rod 6010 near distal end 6010, and used to provide valuable feedback information to the surgeon. Pressure sensor 6005 can let the surgeon know when the pressures seems to have maximized, whether used for the insertion of an acetabular cup, or any other implant including knee and shoulder implants and rods used to fix tibia and femur fractures. When pressure sensor 6005 is not showing an advance or increase in pressure readings and has plateaued, the surgeon may determine it is time to stop operation/impacting. An indicator, for example an alarm can go off or a red signal can show when maximal peak forces are repeatedly achieved. As noted above, the incorporated patents describe a presence of a pressure sensor in an installation device, the presence of which was designed as part of a system to characterize an installation pulse pattern communicated by a pulse transfer assembly. The disclosure here relates to a pressure sensor provided not to characterize the installation pulse pattern but to provide an in situ feedback mechanism to the surgeon as to a status of the installation, such as to reduce a risk of fracturing the installation site. Some embodiments may also employ this pressure sensor for multiple purposes including characterization of an applied pulse pattern such as, for example, when the device includes automated control of an impacting engine coupled to the hammer. Other embodiments of this invention may dispose the sensor or sensor reading system within a handle or housing of the device rather than in the central rod or shaft.

### Discussion

The FR curve represents a very important piece of information. To the surgeon the FR curve should have the same significance that a topographical map has to a mountaineer. Knowing the resistive forces involved in any particular cup/cavity interaction is desirable in order to know how much force is necessary for insertion of the cup. We believe that in vitro, all cup/cavity interactions have to be studied and qualified. For example it is important to know if the same 62 mm Continuum cup we used in this experiment is going to be used in a 40 year old or 70 year old person. The variables that will determine FR include bone density which determines the spring like quality of bone that provides compression to the cup, the geometry of the cup, an amount of under reaming, and the surface roughness of the cup. Once the FR for a particular cup and bone density is known, the surgeon is now armed with information he/she can use to reliably insert the cup. This would seem to be a much better way to approach cup insertion than banging clueless on a an impaction rod with a 4 lbs mallet. Approaching FR with an eye for the B range will assure that the cup is not going to be poorly seated with risk of pullout or too deeply seated with a risk of fracture.

We have contemplated approaching FR with both vibratory (BMD3) insertion and controlled (BMD4 impaction) among other devices. Each of these systems has advantages and disadvantages that continue to be studied and further developed.

For example, we believe that vibratory insertion with the current BMD3 prototypes have the clear advantage of allowing the surgeon ease of movement and insertion. The surgeon appears to be able to move the cup within the cavity by simple hand pressure to the desired alignment. This provides the appearance of a frictionless state. However, to date we have not quite been able to achieve higher forces with the BMD3 tools. So far we have been able to achieve up to 150 lb. (pneumatic), 250 (auto hammer), and 400 lb. (BMD/BE) in our vibratory prototypes. This level of applied force provides submaximal level of insertion and pull out force. We believe that ultimately, higher forces can be achieved with the vibratory BMD3 tools (500 to 900 lbs) which will provide for deep and secure seating.

With regards to this concern, we have contemplated a novel approach to address the current technological deficits. We propose a combination of BMD3 vibratory insertion with controlled BMD4 impaction. The BMD3 vibratory tool (currently at 100 lbs. to 400 lbs) is used to initiate the first phase of insertion allowing the surgeon to easily align and partially insert the prosthesis with hand pressure, while monitoring the alignment with the method of choice (A-frame, navigation, C-arm, IMU). The BMD4 controlled impaction is then utilized to apply quantifiable packets of force (100 lbs. to 900 lbs) to the cup to finish the seating of the prosthesis in the B range of the FR curve. This can be done either as a single step fashion or “walking up the FR curve” fashion.

Alternatively, BMD4 controlled impaction can be utilized to insert the cup without the advantage of BMD3 tool. The BMD4 technique provides the ability to quantify and control the amount of applied force (TMIF) and provides coaxiality to avoid undesirable torques during the impaction. It is particularly appealing for robotic insertion where the position of the impaction rod is rigidly secured by the robot.

We have contemplated that the BMD4 controlled impaction can be utilized in two separate techniques.

The first technique involves setting the impaction force within the middle of the B Cloud where 74% to 90% insertion and 51% to 88% extraction forces could be expected, and then impacting the cup. The BMD4 tool acts through the slide hammer mechanism to produce a specific amount of force (for example 600 lbs) and deliver it axially. This can be considered a single step mechanism for use of BMD4 technique.

The second method involves “walking the forces” up the FR curve. In this system the applied force (TMIF) is provided in “packets of energy”. For example, the BMD4 gun may create 100 lbs packets of force. It has an internal pressure sensing mechanism that allows the tool to know if insertion is occurring or not. A force sensor and a corresponding algorithm within the BMD4 tool is described herein. The force sensor monitors the measured impact force (mIF) and the corresponding change in mIF within the system. As we have described before, when impacts are applied to an “inelastic” system, energy is lost at the interface as insertion occurs and heat is produced. This loss of energy is measured and calculated in the (change) or slope of mIF. Consecutive mIF s have to be measured and compared to previous mIFs to determine if insertion is occurring. As long as insertion is occurring impactions will continue. When the change in mIF approaches zero, insertion is not occurring, there is no dissipation of energy within the system The slope or (change) in mIF has approached zero. At this point the cup and cavity move together as a rigid system (elastic), and all the kinetic energy of TMIF is experienced by the cup/cavity system and mIF is measured to be the same as TMIF. When insertion is not occurring mIF has approached TMIF and change in mIF has approached zero.

At this point the next step is taken and TMIF is increased, for example by a packet of 100 lbs. The subsequent mIF measurements are taken and if the slope (change) in mIF is high, insertion is occurring with the new TMIF, therefore impacts should continue until the change in mIF approaches zero again.

Conversely, if an increase in TMIF results in an increase in mIF but not the change (slope) in mIF, we know the cup is no longer inserting and has reached its maximum insertion point. We should point out that when the cup stops inserting, this also the point where FR exceeds TMIF. In this manner, we have contemplated an algorithm that allows for monitoring of the forces experienced in the system. Based on this algorithm, a system is created in which the surgeon can walk the TMIF up the FR curve while being given realtime feedback information as to when to stop impaction.

The general idea is that at some point in time the cup will no longer insert (even though not fully seated). This algorithm determines when no further insertion is occurring. The surgeon will be content to stop impaction in the B cloud range of the FR curve.

We have also discovered that mIF is related to TMIF+FR. The value of TMIF is known. The value of mIF is measured. The FR can be calculated live during insertion by the BMD3 and BMD4 tools and shown to the surgeon as a % or (probability of fracture). This calculation and algorithm could be very significant.

A few words on Alignment:

We have so far proposed that the BMD3 vibratory tool be used to insert the cup under monitoring by current alignment techniques (navigation, Fluoroscopy, A-frame). We have now devised a novel system, which we believe will be the most efficacious method of monitoring and assuring alignment. This system relies of Radlink (Xrays) and PSI (patient specific models) to set and calibrate the OR space as the first step.

As a second step, it utilizes a novel technique with use of IMU technology to monitor the movement of the reamers, tools (BMDs) and impaction rods. This is discussed in a separate paper. Needs to be written up.

Summary and Recommendations for BMD/BE project.

1. We propose a novel system of inserting and aligning the acetabular cup in the human pelvic bone. This technique involves combining aspects of the BMD3 and BMD4 prototypes, initially utilizing BMD3 vibratory insertion to partially insert and perfectly align the acetabular cup into the pelvis. Subsequently switching to the BMD4 controlled impaction technique to apply specific quantifiable forces for full seating and insertion. In this manner we are combining the proven advantages of the vibratory insertion prototype with the advantages of the controlled impaction prototype.

2. We have described a force sensing system within the BMD tool with capacity to measure the force experienced by the system (mIF) and calculate the change in mIF with respect to time or number of impacts. This system provides a feedback mechanism for the BMD tools as to when impaction should stop.

3. We have described the FR curve which is a profile (cup print) of any cup/cavity interaction. And have recommended that this “cup print” for most cup/cavity interactions be determined in vitro to arm the surgeon with information necessary for cup insertion. We feel that every cup/cavity interaction deserves study to determine its FR profile. Once the FR is known, BMD3 and BMD4 tools can be used to intelligently and confidently apply force for insertion of the acetabular prosthesis.

4. We have described two methods for use of BMD4 controlled cup impaction

a. Setting the TMIF to the middle of the B cloud (somewhere between 500 to 900 range for our FR) and producing a single stage impaction.

b. Producing sequential packets of increasing TMIF in order to walk TMIF up the FR curve. (Increasing packets of 100 lbs or 200 lbs)

5. We have also discovered that mIF is related to TMIF+FR. The value of TMIF is known. The value of mIF is measured. The FR can be calculated live during insertion by the BMD3 and BMD4 tools and shown to the surgeon as a % or (probability of fracture). This calculation and algorithm could be very significant in help the surgeon to insert the cup deeply without fracture.

Concept 5 W and 1H:

1. Who: The surgeon; 2. What: Cup insertion; 3. When: When to increase the force and when to stop; 4. Where: PSI and Radlink to set and IMU to monitor alignment and position; 5. Why: Consistency for the surgeon and the patient; and 6. How: FR for every cup/cavity interaction, BMD3 and BMD4 tools.

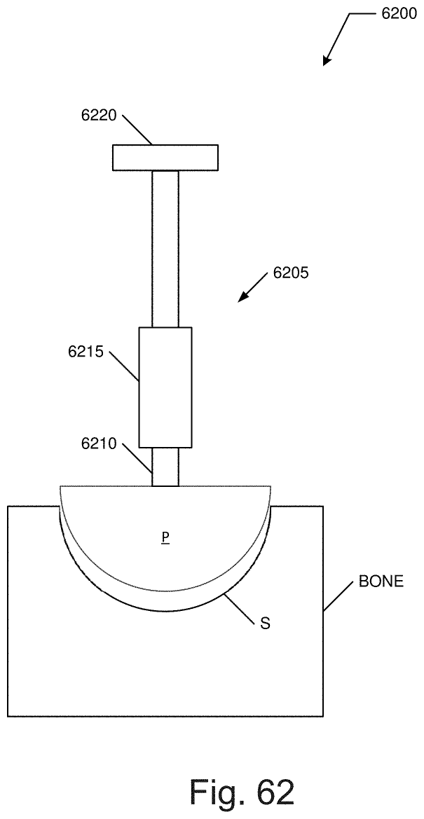

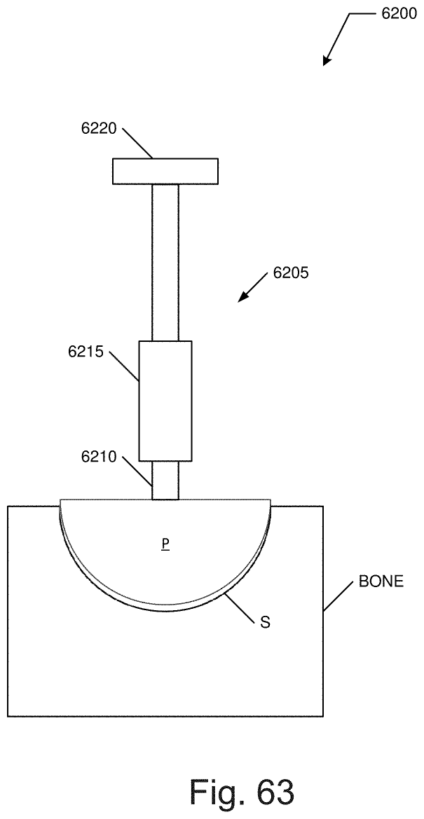

FIG. 62-FIG. 63 illustrate a general force measurement system 6200 for understanding an installation of a prosthesis P into an installation site S (e.g., an acetabular cup into an acetabulum during total hip replacement procedures); FIG. 62 illustrates an initial engagement of prosthesis P to a cavity at installation site S when prosthesis P is secured to a force sensing tool 6205; FIG. 63 illustrates a partial installation of prosthesis P into the cavity by operation of force sensing tool 6205.

Tool 6205 includes an elongate member 6210, such as a shaft, rod, or the like. There may be many different embodiments but tool 6205 may include a mechanism for direct or indirect measurement of impact forces (mIF) such as by inclusion of an in-line sensor 6215. Further, tool 6205 allows for application of an external force applied to tool 6205. In some embodiments, another sensor 6220 may be used to measure this applied force as a theoretical maximum impact force (TMIF). In some cases, the TMIF is applied from outside and in other systems, the application is from tool 6205 itself. In some cases, there system 6200 has a priori knowledge of the force applied or it can estimate it without use of sensor 6220. Depending upon an implementation, various user interface elements and controls may be included, including indicators for various measured, calculated, and/or determined status information.

During operation, as mIF begins to approach TMIF, then system 6200 understands that prosthesis P is not moving much, if any, in response to the TMIF (when it is kept relatively constant). An advantage to the mechanical tools is their ability to repeatably apply a known/predetermined force allowing for understanding of where the process is on an applicable FR curve for prosthesis P at installation site S. For example, in FIG. 63, the mIF, for a constant applied force, is closer to TMIF than in the case of FIG. 62.

The arrangement of FIG. 62-FIG. 63 may be implemented in many different ways as further explained herein for improving installation and reducing risk of fracture.

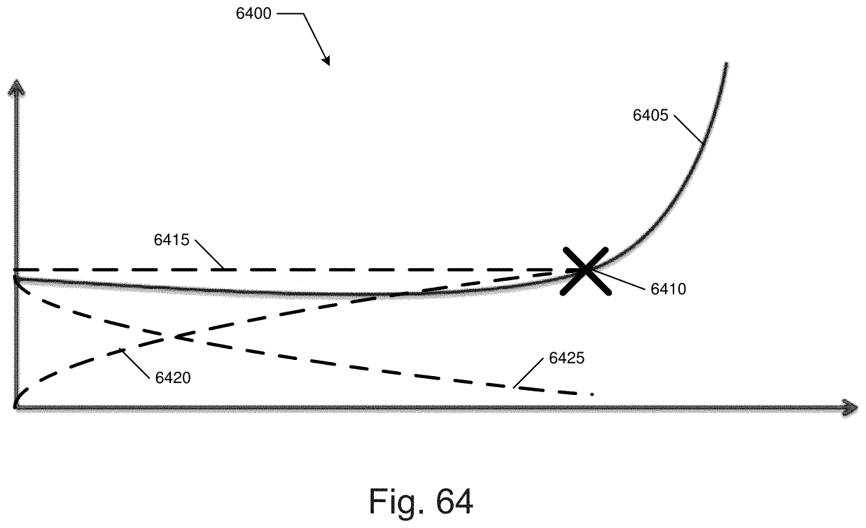

FIG. 64 illustrates a set of parameters and relationships for a force sensing system 6400 including a generalized FR curve 6405 visualizing various applicable forces implicated in operation of the tool in FIG. 62 and FIG. 63. Curve 6405 includes TMIF vs displacement of the implant at the installation site. Early, a small change of TMIF can result is a relatively large change in displacement. However, near the magic spot, the curve starts to transition where the implant is close to being seated and increases in TMIF may result in little displacement change. And as TMIF increases, the risk of fracture increases.

In FIG. 64, a particular state is illustrated by “X” a point 6410 on curve 6405. A particular constant value of TMIF 6415 is applied to the system and prosthesis P moves along curve 6405. A measured Impact Force (mIF) 6420 approaches the value of TMIF 6415 as prosthesis P approaches point 6410. A resultant curve 6425 illustrates a difference between TMIF 6415 and mIF 6420. As prosthesis P approaches point 6410, resultant curve 6425 provides a valuable, previously unavailable quantitative indication of how prosthesis P was responding to applied forces. It may be that the procedure stops at point 6410, or a new, larger value for TMIF is chosen to move prosthesis P along curve 6405. System 6400 provides the surgeon with knowledge of where on curve 6405 the prosthesis P resides and provides an indication of a risk of fracture versus improving seating of prosthesis P. By monitoring resultant curve 6425 in some form, system 6400 understands whether prosthesis is moving or has become seated. Each of these pieces of information is useful to system 6400 and/or the surgeon until completion of the process.

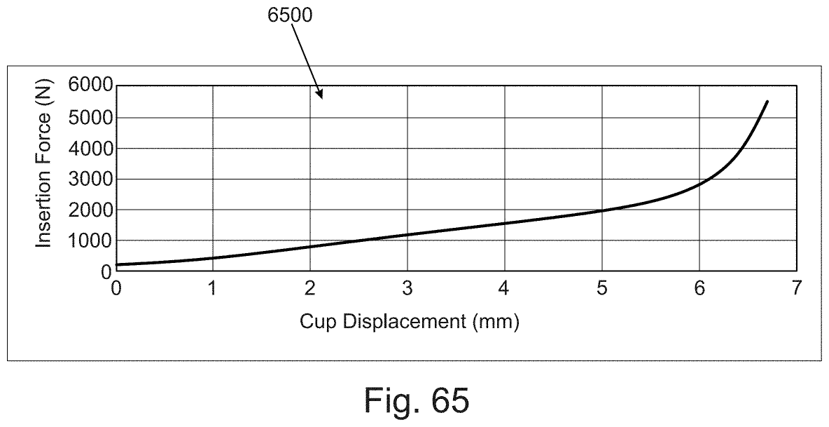

FIG. 65-FIG. 21 illustrate a first specific implementation of the system and method of FIG. 62-FIG. 64, FIG. 65 illustrates a representative plot 6500 of insertion force for a cup during installation. As prosthesis P is being installed by a system, device, process, or tool, each increment of the active installation will have an applicable minimum impact to overcome resistive (e.g., static friction) forces. The impact force required increases as the insertion depth of the cup increases due to larger normal forces acting on the cup/bone interface (see FIG. 65). There is a tension between seating and increased force though, as larger impact forces raise the risk of fracture of surrounding bone. The goal of the surgeon is to reach a sufficient insertion depth to generate acceptable cup stability (e.g., pull-out resistance or seatedness), while minimizing forces imparted to the acetabulum during the process. The process does not want to terminate early as the prosthesis may too easily be removed and the process doesn't want to continue too long until the bone fractures. This area is believed to be in the beginning of the non-linear regime in the plot of FIG. 65, as higher forces begin to have a smaller incremental benefit to cup insertion (i.e. smaller incremental insertion depth with larger forces).

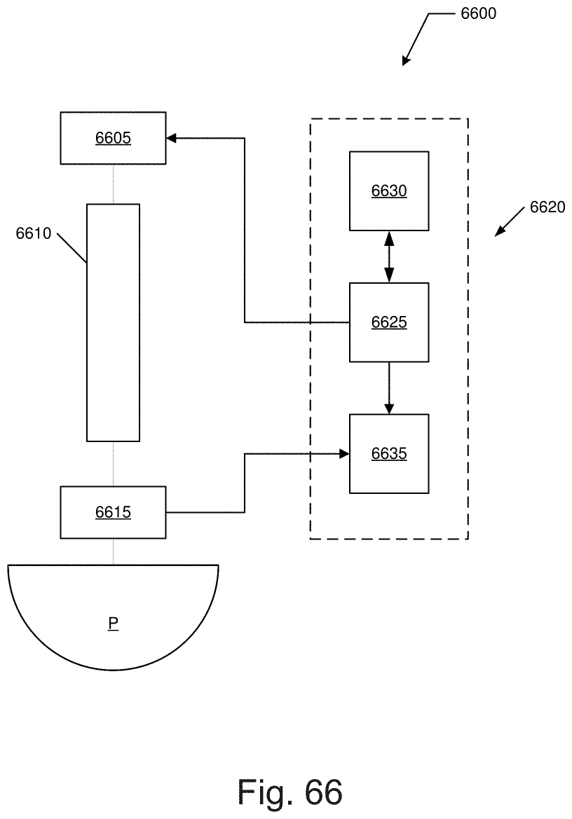

FIG. 66 illustrates a first particular embodiment of a BMDX force sensing tool 6600. Tool 6600 allows indirect measurement of a rate of insertion of an acetabular cup and may be used to control the impact force being delivered to the cup based upon control signals and the use of features of FIG. 66. Tool 6600 may include an actuator 6605, a shaft 6610, and a force sensor 6615. One representative method for force measurement/response would employ such a tool 6600. Similar to the impaction rod currently used by surgeons, tool 6600 would couple to an acetabular cup (prosthesis P) using an appropriate thread at the distal end of shaft 6610. Actuator 6605 would couple to a proximal end of shaft 6610, and create controlled impacts that would be applied to shaft 6610 and connected cup P. The magnitude of the impact(s) would be controlled by the surgeon through a system control 6620, such as a dial or other input mechanism on the device, or directly by the instrument's software. System control 6620 may include a microcontroller 6625 in two-way communication with a user interface 6630 and receiving inputs from a signal conditioner 6635 receiving data from force sensor 6615. Controller 6625 is coupled to actuator 6605 to set a desired impact value.

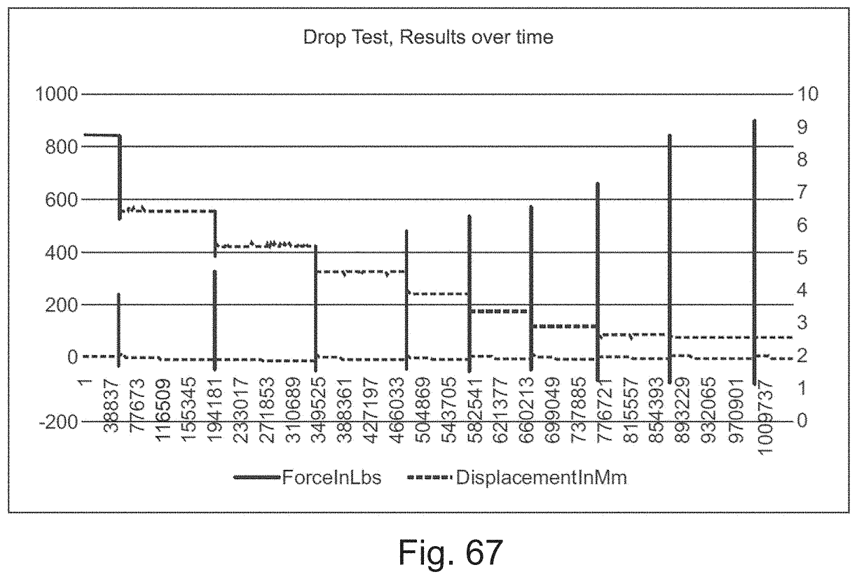

Force sensor 6615 may be mounted between the shaft 6610 and acetabular cup P. Sensor 6615 would be of a high enough sampling rate to capture the peak force generated during an actuator impact. It is known that for multiple impacts of a given energy, the resulting forces increase as the incremental cup insertion distance decreases, see, for example, FIG. 67. FIG. 67 illustrates a graph including results of a drop test over time which simulate use of tool 6600 installing cup P into bone.

This change in force given the same impact energy may be a result of the frictional forces between cup P and surrounding bone of the installation site. For the plot of FIG. 67, the initial impact has a slow deceleration of the cup due to its relatively large displacement, resulting in a low force measurement. The displacement decreases for subsequent impacts due to the increasing frictional forces between the cup and bone, which results in faster deceleration of the cup (the cup is decelerating from the same initial velocity over a shorter distance). This results in an increase in force measurement for each impact. The maximum force for a given impact energy will be when the cup P can no longer overcome, responsive to a given impact force from the actuating system, the resistive (e.g., static friction) forces from the surrounding bone. This results in a “plateau”, where any subsequent impact will not change either the insertion of cup P or the force measured.

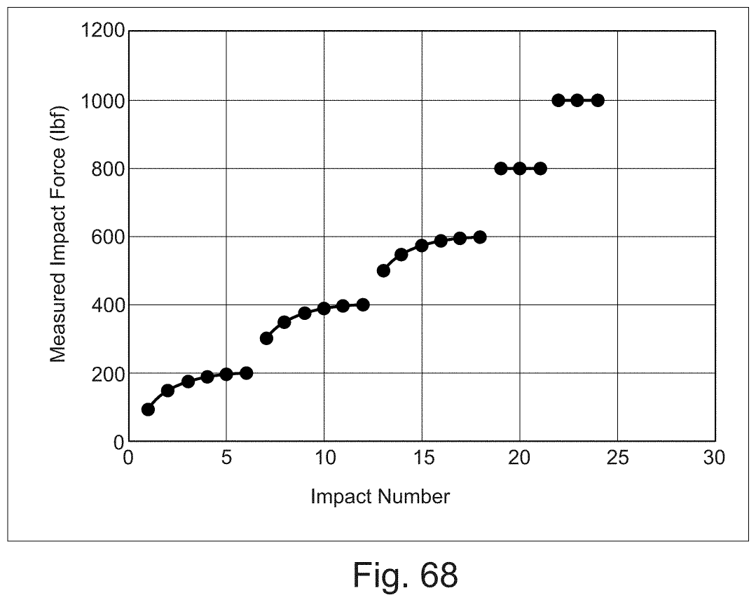

In some embodiments, this relationship may be used to “walk up” the insertion force plot illustrated in FIG. 65, allowing tool 6600 to find the “plateau” of larger and larger impact energies. By increasing the energy linearly, the relationship between measured impact force and cup insertion illustrated in FIG. 67 should hold until the system reaches the non-linear insertion force regime of FIG. 65. When the non-linear regime is reached, a small linear increase in impact energy will not overcome the higher static forces needed to continue to insert the cup. This will result in an almost immediate steady state for the measured impact force (mIF of a force application X is about the same as MIF of a force application X+1). A visual representation of the measured impact force as the impact energy is increased is illustrated in FIG. 68. FIG. 68 illustrates a graph of measured impact force as impact energy is increased. Five impact energy levels are shown, with the last two increases in energy resulting in the cup entering the non-linear portion of the insertion force plot illustrated in FIG. 65.

A procedure for automated impact control/force measurement may include: a) Begin impacts with a static, low energy; b) Record the measured impact force (MIF); c) continue striking until the difference in measured impact force approaches zero (dMIF=>0), inferring that the cup is no longer displacing; d) increase the energy of the impacts by a known, relatively small amount; and e) repeat striking until plateau and increasing energy in a linear fashion until an increase in energy does not result in the relationship shown in FIG. 67. Instead, an increase in energy results in a “step function” in recorded forces, with an immediate steady-state. The user could be notified of each increase in energy, allowing a decision by the surgeon to increase the resulting impact force.

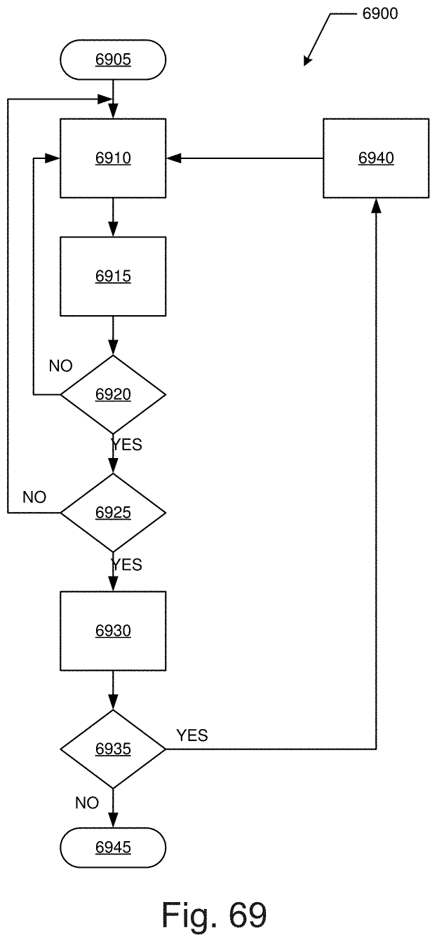

FIG. 69 illustrates a discrete impact control and measurement process 6900. Process 6900 includes step 6905-step 6945. Step 6905 (start) initializes process 6900. Process 6900 advances to a step 6910 to initiate the actuator to impart a known force application with energy X joules. After step 6910, process 6900 advances to step 6915 to measure impact force (MIF). After step 6915, process 6900 tests whether there have been a sufficient number of force applications to properly evaluate/measure a delta MIF (dMIF) between an initial value and a current value. When the test at step 6920 is negative, process 6900 returns to step 6910 to generate another force application event. Process 6900 continues with steps 6910-6920 until the test at step 6920 is affirmative, at which point process 6900 advances to a test at step 6925. Step 6925 tests whether the evaluated dMIF is approaching within a predetermined threshold of zero (that is, MIF(N)−MIF(N−1)=>0 within a desired threshold. When the test at step 6925 is negative, process 6900 returns to step 6910 for produce another force application event and process 6900 repeats steps 6910-6925 until the test at step 6925 is affirmative.

When affirmative, process 6900 advances to a step 6930 and includes a user feedback event to inform a surgeon/observer that the prosthesis is no longer inserting at a given TMIF value. After step 6930, process 6900 may include a test at step 6935 as to whether the user desires to increase the TMIF. Some implementations may not include this test (and either automatically continue until a termination event or the system stops automatically).

In the test at step 6935, the user may choose to have the energy applied from the actuator increased. Process 6900 includes a step 6940 after an affirmative result of the test at step 6935 which increases the current energy applied by the actuator an additional Y joules. After the change of energy at step 6940, process 6900 returns to repeat steps 6910-6935 until the test at step 6935 is negative. At which point, process 6900 advances to an end step 6945 which may include any post-installation processing.

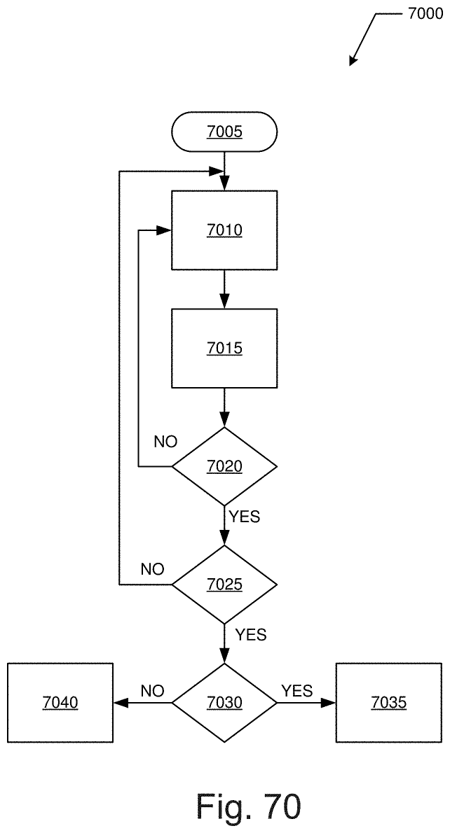

Once the non-linear regime discussed in FIG. 65 is reached, the probability of fracture increases. This is due to the acetabular cup nearing its full insertion depth, with limited incremental displacement from additional blows. This results in larger impact forces that are transmitted to the surrounding bone. Tool 6600 is able to detect when this regime is reached using process 6900, and could generate an alert through the user interface. The implementation of an alert could be performed in a number of different ways. One way would be a warning light and/or tone that would activate when a “step function” increase in measured impact force is detected. More advanced implementations are possible, with the system indicating the increasing probability of fracture as impact energy is increased once a “step function” increase in measured impact force is detected. The increasing risk of fracture could be shown through an LED bar that would illuminate additional lights to correspond to the relative risk, or by computing and displaying a fracture probability directly on the user interface. It should be noted that the cup may not fully seated when the system generates the aforementioned alert. This could be due to cup alignment issues, incorrect bone preparation, or incorrect cup sizing, among other causes. In these instances the system would generate an alert before the cup is fully inserted, allowing the surgeon to stop and determine the cause of the alert. This may be an additional benefit, allowing detection of an insertion issue before larger impact forces are used. A flowchart for one form of warning implementation is illustrated in FIG. 70.

FIG. 70 illustrates a warning process 7000. Process 7000 includes a step 7005-step 7040. Step 7005 (start) initializes process 7000. Process 7000 advances to a step 7010 to initiate the actuator to impart a known force application with energy X joules. After step 7010, process 7000 advances to step 7015 to measure impact force (MIF). After step 7015, process 7000 tests whether there have been a sufficient number of force applications to properly evaluate/measure a delta MIF (dMIF) between an initial value and a current value. When the test at step 7020 is negative, process 7000 returns to step 7010 to generate another force application event. Process 7000 continues with steps 7010-7020 until the test at step 7020 is affirmative, at which point process 7000 advances to a test at step 7025. Step 7025 tests whether the evaluated dMIF is approaching within a predetermined threshold of zero (that is, MIF(N)−MIF(N−1)=>0 within a desired threshold. When the test at step 7025 is negative, process 7000 returns to step 7010 for produce another force application event and process 7000 repeats steps 7010-7025 until the test at step 7025 is affirmative.

When affirmative, process 7000 advances to a step 7030 and includes a warning test event to test whether a first and a last MIF are within measurement error (MIF(0)=MIF(N)?) When the test at step 7030 is affirmative, a warning may be issued. When the test at step 7030 is negative, no warning is issued. There are similarities with process 6900 and process 7000 and some embodiments may combine them.

Improved performance may arise when the device is in the same state before each impact, in that the force applied by the user to the device is relatively consistent. Varying the user's input may influence the measured impact force for a strike, resulting in erroneous resistance curve modeling by the device. In order to minimize the occurrence, the device could actively monitor the force sensor between impacts, looking for a static load before within an acceptable value range. The system could also use the static load measurements directly before a strike as the impact's reference point, allowing relative measurements that reduce the effect of user variation. Even with this step, it is expected that filtering and statistical analysis will need to be performed in order to minimize signal noise.

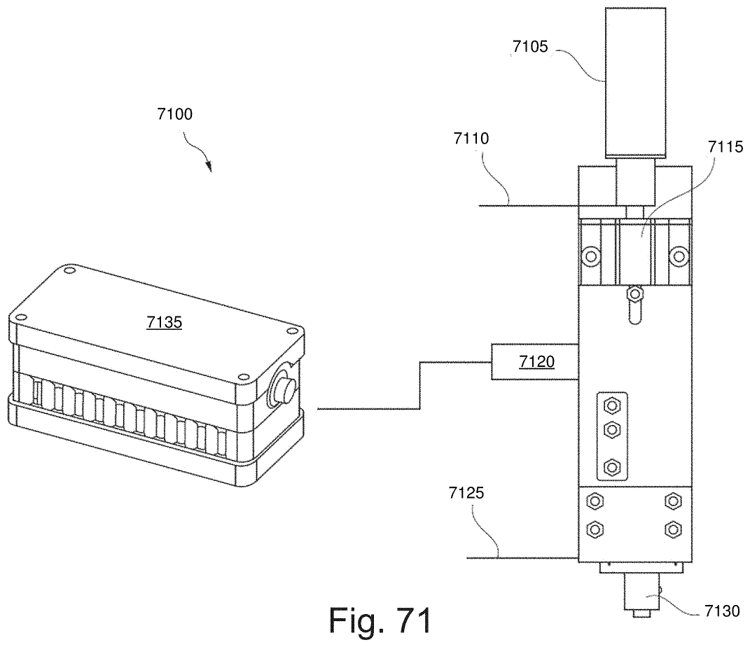

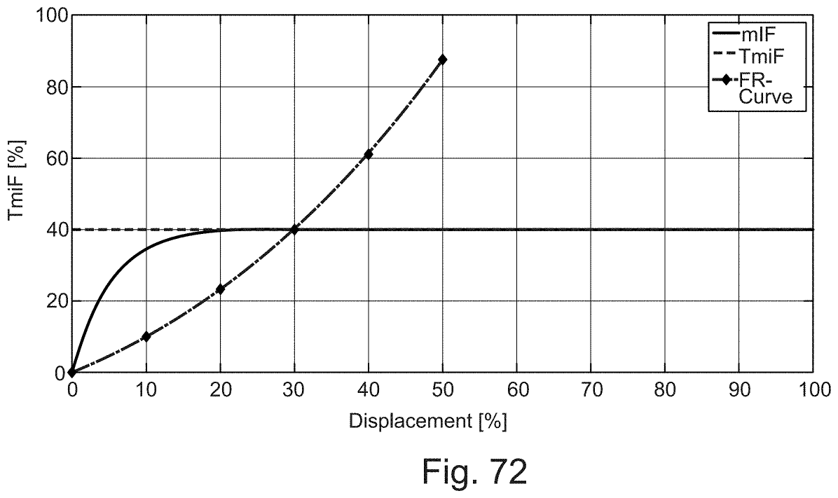

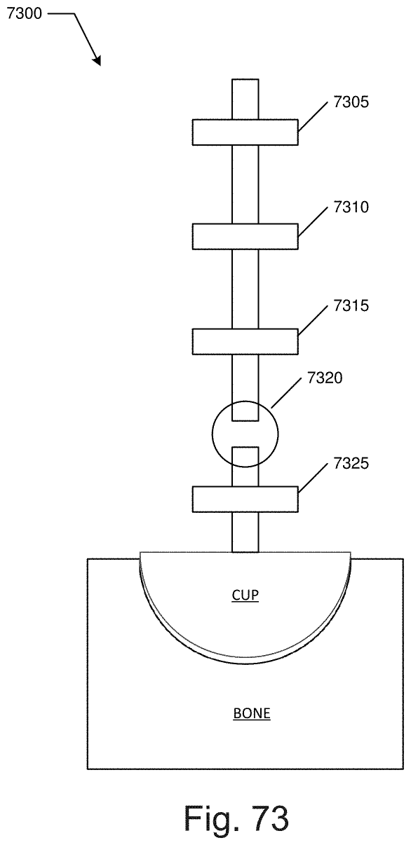

FIG. 71-FIG. 76 illustrate a second specific implementation of the system and method of FIG. 62-FIG. 64; FIG. 71 illustrates a basic force sensor system 7100 for controlled insertion. System 7100 includes a handle 7105, a first force sensor 7110, a shock absorber 7115, a motor 7120, a second force sensor 7125, and impact rod 7130, and a processing unit 7135. A purpose of system 7100 is to use force measurements and estimates to provide cup settlement feedback. A basic configuration of the hardware involved in system 7100 is illustrated in FIG. 71. Important sensors include: Preload sensor 7110, motor current sensor located in PPU 7135; and impaction sensor 7125. Instrumentation of system 7100 either measures or estimates variables illustrated in FIG. 72. FIG. 72 illustrates an FR curve including TmIF and mIF as functions of displacement. FIG. 73 illustrates a generic force sensor tool to access variables of interest in FIG. 72. System 7300, corresponding generally to system 6200 includes a force sensor 7305 (measuring F), a damping mechanism 7310, a current sensor (TmIF estimation and Actuator) function 7315, a vibrating/impacting interface 7320, and a force sensor 7325 (measuring mIF).

The relationship among the three curves in FIG. 72 are able to determine the cup/cavity settlement behavior. mIF can be directly measured by system 7100 as described herein. For example, impaction sensor 7125 may be a force sensor placed in the impacting rod 7130. The impacting rod 7130 receives and transmits impacts directly to the cup. This same impaction force input is sensed by sensor 7125.

TmIF is composed by both preload and actuator force. The preload is measured directly by the force sensor 7110. The actuator force can be estimated by means of current sensing (motor 7120 and PPU 7135) as the torque/force generated by the motor can be related to its electric current.] C. L. Chu, M. C. Tsai, H. Y. Chen, “Torque control of brushless dc motors applied to electric vehicles,” in IEEE International Electric Machines and Drives Conference, 2001, pp. 82-87.

Motor 7120 is connected to PPU 7135 where the current sensor is installed. All measurements shall be properly filtered and handled in real-time before any advanced processing takes place. Both low level and advanced real-time processing are executed in PPU 7135 for each sensor. Sensor 7125 needs less processing since this is the direct measurement of mIF. TmIF needs more processing since it is composed by direct measurement of sensor 7110 and estimated force provided by motor 7120. Force estimation is basically data fusion of brushless DC motor current measurements with its electromechanical mathematical model considering mechanism interactions.

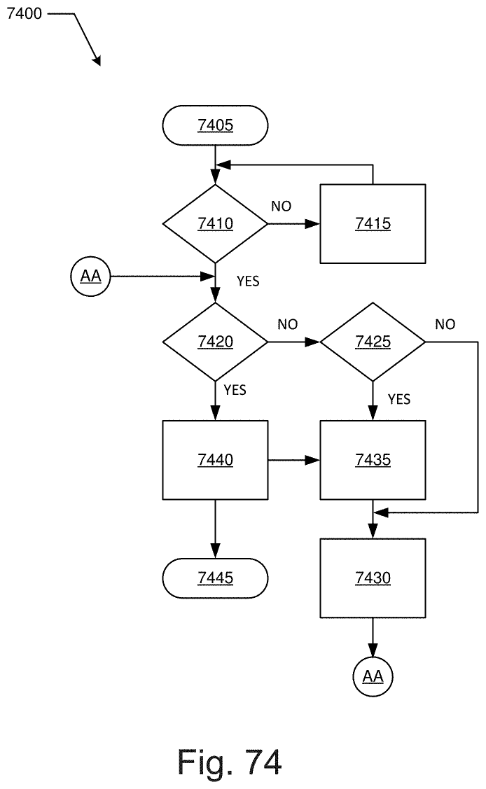

Once mIF and TmIF are internally available (to the PPU), the frequency of the actuating mechanism can be changed as a function of these variables. This allows the tool to track the optimal region (the B-Cloud) of the FR-Curve. It is important to note that mIF steady state value depends on current TmIF. In other words, the B-Cloud can be suitably tracked by the combination of both TmIF and mIF as described in the flowchart of FIG. 74.

FIG. 74 illustrates a B-cloud tracking process 7400 using TmIF and MIF measurements. Process 7400 includes step 7405-step 7445. Step 7405, a start step, initiates process 7400. After start 7405, process 7400 includes a test step 7410 to determine whether TmIF=mIF. When negative, process 7400 performs a controlled action step 7415 and then returns to step 7410. Process 7400 repeats steps 7410-7415 until the test at step 7410 is affirmative, at which point process 7400 performs a test step 7420 to determine whether the B-cloud is achieved. When the test at step 7420 is negative, process 7400 performs a test step 7425 to determine whether to change the preload. When the test at step 7425 is negative, process 7400 performs a controlled action step 7430 and then branches to AA—to the test at step 7420.

When the test at step 7425 is affirmative, process 7400 queries the surgeon at step 7435 as to changing the preload. In response to surgeon consultation step 7435, process 7400 performs controlled action step 7430. Process 7400 repeats steps 7420-7435 until the test at step 7420 is affirmative. When affirmative, process 7400 performs a stop insertion step 7440 and may either ask surgeon at step 7430 and/or conclude process 7400 by performing an end step 7445.

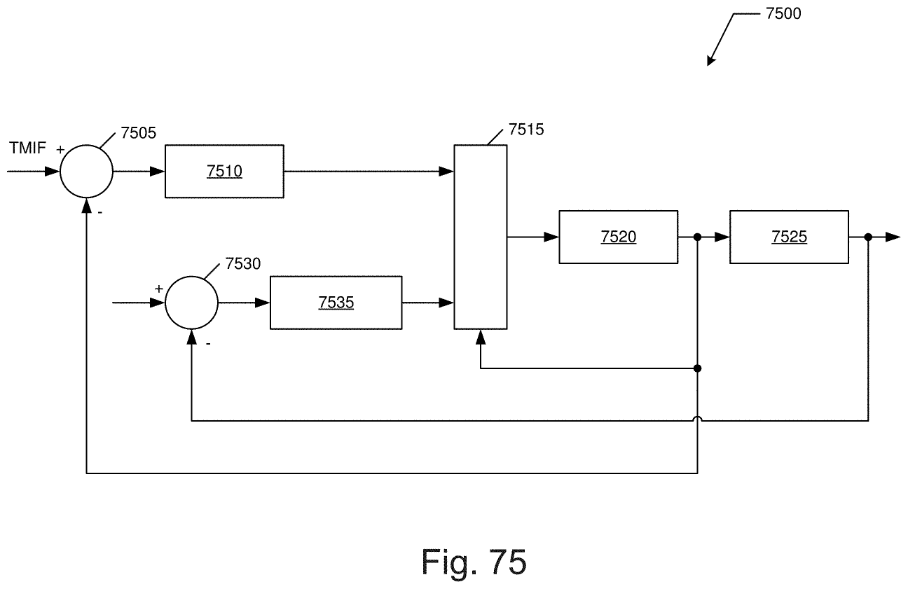

Process 7400 begins when the cup is preloaded against the cavity. It may be triggered by force threshold or button press. Current TmIF and mIF are constantly compared and regulated to be equal according to an internal control system when they are not able to converge easily. The control system is detailed in FIG. 75. FIG. 75 illustrates a control system 7500 for the “controlled action” referenced in FIG. 74.

Control system 7500 includes a set of processing blocks, real objects, computed signals and raw measurement and computed signals selectively responsive to input force and input frequency commands. System 7500 includes a feedback block 7505, a Bcloud regulator block 7510, a control selector 7515, a device/cavity/cup interaction assessment 7520, an FR curve estimator 7525, a feedback block 7530, and a performance pursuit block 7535.

Feedback block 7505 compares TMIF against an output (input force command and mIF) of block 7520. When/If there is an Input Force error at block 7505, Bcloud Regulator provides a first input frequency command f1 in response to the IF error. Feedback block 7530 compares a maximum feasible gain against a cup/cavity gain estimate from FR estimator 7525. When/if there is a gain error, performance pursuit 7535 takes this gain error and produces a second input frequency command. Control selector 7515 accepts both input frequency commands and selects one and provides it to the device/cavity/cup interaction 7520. Interaction 7520 produces input force command and mIF to FR estimator 7525, to selector 7515, and to feedback block 7505.

As the achievement of the B-Cloud is an objective, it is also constantly verified if it was achieved. However, the achievement of the B-Cloud is constrained to the value of the force source measured by TmIF. When the B-Cloud is not achieved, it is evaluated if there is need of pre-load increase or not (i.e. the actuator alone would be able to increase TmIF). In case of additional pre-load needed, the device asks the surgeon to increase the pre-load. The control system keeps running to make mIF track TmIF in an optimized way. The insertion stops automatically when the B-Cloud is achieved for the first time. A reference value inside the B-Cloud can be adjusted by the surgeon if she realizes based on its visual feedback that additional or less insertion force is necessary.

Embodiments described herein include a tool, device, system, apparatus and method for delivering a characterized insertion force (depending upon the embodiment)—that may have different attributes associated with this insertion force as described herein, such as an axiality of application of the insertion force, a quantification of a magnitude of the insertion force, a selectable variability to the magnitude, selective repeatability, when desired, of a desired magnitude for the insertion force. Generally, these insertion forces are sometimes referred to herein as an insertion agency.

There are possible exceptions related to abnormal or unexpected cup/cavity behavior. As a cup/cavity which needs too much pre-load or much more force than some actuators are able to achieve. For this reason the “B-Cloud regulation” block 2610 in FIG. 26 may be implemented in two distinct ways: a BMD3 device alone (curve 2705 in FIG. 27—mIF strong BMD3); or hybrid BMD3/BMD4 devices combined (curve 2710 with “weak” mIF BMD3 switched to BMD4—hybrid or discrete devices).

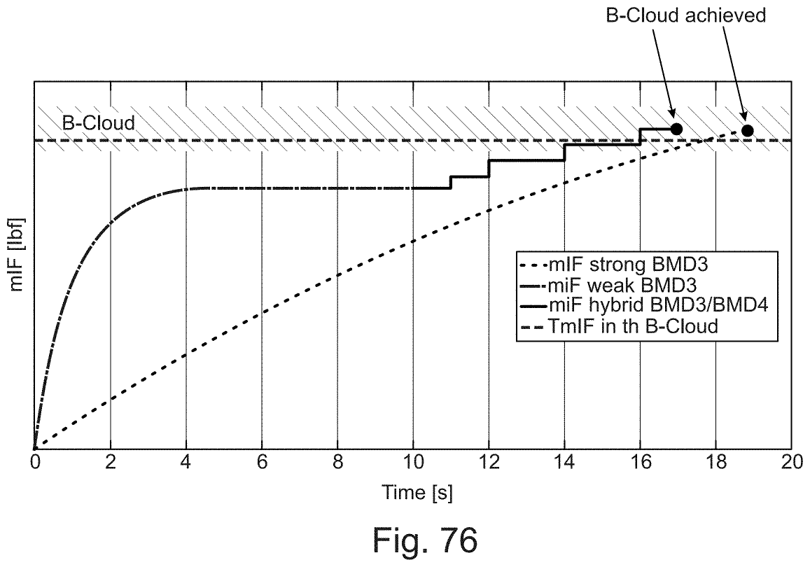

FIG. 76 illustrates possible B-cloud regulation strategies. A value on the B-Cloud is taken as reference for the B-Cloud regulator, this value is expressed by the dashed line in FIG. 76. In the case of a BMD3 able to perform the job alone, it can be achieved smoothly. In the case that BMD3 does not have sufficient power to accomplish the task, it switches to BMD4 which provides incremental impacts proportional to the difference between mIF and TmIF. Progressive BMD4 impacts change its amplitude following K_{BMD4}(m_{IF}−T_{mIF}), while K_{BMD} is a parameter which has to be determined experimentally.

Estimation of the Force Provided by the Motor

A reliable and feasible way to determine the amount of force made available by the actuator is by means of electrical current measurement. The accuracy and sizes involved in our application may make difficult the installation of force/torque sensors for motors and piezo transducers, which are the basic types of actuators used in BMD3 and BMD4 devices. However, electrical current drawn by these actuators is related to the force produced by them. In other words, the force produced can be understood as a function of the electrical current. This idea is largely in engineering. Our proposed solution would make use of estimators (e.g. Kalman filter) which relate the mathematical model of the electromechanical actuator fused with measured values of the electrical current to provide the force output generated in real-time by the actuator

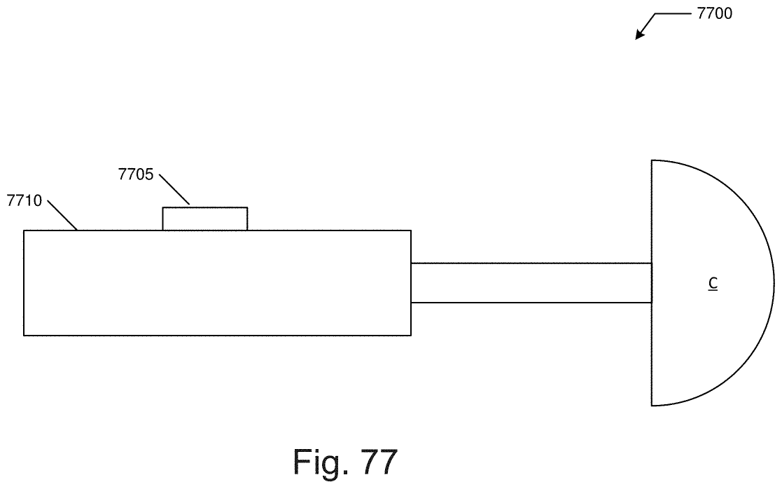

FIG. 77 illustrates a generalized BMD 7700 including realtime invasive sense measurement. BMD 7700 includes one or more micro-electro-mechanical systems (MEMS) 7705 to measure realtime invasive sense measurement for BMD 7700. MEMS 7705 are secured to BMD 7700, such as by for example, an attachment or other coupling to a handle 7710 of BMD 7700. As illustrated, BMD 7700 includes an acetabular cup C for installation, though other systems may be used for different prosthetics.

During a procedure, MEMS 7705 provides realtime parametric evaluation of relevant information that may be needed or desired by an operator of handle 7710. For example, an orientation and seatedness of cup C may be evaluated in realtime to allow the operator to suspend operation when a desired orientation and/or seatedness has been achieved. MEMS 7705 may evaluate orientation, displacement depth, seatedness, using a range of potential systems, including force, acceleration, vibration, acoustics, and other information. Just as an interaction between cup C and an installation site may produce an FR curve as described herein, various interactions of BMD 7700 or one or more components of BMD 7700 (e.g., cup C) with the installation site may produce characteristic profiles or “prints” that change during the realtime operation. Monitoring these parametric prints in true realtime may provide the operator with helpful information that is not available with a series of pre-process measurement and post-process measurement.

The force parameter has been described herein. Other parameters of acceleration, vibration, acoustic, and the like information may provide helpful information as well by including appropriate sensing structures for acceleration, vibration, acoustic, and the like. In the case of an installation depth of an acetabular cup, these parameters may help the operator to identify and differentiate between the three zones: too little seatedness zone, sweet zone, and fracture-risk zone. The specifics by which these zones are detected and identified are likely to be different however.

BMD 7700, by appropriate selection of multiple systems in MEMS 7705, may improve performance by providing a logical product of different parametric evaluations. That is, while any single parameter of force, acceleration, vibration, acoustic, or the like may offer improved performance, having multiple different sensors all operating in true realtime to cross/double check can offer improved performance.

In some cases, a system may not identify that the prosthesis is in the sweet zone unless multiple parametric systems concur. In other cases, it may be that a first to detect a fracture-risk zone may result in suspension or termination of the installation process. Or that all systems must indicate adequate seatedness before stopping (possibly adding a further condition of providing no fracture risk detection).

Even without automatic detection of these zones, the combined information may useful to the operator in evaluating how to proceed with the installation to help maximize the desired orientation and seatedness without unnecessarily risking fracture.

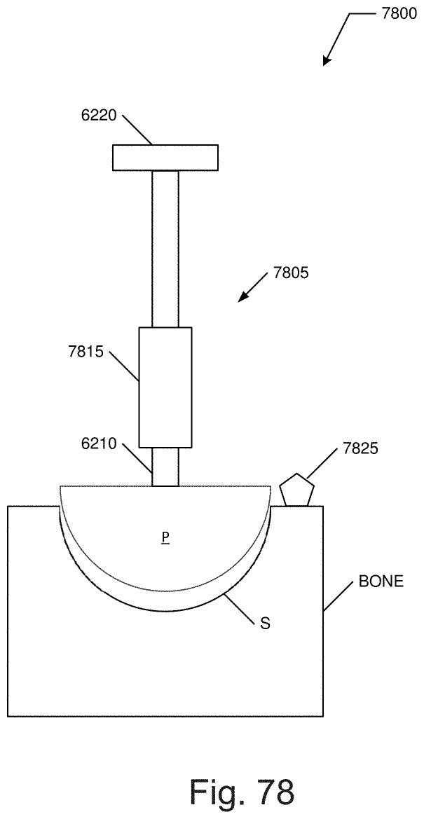

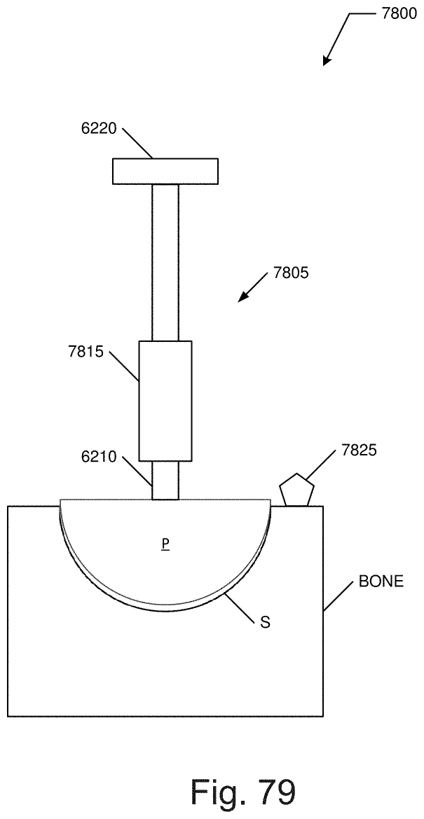

FIG. 78-FIG. 79 illustrate an alternative general force measurement system 7800 for understanding an installation of a prosthesis P into an installation site S (e.g., an acetabular cup into an acetabulum during total hip replacement procedures); FIG. 78 illustrates an initial engagement of prosthesis P to a cavity at installation site S when prosthesis P is secured to a force sensing tool 7805; FIG. 79 illustrates a partial installation of prosthesis P into the cavity by operation of force sensing tool 7805. System 7800 generally conforms to system 6200 with the inclusion of a system 7825.

Tool 7805 includes an elongate member 6210, such as a shaft, rod, or the like. There may be many different embodiments but tool 7805 may include a mechanism for direct or indirect measurement of impact forces (mIF) such as by inclusion of an optional in-line sensor 7815. Further, as illustrated tool 7805 allows for application of an external force applied to tool 7805. In some embodiments, another sensor 6220 may be used to measure this applied force as a theoretical maximum impact force (TMIF). In some cases, the TMIF is applied from outside and in other systems, the application is from tool 7805 itself. In some cases, system 7800 has a priori knowledge of the force applied or it can estimate it without use of sensor 6220. Depending upon an implementation, various user interface elements and controls may be included, including indicators for various measured, calculated, and/or determined status information.

System 7825 is illustrated as a structure installed proximate the cavity of installation site S of the bone. System 7825 may include a set of sensors selected from a group including one or more combinations of sensors for vibrations, pressures, shears, torsions, accelerations, motions, displacements, proximities, impulses, shocks, deformations, acoustics, sounds, temperatures, optics, currents, voltages, and the like. System 7825 may provide an additional, or alternative, determination of a status of insertion for prosthesis P. This determination may be used cooperatively, alternatively, confirmatorily, supportively, comparatively, or in some other fashion with respect to any other determinations from tool 7805 or system 7800 for indicating, determining, calculating, measuring, assessing, evaluating, quantifying, or measurement of the status of insertion for prosthesis P.

As described herein, the status of prosthesis may include an assessment of the current insertion zone for prosthesis P such as described herein in reference to FIG. 50-FIG. 55, FIG. 61, FIG. 62, FIG. 65, FIG. 67, FIG. 68, FIG. 72, and/or FIG. 76 (e.g., “poor seating,” “magic zone,” or “fracture zone”).

The one or more sensors of system 7825 may be installed at one location of system 7800 (e.g., all on the bone of installation site S, or all on tool 7805) or may be distributed among different components of system 7800.

For some embodiments, a difference between system 6200 and system 7800 is that system 7800 at least senses properties, characteristics, first or higher ordered derivatives over time of sensed parameters, and the like relating to bone at the implant site S consequent to insertion of prosthesis P into installation site S while system 6200 evaluates forces, displacements, motions, first or higher ordered derivatives over time of these, and the like of insertion of prosthesis P into the bone.

For system 7800, a set of output from the set of sensors of system 7825 may provide discernable profile signatures that identify a particular status of prosthesis P at any particular time. This may be a realtime status during continuous operation of tool 7805 when processing the prosthesis. This status, when quantified, may be considered an insertion metric or a measurement of a quality of insertion.

In some embodiments, and in some implant scenarios based upon a relationship between prosthesis P and installation site S, the insertion metric may also strongly correlate, represent, indicate, or the like, to a fixation (or seatedness) metric. That is, it may also represent a quality of fixation.

For example, in the case of an acetabular cup inserted into a reamed cavity in a hip bone, when the resistive forces include prosthesis-bone forces between the hoop, rim, and other non-dome portions of the cup, the insertion metric may also more strongly represent a seatedness metric (e.g., how much force it may require to pull or extract prosthesis P from installation site S after press-fit installation.

Systems 6200 and system 7800 may each determine, quantify, and/or indicate one or more of these metrics. The indication may provide a simple go/no go guidance as to whether prosthesis P is in the magic zone/B-cloud (e.g., go) or in the fracture zone (e.g., no go). A simple signal may provide these indications, for example green light for go and red light for no go.

During operation, as mIF begins to approach TMIF, system 7800 understands that prosthesis P is not moving much, if any, in response to the TMIF (when it is kept relatively constant). An advantage to the mechanical tools is their ability to repeatably apply a known/predetermined force allowing for understanding of where the process is on an applicable FR curve for prosthesis P at installation site S. For example, in FIG. 79, the mIF, for a constant applied force, is closer to TMIF than in the case of FIG. 78.

The arrangement of FIG. 78-FIG. 79 may be implemented in many different ways as further explained herein for improving installation and reducing risk of fracture.

Some embodiments demonstrate that when a prosthesis is press fit into bone, a resistive force FR is encountered. This resistive force increases as the prosthesis is inserted deeper into a cavity. On a force vs. depth of insertion graph this resistive force (FR) may have a characteristic hockey shape (exponential curve).

Some embodiments have described three zones on this curve. The “poor seating zone”, the “sweet spot”, and the “fracture zone”. In the fracture zone additional insertion forces may lead to minimal gains in (quality of fixation/pull out force). This may indicate that there is a sweet spot on the curve where good/adequate fixation is achieved (even without full seating of the prosthesis) and that the surgeon should not continue to impart forces to the cup/cavity interface for minimal insertion depth gain and concurrent unnecessary increase in fracture risk. On the other hand there is risk of not seating the prosthesis fully enough which can lead to poor initial fixation and eventual loosening.

Noted herein that both poor initial seating and occult fractures at the time of impaction can lead to the same effect of loosening of the prosthesis and failure of important bone ingrowth leading to subsequent potential problems such as infection or osteolysis. The cost of these problems to society has not been evaluated or contemplated so far but is expected to be in the multiple billions of dollars.

Some embodiments having included a discussion of a more sophisticated inserting process where the prosthesis is installed into bone with a) vibratory techniques, b) controlled impaction techniques, and/or c) a hybrid version using both types of techniques.

Relevant here is a prior art problem associated with how a surgeon impacts a prosthesis into bone or (a prosthesis into a prosthesis). This may be described as a weak (open loop) system where there is no technological feedback to the surgeon other than his/her own auditory and tactile senses which may be impaired during the procedure by the environment of the procedure room.

Some embodiments “close” this loop in the process of PRESS FIT fixation by measuring parametric values (such as force, acceleration, vibrations) in the system which includes the bone, the prosthesis, the impacting tool and the surgeon. Some embodiments may include a method to use measurement of force within this system and the processing of its derivatives to accurately assess an insertion metric that may under various conditions reflect a quality of fixation of the implant into the bone (or of a mechanical join of one component of a modular prosthesis to another component of that modular prosthesis). This gives the surgeon a technological tool to assess when good and adequate fixation has been achieved while reducing, minimizing, and/or eliminating risks of poor fixation or fracture on the margins of the ‘sweet spot’.

Some embodiments of system 7800 may expand on the idea by including a measurement of vibrations to assess the metrics and quality of insertion and/or fixation as an adjunct, or alternative, to measurement of force.

Currently in orthopedics some surgeons may use their tactile and auditory senses to estimate when a prosthesis is properly seated. And as noted herein, this method is inadequate. This problem occurs most notably with press fit fixation of femoral and humeral prosthesis in hip and shoulder replacement, as well as acetabular component fixation.

Some embodiments of system 7800 contemplate that with the use of appropriate sensors that may be incorporated in system 7800, for example a MEMS module attached to a mechatronic handle-insertion tool, independently attached to bone, and/or a discrete system in the vicinity of the procedure, to establish, measure, estimate, quantify, evaluate, assess vibrational responses of bone such as may be derivable through sensing of “frequency spectra”. This can be accomplished for example by using “shock accelerometers” or other forms of ‘vibrational sensors, including sound or acoustic sensors. The frequency spectra from shock accelerometers, for example, may be produced for each of the stages of the FR curve (e.g., poor seating, sweet spot, and fracture zone). Evaluation of these frequency spectra or other sensor output can assist the surgeon in determining how to proceed with installation of an implant/prosthesis at any given time. For example, should the magnitude of force be increased and should the application of force be stopped or suspended. Similarly other vibrational characteristics of bone during cup cavity insertion (including changes in the pitch of sound) can assist the surgeon technologically on the assessment of the various metrics of installation and/or fixation, in a press-fit situation. In this manner “peak forces” that typically lead to high fracture risk without attendant enhancement of quality of fixation may be avoided.

Assessment of vibrations in bone can provide a guide map for the surgeon on where on an FR curve a prosthesis may find itself at any moment of an installation process, which may provide a technological assessment of when adequate fixation has been achieved without unnecessarily applying peak forces that risk fracture of the cavity, and subsequent loosening and (potential additional problems such as infection and osetolysis). Measurement of vibrational spectra in an inserting tool or directly in bone may be used to assess quality of insertion and/or (quality of fixation) with press fit technology.

Other procedures besides cup installation (e.g., installing a different type of prosthesis), other processes other than prosthesis installation (e.g., assembling a modular prosthesis), and other invasive operations (e.g., bone preparation), and other medical interventions that do not relate to prosthesis preparation, installation, and assembly may all benefit from providing true realtime analysis and feedback.

Feedback from a MEMS system may be accomplished by one or more of a display or indicator on or integrated with the device, and/or an associated module in communication with the MEMS system/display, a robot or navigation system in communication with the MEMS system and/or an associated module.

The description herein includes a discussion of use of intracorporeal sensors that may be installed for various tasks. For example, there are a set of sensors disposed on an implant (FIG. 15), metabolite sensors (FIG. 43), and a force sensor in FIG. 49, among other sensors. As illustrated, these sensors may be affixed to an implant or tissue, among other intracorporeal uses. Described below is an extension of these ideas for in situ sensing in other contexts.

In general, embodiments of the present invention for in situ sensing includes fixation of a set of one or more physical references within the body (intracorporeal fixation) to tissue of interest. These references are configured to be sensed outside the body, either actively or passively, depending upon configuration, implementation, and application.

The fixation may be mechanical, such as a reference tag incorporated into a fastener, screw, plug, and the like, in some instances the mechanical fixation may be accomplished by a suture element fixing the reference tag to the tissue of interest, or may be an adhesive that fixes the reference tag into position. The reference tags may be incorporated into various locations of the mechanical system (e.g., a proximal, medial, or distal end of a fastener). The fixation structure may position the reference tag above the tissue surface, at the tissue surface, or beneath the tissue surface.

The reference tags may be passive, active, or a hybrid and preferably accessible extracorporeally by a counterpart system. A passive system includes reference tags providing desired intracorporeal information in response to an external operation. An active system includes reference tags providing desired intracorporeal information independent of external operations. A hybrid system includes reference tags that share features of the passive and active systems.

Described below is a particular context for an implementation of an embodiment of the present invention for externally accessible intracorporeally fixed markers. This context includes replacement of a hip or a shoulder. Sometimes the replacement is in response to degeneration of the joint wherein biomechanics of the afflicted joint is different from a complementary non-processed joint (e.g., one diseased shoulder or hip compared to the other non-diseased shoulder or hip). The biomechanics may be impacted by a length and an offset of the replacement. Mismatches in any of these attributes between the replacement joint and the non-processed joint may cause adverse response to the procedure. This context may be simpler than other contexts because hip repair requires only X & Y (or distance and angle) conformation between the repair and the reference joint. Freedom of motion/movement of the joint components are constrained within a plane so the variables in repair are fewer. Other repairs may be simpler (one degree of freedom of motion) or more complex (three, or more, degrees of freedom of motion) with freedom of motion including motion in X, Y, Z directions, or rotations about these directions, for example. Some embodiments contemplate a minimum number of reference tags equal to the freedoms motion). Other embodiments may use multiple numbers of reference tags per degree of freedom of motion. Possible advantages include more accuracy/precision and/or provision of statistical summary of reference locations.

Matching the replacement joint to the non-processed joint is not a simple matter and has many opportunities for error. While the surgeon begins the procedure with a plan that has resulted from pre-operative imaging and assessment. That plan includes a desired length and an offset for the replaced joint (which may be expressed in Cartesian or Polar coordinates, or other reference system).

This length and offset (or angle and magnitude equivalence) is affected by the preparation and the size/configuration of the implant(s) to be installed. In some instances, one component of the joint replacement is installed, and then complementary components are tested in a sort of trial and assessment. In the prior art, this assessment includes successive images after each trial to evaluate how close the current status of the replacement has met the plan. Different imaging techniques may be used, such as X-Ray, fluoroscopy, among others.

Each independent image sometimes requires that a portable imaging system be brought into the operating room each time an assessment it to be made. The assessment is subjective as to each image—one or more anatomical landmarks are identified and then measurements are made to ascertain the characteristics of the replacement and compare it to the plan. Unfortunately, these landmarks are not sharp, pinpoint structures. Different observers will determine a location for them at different positions, usually close to, but different from, another person's determination of those positions. Further, the same person may not always determine the same location for the same landmark. And that is on a per image basis—with each successive image taken during the assessment process incorporating these subjective variances.

It would be desirable should these assessments be performed more quickly and more objectively. An embodiment of the in situ sensing invention may be adapted to address this problem and provide a range of possible solutions as discussed herein.

Total hip arthroplasty (THA) has developed into one of the most successful and widespread orthopedic operations, providing pain relief and restoring function in patients with severe arthritis affecting the hip joint. During this operation, a surgeon replaces damaged bone and cartilage with a prosthetic femoral stem and cup. Since the inception of THA, the method has benefitted from improvement in prosthesis materials and design, as well as refinement of surgical techniques. During THA, the diseased femoral head and acetabular portions are removed and replaced with implants to restore function to the hip joint. One of the intraoperative challenges of THA is correcting limb length inequality without compromising stability. Discrepancy in leg length is often considered to be a problem after THA and can adversely affect an otherwise excellent outcome. Furthermore, it has been associated with patient dissatisfaction and remains of the most common reasons for litigation against the orthopedic community. Various methods have been sought to minimize limb length discrepancy; however, no method has provided an accurate, easy and reproducible way for surgeons to monitor in real time leg length changes during the procedure.

Conventional surgical handwork requires competencies such as dexterity or fine motor skills, which are complemented by visual and tactile feedback. Many conventional intraoperative techniques have been developed to measure changes in leg length by measuring reference markers placed on the femur and pelvis which are routinely checked during THA. However, these techniques are fraught with inaccuracies due to reliance on stable fixation of large pins and screws to bone, which is unlikely due to the fact that the pins are directly in the way of the surgeon and assistants who tend to push and pull on them. As well, these techniques are dependent on the ability to exactly reposition the original relationship of the femur to the pelvis during the operation.

Advanced technology in THA has involved the development of computer assisted and robotic surgery which attempts to aid the surgeon with obtaining optimal goals during surgery, including enhanced ability to obtain desired leg length and hip offset.

Computer navigation in orthopedic surgery has emerged over the last several decades as an independent field that enables computer tracking systems and robotic devices to improve visibility to the surgical field and increase application accuracy. Computer assisted surgery involves three major components: (i) a therapeutic object, the patient's bone, which is the target of treatment, (ii) a virtual object, which is the virtual representation in the planning and navigation computer, and (iii) a so-called navigator that links both objects. The central element of the computer navigation and robotic systems in orthopedics is the navigator. It is a device that establishes a global, three-dimensional (3-D) coordinate system (COS) in which the target (bone) is to be treated and the current location and orientation of the utilized end-effectors are mathematically described.

End effectors are passive surgical instruments but can also be semi-active or active devices. One of the main functions of the navigator is to enable the transmission of positional information between the end effector, the target, and virtual object. For robotic devices, the robot itself plays the role of the navigator, while in surgical navigation, a position tracking device is used.

For the purposes of establishment of a computer system use in orthopedic surgery three key procedural requirements have to be fulfilled. The first is the calibration of the end effectors, which means to describe the end effectors geometry and shape in the COS of the navigator. For this purpose, it is required to establish physically a local coordinate system COS at the end effector. When an optical tracker is used, this is done via rigid attachment of three or more optical markers onto each end effector.

The second is registration, which aims to provide a geometrical transformation between the target (patient's bone) and the virtual object in order to display the end effector's localization with respect to the virtual representation, just like the display of the location of a car in a map in a GPS-based navigation system. The geometrical transformation can be rigid or non-rigid. In the literature a wide variety of registration concepts and associated algorithms exist.

The third key ingredient is referencing, which is necessary to compensate for possible motion of the navigator and/or the target object during the surgical actions. This done by attaching a so-called “dynamic reference base” DRB holding three or more optical markers to the target object or immobilizing the target object with respect to the navigator.

Registration closes the gap between the virtual object and the target object. The navigator enables this connection by providing a global coordinate space. In addition, it links the surgical end effectors, with which the procedure is carried out, to the target object that they act upon. For robotic systems used in orthopedic surgery such as MAKO systems and Blue Belt Technologies, the robot itself is the navigator.

These systems mostly use optical tracking of objects using operating room compatible infrared light that is either actively emitted or passively reflected from the tracked objects. Reference bases (Markers) are therefore attached to surgical end effectors which hold either light emitting diodes (LED, active) or light reflecting spheres or plates (passive). These reference bases (markers) are also attached to bone in the following manner. A screw or pin (Schantz screw) is placed into bone, which is then attached to a clamp, which is then attached to a senor/marker/reference base.

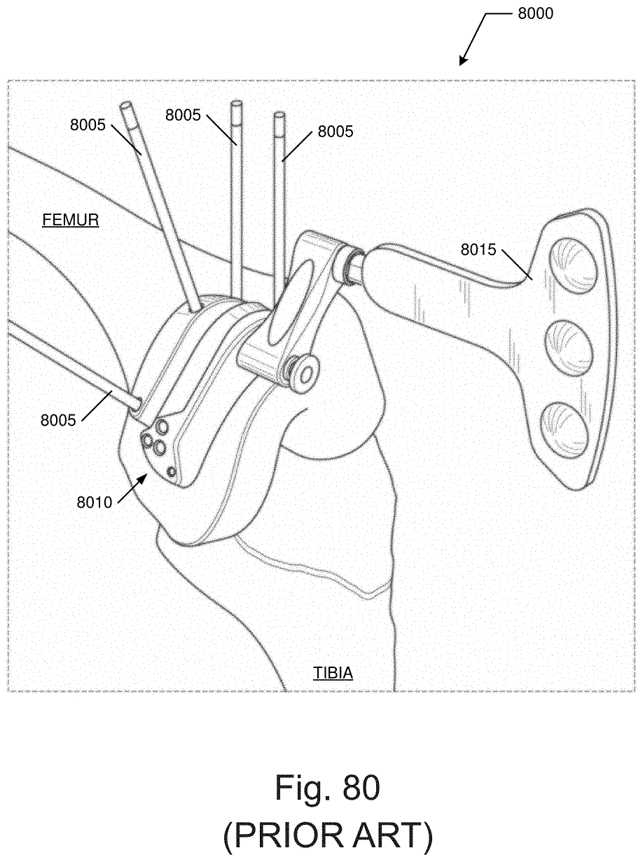

FIG. 80 illustrates a conventional sensing implementation system 8000 implementing a set of Schantz screws 8005 fixated to bone (e.g., a femur), which may be coupled, directly or indirectly, to a clamp 8010 and then attached a sensor, marker, reference base or optical tracker 8015.

These systems therefore produce a coordinate system in the OR space in which the position of the surgical tools and the patient's bone in relation to each other are precisely known, presenting greater detail, three dimensional views and sight of internal structures which are invisible to the naked eye.

Despite its touted advantages of increased accuracy surgical navigation has yet to gain general acceptance among orthopedic surgeons. The barriers to adoption appear to be intrinsic to the technology itself, including intra-operative glitches, unreliable accuracy, frustration with intra-operative registration, and line of sight issues, bulkiness of the equipment and extra time required for the add ons. These findings suggest significant improvements with technology will be required to improve the adoption rate of surgical navigation and robotic surgery.

Reference: Computer-Assisted Orthopedic Surgery: Current State and Future Perspective Guoyan Zheng, Lutz P. Nolte.

The concept of navigation in orthopedic surgery, in particular THA, was developed as a panacea to provide a solution for (a) cutting bone more accurately, (b) obtaining exact alignment of implants and (c) obtaining perfect leg lengths, by providing greater visibility of the patient's anatomy. However, at least with respects to leg length assessment, the concept that better visualization of the anatomy leads to better reproducibility of the leg lengths maybe flawed and spurious. It may be excessive (overkill) to have to determine the exact relationship of the patient's bone, cutting tools, implants and a virtual (desired) plan in the coordinate system of the OR space, when all that is needed is the ability to quickly and accurately determine the distance between two reference points in the OR, such that ΔX and ΔY can be continuously monitored.

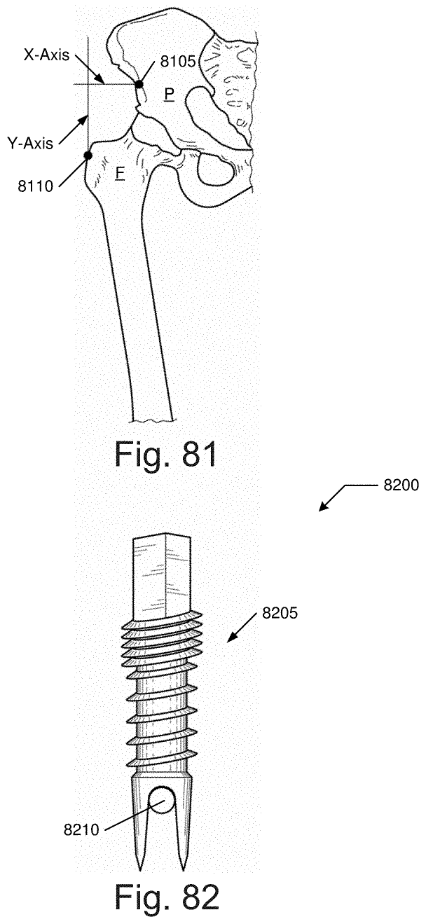

In general, orthopedic surgeons are concerned with three distinct issues during hip replacement surgery: (i) proper seatedness (or the ability to obtain a good pull out force, extraction force, and/or optimal interference fit), (ii) obtaining proper alignment (for example the desired amount of inclination and abduction angle for the acetabular cup), and (iii) obtaining proper or desired leg length and offset in THA. FIG. 81 illustrates two fixed points (a first point 8105 on the pelvis P and a second fixed point 8110 femur F) that may be used to measure changes in leg length (y-axis) and offset (x-axis).

In some of our previous applications we identified a significant metric that navigation does not address, which includes an assessment of the quality of seatedness of the prosthesis, or how to quantitatively assess the pull-out force, extraction force of an implant in vivo and in real-time during installation of the implant. Related U.S. patent applications (including U.S. Ser. No. 15/284,091, U.S. Ser. No. 15/234,782, U.S. Ser. No. 15/592,229, and U.S. Ser. No. 15/687,324, hereby expressly incorporated by reference thereto) concerned themselves with how to obtain optimal interference fit for cement-less arthroplasty.

An additional group of applications (including U.S. Ser. No. 15/055,942, U.S. Ser. No. 15/235,094, U.S. Ser. No. 16/050,662, and U.S. Ser. No. 16/375,736, hereby expressly incorporated by reference thereto) describe a simple process of calibrating the OR coordinate space and continuously monitoring both the target bone (acetabulum) and end effector (cutting tools or acetabular cup) with inertial measuring unit (IMU) technologies.

Further, orthopedic surgeons are interested in two specific measurements in THA (Leg length and Offset) and wish to obtain these with values as efficiently as possible. Restoring or maintaining equivalent leg lengths is an important goal of total hip arthroplasty THA. Leg length inequality after THA has long been recognized as a complication of the procedure. Postoperatively, 32%-41% of patients notice a difference in leg lengths and up to 45% require use of a shoe lift. The vast majority of patients who undergo THA have less than a 10 mm discrepancy postoperatively. The difference in leg lengths causes uneven loading of hip joints as well as the lumber spine, and is related to hip and back pain, as well as poor gait.

Recreating hip offset, the difference between the center of rotation of the femoral head and the axis of the femur, is another consideration in THA, as appropriately increased offset has been associated with greater abductor muscle strength, improved pelvic mobility, superior gait, and higher patient-reported outcomes.

Both of these metrics (leg length and offset) are typically measured by one of three methods: 1 visual and physical gauges, 2. computer navigation and robotic surgery as described above, 3. Direct anterior approach and use of intra-operative fluoroscopy.

Computer navigation and robotic surgery, as noted above, have not gained general adoption by orthopedic surgeons due to significant add-ons to the operation with landmarking, registration, application of markers and tracking with optical equipment, which makes the operation more cumbersome, expensive, more complicated, and which on many occasions does not provide the accuracy promised. Typical added time to the operation is from 30 minutes to 120 minutes.

Direct anterior approach THA has become increasingly popular and provides the opportunity to utilize intraoperative fluoroscopy to assess leg length and offset. However, this is a less natural operation for most orthopedic surgeons and associated with steep learning curve and difficulty with placement of the femoral component, leading to a high number of complications associated with the femoral component (loosening and or fracture). As well intraoperative fluoroscopy exposes the patient and the surgeons to excessive amounts of radiation. Use of fluoroscopy also requires movement of C arm machine in and out of the operative site for spot checks of component position. This technique is reliant on the surgeon's ability to visually reproducibly pick the same landmarks on the femur and pelvis (every time the machine is brought in and out of the operative site). This process is prone to introduction of errors related to intra-observer reliably issues and variance, with potential for compounding errors with each assessment.

Conventional intraoperative techniques utilize pins, sutures and calipers to measure changes in leg length intraoperatively based on the distance between two reference points marked on the pelvis and the femur. While these techniques are practical, they are prone to error due to femur and pelvis repositioning, removal and reapplication of pins and calipers, all of which can lead to substantial errors in assessing the leg length and offset as the fixed reference points (sutures, pins, calipers) are not perfectly fixed, cumbersome and prone to displacement during the surgical procedure.

In this application we have contemplated a new and simple system to evaluate leg length discrepancy and hip offset. The system described may not concern itself with complicated navigation systems and mathematical algorithms that attempt to reproduce the exact contour, position and shape of the target bone in the OR coordinate space. In fact, our concept is to keep things simple and only to obtain the information we need as quickly and efficiently as possible. Therefore, establishing a coordinate system in the OR space and use of complicated mathematical algorithms appears extraneous.

Surgeons generally are fully aware of the leg length discrepancy and offset prior to the operation by simply viewing (assessing) the diseased and unaffected hips on a simple pelvis X-ray. For example, the surgeon may decide pre-operatively that she wants to increase leg length by 3 mm and offset by 5 mm, or that she may want to keep the leg length and offset exactly the same (i.e. a delta of zero for both leg length and offset maybe desired). Therefore, the surgeon is only interested in the difference (delta), that occurs in leg length and offset, between the preoperative diseased hip and the postoperative replaced hip; and not the absolute values of the lengths of the femur.

The new system described herein provides the ability to accurately reproduce the desired leg length and offset with minimal added time (one to two minutes) and with no interruption to the flow of the operation, no bulky and cumbersome equipment in the way of the surgeon, no significant increase in cost of purchasing capital equipment (cost of MAKO robot $1 Million), and yet to accomplish this with simplicity, accuracy and precision.

First, surgeons always preoperatively assess the X-rays to decide whether they desire to change leg lengths with THA operation. That is, they decide whether they want to keep the leg lengths the same or alter them. Once that decision is made, the surgeon does not need to know the exact (absolute value) of leg length (for example it is irrelevant for the surgeon to know that the length of one femur is 80 cm and the other 81 cm), but rather the surgeon needs to know the delta (A) difference between preoperative and postoperative leg lengths (and offset) when the implants are installed. In other words, how did the leg length change after the surgeon cut and removed the diseased femoral neck and head and installed the femoral stem, femoral head and the new acetabular cup.

Previous conventional and navigation techniques require the use of large pins to be attached to bone and subsequently attached to trackers, calipers and/or sutures with clamps, with attendant limitations described above, such as obstruction of the surgical field, susceptibly of loosening of screw to bone and loosening of the clamp holding the trackers to the screw, all of which introduces measurement errors.

All of the limitations noted above may be eliminated when the two reference markers can be rapidly, securely and stealthily established on the femur and pelvis (less than a minute); and their relationship to each other continually transduced and monitored by the surgeon throughout the THA operation. Essentially, the reference markers/sensors can be securely and quickly attached to any two bones within the patient's body, and the spatial relationship between these markers can be accurately determined and monitored. Therefore, the leg length and offset changes (delta) can be known in real time, and shown live to the surgeon during the operation, with minimal added time and cost and with no added cumbersome bulky equipment, which adds to complexity of surgery, provides too much information, and increases the risk of contamination and infection.

Considering the advance of two essential technologies: (i) the arrival of progressively more miniature and inexpensive sensors and, (ii) superfast networking kits, we anticipate that the two reference markers used for the pelvis and femur (or any other combination of two bones in the human body) can be used as sensors embedded in and housed securely in an anchor such as screw, pin and/or a hook and will only improve with advancement of these and related/similar technologies. This screw/sensor combination provides the utility of easy application with a drill or hand held screw driver as is usually done when applying anchors to the proximal humerus for rotator cuff surgery, within seconds. It also has the utility of being perfectly secure, miniature and completely out of the surgeon's way and workflow. Another advantage of the screw/sensor is that the sensors can be applied in close proximity to each other (for example superior acetabulum to lateral edge of the greater trochanter—10 cm) to prevent loss of precision and accuracy. These screw/sensors can be rapidly applied and later removed to provide leg length and offset information, without unnecessary demand for complex mathematical equations and algorithms to determine OR coordinate space.

The screw/sensor combination can be left in place, removed, or designed to be absorbable/resorbable.

Ultimately, the surgeon currently has no way to continually monitor leg length and offset changes during THA surgery. With current techniques, every time the surgeon wants to know what leg length and offset change have occurred, the surgeon has to bring a big bulky equipment into the surgical field (e.g., robot, computer navigation system, or fluoroscopy) and take a picture/image (X-ray or optical tracking) and depend on some secondary calculation (measurement of leg length on X-ray or fluoroscopy screen) or (secondary processing with mathematical algorithms within a navigation system) to determine changes in length and/or offset (position of the femur in relation to the pelvis) in the X and Y planes.

A proposed solution includes a set of tags that disposes one or more devices within a body (e.g., screw, hook, pin, fastener or the like), the body mechanically engaging a portion of tissue, such as with threads, barbs, annular rings, and the like. Alternatively there may be some other mode securely fixing the tag to tissue (which may include bone) with suture and or adhesive. This arrangement may allow non-interventional realtime/near realtime calculation of leg length and offset delta (change) without any bulky equipment or added time or disruption of workflow to the surgery (anchoring).

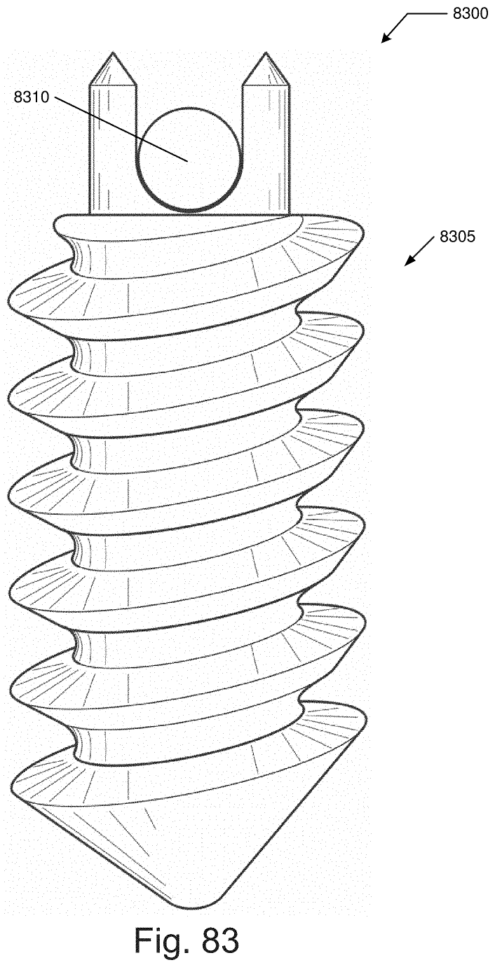

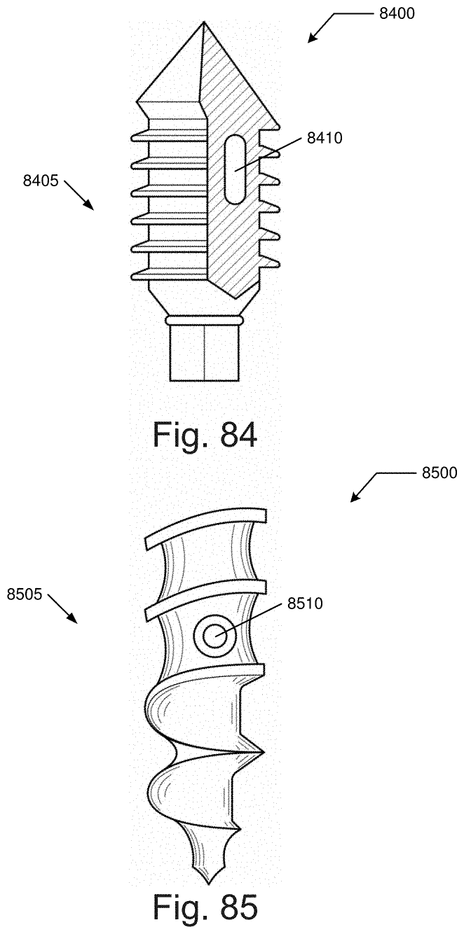

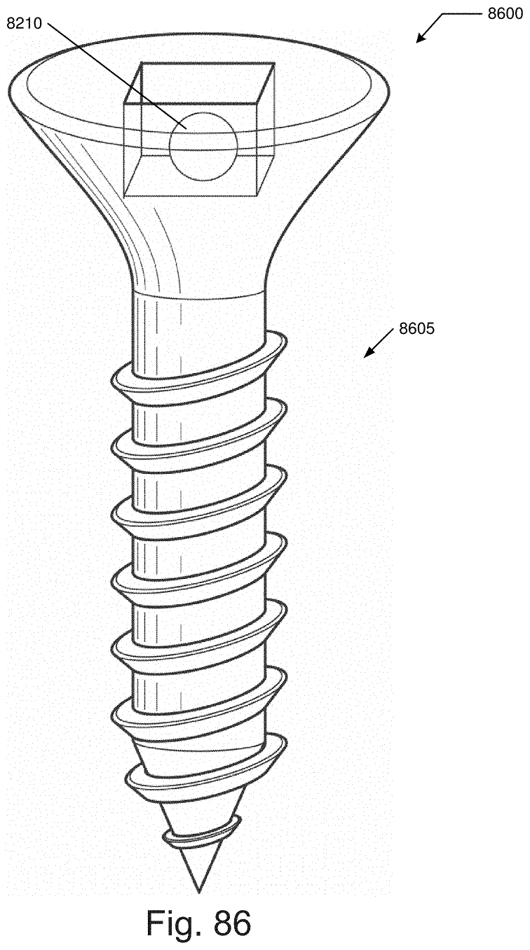

FIG. 82-FIG. 86 illustrate various types of tags and fixing modalities including screws, barbs, hooks, nails, plugs, fasteners, anchors, and threaded pins that include sensors, references, or other passive/active component. FIG. 82 illustrates a tag 8200 having a body 8205 coupled to an active/passive device 8210. FIG. 83 illustrates a tag 8300 having a body 8305 coupled to an active/passive device 8310. FIG. 84 illustrates a tag 8400 having a body 8405 coupled to an active/passive device 8410. FIG. 85 illustrates a tag 8500 having a body 8505 coupled to an active/passive device 8510. FIG. 86 illustrates a tag 8600 having a body 8605 coupled to an active/passive device 8610. Surgeons have access to many different fasteners and fastening/fixing systems that will include a body that may be adapted to include an active/passive device (examples of active/passive devices are further described herein). The body may deliver/present the active/passive device above, at, or below a surface of body tissue (e.g., bone, muscle, and connective tissue) to which the body is affixed. The fixing may be performed mechanically such as by use of a thread, helical or annular rings, and the like, use of suture material, and/or adhesive. In many implementations the fixation of the body delivers and attaches the reference/tag intracorporeally to subcutaneous tissue (such as through an incision, portal, or other subcutaneous access) and the active/passive device is accessible extracorporeally for different uses via different active, passive, or hybrid systems.

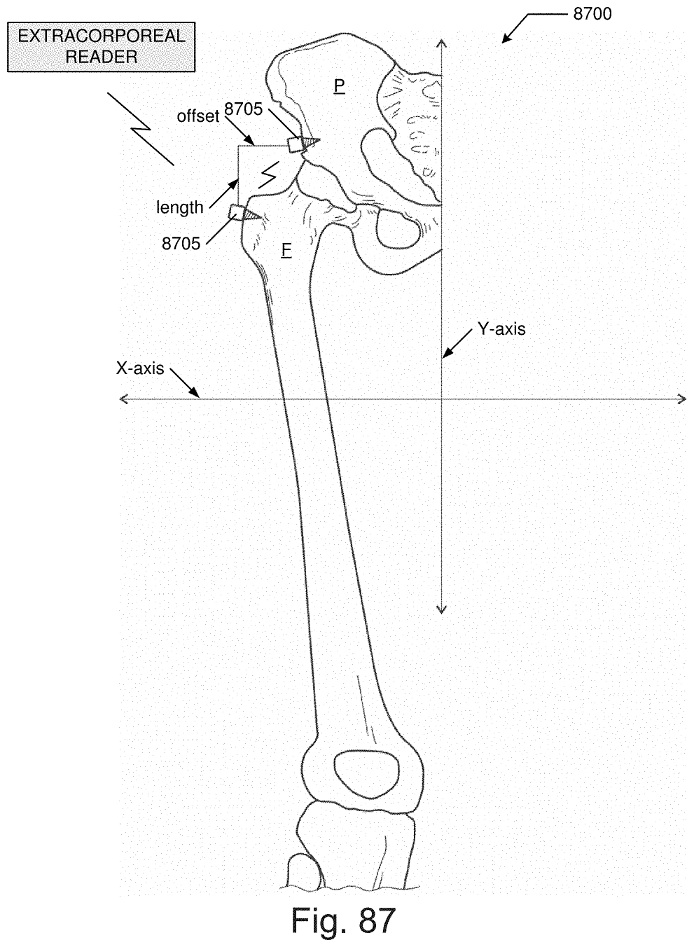

FIG. 87 illustrates a set of tags, each tag 8705 having an active/passive device (e.g., a sensor) incorporated within a body and applied to pelvis (P) and femur (F) to provide realtime or near realtime evaluation of leg length (e.g., delta/distance on Y-axis) and offset changes (e.g., delta/distance on X-axis) during THR. Illustrated in FIG. 87 include use of a set of tags that determine relative X and Y changes in realtime during a procedure and provide this information outside the body to the surgeon. As illustrated, one tag is fixed in place to pelvis P and one tag is fixed to femur F so that relative (as opposed to absolute) position changes of femur with respect to pelvis P may be easily monitored in realtime during a procedure. Further, these position changes are objective and not subject to observation errors/differences by different people at different times. This permits better measurement and conformance with pre-operative plans which in turn allows a surgeon to produce better outcomes for her patients. There are different technologies that may be used for the tags and fixation modalities for the bodies/devices that determine relative distance/distance changes and communicate this information extracorporeally. The solution illustrated in FIG. 87 does not require repetitive identification and use of imprecise anatomical landmarks that contribute to inaccurate assessment of offset/length.

Various type of tags may be utilized including threaded screws, barbed hooks, and straight and threaded pins with devices embedded within the structure of the tags as shown. The devices can be embedded within the head of the screw, in the body of the screw, on the face of the screw or on the tip of the screw or tag, depending on the functionality required. Devices may be embedded within suture material or attached to suture material for direct fixation to bone (or other rigid or soft tissue). Similarly, tags may be manufactured with adhesive surfaces that attach securely to bone (tissue).

Various distance and proximity devices may be incorporated into the tags. Proximity sensors sense when an object is within the sensing area where the sensor is designed to operate. Distance sensors sense distance from the object and tag interface (e.g., an extracorporeal reader, measuring device, or the like) through outputting a signal or data, which may be in the form of ultrasonic waves, laser, infra-red, LED, sonar and magnetic, and the like.

Active ultrasonic or sonar sensors may emit high frequency sound waves toward a target object and measure a time it takes for the sound waves to bounce back and use this technique to measure the distance between. Passive ultrasonic or sonar “sensors” or devices may respond to an external stimulus or reflect applied energy, or include a tag having a wholly or partially structure that is opaque to applied energy (e.g., a lead device in an X-ray imaging environment).

IR infra-red sensors may work through a principle of triangulation, or measuring the distance based on the angle of the reflected beam.

Laser distance sensors LIDAR emit laser light at the target object and as the light is reflected back the distance is calculated by using the relationship between constant speed of light in air and the time between sending and receiving the signal.

LED Time-of-Flight Distance sensors measure the elapsed time it takes for a wave pulse to reflect off and object and return to the sensor. It is capable of producing a 3D image in the X,Y,Z planes with a single snapshot by measuring the time it takes for light to travel from the emitter to the receiver.

These distance determining devices, variations or combinations thereof, may be incorporated within the tags to allow rapid, real-time non-interventional extracorporeal evaluation of the leg length and offset changes during the total hip operation. In other words, changes in the X and Y planes between the femur and pelvis can be accurately and continually monitored with little effort and time added to the THA procedure.

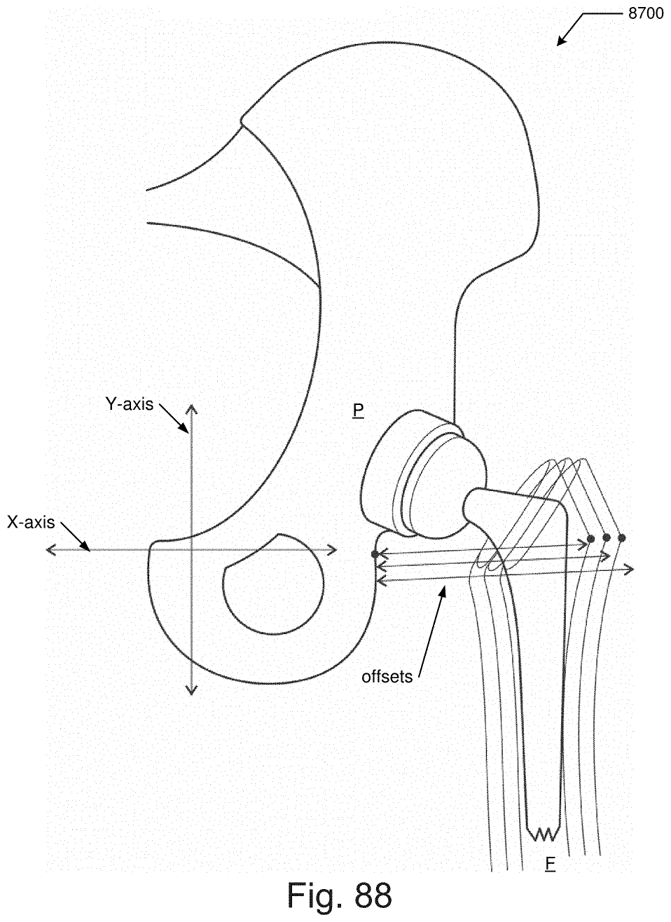

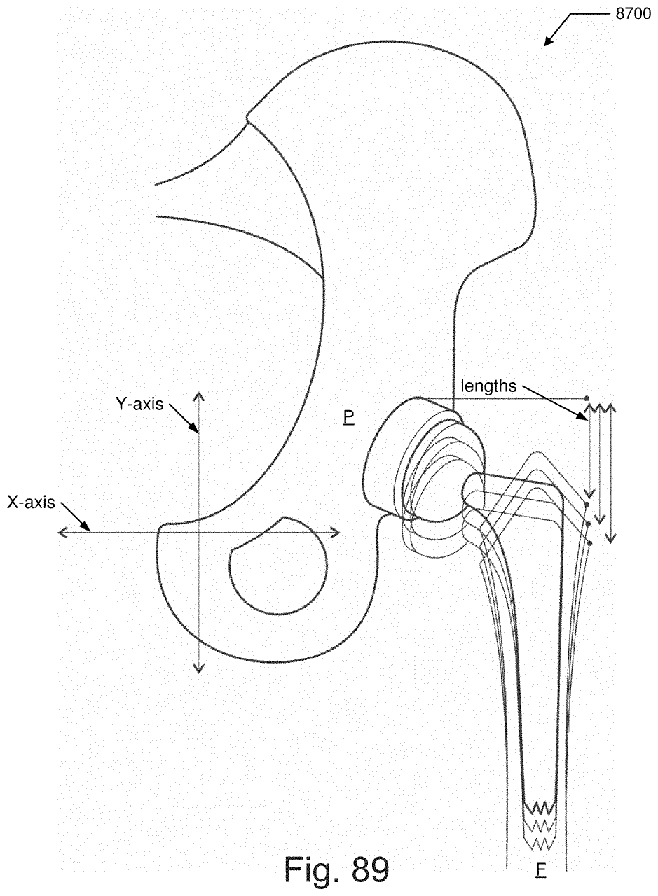

FIG. 88 illustrates measurement of different “offset” distances or deltas X in the X axis and FIG. 89 illustrates measurement of different “leg length” distances or deltas Y in the Y axis.

In a simple format, the tags may be applied in line with each other and the delta in the Y and X planes can be measured directly from the devices with minimal secondary calculations.

However, integrated circuits/microelectronics, incorporated (added) to the tags, may calculate the change in X and Y positions between two tags simultaneously, through mathematical and geometric algorithms. Similarly, angular changes between two or more tags may be calculated simultaneously through mathematical and geometric algorithms. This information may be made available non-interventionally to the surgeon live (nonstop) and in real time, through a software/hardware display, throughout the course of the operation without the need for disruptive movements of large bulky equipment in and out of the operative site or other suspension of the procedure to operate manually various measurement or monitoring systems.

In some embodiments more than two tags, for example, four or five tags, may be utilized for data fusion and averaging for increased accuracy. In some embodiments more than one type of device may be utilized in a set of tags, such as infra-red (IR) and ultrasonic tags together to increase accuracy.

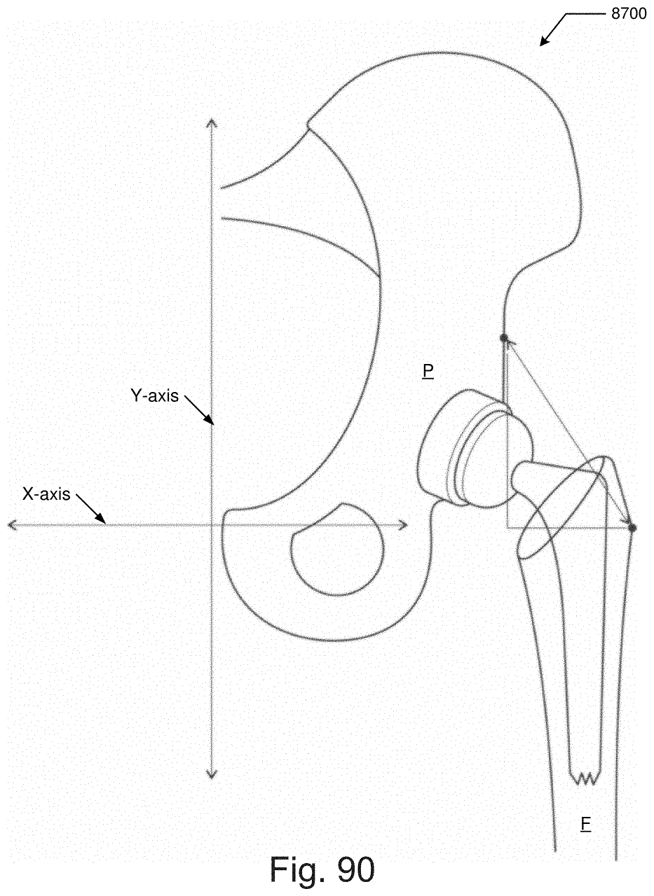

FIG. 90 illustrates integrated circuits and microelectronics incorporated within the tags calculating changes (delta) in the Y and X locations between the two tags simultaneously through mathematical and geometric algorithms.

These tags may be fixed onto or into bone, as described in U.S. patent application Ser. No. 15/592,229 and shown in FIG. 29 (sensor 2925) may be utilized to measure force, acceleration, vibration, sound, acoustics, heat, velocity and impulse.

Similarly, the same screw/sensor can be incorporated into bone, as described in U.S. patent application Ser. No. 16/375,736 as shown in FIG. 43 (sensor 4315) to measure any inflammatory metabolites (mast cells, macrophages, cytokines, chemokines, histamine, and the like) as well as cells (such as bacteria) and metal debris such as cobalt, chromium, and titanium.

Various arrangement of screw/sensors can be advantageous. For example, when using a threaded screw as the anchor, the sensor maybe best utilized when it is embedded in the screw head, or within the substance of the screw, inside the screw, or at the face of the screw or at the exact tip of the screw.

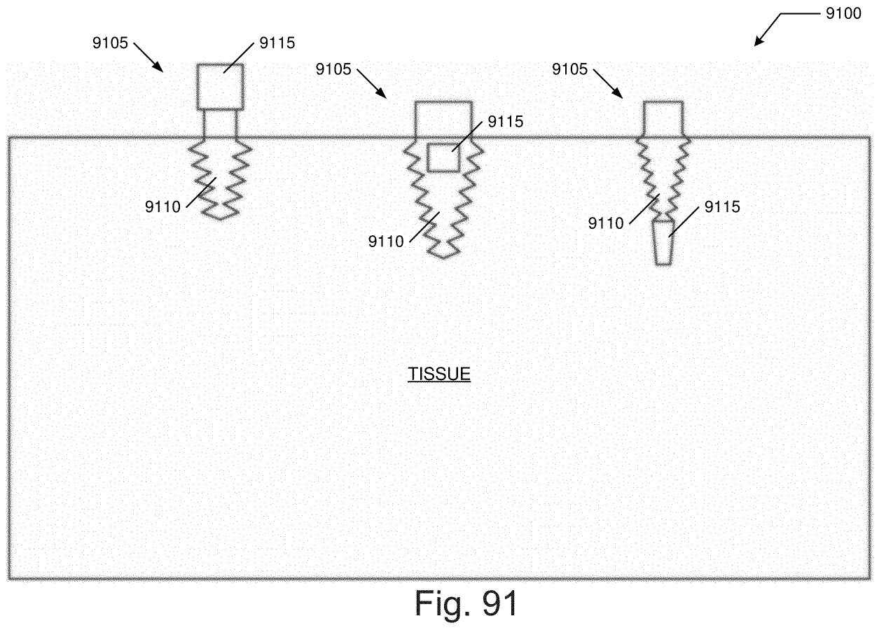

FIG. 91 illustrates a system 9100 including a set of tags 9105, each including a threaded body 9110 coupled to one or more active/passive devices 9115. Body 9110 may be similar to rotator cuff anchors that may be rapidly applied to, and removed from, tissue (e.g., bone) with a drill or hand screwdriver. The threaded screw/sensor may be utilized when rapid application and removal is required. This can be accomplished with a drill.

When the tag is intended to stay in bone without plans for removal a barbed and hooked body maybe desired. Similar to the threaded screw design, the sensors may be useful when housed at the top of the anchor, within the anchor or at the tip of the anchor. Alternatively, the tag may be useful as incorporated within a pin or Schantz screw.

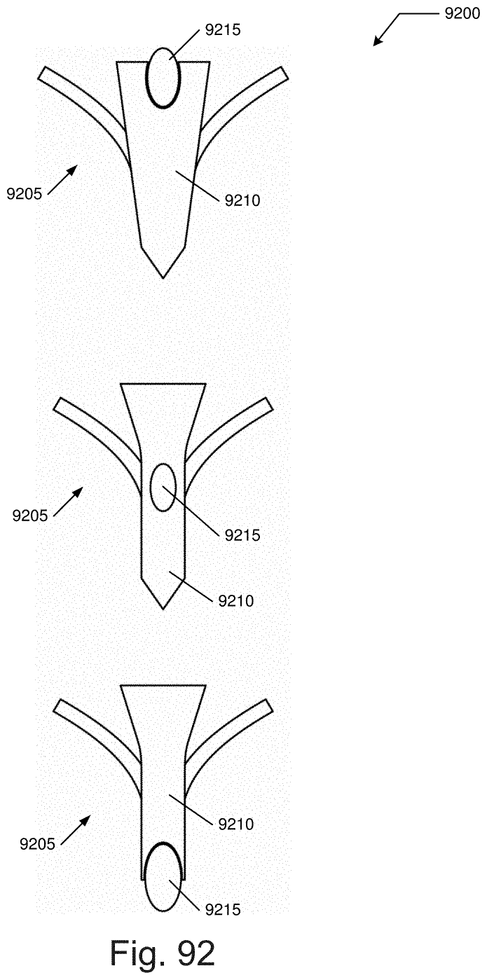

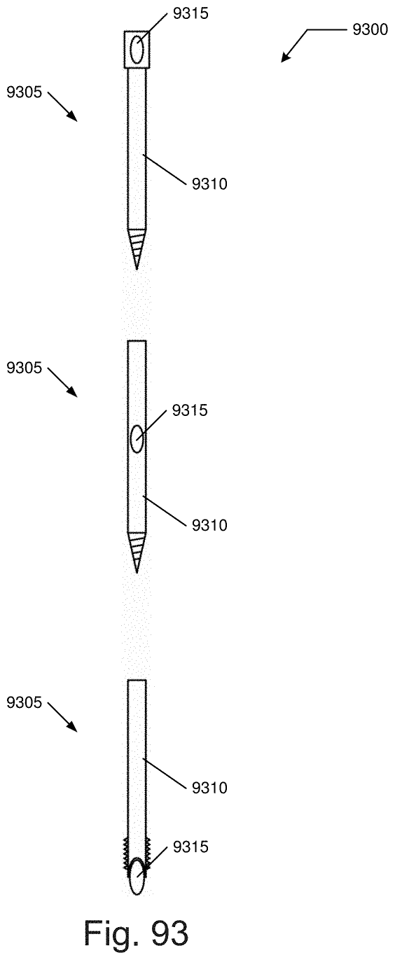

FIG. 92 illustrates a system 9200 including a set of tags 9205, each including a barbed body 9210 coupled to one or more devices 9215 disposed at different locations relative to the bodies, and FIG. 93 illustrates a system 9300 including set of Schantz tags 9305, each including a threaded Shantz body 9310 coupled to one or more devices 9315, the tags which may be permanent or absorbable.

It is noteworthy that all previous optical sensors/systems utilized in orthopedics with computer navigation and robotics have required that initially a screw or threaded pin (Schantz screw) be applied to bone and subsequently through a clamp be attached to an optical tracker. This requires bulky large pins within the wound that frequently get in the surgeon's way of performing the operation. Additionally, the threaded pins in bone and the clamps holding the trackers are subject to being manipulated and leaned on by the surgeon and assistants, which can lead to loss of reference point and erroneous input and registration process.

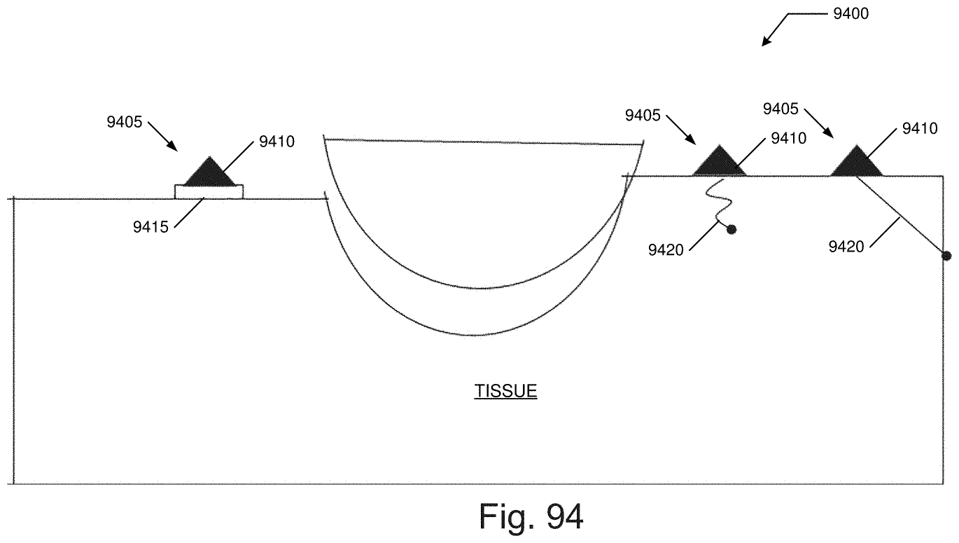

FIG. 94 illustrates a system 9400 including a set of tags 9405 including alternative tag fixation modalities attaching a body 9410 to tissue, the alternative fixation modalities including a layer of adhesive 9415 or strand of suture 9420.

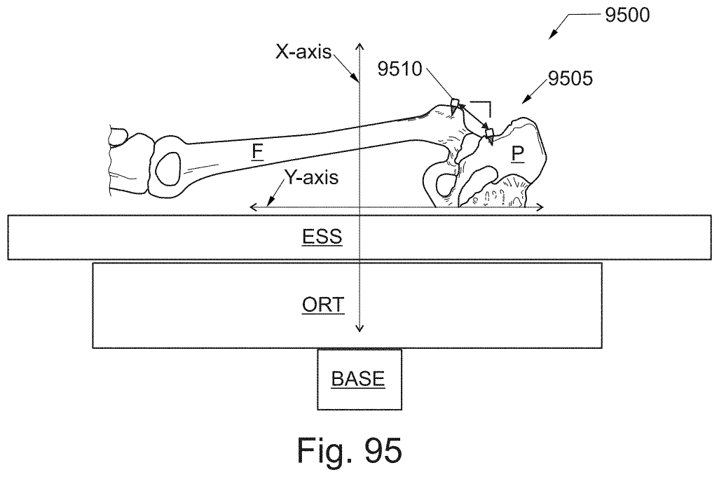

FIG. 95-FIG. 97 illustrate a concept view of a set of implementations for realtime systems for externally accessed non-interventional intracorporeal tags, such as may be used for a THA/THR procedure. For these illustrations, a patient is in “lateral decubitus position” on an operating room table (ORT). This means the patient is lying on one side (e.g., a left side) with the other hip (e.g., the right hip) pointing upwards towards the ceiling (for simplicity, the figures do not include the left pelvis portion and leg)—effectively illustrating a partial side view. Some surgeons perform a THR in supine position where the patient is lying flat on his back on the ORT, face toward the ceiling (not illustrated). As noted elsewhere herein, pelvis P is restrained by the joint itself as to a plane (e.g., the Z plane). Thus in this lateral decubitus position, the surgeon effectively may only make the leg longer/shorter (Y-axis changes towards the left or right in the views) or wider/narrower (X-axis changes towards the top or bottom in the view) but not more posterior and/or anterior (in and out of the plane of the page). The visible axes in the figures would be different for a supine THA procedure.

FIG. 95 illustrates a first implementation of a system 9500 for realtime externally access to a set of intracorporeal tags 9505. Set 9505 includes one or more tags (two in FIG. 95) that are fixed to internally accessed locations on pelvis P and femur F. These locations may be convenient for the surgeon and become objective invariant reference location points throughout the procedure as opposed to subjectively evaluated imprecise variable anatomical landmark locations. Disposed on, or integrated into, ORT is an extracorporeal sensing system (ESS), an example of a tag interface, that externally (extracorporeally) accesses set 9505 to provide the desired information (in this case relative length and offset changes between set of sensors 9505). The manner of external access of this desired information depends upon the technology of the set of tags (active/passive/hybrid systems which may include local processors and communications subsystems). ESS may be implemented, for example, as part of a disposable pad disposed on or within a top surface of ORT (other implementations are possible). Set of tags 9505 provide direct and continuous positional information which allows non-interventional monitoring of realtime/near realtime changes to length and offset during the THA/THR procedure.

While the tag interface, illustrated as ESS in FIG. 95, is incorporated or associated with a disposable pad disposed on an operating room table. The tag interface may be disposed in other structures besides, or in addition to, the disposable pad. For example, the tag interface may be disposed on a railing of the OR table. For example, the tag interface may be disposed in an electronic device (e.g., an iPhone sitting on a desk, a laptop computer, or electronically visually displayed in a wearable glasses or head-mounted display). The set of tags and tag interface may be implemented to establish a status of the leg offset and leg lengths in different ways, using different technologies. As described further herein, the set of tags may employ active/passive/smart/dumb components to establish the status directly or establish underlying data from which the status may be calculated, derived, or established. The status or underlying data is communicated to the interface which may collect/compile/gather the status or underlying data.

Other implementations of the present invention may include an indicator, display, or user interface to present the status information to the surgeon in a simple, efficient, non-intrusive manner. The indicator/display may be a part of the tag interface or an additional system. For example, various feedback methods could be employed to present and allow assessment/correction of the realtime status. This can be done for example through visual, audio and tactile feedback. A surgeon may have realtime information about the offset and length parameters instantaneously available through tag interface and optional status indication/visualization/display systems. Thus, for every adjustment where the surgeon may produce a change to the offset and/or the length; visual, auditory and tactile information can be immediately and instantaneously made available to the surgeon through the tag interface/status indicator.

This information is transferred wirelessly and made available to the surgeon: (i) visually through wearable computer glasses (for example, smart glasses, head-mounted displays, augmented reality visualization systems, and the like) equipped with infographics (graphic visual representation of information); (ii) through tactile vibrations and haptic technology using wearables such as watches, earrings, necklaces and/or clothing; and/or (iii) through auditory feedback using earpiece/audiblization technologies such as headphones, earbuds, speakers, transducers, and other electroacoustic transducers, converting electrical signals into sound.

Hence, the surgeon may be instantaneously aware and conscious of the current status (offset and/or length) and understand what adjustments may be desired to correct or alter the status during the surgery in order to achieve the preoperative plan.

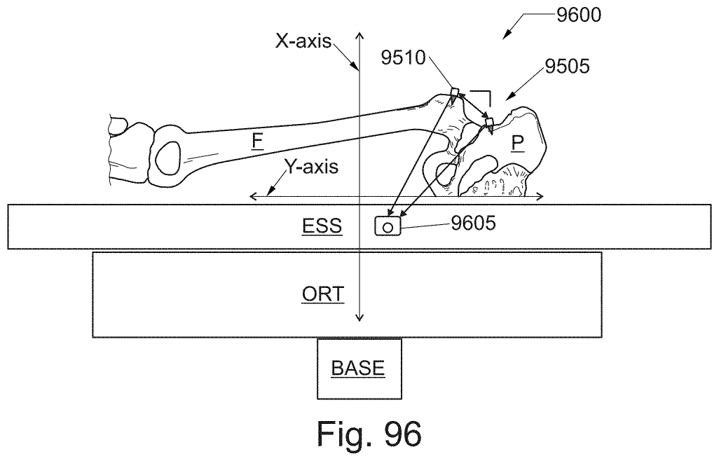

FIG. 96 illustrates a second implementation of a system 9600 for realtime externally access to a set of intracorporeal tags 9505. System 9600 is similar to system 9500 with the following adjustments. Set of tags 9505 may be “dumb” and read by a sensing/monitoring subsystem including one or more sensing devices 9605 embedded in a disposable pad implementation of ESS.

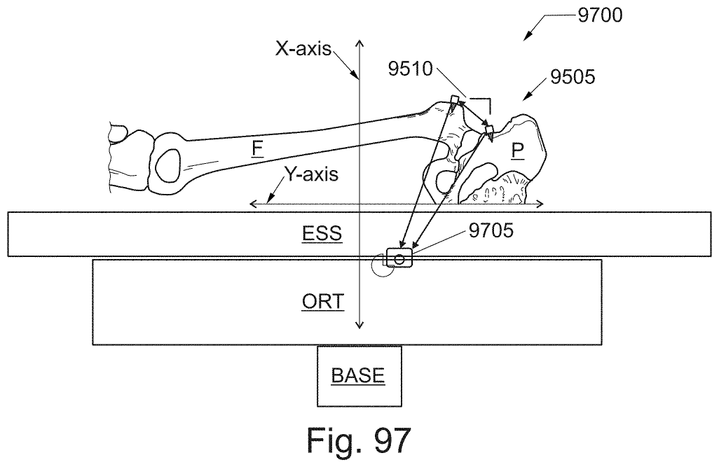

FIG. 97 illustrates a third implementation of a system 9700 for realtime external access to a set of intracorporeal tags 9505. System 9700 is similar to system 9500 and system 9600 with the following adjustments. Set of tags 9505 may be “dumb” and read by a sensing subsystem including one or more sensing devices (e.g., chips) 9705 attached/clamped to ORT as part of an implementation of ESS.

In operation, a surgeon fixes a set of tags to externally-accessible portions of pelvis P and femur F (easily and conveniently accessible locations, may even be deemed to be otherwise arbitrary placement, that do not interfere with the THA procedure without regard to specific anatomical landmarks (e.g., teardrop and trochanter)). An ESS is disposed in appropriate proximity to the set of tags, what is appropriate is determined by the technology employed by the set of tags and ESS. The set of tags is non-interventionally accessed externally in realtime to provide desired status information produced by the set of tags, in this case to length and offset.

The tags described in this application may be anywhere in size between 20 mm to 1 mm or even much smaller (such as 1 micrometer or 1 nanometer) as miniaturized sensors continue to develop over time. A small threaded tag can be easily and quickly applied (and removed) within the surgical field to allow continuous and accurate monitoring of leg length and offset between the pre-operative diseased hip and the hip replaced with implants. This can be done in an unobtrusive, efficient, and inexpensive manner, without the need for bulky equipment, complex mathematical algorithms to establish a coordinate system in the OR space, purchase of million dollar capital equipment such as robots, and application of large screws and clamps to bone. The status information is not limited to leg length/leg offset implementations. As noted here, status may also include, among other intracorporeal status, force response for fixation/seatedness or metabolite detection, among other status information some of which are detailed herein.

These reference tags may have additional uses. ACL reconstruction is one of the most common operations performed by orthopedic surgeons. Every year more than 300,000 ACL reconstructions are performed in the US alone. Previous reports suggested that the success rate of ACL reconstruction was in the 95% range, however, over the last several years it has become apparent that the success rate is significantly lower and potentially no higher than 60% to 70%. The consequences of failed ACL reconstruction are significant and include instability, pain and stiffness. Many patients may develop post-traumatic arthritis ultimately requiring additional surgeries such as total knee replacement. Some will require multiple surgeries for revision ACL reconstruction to obtain a stable knee. In general ACL reconstruction has not been as successful a surgery as we have believed it to be. Finally, new studies suggest that not reconstructing a torn ACL, even when it appears unnecessary in a sedentary patient, will cause development of un-physiological forces against the femoral condyles, leading to post traumatic arthritis. Therefore, the cost of failed ACL and ligament reconstructions in general are immense and affect both the individual (pain and suffering) and society (cost).

Some believe that a significant cause of failed ACL reconstruction is poor tunnel positioning. The inability to consistently choose the correct insertion sites and non-anatomical tunnel placement (technical misplacement by the surgeon) may account of up to 70% of ACL failure cases.

The ACL, as an example, is an elegant ligament that has two (or more) bundles within its substance that provide different mechanical properties throughout a range of motion, as well, during loading and unloading of the extremity. These two bundles commonly referred to as the anteromedial (AM) and posterolateral (PM) bundles have distinct anatomical bony landmarks (insertion sites or footprint) inside the joint, upon which they attach on the femur and tibia.

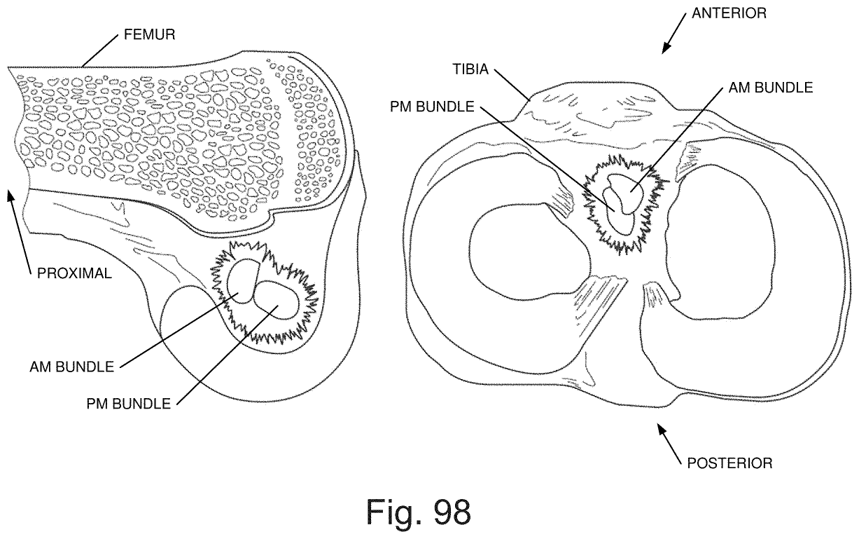

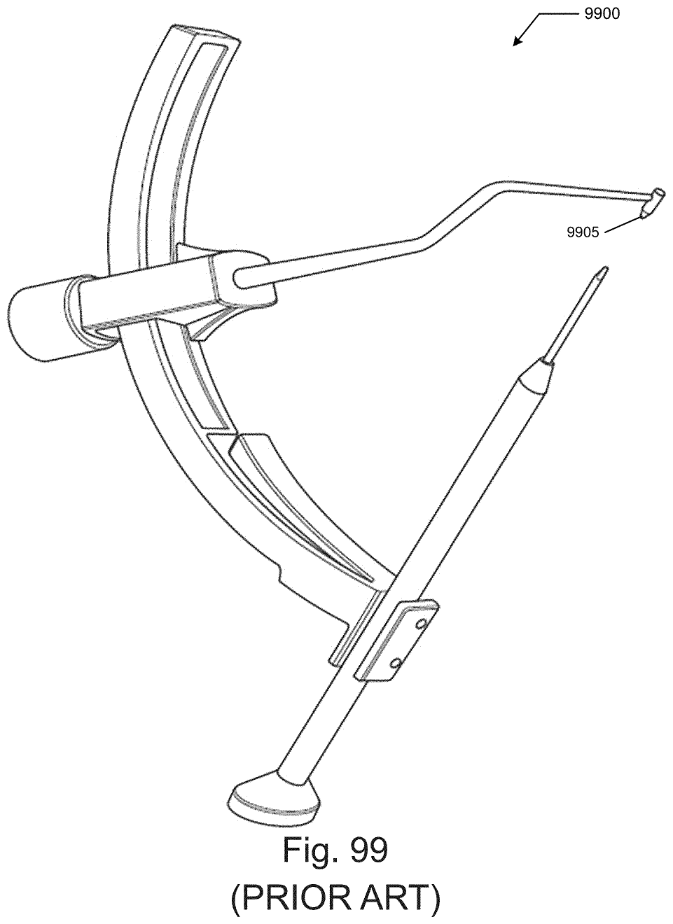

FIG. 98-FIG. 116 illustrate additional uses and implementations of anatomical locator tags. FIG. 98 illustrates femoral and tibial attachment sites (footprints) of the anterior cruciate ligament anteromedial (AM) and posterolateral (PL) bundles.

These insertion sites (footprints) are distinctly different and highly specific for individual people. For example, the AM and PM bundles together may have a 20 mm footprint on the tibia in a 5′3″ female soccer player, while the same two bundles may have a 13 mm footprint in a 6′1″ male basketball player.

Currently, when a surgeon does an ACL reconstruction, the only tools at her disposal for identification of the ACL insertion sites is the arthroscope and the typical “ACL guides”, which are carpentry inspired hand held tools that assist the surgeon in geometric tunnel placement.

FIG. 99 illustrates a conventional anterior cruciate ligament tibial ACL guide 9900. The guide is a mechanical tool that is configured by the surgeon. An ultimate purpose of guide 9900 is to help the surgeon define a tunnel path that terminates at a location of a guide tip 9905. A challenge for use of guide 9900 is accurate positioning of tip 9905 at the appropriate footprint for the AM and PM bundles.

The surgeon is asked to work inside the joint, visualize the joint with a 30° angle arthroscope from within, without any cues from outside bony landmarks, and to accurately determine the insertion sites of the ligament. Frequently, to make matters worse the visual field obtained during arthroscopy is variable. This is because the positioning of the “portals” (the keyhole incisions through which instruments are inserted) may themselves cause limitations of field of view, which may occur even when the portals are offset by 5 mm. A second factor that obstructs field of view is the soft tissue remnants of the torn ligament. Typically maintaining the soft tissue remnants as a “biological cover” or “soft tissue skirt” is an advantage for healing of the grafted ligament but these remnants are frequently completely removed in order to be able to see the bony landmarks that identify the insertion sites. Thirdly, bleeding conditions may obstruct insertion sites.

Therefore, the surgeon is asked to navigate inside a joint with an arthroscope and a mechanical ACL guide, with variable visibility conditions, and asked to find specific landwards which identify the insertion sites to the ACL bundles, drill a guide wire in the center of the footprint/insertion site and then over drill to create the bony tunnels within which the graft is passed.

When viewing the knee joint with an MRI or CT scan the insertion sites are clearly visualized and their specific relationship to other bony landmarks in and around the joint are clearly seen. However, in the operating room and during surgery, these cues are not present. Currently there is no system that allows the surgeon access to these cues, so that a more precise operation can be performed. There may be advantages to having efficient global anatomical cues or some method of “assistive positioning system” made available to the surgeon for enhanced performance of ACL surgery.

Current techniques in ACL and other ligament reconstruction are not supported by assistive technologies that can accurately, efficiently and quickly detect the insertion sites. The act of searching inside the joint for a landmark without an assistive “positioning” device can be analogized to mountaineering without awareness of global cues that maybe important to survival. What is currently done in surgery is akin to asking a mountaineer to find a specific spot on the mountain using nothing but tress and rocks as guides, without the benefit of a compass or any technology that provides a complete sense of the scope and size of the mountain. For example, without a compass or a GPS system, the mountaineer is significantly limited in her ability to find locations and assess distance, and therefore room for error may be large. A similar situation exists for the arthroscopic surgeon performing ACL surgery. Once the surgery is started the outside cues and landmarks and their relationship with the inside bony landmarks are not available to the surgeon, and therefore quick identification of the insertion sites becomes difficult if not impossible. Soft tissues have to be aggressively debrided and removed to identify the landmarks that represent the insertion sites.

Another analogy is that of a pilot flying only using visual cues, without the benefit of radar or GPS. Arthroscopic ACL surgery in some ways is similar to flying a plane in bad weather without radar because the visual cues in arthroscopy are many times obscured by poor portal placement, bleeding and soft tissues. Just as a pilot could greatly increase the chance of safe and proper flight with radar and global positioning systems (GPS), the surgeon can greatly increase the chance of safe and proper surgery with a positioning system that is efficient and nimble.

Furthermore, the surgeon adds significant time to the surgery cleaning and debriding the bone and soft tissues that obstruct direct visualization of the landmarks that identify proper tunnel position.

Despite these efforts, surgeons are frequently not sure whether they have picked the right spot (insertion site) for the tunnels. This maybe one reason even very experienced surgeons have a fair number of failures themselves. In other words, the process of choosing the insertion sites and tunnel placement in ligament reconstructive surgery is far from standardized. Additionally, the technology is nowhere close to being able to customize the tunnel placements with our current thinking, despite the fact that many of the insertion sites appear to be individual patient specific (i.e. the tibial ACL insertion sites 5′3″ female soccer player vs. 6′1″ male basketball player).

A reason for non-standardization of ACL tunnel positioning includes the fact that no tool exists to quantitatively assist the surgeon in detecting the proper ACL insertion sites. Therefore, the same surgeon (experienced or not) is very likely placing the tunnels in different places every time the same surgery is performed. In other words, despite best attempts, the same surgeon is choosing different insertion sites with each surgery. Furthermore, because the process of tunnel creation is not standardized, surgeons do not have a means of accurately measuring what was done. Therefore, they cannot know (or have means of assessing) which one of their actions has led to failure and which to successful outcome.

This application includes a proposal for a system that allows the surgeon to properly control for the variable of tunnel positioning and therefore be able to analyze possible tunnel positioning errors as cause of failure with respect to instability, stiffness and pain. For example, when the tibial tunnel for ACL reconstruction is placed too posteriorly, the knee may be loose and unstable and when the tibial tunnel is placed too anteriorly the knee may become stiff. The ability to quantitatively evaluate the tunnel placement variables with outcomes (stiffness, pain, instability) provides for better understanding of the procedure, better outcomes, and potential for improvement of the technique over time.

Previous attempts to solve this problem have entailed incorporation of existing technologies similar to GPS. Computer navigation surgery has been employed in orthopedics for several decades as discussed herein. This technology has gained more acceptance in arthroplasty procedures than arthroscopic procedures, nonetheless, it has been extensively researched in ACL reconstruction. Use of robotics and computer navigation has not gained wide adoption in ACL or ligament reconstructive surgery.

First, the process of establishing an operating room wide three dimensional (3D) coordinate system (COS) in the OR space (discussed herein) is time consuming and adds a significant number of steps to an already complex procedure that is constrained by tourniquet time (time in general—the longer the operation the higher chance of infection). Secondly, with navigation, there is a requirement to move big equipment in and out of the surgical field, including computers, cameras, robots to take snap shots of positions of the instruments and implants in relation to bone. These technologies are time consuming and bulky and have not been widely adopted by orthopedic surgeons because they have added significant cost, extra time and steps, without providing meaningful difference in the outcome.

An alternative assistive positioning system may be advantageous with respect to currently available bulky robotics and navigations systems that are inefficient/time-consuming/and complicated, that can provide quick and accurate positional cues for the surgeon, without the bulk, extra steps, extra time, and likely extra cost.

This system may identify the tunnel positions, without the need to rely or be concerned about the surgeon's experience, eye-hand coordination, precise portal placement, visualization issues such as bleeding.

The technique may also allow the surgeon to leave intact much of the soft tissue remnants as “vascular skirts” for the grafts that enhance healing, without having to remove them to identify the landmarks.

Some implementations in this application enhance/extend systems and methods presented and described in the incorporated parent application where various sensors (reference tags) in bone provided information about the A (delta) change in distance and angle between fixed reference tags in bone, among other innovations. This concept has useful application in determining leg length and offset differences in hip replacement surgery.

The description herein includes a local positioning system (LPS) for ACL reconstruction and ligament surgery in general, where precise positioning information about the insertion sites of ligaments within the joint are made available for the surgeon in order to perform a better more reliable surgery. Sometimes this concept is referred to herein as a BMD LPS-ACL system and method.

As discussed, there are benefits to having (sensor-embedded-screws or other structures) as reference tags in bone that allow detection of the changes in distance and angle between fixed points in bone and to provide fixed, objective reference locations. Herein is a discussion including a system and a method that uses sensor-embedded-screws to determine exact location of ligament insertion sites/footprints and tunnel positioning. Also included herein is a discussion of a hand held tool that may (in and of itself) provide an ability to accurately find the insertion sites/footprints of the ACL bundles on the femur and tibia, to provide for precise tunnel positioning, and allowing every surgeon to perform better surgeries regardless of experience level.

Since there appears to be so much difficulty and variation in finding these footprints, and since it is known that many of the ACL failures occur due to poor tunnel placement, there may be advantages to availability of a tool that automatically and quickly finds ligament footprints and assists with tunnel positioning.

The benefits of a tool that automatically and precisely identifies relevant anatomical features such as the ACL foot prints (i.e. AM and PL footprints on the tibia and femur) could be tremendous; including (a) one could actually measure what has been done so tunnel placement may be isolated as a variable and assess its effects on outcome; (b) an ability to measure “what was done” lends itself to better understanding of the process through cognitive technologies and machine learning; (c) operative time and infection risk would be decreased, where less time is spend debriding soft tissues and bone to find the insertion sites; (d) healing will be enhanced, including maintenance of soft tissue remnants as vascular skirts to enhance healing of the grafted ligament to bone; (e) tunnel placement in ACL reconstruction (and all ligament surgery in general) could become (i) standardized and (ii) customized (i.e. patient specific); and (f) double bundle ACL reconstructions may become more common place providing better kinematics for some patients, particularly athletes and others applying significant stresses to their connective tissue and joints.

The BMD LPS-ACL System and Method

An implementation may include special structures (sometimes referred to herein as reference tags or anatomical tags) be applied to certain bony landmarks about the knee or joint of interest. It is well known that microchips, electronics and microcontrollers have been undergoing a miniaturization revolution over the last decade. This miniaturization will continue to such a level where screw/sensors in the order of 1 mm-5 mm or even smaller can be percutaneously applied to bony landmarks under simple sterile techniques, in clinic with a minor procedure that takes less than five minutes to perform; similar in scope to suturing a small wound or an injection. Any anatomical tags added in a pre-operative clinic may be used for pre-operative assessment and then during the procedure where more anatomical structures are accessible, the initial set may be supplemented or replaced as needed or desired.

The current implementations may be implemented with a range of current and future-developed active and passive sensing technologies, and as such the specifics of the actual sensing technologies may be considered secondary to the use of a set of sense-aware anatomical reference tags (active, passive, or a combination thereof).

Subsequently, an imaging study of choice is conducted with a CT scan, X-ray, MRI or Ultrasound. Once imaging is completed, the anatomical tags, that were previously attached to bone (or other subcutaneous tissue), provide the function of a common fixed reference frame for the knee joint. For certain procedures it may be appropriate to fix some or all of a set of tags to a surface of the skin with an understanding that such tags may be subject to contact/displacement/removal and shifting based on skin manipulation.

In this dormant stage, the tags function as visual/optical markers which provide an ability to for a technician in a laboratory to digitally, visually and optically measure distance and angle relationships from multiple reference points to the bony landmarks that identify the insertion site/footprints of the ligament to be reconstructed. For example, the distances and angular relationships from the three reference tags applied to the proximal tibia and the insertion sites of the AM and PL bundles of the ACL on the tibia can precisely and accurately measured.

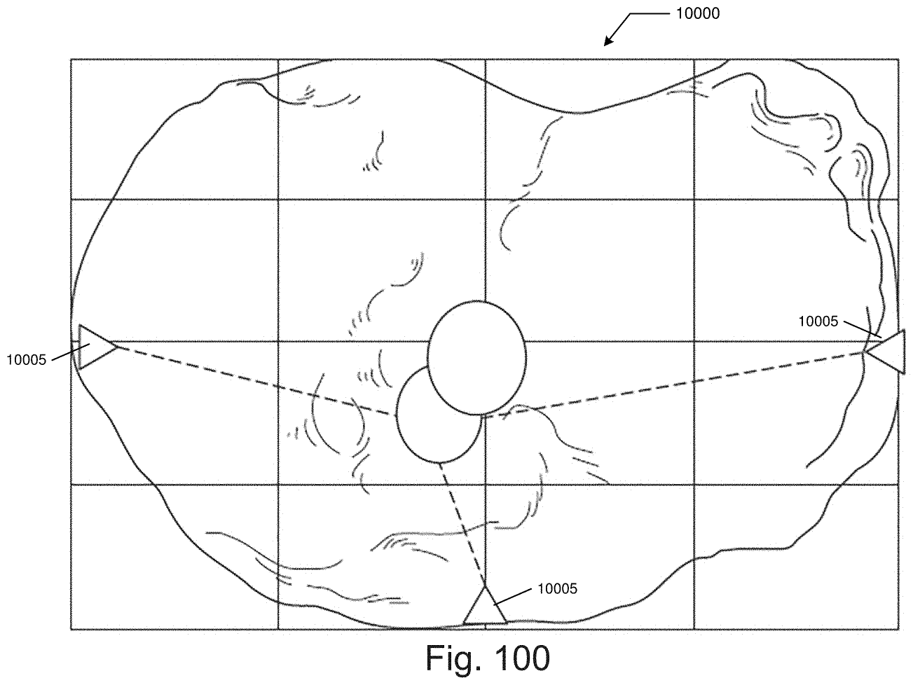

FIG. 100 illustrates a system 10000 depicting a relationship of each of three anatomical tags 10005 to the anteromedial AM bundle attachment of ACL on tibia. The patient is subsequently taken to surgery. At this point, the tags are safely buried under the skin and dormant, resistant to displacement before the actual surgery. During arthroscopic surgery, the tags sensing technology or tag location interface (e.g., distance and/or angles) are activated to precisely identify the insertion sites of the ligament to be reconstructed such as by triangulation.

A variety of distance, location and proximity sensing technologies are available that can be deployed within the anatomical tags to replicate and reproduce the distance and angle relationships between the tags and the insertion sites/footprints.

For example, a patient that is known to have an ACL tear is seen in clinic pre-operatively. In order to have a special tool that automatically finds the ACL footprints during surgery, three or four reference tags are applied to the distal femur and proximal tibia.

The reference tags (e.g., screw/anchor/fastener and the like with active, passive, or hybrid technology) may have a particular sensing technology incorporated within or operational in cooperation with, and may be miniaturized (possibly to 1 mm to 5 mm in size or even smaller) so they may be easily inserted subcutaneously into bone and buried under the skin. They are inserted into bone through a small incision with an insertion tool which may utilize pneumatic, electronic, and or manual force.

The patient is sent for an imaging study of choice (CT, MRI, Ultrasound, X-ray), with the reference tags already applied. Imaging software allows precise distance and angle measurements from the tags to the ligament insertion sites. The anatomical tags provide objective and invariant/accurately reproducible reference locations that may be used pre-operatively for planning, during surgery for implementation of the plan, and/or post-operatively (when post-operatively retained/affixed to the bone) to monitor for loosening or other degradation or reinjury of the repair, all using the same objective reference locations.

For example, the precise distance and angular measurements from the three tibia reference tags to the ACL (AM) and (PL) tunnel insertion sites is taken and recorded. Similarly, the precise distance and angular relationships between the femur reference tags and the ACL (AM) and (PL) insertion sites is taken and recorded. These measurements can be taken with up to 0.1 mm accuracy. At this point a fixed common reference frame between the tags and the insertion sites is established.

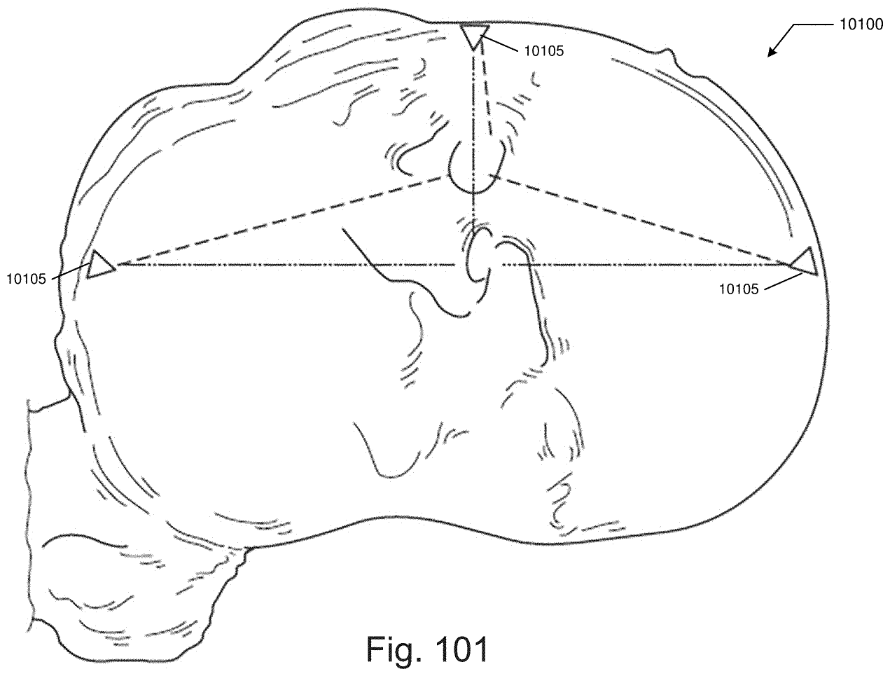

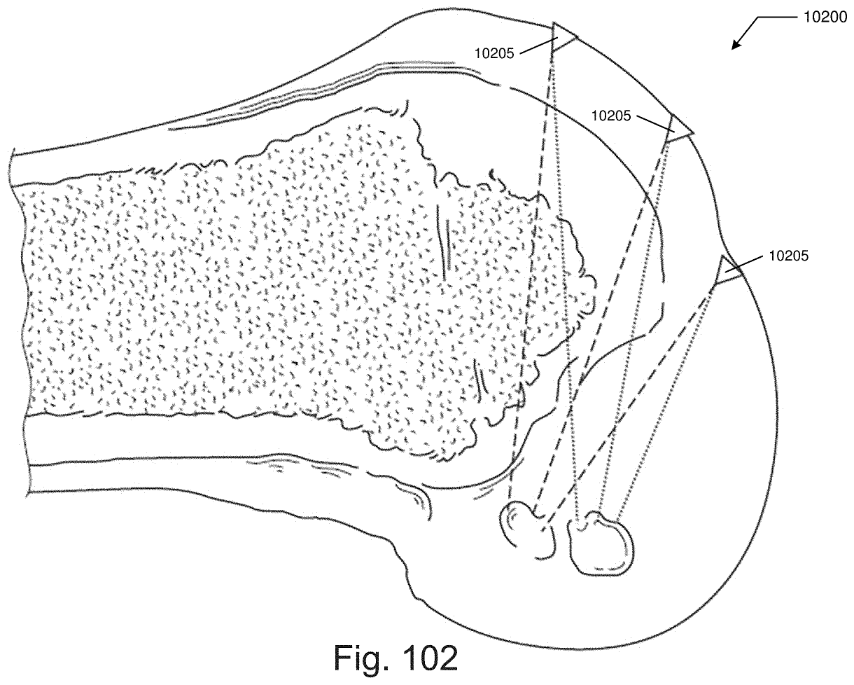

FIG. 101 illustrates a system 10100 depicting a relationship between three tags 10105 on the tibia and the anteromedial (AM) and posterolateral (PM) bundle attachments sites (footprints) of ACL on the tibia. FIG. 102 illustrates a system 10200 depicting a relationship between three tags 10205 on the femur and the anteromedial (AM) and Posterolateral (PM) bundle attachments sites (footprints) of ACL on the femur.

As illustrated in FIG. 101 and FIG. 102 illustrate plan views (e.g., “overhead”) of the bundle footprints, that is, two-dimensional positioning. The anatomical tags are not limited to two-dimensional positioning (e.g., just a location of openings of the bundle tunnels), but may also be used for three-dimensional location of the entire length of the tunnel.

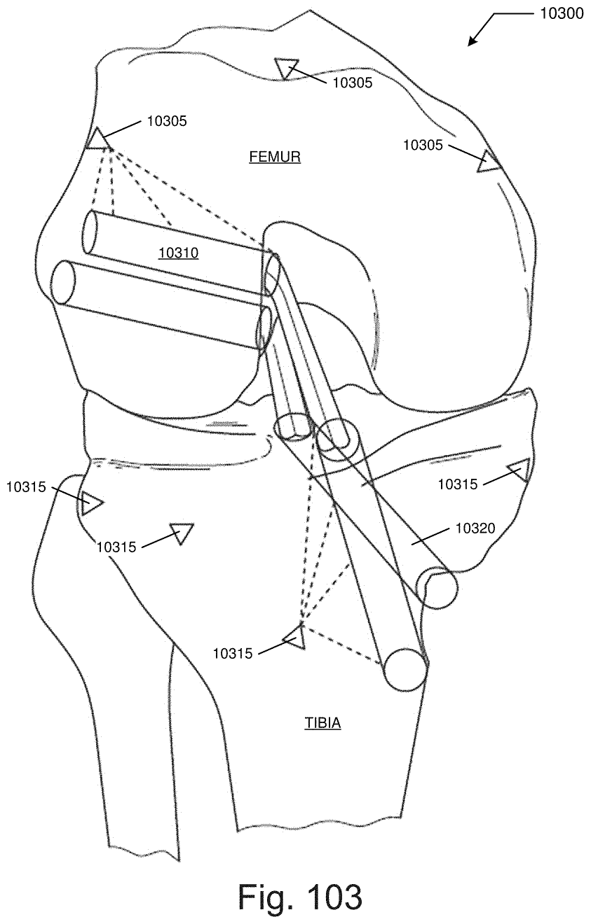

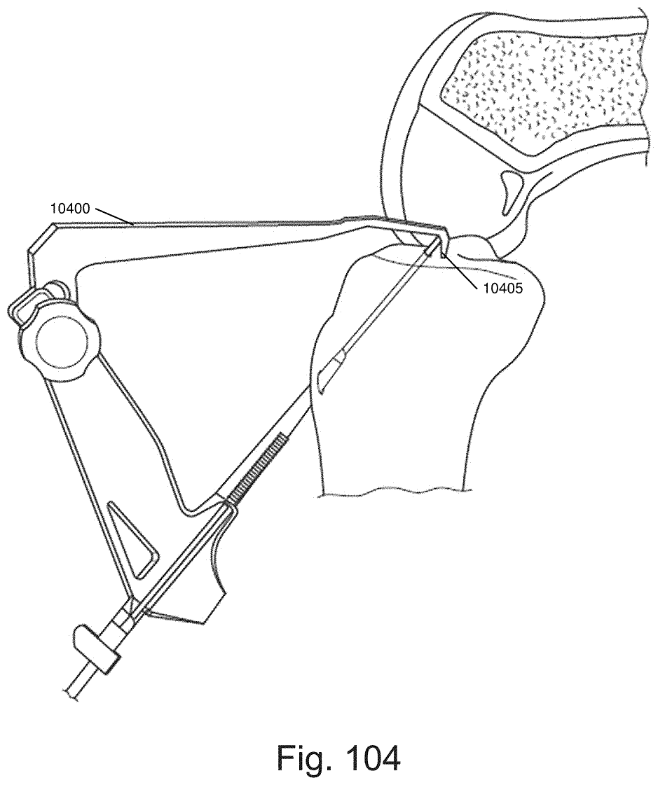

FIG. 103 illustrates a system 10300 including three-dimensional relationship positioning between three tags 10305 on the femur and femoral anteromedial (AM) tunnel and three-dimensional relationship positioning between three reference tags on the tibia and the tibial anteromedial (AM) tunnel.

During surgery, the tags are active/operational (including passive operational anatomical tags), and depending on the technology chosen the distance and angular relationships between the tags and the insertion sites are recreated and replicated inside the joint to assist the surgeon in detecting the ligament (ACL) insertion sites.

For example, any one of distance and motion sensing technologies may include infrared, ultrasonic, capacitance, inertial and/or magnetic, or other, sensors that may be used alone or in combination to reproduce the distance and angular relationship between the fixed reference tags and the insertion sites.

The BMD LPS-ACL Guide

The typical ACL tibial and femoral guide, see for example system 9900, used for identifying insertion sites are generally shaped to have a central targeting hole for the tip of the guide wire and surrounding sharp prongs that engage the bone. Under direct visualization, when the surgeon finds the desired insertion site, she hooks the prongs on to insertion site and drills a guide wire towards the tip of the ACL guide. This is done before over drilling the actual tunnel with a larger cannulated reamer, which typically ranges between 8 mm to 10 mm in diameter.

FIG. 104 illustrates an enhanced tibial ACL guide 10400 (including modifications to guide 9900) used to determine the eventual tunnel and footprint placement. BMD LPS-ACL guides 10400 are adapted to have a special sensor tip 10405, (similar to a miniature mine detector) and incorporated with electronic circuitry and mini display units for visualization of the ligament insertion sites in cooperation with the embedded anatomical tags.

The BMD LPS-ACL guide includes two features: (i) a capacity to replicate exact distance and angular relationships between a fixed common reference frame in bone and the insertion sites for the ACL tunnels; and (ii) incorporated electronic circuitry as needed with a capacity to transfer information for interpretation and display. Information and data within the sensors, microchips, and tags, as well as there relative interactions (e.g., distance/position/location) may be, either directly visualized through electronics and mini display unit incorporated within BMD LPS-ACL guide 10400; or wirelessly conveyed to a central processing unit through wireless communication systems (Internet, WAN, LAN, and the like) for interpretation and display on a computer screen for direct realtime (when desired) surgeon viewing.

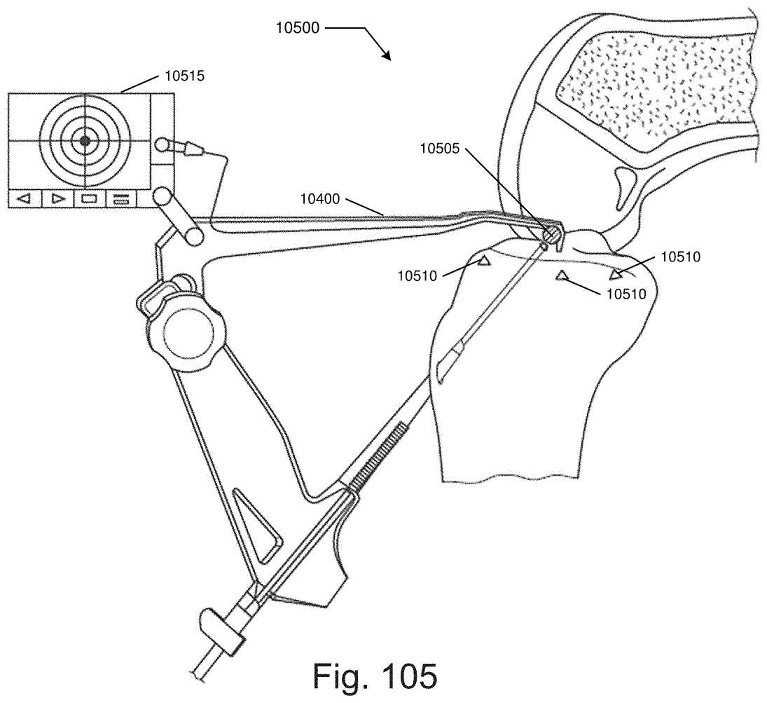

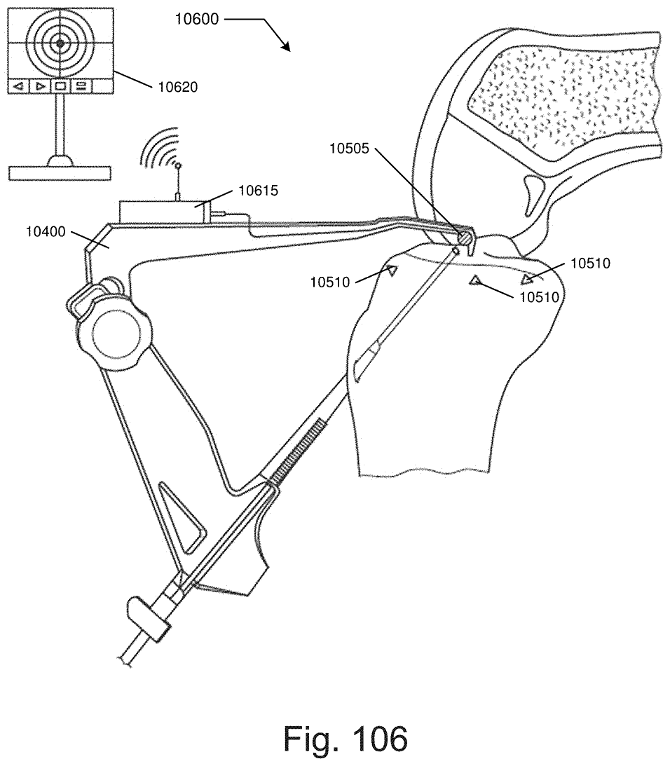

FIG. 105 illustrates an LPS-ACL guide 10500, an all-in-one unit and FIG. 106 illustrates an LPS-ACL guide 10600 with multiple cooperative components (e.g., separate monitor). Guide 10500 and guide 10600 include system 10400 with a location sensor 10505 and additional local positioning equipment customized for the intended application to define and produce desired bone tunnels. In this case, the femur and tibia.

Location sensor 10505 cooperates with a set of anatomical tags 10510 affixed to bone around the site of the procedure (this set is sometimes referred to herein as a constellation helping to highlight that three-dimensional locations of the tags can be important). A set of anatomical tags 10510 employ the same or compatible technology with location sensor 10505 to enable sensor 10505 to be positioned in 2D or 3D solutions with respect to tags 10510, such as accurate locations of the connective tissue bundles. Some or all of tags 10510 may have been pre-operatively installed and a detailed study conducted to determine desired locations that may be monitored/sampled by location sensor 10505 which in turn defines a precise location of a processing or preparation tool, for example, a drill bit, burr, or bone sculpting tool within the bone.

A feedback system is employed, such as an “on tool” display 10515 or a remote display 10620 receiving (wired or wirelessly) relative position information of tip 10505 with respect to the constellation of anatomical tags 10510.

The BMD LPS-ACL guides used for creation of the tibial and femoral tunnels will therefore be armed with sensors at their target tips. Similar in concept to mine detectors which identify mines with sound, visual effects and vibration, when scanned over the ground, the BMD LPS-ACL guide precisely identifies the exact location of the insertion sites when the sensor tipped BMD LPS-ACL guide scans over the targeted structures. The insertion sites can be found with ease and confidence regardless of visualization issues and surgeon experience. No time will be wasted debriding inside the joint to identify landmarks. Soft tissue remnants important to healing will be spared. Finally, surgeons will have the ability to document exactly what was done so they can learn from their mistakes and to know how to optimize outcomes.

The sensors within the tips of the BMD LPS-ACL guides are programmed to indicate when the precise angle and distance relationships between tags 10510 and the desired ACL insertion sites/footprints are reached. The precise location of the footprints in relation to the BMD sensor tipped LPS-ACL guide can be shown graphically or visually on a screen. The insertion site can be identified as a target with surrounding circles, or with color graphics (green meaning the correct spot is reached, the surgeon should dock and drill) (red and yellow providing a sense whether the surgeon is close or far). Similarly, tactile, vibratory or auditory responses could be utilized to show that the proper insertion site has been reached.

The distance and angular relationship from each tag to the insertion sites may be incorporated into data fusion algorithms to optimize the accuracy of the location of the footprints. For example, the set of tags may include more than three tags such that multiple checks on angle/distance are possible to provide a more robust solution than relying on fewer single tag-tag measurements.

Therefore, during surgery, as the BMD LPS-ACL sensor tipped guide is activated and “turned on” and scanned over the insertion sites/footprints, when the exact location of the footprints is found, the sensor tipped BMD LPS-ACL guide provide a visual, auditory or tactile response, at which point, the surgeon knows that she is hovering over the insertion site, and therefore the ACL guide is docked on to the bone.

The desired location may have been predetermined from post-clinic imaging analysis of pre-surgery clinically installed anatomical tags. Then those exact same reference tags used in the imaging analysis may be used by location sensor to precisely locate the bone process/preparation/sculpting implement at the exact location and enable close implementation of the presurgery plan.

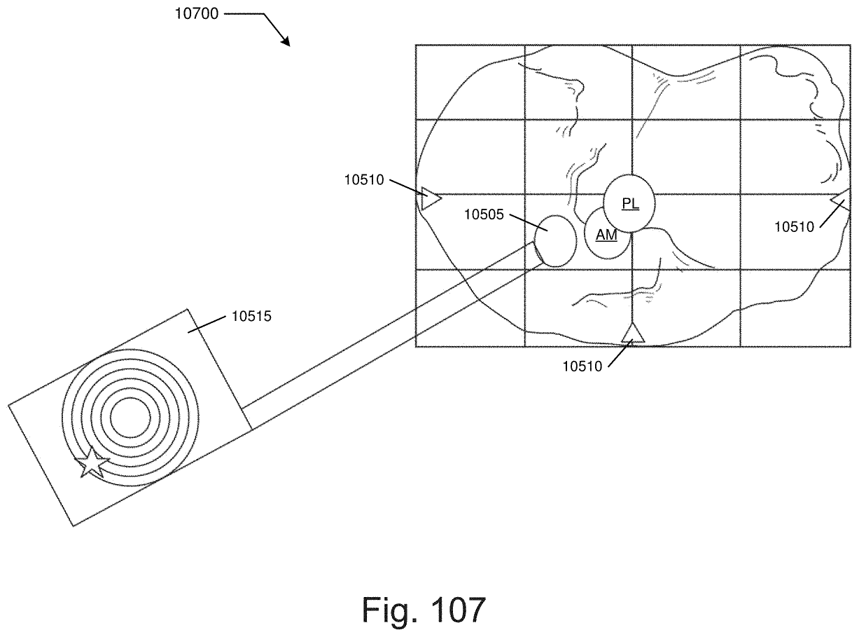

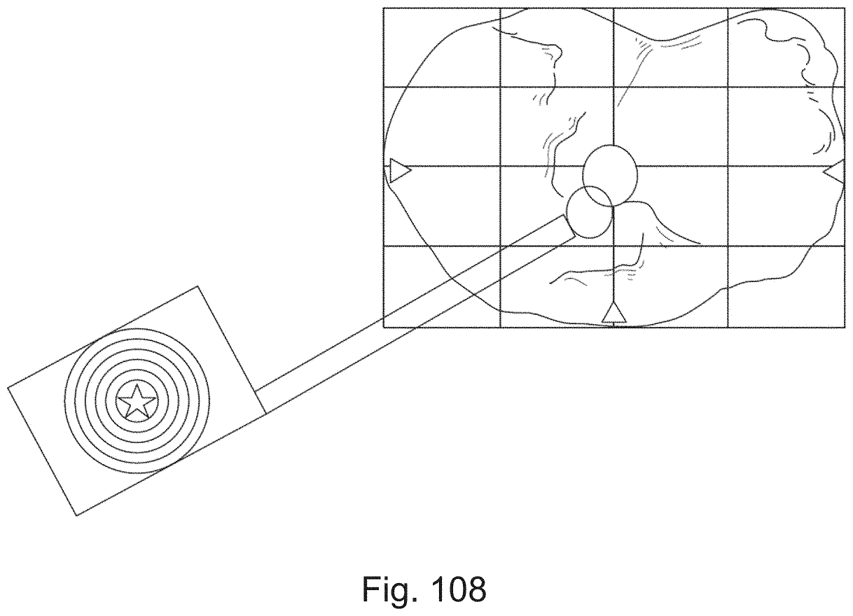

FIG. 107 illustrates a generalized diagram of a system 10700 including a LPS-ACL guide sensor tip 10505 indicating discrepancy between its position and the AM bundle attachment site (footprint) of ACL, indicated by star off center from bulls' eye on display 10515. FIG. 108 illustrates LPS-ACL guide sensor tip hovering over the AM bundle attachment site (footprint) of ACL, indicated by star centered over the bulls' eye.

In operation, the center of the bulls' eye represents the target position for the location of sensor 10505. The “star” represents the actual relative location of the tip relative to the desired location. In FIG. 107 the tip is not properly positioned, and the star indicates the direction and magnitude of the mispositioning. In FIG. 108, the tip is properly positioned, and the star indicates that to be the case by laying on the bulls' eye.

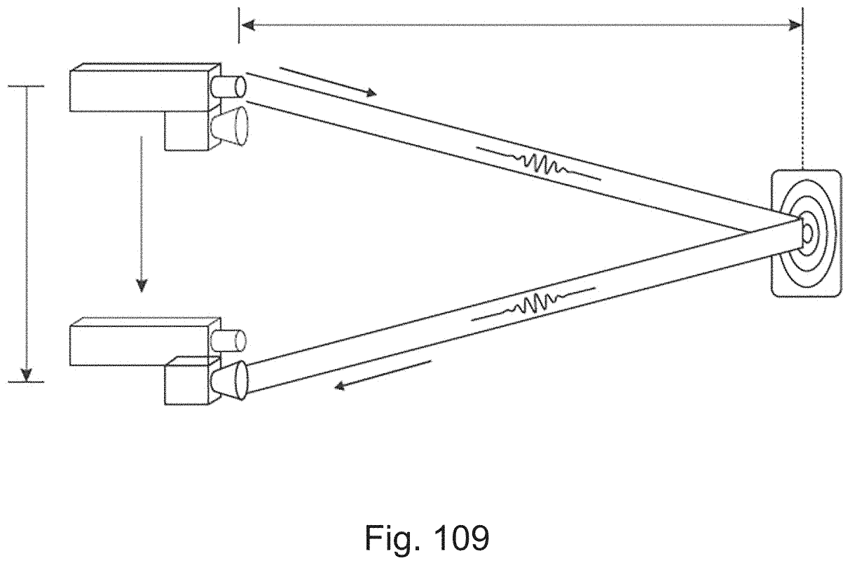

FIG. 109 illustrates time of flight measurements that may be used to determine distance (speed of light and sound are known). Standard technique for drilling of the guide wire over-drilling of the tunnel with appropriately sized cannulated drill is then performed. The distance and angular relationships between the fixed bony reference tags and the insertion sites on the femur and tibia can now be determined with any of the particular distance or proximity sensing technology chosen.

Different distance and proximity sensing technologies may be used in the BMD LPS-ACL guides depending upon the application, anatomical tag technology, and other design considerations of the desired solution. A variety of different types of distance and proximity sensors can be incorporated into the BMD LPS-ACL guide to determine the exact location of the ACL insertion sites within the joint.

For example, magnetic sensors convert distance measurements to different strength magnetic fields and have some advantage because there are no line of sight issues. Time of flight of flight sensors employ techniques that measure the round-trip time of an artificial light or sound signal to resolve the distance between two objects. speed=distance/time (s=d/t) and therefore d=s·t/2. The speed of sound and light are known. Measuring time of flight back and forth enables calculation of distance.

Light detection and ranging (LIDAR) works through measurement of time for narrow beam of pulsed light to reach an object and reflect back to a sensor. Infra-red distance sensing works through measuring angle of reflection of infra-red beam; distance is calculated using triangulation. Ultrasound works though measurement of time for sound waves to reach an object and reflect back to a sensor.

A possibility for the imaging technique to be used with a BMD LPS-ACL guiding system is the CT scan which shows the bony anatomy of the insertion sites accurately.

The first step in the BMD LPS-ACL method involves obtaining accurate distance and angular relationships between insertion sites and fixed reference bony tags. These distance and angular relationships are measured visually/optically/digitally (e.g., with software) by viewing the imaged scans of the MRI, CT, X-ray or ultrasound.

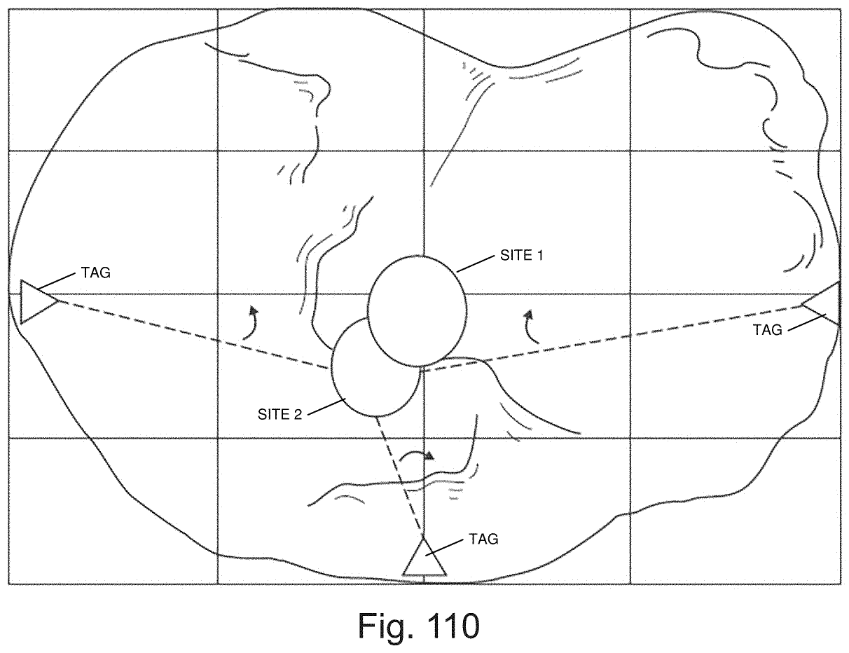

FIG. 110 illustrates an example of distance and angular relationship measurements between a constellation of anatomical tags and desired insertion sites, in this example the footprint of the anteromedial AM bundle of ACL on the tibia.

At the time of surgery, these distance and angle relationships are then transcribed and reproduced within the patient's knee joint using any variety of distance and/or proximity sensors alone or a combination.

In the case of ultrasound, infrared, laser sensors and the like, a measured distance in millimeters on an imaging study is reproduced inside the joint with the same unit in (millimeters), in order to pinpoint the insertion site.

In the case of magnetic sensors, the distance in measured in millimeters on the imaging study is converted to a commensurate level of magnetic field strength to determine the exact position of the insertion site.

In some implementations, surgeons performing ACL surgery may be able to reproduce perfect tunnel positioning for each individual patient, ultimately realizing standardization and customization techniques for tunnel placement in ACL reconstruction. Since the surgeon is able to very closely implement the desired plan, post-operative assessment allows the surgeon to evaluate the merits of the desired plan as actually implemented. This allows the standard of care to be improved over time, something that is extremely difficult, if at all possible, using current technologies and installation systems.

With respect to a sensing system that may be desired for the BMD ACL method, tracking systems may employ an emitter/sensor pair to determine a position of an object as well as measurement techniques such as time of flight (ToF) and an Angle-of-Arrival (AoA). These techniques enable determination of distances and angles based on absolute time measurements.

In contrast, time-difference-of-arrival (TDoA) allows for calculating the difference in distances between the target and two reference stations by using the difference in arrival time. Ultra-Wide Band (UWB) and high frequency electromagnetic impulses may provide reliable solutions for localization in some instances.

Infrared signal technologies use time differences to various reference antennas and triangulation for localization purposes.

Ultrasonic based approaches use measurements of the propagation time to enable the computation of distances and ensure 3-D tracking.

Additionally, a large variety of different electronic sensors are available to measure distance, displacement and position; and can be considered. These include inductive sensors, capacitance sensors, laser distance sensors (LIDAR), magneto-inductive sensors, confocal sensor, and draw-wire sensors.

A variety of other local positioning technologies may also be used to estimate an object's position. These include Wi-Fi technology, Radio Frequency Identification technology (RFID), Bluetooth technology, and other vision and camera technologies.

Regardless of the specific technology utilized, many object tracking systems use an emitter/sensor pair to determine the position of the object. In some situations, the emitters are active and/or passive. In some cases, the sensors are active and/or passive. In certain situations, the (i) emitters, (ii) sensors, and/or (iii) combined emitter/sensor pairs are embedded in the bony tags (anatomical tags). In some cases, emitters, sensors and (combined emitter/sensor pairs) are called nodes or beacon nodes, which collectively comprise a local positioning system.

Some of the mentioned techniques may be limited due to disturbances, multipath fading, lighting and line of sight issues. Certain technologies are however less sensitive to line of sight concerns.

Magnetic field systems have been utilized for distance measurements and have an advantage for some implementations of not being limited by line of sight issues. Each distance and/or motion sensing technology may have its own advantages and disadvantages. Certain technologies may be more amenable to be enabled in BMD LPS-ACL surgery. Some of these technologies can be used alone or in combination.

Regardless of the distance sensing technology utilized, the general concept of the BMD LPS-ACL system is essentially the same, where a local positioning system is developed by establishing a local common reference frame about the joint. This is accomplished by attaching “sensor enabled reference tags” to the bone.

The term local positioning is distinguished from a “global” positioning system, global in the sense that all relevant objects in the operating space are tied to a common reference frame with the patient and tools having absolute positions in this common reference frame. In contrast, for some implementations of the present solution, a relative position of the patient and tools is all that is needed, and some may determine that is what is actually important. The local positioning system enables a simpler and less expensive (resources and time), and more efficient and accurate solution.

Imaging studies such as CT, MRI, Ultrasound and X-ray are then obtained with reference tags in place, which enable exact distance (and angular relationship) measurements between the reference tags and the (insertion sites/footprints) of the ligament.

The tags allow multiple measurements of distance and angular relationships between tags and insertion sites or (anatomical landmarks of interest). A constellation of at least two or three reference tags (and sometimes many more maybe utilized).

In one embodiment, special sensor tipped BMD LPS-ACL guides akin to mine detectors with incorporated electronics and display units are developed, which are then programmable to graphically and numerically show the surgeon when the exact location of the bony landmark (insertion site) is achieved. This process occurs as the surgeon scans the sensor tipped BMD LPS-ACL guide over the insertion sites, inside the joint, over the top of the tibia and over the lateral condylar notch of the distal femur, assessing the proximity of the tip of the guide to the insertion sites.

When the BMD LPS-ACL sensor tips “light up” or graphically indicates that the intended target has been reached, a set of prongs of the BMD LPS-ACL guide are docked onto the bone and standard drilling of the guide wire and over-drilling of the tunnel are performed.

In this manner, accurate and reliable ACL tunnel placement can be accomplished and the poor tunnel positioning as the cause of ACL failures can be eliminated once and for all.

For example, the AM insertion site (footprint) for the anteromedial ACL bundle is determined to be 5.4 mm from reference tag 1, 3.3 mm from reference tag 2 and 7.8 mm from reference tag 3. The tip of the sensor is programmed to vibrate, make noise or show a green light, among other possible indications, when all these measurement requirements are achieved, as the BMD LPS-ACL sensor tipped guide is scanned over the top of the tibia.

Additionally, a graded targeting system can be shown visually to assist the surgeon in approaching and docking the BMD LPS-ACL guide directly over the footprint. The targeting system may be fashioned to have a “bulls-eye” or a color scheme (green, yellow, red). Once the insertion site is detected, the BMD LPS-ACL guide prongs can be docked onto the bone and standard guide wire drilling and over drilling can be accomplished.

In this manner all four footprints for the ACL (AM and PL insertion sites on the tibia and femur) can be quickly and automatically pinpointed with the BMD LPS-ACL guiding system and method.

This is done without the need to waste time debriding soft tissue or bone (notchplasty), and without the use of expensive and bulky equipment.

Any of the sensing technologies discussed above, alone or in combination, can be potentially optimized for the BMD LPS-ACL method; and utilized with any of the imaging technologies. For example, at this time it may be possible that miniaturized metallic chips within fasteners may be attached to bone and used in conjunction with CT, ultrasound and radiograph imaging to assess the exact location of the insertion sites.

In some cases, the tags may be made without metal so as to not cause artifacts with MRI imaging. Alternatively, solution software for MRI can be created to offset and mitigate against any metal artifact that maybe created by metallic chips.

Another embodiment of BMD LPS-ACL guide may involve the use of magnetic field-based systems which may have applications for some no line of sight scenarios. Magnetometer sensors with acceleration and angular rate measurements from inertial measuring units (IMU) can be used to measure distances without concern for line of sight issues. Alternatively, extremely low frequency (ELF) magnetic fields or distributed magnetic local positioning (DMLP) systems can be utilized for distance measurements without concern for line of sight issues.

In another embodiment the combination of IMU and magnetic field-based systems (IMU/Magnetometer Sensor Fusion Technologies) can also be used for accurate positioning in some Non-line of Sight scenarios.

For reference, “Accurate 3D Positioning for a Mobile Platform in Non-Line-of Sight Scenarios Based on IMU/Magnetometer Sensor Fusion” Hendrik Hellmers, Zakaria Kasmi, Abdelmoumen Norrdine, Andreas Eichhorn is incorporated by reference herein.

As an example, beacons which create ELF magnetic fields and have excellent characteristics for penetrating line of sight obstructions, may be installed around a periphery of a knee joint about the tibial plateau and femoral condyles (anatomical tags). In dormant phase they act as a fixed reference points on bone for distance and angular measurement purposes on the imaging studies.

In an active phase, when deployed during surgery, the beacons emit magnetic fields. The larger the number of beacons, the wider coverage volume around the knee joint, with greatly reduced eddy field noise. A small sensor at the tip of the BMD LPS-ACL guide determines the position of the sensor in relation to the reference tags (beacons producing ELF magnetic fields). The sensor unit on the tip of the BMD LPS-ACL guide samples the local magnetic field (a vector quantity) and distinguishes the components of the field produced by individual beacons. Measurement of the fields from the several beacons along with known beacon locations and field shapes, allow the sensor to solve for its position an attitude, which provides a drift-free position information in a common reference frame. In this manner, insertion sites of the ACL bundles can be accurately determined without over reliance on the surgeon's experience and mechanical tools.

As noted earlier, the node beacons fixed in bone, which emit magnetic fields can also incorporate within, additional technology to produce a (combination sensor/emitter) anatomical tag. Similarly, the sensor at the tip of the BMD LPS-ACL guide can incorporate within beacon emitters to produce a (combination sensor/emitter) anatomical tag. Regardless of whether the tag is fixed in bone or mobile, the tag may be configured to be sensing, emitting or performing both sensing and emitting functions.

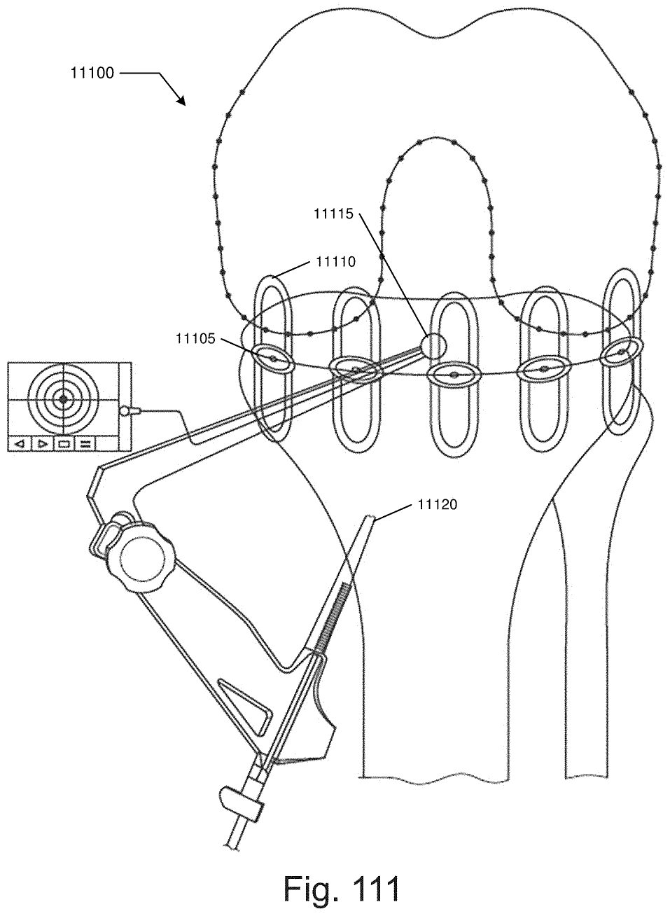

FIG. 111 illustrates an LPS-ACL guide 11100 using a set of magnetic distance sensors using a distributed magnetic local positioning system (DMLP) to identify insertion sites. Multiple beacons 11105 produce extremely low frequency ELF magnetic fields 11110 provide for a distributed magnetic local positioning system (DMLP). A sensor 11115 at the tip of the BMD LPS-ACL guide samples the local magnetic fields produced by individual beacons. Measurement of the fields from several beacons, along with known beacon location and field shapes allow the sensor to solve for its position and attitude to position a tunnel drill guide 11120 as earlier described using different technologies for the anatomical tags.

Eddy field noise and ferromagnetic distortion provide challenges for use of magnetic fields; however, current technology allows for development of better signal architecture and solution algorithms which may mitigate against ferromagnetic distortion and reduced eddy field noise, and as well to provide greater efficiency.

In U.S. patent application Ser. No. 16/596,410, incorporated herein by reference for all purposes, a 3D sculpting apparatus was described for preparation of bone tunnels in ACL surgery (See, for example FIG. 18 and associated text for a machine creating a desired profile on a bone tunnel—for example FIG. 15). An automated/semiautomated surgical apparatus produced a “profiled” bone tunnel in the tibia and initiated preparation of a second profiled bone tunnel in the femur. Apparatus included a bone preparation implement (e.g., a high-speed burr or the like) having a mechanical coupling (direct or indirect) between a controller (e.g. a machine having a stored program computing system including processor executing instructions from a memory including a user interface to set user options and parameters). Use of a bone sculpting apparatus allows a surgeon to prepare non-cylindrical bone tunnels which may enhance the effectiveness of the procedure.+

There are automated assistive surgical devices which may fill the role of a component of the apparatus, such as robotic assisted surgical platforms (e.g., MAKO, da Vinci, Verb, Medtronic, TransEnterix, Titan Medical systems, NAVIO blue belt, and the like). These platforms provide positional control/limitation of surgical implements operated by a surgeon, such that the robotic tools (some of which utilize custom software and CT data) resist the movements by the surgeon that may attempt to deviate from a planned procedure, bone preparation, or other processing. These platforms are often installed into a known “global” reference frame shared by the patient so precise position control/limitation may be imposed. Installing bone preparation tool (e.g., a high speed rotating burr or the like) the surgeon may operate the platform to form a precisely profiled bone tunnel as described herein (e.g., profiled tunnels). A profiled tunnel may be initiated from a bit-prepared cylindrical tunnel and then profiled from there or apparatus may prepare the entirety of the profiled bone tunnel.”

It is conceivable that similar “automated” bone preparing techniques can be created with the use of anatomical tags implementing a local positioning system as opposed to a global solution with a variety of local positioning systems described in this application, that allow for precisely prepared bone tunnels for ligament surgery. The tags may, in this and similar machine-assisted/implemented procedures, serve an additional or alternative role as a geofence locator that may be used to constrain a range of motion for the bone implement.

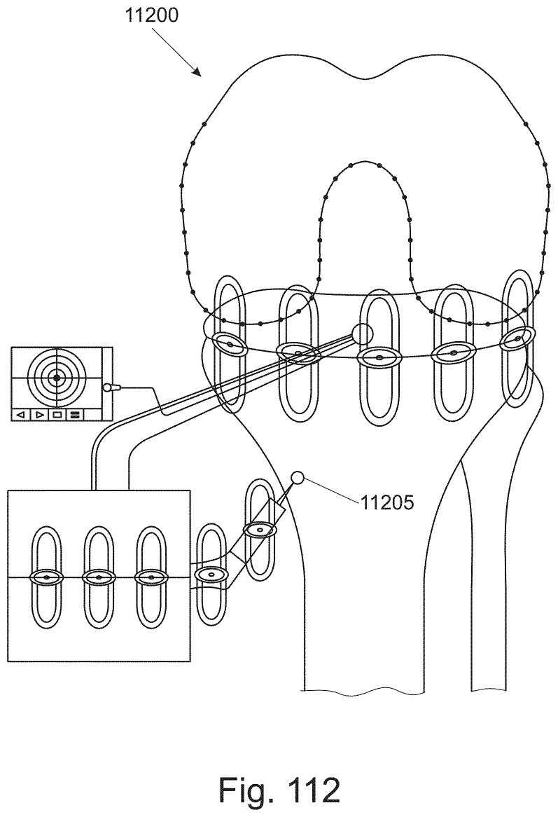

FIG. 112 illustrates a preparation system 11200 using a constellation of anatomical reference tags for positioning a bone preparation implement, such as the 3D sculptor and the like similar to FIG. 11 in placement and use of tags (e.g., magnetic beacons). FIG. 112 is similar to FIG. 111 except that in FIG. 111, the local positioning systems is used to position a guide. In FIG. 112, the local positioning system 11200 is used to actively control or supervise an implement 11205 that directly removes bone or other tissue.

As an example, FIG. 112 illustrates an embodiment of an automated bone preparation device utilizing beacons with extremely low frequency (ELF) magnetic fields and distributed magnetic local positioning system, incorporated within the “robotic device”, as well in bone, to produce a local positioning system that enables the automated device to prepare bone using a stored program computing system including processor executing instructions from a memory including a user interface to set user options and parameters.

As noted above, reference tags may be applied to bone to allow detection of changes (delta) in distance and angles between fixed points in bone (leg-length and offset) and assist in placement of bone-preparation implements. The use of anatomical tags (in dormant imaging and active surgical phases), used for detection of tunnel position and ligament footprints in ACL surgery, can be similarly utilized in arthroplasty surgery, to determine exact location and alignment of implants (prosthesis) in bone.

In the arthroplasty scenario, the patient is seen in clinic, and under sterile technique, miniaturized reference tags are inserted in bone. For example, for the knee, the reference tags would be inserted in the lateral and medial femoral condyles, as well as the proximal tibia, where bone is subcutaneous. For the hip, the reference tags would be inserted in the iliac crest of the pelvis and the greater trochanter of the proximal femur. Imaging studies are then obtained such as X-ray, CT, MRI or Ultrasonic scans with reference tags in place and dormant.

In the laboratory, as a first step, the computer scientist, through a variety of data processing techniques and software, superimposes a “virtual implant” in the desired position and alignment in bone, within the scanned image. For example, this is the same method that is currently used to produce patient specific instrumentation (PSI) with use of CT and MRI scans. Similarly, this method is used in the virtual pre-plan positioning of implants in MAKO robotic arthroplasty.

As a second step, the distance and angular relationships of the optimally positioned virtual implant with the bone reference tags is measured and recorded as the ideal implant position.

In some implementations, anatomical reference tags (e.g., with sensors, emitters or sensor/emitter combinations) may be configured to be incorporated within (and/or on the surface) of implants and prosthesis. Addition of reference tags to implants could be done during the fabrication process with additive and or subtractive manufacturing processes. The addition of anatomical tags on implants, in conjunction with anatomical tags in bone, allows for creation of a distributed local positioning system (DLPS) for joint arthroplasty, similar to what is described in the body of this application for ACL reconstruction, which can show the exact position and alignment of the implant in relation to bone, in real-time fashion, during the surgical procedure. This technique can provide unsurpassed levels of precision and accuracy in implant alignment and positioning in joint arthroplasty, allowing the surgeon to visualize an entire structure of the implant in relation to bone (live and in real-time) sometimes available in a related format in current robotic and navigation systems, but without the intra-operative glitches, unreliable accuracy, frustration with intra-operative registration and line of sight issues. This process can be accomplished with no interruption to the flow of the operation, no bulky and cumbersome equipment that has to be moved in and out of the way of the surgeon, no significant increase in cost of purchasing capital equipment (cost of MAKO robot ˜$1 Million or more).

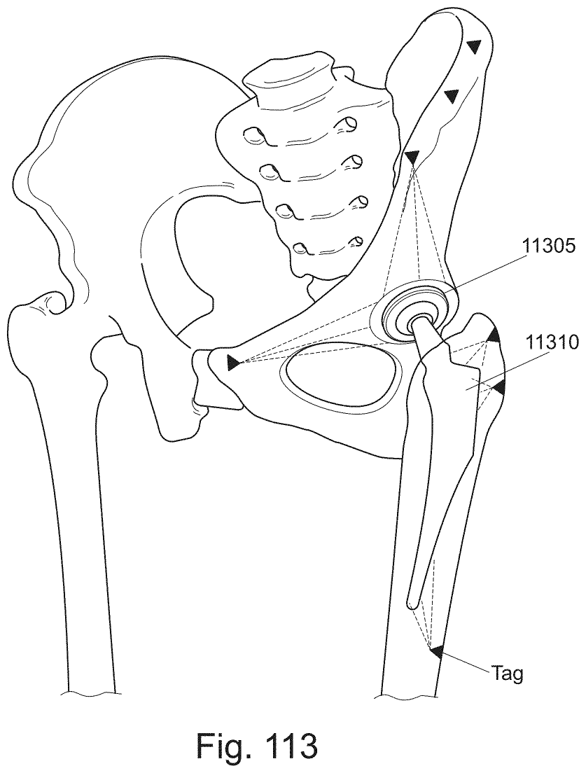

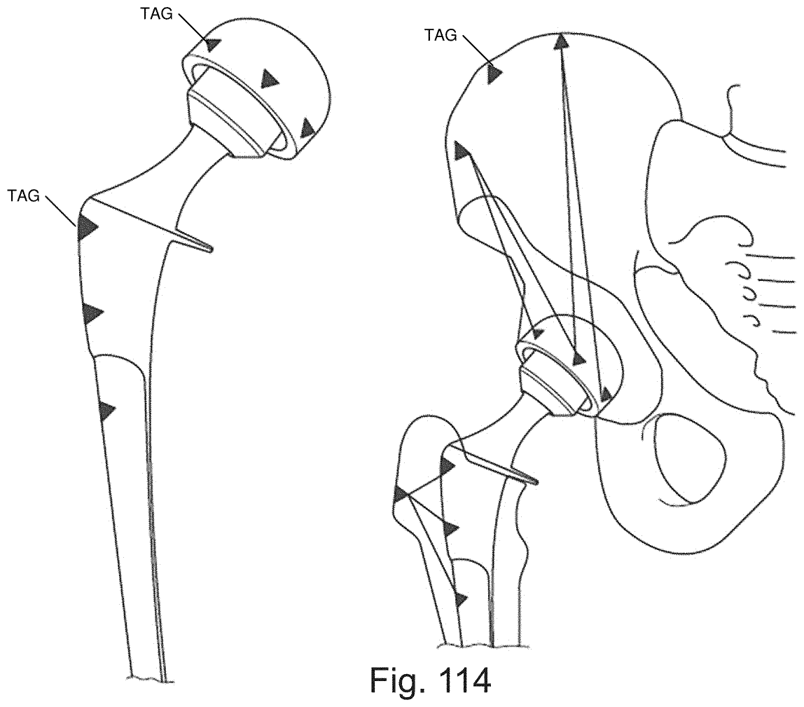

FIG. 113 illustrates a reference virtual implant position as discussed above, determined pre-operatively using clinically installed anatomical tags (still present). FIG. 114 illustrates an LPS measurement of actual implant position(s) using installed anatomical reference tags.

In FIG. 113, a virtual acetabular cup 11305 and virtual femoral stem 11310 are optimally positioned in the imaging study. The angular and distance relationships between the bone tags and ideal implant position is measured and recorded. This is considered the dormant reference tag phase (in vitro). This information will be used later, in the active reference tag phase (in vivo), to provide a reference for comparison to the actual implant position measured, in some cases measurements made using local positioning, during surgery.

In FIG. 114, reference tags incorporated within the implant, in conjunction with reference tags in bone, provide exact position of the implant during surgery, allowing for comparison of (actual implant position) with (virtual implant position) acquired during the dormant phase.

Therefore, during surgery when the surgeon places the implant in bone, because the relationship of the reference tags with the (virtual) ideal implant position is known and recorded in vitro, and the relationship of the reference tags with the actual implant position can measured in vivo; the differential between actual position and ideal position of the implant can be conveyed to the surgeon in real-time fashion. This method provides the surgeon an ability to assess implant position in a live fashion, which allows for second to second adjustments of implant alignment during the installation process, without any disruption to the flow of the operation.

For example, the computer scientist may position the virtual acetabular cup at 45 degrees of abduction and 20 degrees of anteversion. During surgery the surgeon may place the actual cup in 35 degrees of abduction and 15 degrees of anteversion. The reference tags, through variation of distance sensing technologies described in this application, would provide the delta or (error) in position and alignment of the actual implant (in relation) to the ideal implant.

In the above example, the surgeon can see that she needs to increase abduction by 10 degrees and increase anteversion by 5 degrees to obtain the desired 45 degrees of abduction and 20 degrees of anteversion, as planned. She can correct alignment as the installation process is ongoing, by making second to second adjustments through live streaming information never previously available.

The combination of this technology with previously described vibratory insertion technologies (see, for example, U.S. Pat. Nos. 9,168,154, 10,610,379, 10,245,162, 10,413,425, 10,245,160, 10,478,318, and 10,729,559) allows “floating” or placing the prosthesis precisely in the desired position, without use of impacts, in perfect alignment, with unsurpassable haptic freedom to adjust implant position and other manipulations of a prosthesis.

Fundamentally regardless of mathematical algorithms and distance sensing technology utilized, this process, in ligament reconstruction, is accomplished through establishment of a local positioning system with reference tags in bone and reference tags on guiding implement (LPS-ACL guide). In arthroplasty procedures, the process is accomplished between reference tags in bone and reference tags on implants

In some arthroplasty embodiments, for example, during the dormant and pre-planning stage, the relationship of the reference tags to well-known and identifiable anatomical landmarks of interest in the joint can be measured and recorded. During the surgical phase, when the reference tags are active, the distance and angular relationships from the reference tags to the well-known anatomical landmarks can be further enhanced by addition of secondary reference tags to these landmarks, once surgical exposure has been achieved. Some of these landmarks are not subcutaneously accessible in clinic. However, they can be accessed accurately utilizing the “clinic applied” reference tags and information obtained in the dormant phase with the imaging studies. In this manner a much more robust local positioning system is created once the surgical site is exposed.

For example, in hip replacement surgery, once the hip is surgically exposed, preliminary “clinic applied” reference tags can guide hand held sensor tips to “anatomical landmarks of interest” previously inaccessible in clinic, such as the lessor trochanter. Addition of these “secondary reference tags” allows enhancement of the local positioning system, and therefore allows more precise and accurate leg length and offset measurements between pelvis and femur. This method provides a “redundancy” or “double check” system for positioning of the implants.

For example, in the dormant phase, the relationship of reference tags on the pelvis (iliac crest) to the lessor trochanter of the proximal femur is measured. During surgery, after the application of the secondary reference tags (on the lessor trochanter—previously inaccessible in clinic), and installation of the acetabular and femoral implants, two different methods are available to assess implant position. First, the virtual (ideal) implant vs. actual implant position can be assessed by mathematical comparison of fixed reference tags in bone and mobile reference tags on the implant. Second, the absolute value of pre and post op leg length and offset are available by comparison of pre-operative distance from pelvis reference tag to lessor trochanter (chosen on imaging scan) to post-operative position of pelvis reference tag to lessor trochanter (secondary tag).

This method is a double check system for assessing the positioning of the implant. Therefore, the reference tags can help the surgeon's implant positioning by several means including: (i) allowing a live, real-time assessment and comparison of the “virtual ideal position” determined in the (dormant phase) with the “actual implant position” determined in the (active phase); and (ii) allowing a redundancy double check system that allows comparison of leg length and offset measurements after implant installation (active phase) with those before implant installation (dormant phase).

In another embodiment, reference tags may be applied to tools that are used to fit, install, or impact prosthesis into place. In patents incorporated above, a “mechatronic handle” was described, a tool designed to install prosthesis into place with vibratory force while monitoring an alignment of the implant with an inertial measuring unit IMU. The reference tag local positioning technology can be used in a tool like the “mechatronic handle”, alone, or in addition to the IMU technology described to allow real-time live monitoring of the implant position and alignment relative to bone during implant installation. In other embodiments, the reference tag local positioning system can be used in any impaction rod or insertion tool to properly position an implant.

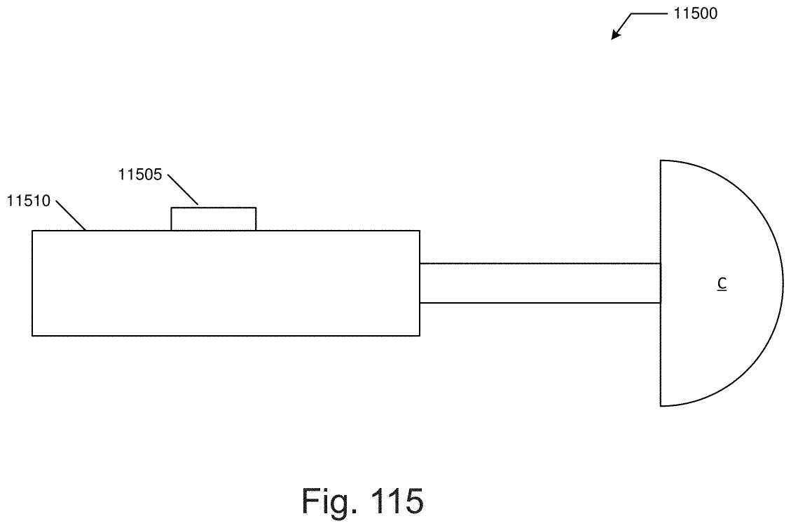

FIG. 115 illustrates a generalized BMD including an anatomical tag local positioning system. A Behzadi Medical Device (BMD) has been described using an installation system having an IMU (inertial measuring system) to determine/set a desired installation orientation for an implant during or after installation. The BMD of FIG. 115 includes a local positioning system compatible with installed reference tags (like the beacons and other anatomical tags) so that the BMD may interact with the reference tags to set the desired installation parameters. The reference tag positioning system may be in addition to, or in lieu of, the IMU technology described in the previous solution.

In another embodiment, reference tags have utility in long term monitoring of implants in bone. One of the major problems in press fit arthroplasty still remain aseptic loosening and subsidence which frequently presents late, years after implantation, and is diagnosed by radiographic comparison of X-rays obtained over the life of the implant. For example, a total knee, hip or shoulder prosthesis may subside over time by 1 mm, 2 mm, or 3 mm as it loosens and “debonds” from bone. On some occasions subsidence of implants stabilizes and does not lead to clinical failure. On some occasions aseptic loosening leads to clinical failure. Subsidence and loosening of implants can be more accurately and granularly measured by distance and angular measurements between bone reference tags and implant embedded reference tags. Therefore, reference tags have utility in post-operative monitoring, particularly for diagnosis of aseptic loosening and subsidence. It is conceivable that reference tags applied to bone and implant, installed in a patient, will have specific identification numbers (ID). It is also conceivable that orthopedic surgeon's offices and/or certain places where there is a large community of patients with implants would employ reference tag scanning systems, depending on the sensing technology utilized, to read any changes in position of the implants in relation to bone (bone embedded reference tags). This can simply occur by the patient walking under the scanning system or simply being present in a room with a scanning system. While such an automated system may not actually diagnose a medical condition, such automatic scanning of constellations may provide a recommendation to the patient to consult with a qualified physician to determine whether a problem exists with the implant/prosthesis. In this manner the diagnosis of subsidence and loosening can be made much more accurately than current X-ray techniques, without any extra effort by the orthopedic surgeon.

We also anticipate the in the future “auto-localization algorithms for local positioning systems” will provide new methods to calculate inter beacon (reference tag) distance, angular relationships and positions based on mathematical linearization and trilateration techniques, without the need to obtain preliminary measurements on imaging studies. In this embodiment the need to create a fixed local framework with the initial imaging study is dropped. All that the surgeon needs to do is to obtain exposure and apply reference tags to certain anatomical landmarks of interests. The mathematical calculations and algorithms occurring individually and collectively within the beacons (reference tags) themselves provide an autonomous local positioning system, which will provide exact position and alignment information about the implant relative to the bone.

See, for example, “Auto-localization algorithm for local positioning systems” J. Guevara A. R. Jimenez, J. C. Prieto, F. Seco, hereby incorporated by reference.

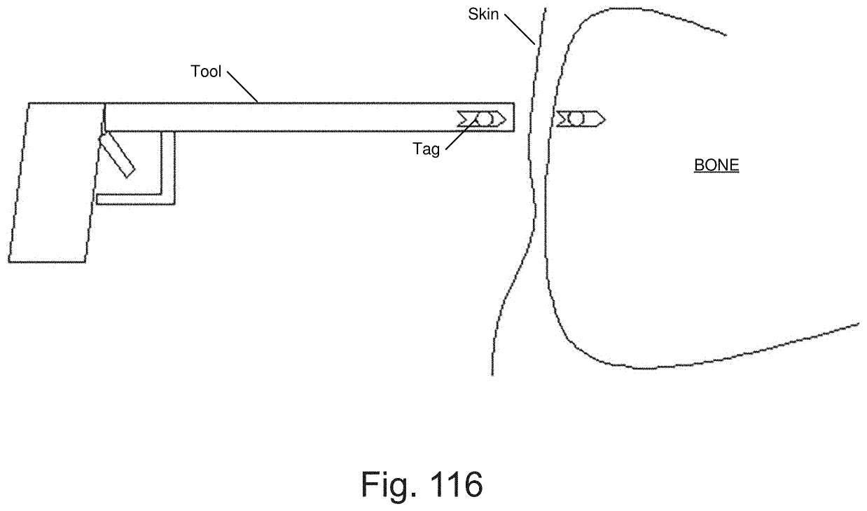

FIG. 116 illustrates a generalized BMD including an anatomical tag installation function. As noted herein, reference tags may be installed preoperatively in clinic under sterile condition with a mechanized or manual tool, with minimal trauma similar to an injection. A conceptual version of a reference tag inserter is presented in which a “gun” installs a reference tag subcutaneously (e.g., through the skin or a small incision made in the skin. The reference tag is installed in tissue, such as subcutaneous bone or connective tissue. In some cases reference tags may be installed in other tissue such as muscle or adipose tissue, or in some cases applied to a surface of the skin depending upon the application and an amount of time that the reference tags are desired to be in place. The inserter may, in some implementations, include a local positioning system that interacts with installed reference tags to help position additional reference tags in the preoperative clinic process.

In a constellation, it is not the case that all reference tags are homogenous or are operational for the same function. Some reference tags may be used for the dormant phase before surgery, the active phase during surgery, and/or the post-operative phase for monitoring the quality of the installation or other desired parameter or condition.

An initial set of reference tags may be selected that are used to install during clinic, such as for the imaging and virtual prosthesis planning. During surgery when the subcutaneous tissue and bone is exposed, additional reference tags may be installed (and when desired the clinically installed reference tags may help position the additional reference tags at desired locations to aid the procedure. These reference tags installed during the procedure may have additional functionality or additional location services the inclusion of which may have hindered the installation during the pre-operative clinic phase (e.g., size or desired locations inaccessible during clinic).

When post-operative monitoring is desired, a special type of reference tag may be installed that is tailored for such use. The post-operative reference tags may also be positioned at strategic locations to simplify detection of loosening, for example. The different reference tags may define different constellations, which can simplify long-term post-installation monitoring of a reduced number of long-term reference tags.

In some instances, a set of tags may be predefined and installed on a foundation, such as a flexible film or surgery-compatible material (including bio-absorbable). These tags of the set may define a constellation having a predetermined relationship with each other. The foundation may be applied (e.g., adhesive, suture, and the like) to the tissue as a set once the installation site is exposed which may save some time in installing tags, including some secondary tags used to enhance primary tags preoperatively installed in the clinic. Further, having a predetermined relationship among these secondary tags applied to this foundation may help with calibration of primary tags, additional tags, and/or all tags of the system. In some cases, having a set of tags that maintain their predetermined pre-installed relationship (in 2D or 3D space as appropriate) after installation may improve the efficiency and accuracy of an implementation of a local positioning system.

The system and methods above have been described in general terms as an aid to understanding details of preferred embodiments of the present invention. In the description herein, numerous specific details are provided, such as examples of components and/or methods, to provide a thorough understanding of embodiments of the present invention. Some features and benefits of the present invention are realized in such modes and are not required in every case. One skilled in the relevant art will recognize, however, that an embodiment of the invention can be practiced without one or more of the specific details, or with other apparatus, systems, assemblies, methods, components, materials, parts, and/or the like. In other instances, well-known structures, materials, or operations are not specifically shown or described in detail to avoid obscuring aspects of embodiments of the present invention.

Reference throughout this specification to “one embodiment”, “an embodiment”, or “a specific embodiment” means that a particular feature, structure, or characteristic described in connection with the embodiment is included in at least one embodiment of the present invention and not necessarily in all embodiments. Thus, respective appearances of the phrases “in one embodiment”, “in an embodiment”, or “in a specific embodiment” in various places throughout this specification are not necessarily referring to the same embodiment. Furthermore, the particular features, structures, or characteristics of any specific embodiment of the present invention may be combined in any suitable manner with one or more other embodiments. It is to be understood that other variations and modifications of the embodiments of the present invention described and illustrated herein are possible in light of the teachings herein and are to be considered as part of the spirit and scope of the present invention.

It will also be appreciated that one or more of the elements depicted in the drawings/figures can also be implemented in a more separated or integrated manner, or even removed or rendered as inoperable in certain cases, as is useful in accordance with a particular application.

Additionally, any signal arrows in the drawings/Figures should be considered only as exemplary, and not limiting, unless otherwise specifically noted. Combinations of components or steps will also be considered as being noted, where terminology is foreseen as rendering the ability to separate or combine is unclear.

The foregoing description of illustrated embodiments of the present invention, including what is described in the Abstract, is not intended to be exhaustive or to limit the invention to the precise forms disclosed herein. While specific embodiments of, and examples for, the invention are described herein for illustrative purposes only, various equivalent modifications are possible within the spirit and scope of the present invention, as those skilled in the relevant art will recognize and appreciate. As indicated, these modifications may be made to the present invention in light of the foregoing description of illustrated embodiments of the present invention and are to be included within the spirit and scope of the present invention.

Thus, while the present invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the present invention. It is intended that the invention not be limited to the particular terms used in following claims and/or to the particular embodiment disclosed as the best mode contemplated for carrying out this invention, but that the invention will include any and all embodiments and equivalents falling within the scope of the appended claims. Thus, the scope of the invention is to be determined solely by the appended claims.

1. A system for a body, the system providing extracorporeal data regarding a status of an anatomical structure within the body, comprising:
a set of subdermal reference tags;
a tag interface, disposed outside the body and in communication with said set of tags, configured to support a local positioning system; and
wherein said local positioning system assists in location of an implement relative to one or more anatomical elements of the anatomical structure; and
wherein said local positioning system is obscured from direct line of sight access.

2. The system of claim 1 wherein the anatomical structure includes a pelvis and a femur coupled to said pelvis.

3. The system of claim 1 wherein said set of tags include one or more devices selected from group consisting of a passive sensor, an active sensor, a passive tag, an active tag, a passive reference, an active reference, and combinations thereof.

4. The system of claim 1 wherein the anatomical structure includes a knee joint.

5-21. (canceled)

22. The system of claim 1 wherein said local positioning system does not include an operating room-centric coordinate reference frame into which said local positioning system is mapped.

23. The system of claim 1 further comprising an assistive positioning system including said local positioning system.

24. The system of claim 23 wherein said assistive positioning system does not include a portable imager or a robot.

25. The system of claim 1 wherein said tag interface is configured to provide realtime data of the status of the anatomical structure within the body during a medical procedure referencing the anatomical structure.

26. The system of claim 1 wherein the anatomical structure includes a location of a ligament footprint.

27. The system of claim 1 configured for installation of an implant at an implant location relative to the anatomical structure wherein said local positioning system is configured to provide a guide for said implant location.

28. The system of claim 1 wherein at least a subset of said set of subdermal reference tags are configured for clinical non-surgical advance subdermal installation.

29. The system of claim 1 further comprising a flexible foundation supporting a preinstalled set of reference tags, some or all of which may have a predetermined relative location with respect to each other, configured for installation as part of said local positioning system.

30. A method providing extracorporeal data regarding a status of an anatomical structure within a body, comprising:
installing subdermally a set of reference tags producing a set of installed tags;
establishing, in communication with said set of installed tags, a local positioning system including an extracorporeal tag interface communicated to said set of installed tags; and
locating, assisted by said local positioning system, an implement relative to an anatomical element of the anatomical structure.

31. The method of claim 30 wherein said set of installed tags is visually obscured from said extracorporeal tag interface during said locating said implement step.

32. The method of claim 30 wherein said locating said implement step is performed within an operating room and wherein said installing step includes pre-installing subdermally, during a presurgical clinical visit outside of said operating room, at least a subset of said reference tags.

33. The method of claim 32 wherein said set of installed tags include said subset of said reference tags.

34. A method for a surgery including a use of extracorporeal data regarding a status of an anatomical structure within a body, comprising:
configuring a local positioning system referencing a set of subdermal reference tags positioned in a preconfigured relationship to the anatomical structure, said local positioning system including an extracorporeal tag interface communicated to said set of subdermal reference tags;
producing extracorporeally, from said extracorporeal tag interface, a set of location information from said set of subdermal reference tags regarding a relative position of the anatomical structure within a local position reference frame including said set of subdermal reference tags, the anatomical structure, and said relative position; and
monitoring said set of location information during the surgery without use of portable imagers or robots.

35. The method of claim 34 wherein the surgery includes a cooperation of subdermal tissue with a structure, the method further comprising:
adding the portion of tissue and the structure to said local position reference frame wherein said set of location information includes a relative position between the portion of tissue and the structure; and thereafter cooperating the portion of tissue with the structure using said set of location information in realtime.

36. The method of claim 34 wherein said monitoring step is performed within an surgical environment and wherein said configuring includes a preinstallation of at least one of said subdermal reference tags outside said surgical environment.

37. The method of claim 35 wherein said portion of tissue includes a pelvis and a cooperating femur and wherein said structure includes a prosthesis joint disposed between said pelvis and said cooperating femur.

38. The method of claim 35 wherein said portion of tissue includes a knee joint and said structure includes a ligament guide.
